(19) United States
(12) Patent Application Publication
(10) Pub. No.: US 2025/0054573 A1
(43) Pub. Date: Feb. 13, 2025

(54) METHOD OF CHARACTERISING A DNA SAMPLE

(71) Applicants: Cambridge Enterprise Limited, Cambridge (GB); FUNDACION DEL SECTOR PUBLICO ESTATAL CENTRO NACIONAL DE INVESTIGACIONES ONCOLOGICAS CARLOS III (F.S., Madrid (ES)

(72) Inventors: Geoffrey John Macintyre, London (GB); Florian Markowetz, London (GB); Ruben Matthias Drews, London (GB); Bárbara Hernando Fuster, London (GB)

(21) Appl. No.: 18/697,408

(22) PCT Filed: Oct. 3, 2022
(86) PCT No.: PCT/EP2022/077473

§ 371 Date: Mar. 29, 2024

(30) Foreign Application Priority Data
Oct. 4, 2021 (GB) 2114203.9

(51) Int. Cl. G16B 20/00; G16B 40/20; G16H 10/60; G16H 50/20

(52) U.S. Cl. CPC G16B 20/00; G16B 40/20; G16H 10/60; G16H 50/20

(57) ABSTRACT

A method of characterising a DNA sample obtained from a tumour, the method including the steps of: (a) obtaining a tumour copy number profile for the sample, (b) quantifying a set of copy number features of the copy number profile, and (c) determining exposure to one or more signatures of chromosomal instability based on the quantified features. A copy number feature is a metric that characterises a copy number event in a copy number profile. The set of features does not comprise the absolute copy number of segments in the copy number profile. The signatures of chromosomal instability have been obtained by quantifying the set of copy number features in a plurality of tumour samples, and identifying one or more mutational signatures likely to result in the copy number profiles of the plurality of tumour samples. Methods of characterising types of chromosomal instability present in samples, providing a prognosis, identifying a drug target, or identifying a therapy for a subject are also described.

### FIELD OF INVENTION

The present invention relates to a method for characterising a DNA sample by determining the presence of copy number signatures associated with different types of chromosomal instability. It is particularly, but not exclusively, concerned with a method for determining whether a tumour has one or more deficiencies associated with chromosomal instability, such as impaired homologous recombination and replication stress, and to methods for identifying a treatment based on the presence of copy number signatures in a DNA sample from a tumour.

### BACKGROUND TO THE INVENTION

Chromosomal instability (CIN) is the process of accumulating numerical and structural changes in DNA. CIN is a hallmark of cancer and over time manifests as changes of whole chromosomes or parts of chromosomes. A stable state where large-scale chromosomal aberrations are tolerated and do not change over time is called aneuploidy and is seen as a product of CIN.

Consequences of CIN are complex and include recurrent loss or amplification of driver genes [9-11], highly complex focal rearrangements, formation of extrachromosomal DNA and micronuclei, activation of innate immune signaling [Bakhoum et al., 2018], as well as associations with disease stage [Raghvendra et al., 2020, Vargas-Rondon et al., 2018], metastasis [Bakhoum et al., 2018b], poor prognosis [Bakhoum et al., 20111], and therapeutic resistance [Lee et al. 2011]. Causes of CIN are equally complex and include mitotic chromosome missegregation, homologous recombination defects, telomere crisis, and breakage fusion bridge cycles.

Despite the diversity of causes and consequences, CIN is generally used as an umbrella term. Measures of CIN either divide tumours into broad categories of high/low CIN [Birbak, 2011], or are restricted to a single aetiology like homologous repair deficiency [Davies et al., 2017], or are limited to a particular genomic feature like whole chromosome-arm changes [Cohen-Sharir et al., 2021]. As a result, there is no systematic framework to comprehensively characterise the diversity, extent and origins of CIN, and to define how different types of CIN relate to clinical phenotypes.

Therefore, there is still a need for improved methods for characterising chromosomal instability.

### STATEMENTS OF INVENTION

The present inventors devised a robust analysis framework for chromosomal instability in human cancers. In particular, the inventors identified that patterns of chromosomal instability could be robustly characterised using a limited set of fundamental copy number features that does not include the absolute copy number of segments. The inventors further demonstrated the relevance of the approach in a pan-cancer analysis of 7,880 high-quality samples. The inventors thereby identified a compendium of copy number signatures that characterise different types of CIN and their aetiologies across 33 cancer types, and are supported by a wide array of independent data sources. The inventors then demonstrated the biological relevance of the pan-cancer signatures identified by using the signatures to predict drug response and to identify new drug targets. In particular, associations between the signatures and targeting of 40 genes were identified, supported by both drug and genetic perturbations. This demonstrates the use of the signatures in identifying existing therapeutic strategies for cancers. Further, associations between the signatures and targeting of 104 genes with druggable structures but without known targeted therapies were also identified. This demonstrates the use of the signatures in the drug design process, by identifying drug targets for cancer therapeutics. The inventors further showed how the new framework refines the understanding of impaired homologous recombination (IHR), one of the most clinically relevant types of CIN. In particular, the inventors identified three distinct signatures of IHR: a signature of IHR alone, a signature of IHR plus replication stress, and a signature of IHR plus replication stress and impaired damage sensing and nucleotide excision repair (NER). Finally, the inventors demonstrated that these signatures could be used to predict sensitivity to platinum-based therapy in multiple cancers including ovarian and oesophageal cancer.

Thus, according to a first aspect, there is provided a method of characterising a DNA sample obtained from a tumour, the method including the steps of:
(a) obtaining a tumour copy number profile for the sample;
(b) quantifying a set of copy number features of the copy number profile, wherein a copy number feature is a metric that characterises a copy number event in a copy number profile, and wherein the set of features does not comprise the absolute copy number of segments in the copy number profile; and
(c) determining exposure to one or more signatures of chromosomal instability, wherein the signatures of chromosomal instability have been obtained by quantifying the set of copy number features in a plurality of tumour samples, and identifying one or more mutational signatures likely to result in the copy number profiles of the plurality of tumour samples.

The present inventors have identified that not including a feature representing the copy number of a segment advantageously avoided redundancy if signatures for the same aetiology appear across different ploidy background. For example, a single copy loss on a whole-genome duplicated (WGD) background versus a deletion in a non-WGD background would result in two different copy number states and therefore be encoded by two different signatures, even though they might be caused by the same mutational process. This therefore resulted in more robust, and more biologically relevant signatures identified using these copy number features.

The method may have any one or more of the following features.

The one or more signatures of chromosomal instability may each individually be associated with one or more processes causing chromosomal instability, and exposure to the one or more signatures may be indicative of the presence of the respective process(es) in the sample. The inventors have identified that not including the absolute copy number of a copy number alteration event as a feature used to characterise a copy number profile unexpectedly improves the signatures that are derived using copy number features. In particular, it avoids the artificial splitting of signatures by ploidy status when the signatures in fact represent the same mutational process.

Quantifying a set of copy number features of the copy number profile may comprise quantifying, for each copy number event in the copy number profile, one or more features selected from: segment size, copy number change-point, breakpoint count per predetermined length of sequence and/or per chromosome arm, and number of segments with oscillating copy number. The set of copy number features may include all of the following features: segment size, breakpoint count per predetermined length of sequence, copy number change-point, breakpoint count per chromosome arm, and number of segments with oscillating copy number. The set of copy number features may consist of said features or said features and one or more allele-specific features. This set of copy number features was found by the inventors to be sufficient to describe known patterns of chromosomal instability (see Table 2) including amplification (captured by the segment size (SS), copy number changepoint (CNC), and breakpoint per 10 MB (BP10) features), aneuploidy (captured by the SS and CNC features), breakage-fusion-bridge (captured by the CNC and breakpoints per chromosome arm (BPARM) features), chromothripsis (captured by the CNC, BP10, BPARM and number of segments with oscillating copy number (OSC) features), complex genomic rearrangements (captured by the SS, CNC, BP10, BPARM and OSC features), deletions (captured by the CNC feature), extrachromosomal DNA (captured by the SS and CNC features), homologous recombination deficiency (captured by the SS, CNC, BP10, BPARM and OSC features), loss of heterozygosity (captured by the SS, CNC and BPARM features), micronuclei (captured by the SS and CNC features), tandem duplications (captured by the SS, CNC, BPARM and OSC features), and whole genome duplications (captured by the CNC feature). Thus, this set of features may represent a compact yet robust and complete set of features to describe copy number profiles. Further, the present inventors have identified that the use of such a compact set had several advantages compared to the use of a more extended set that may contain some redundancies (or even noise) in the information captured by each feature. In particular, the use of a compact set of features with definitions that do not overlap means that each feature is more directly interpretable and the information it captures is not diluted across a plurality of features. Additionally, the use of additional features (potentially uninformative in relation to CIN) may lead to the identification of artefactual signatures that do not represent true biological differences. Such artefactual signatures may be difficult to identify and automatically remove, leading to less interpretable and reproducible results.

The segment size may be quantified for each segment in the copy number profile. The copy number change-point may be quantified for each segment in the copy number profile. The copy number change-point may be quantified relative to the left (upstream) neighbouring segment. The breakpoint count per predetermined length of sequence may be quantified for each set of segments that falls within a predetermined length of sequence. The breakpoint count per chromosome arm may be quantified for each set of segments that falls within a chromosome arm. The number of segments with oscillating copy number may be quantified for each set of segments that comprises the maximum number of contiguous segments oscillating between two different absolute copy numbers.

The breakpoint count is preferably quantified over two separate length of sequences: a predetermined length of sequence and the length of the chromosome arm for each chromosome arm represented in the copy number profile. The predetermined length of sequence is preferably shorter than the shortest chromosome arm represented in the copy number profile. For example, the breakpoint count per predetermined length of sequence is advantageously quantified over a predetermined length of sequence between 8 and 12 MB, preferably about 10 MB. Such values may be particularly advantageous when looking at human copy number profiles. Indeed, the present inventors have recognised that clusters of copy number alterations (CNAs) can come in various sizes, e.g. short tandem duplications with less than 10 kb in length each, long tandem duplications with lengths of over 100 kb each, and up to large-scale transitions and chromothriptic events which can span multiple dozens of mega bases. Their respective cluster sizes can be in the tens of kb up to whole chromosome arms. The present inventors have further recognised that the set of copy number features would ideally comprise features that are able to capture all of these events. However, the inventors further recognised that the smallest chromosome arm on a human genotyping array such as the SNP6 is 12.8 Mb in length, such that having window sizes that are large than this may lead to a skewed breakpoint density on smaller chromosome arms. Conversely, if the window size is too small then it may not be able to capture medium size clusters. A further feature could be included for this but this would increase the complexity of the scheme and potentially lead to some redundancy in the features. Thus, the inventors identified the combination of a breakpoint count per 8-12 MB (preferably about 10 MB) and a breakpoint count per chromosome arm to ideally capture the patterns of copy number alterations expected in e.g. a human genome.

The use of features such as the breakpoint count per predetermined length of sequence, the breakpoint count per chromosome arm, and the number of segments with oscillating copy number advantageously capture phenomena that manifest themselves over multiple segments or long stretches of DNA. This is not possible when using only features that characterise single segments. Thus, this results in a more complete (and more biologically relevant) characterisation of the CIN processes that are active in a sample.

The set of features may further comprise an allele-specific feature. An allele-specific feature may advantageously enable the detection of copy-neutral loss of heterozygosity. An allele-specific feature may for example be the proportion of the major allele over both alleles for a portion of a sequence. However, inclusion of an allele-specific feature may restrict applicability of the method to sequence data that has allele-specific resolution (e.g. WGS, SNP 6.0 arrays). Therefore, an allele-specific feature (e.g. a loss of heterozygosity feature such as the proportion of the major allele over both alleles) may advantageously not be included in order to increase the scope of applicability of the method to sequencing data that has been obtained using a variety of technologies such as genotyping arrays, single cell sequencing, shallow whole genome sequencing, etc.

Quantifying the set of copy number features may comprise using unrounded copy number segments. Quantifying the set of copy number features may comprise collapsing and merging near diploid segments to a diploid state, wherein near diploid segments are segments that have a copy number within a predetermined distance from 2. The predetermined distance may be 0.1. Thus, quantifying the set of copy number features may comprise assigning a copy number of 2 (i.e. collapsing) to any segment that has a copy number within predetermined boundaries (such as e.g. above 1.9 and below 2.1), and merging any contiguous segments that have the same copy number as a result of the assigning. This may advantageously avoid including signal from segments that are likely normal diploid segment and which could act as noise in the process. Unrounded copy number segments may be segments whose copy number has not been rounded to the near integer. Thus, the method preferably uses copy number profiles that have not been rounded and/or wherein the only rounding that is performed relates to the collapsing or near diploid segments. The inventors have identified that using unrounded copy numbers enabled them to use more of the information present in copy number profiles (in terms of number of segments that would otherwise be merged but also in terms of the copy number information that is contained in said profile for each segment.

Rounding of copy numbers for segments may be performed in order to remove noise in a copy number profile, at the cost of loss of information. The present inventors have further identified that the additional noise that is associated with the use of unrounded copy number segments could be compensated at least in part, with minimal loss of information relevant to CIN by merging segments with minor deviations from the “normal” copy number state.

Quantifying the set of copy number features may comprise quantifying a feature selected from segment size and copy number changepoint, and wherein said feature is not quantified for diploid segments. Ignoring normal diploid segments when quantifying the segment size and/or copy number changepoint feature(s) may advantageously avoid inflating the quantifications for such segments which are expected to be more numerous and do not capture information in relation to chromosomal instability for these features. This was shown by the inventors to result in refined signature exposures (likely to be more reflective of true biology), with samples with similar exposures having more similar exposures (as quantified by cosine similarity) than if the normal segments had not been ignored. Indeed, the present inventors found that normal diploid segments influenced signature activity even on strongly rearranged genomes and that it would therefore be advantageous to remove them, particularly when looking at signatures derived from multiple cancers where many samples may have less rearranged genomes. Quantifying the set of copy number features may comprise quantifying a feature selected from breakpoint count per predetermined length of sequence and breakpoint count per chromosome arm, and wherein said feature is quantified for all segments including diploid segments. The inventors have identified that although breakpoint count features are impacted by the overrepresentation of diploid segments, this impact is not as problematic as e.g. that for a copy number changepoint feature since a changepoint feature may quantify two events for each aberrant segment surrounded by diploid segments (whereas only one relevant change of copy number has occurred), whereas a breakpoint feature quantifies breakpoints as relevant events where DNA has been broken and repaired, and these are not therefore double counted by counting both breakpoints in the case of an aberrant segment surrounded by diploid segments. The copy number changepoint feature may be quantified by ignoring the first segment of any chromosome if said segment is a normal segment (where a normal segment in this context refers to a diploid segment). Indeed, the changepoint feature describes the difference in absolute copy number from a segment to its neighbouring segment, which may be the segment on the left (i.e. preceding segment), in which case the very first segment of each chromosome may not have a neighbouring segment to the left. The changepoint feature may be quantified by subtracting 2 from the absolute copy number of the first segment of any chromosome if said segment is not a normal segment. This assumes that the (non-existing) previous segment was a normal segment (copy number 2) if the segment is not a normal segment.

Quantifying the set of copy number features may comprise obtaining one or more summarised measures for each copy number feature across the copy number profile. The one or more summarised measures may comprise the sum over all copy number events for which a feature has been quantified of the posterior probabilities of each feature value belonging to each of a set of predetermined distributions. The one or more summarised measures may have been identified empirically by quantifying the copy number features in a plurality of tumour samples. A set of predetermined distributions may comprise one or more distributions, such as e.g. between 1 and 30, between 1 and 25, between 3 and 25. A predetermined distribution may also be referred to herein as a “component”. Each copy number feature may be associated with a plurality of components, and a summarised measure across the copy number profile may be obtained for each such component. The use of summarised measures that have been identified empirically using relevant data means that the features are truly reflective of biological processes rather than arbitrary categories that while convenient to manipulate, may not be biologically relevant. For example, a set of predetermined distributions may be identified by applying mixture modelling on a data set comprises the quantified set of copy number features for a plurality of tumour samples. This enables the identification of the true states of the copy number features present in tumours, and hence the quantification of the evidence for the presence of these states in a new sample to be analysed. The use of posterior probabilities of each feature value belonging to each of a set of predetermined distributions advantageously enables the method to deal with uncertainty in the assignment of a segment to a state (i.e. uncertainty in whether the segment provides evidence for the presence of a particular category of CIN events characterised by one of the distributions for a copy number feature). This means that the method is by design able to deal with inevitable noise in the data, resulting in a more accurate characterisation of the sample. The set of predetermined distributions for a copy number feature may be a set of Gaussian distributions for any feature that is quasi-continuous. The set of predetermined distributions may be a set of Poisson distributions for any count feature. A set of predetermined distributions may have been obtained using a mixture modelling technique. Count features may be selected from breakpoint count per predetermined length of sequence and/or per chromosome arm, and number of segments with oscillating copy number. A quasi-continuous feature may be any feature that is not a count feature, such as e.g. segment size and copy number changepoint. For example, the segment size feature may be quantified by obtaining a sum-of-posterior probabilities (summed across copy number events) for each of a plurality of Gaussian distributions, such as e.g. between 20 and 25 Gaussian distributions (e.g. 22 Gaussian distributions). As another example, the copy number changepoint feature may be quantified by obtaining a sum-of-posterior probabilities (summed across copy number events) for each of a plurality of Gaussian distributions, such as e.g. between 5 and 15 Gaussian distributions (e.g. 10 Gaussian distributions). As another example, the breakpoint per predetermined sequence length feature may be quantified by obtaining a sum-of-posterior probabilities (summed across copy number events) for each of a plurality of Poisson distributions, such as e.g. between 1 and 5 Poisson distributions (e.g. 3 Poisson distributions). As another example, the breakpoint per chromosome arm feature may be quantified by obtaining a sum-of-posterior probabilities (summed across copy number events) for each of a plurality of Poisson distributions, such as e.g. between 1 and 10 Poisson distributions (e.g. 5 Poisson distributions). As another example, the number of segments with oscillating copy number feature may be quantified by obtaining a sum-of-posterior probabilities (summed across copy number events) for each of a plurality of Poisson distributions, such as e.g. between 1 and 5 Poisson distributions (e.g. 3 Poisson distributions). The precise number of distributions used may depend at least in part on the number and diversity of samples used to obtain the signatures. In the examples provided herein, the inventors used a very large number of samples from a wide variety of tumour types, enabling them to provide a nuanced picture of the behaviours of copy number features in cancer, resulting in a higher number of components identified than e.g. in Macintyre et al., 2018. The parameters of each of the predetermined distributions (such as e.g. mean and variance for a Gaussian distribution, A for a Poisson distribution) may have been determined as part of the process of obtaining the signatures of chromosomal instability. The parameters of each of the predetermined distributions (such as e.g. mean and variance for a Gaussian distribution, A for a Poisson distribution) may have been determined by fitting mixture models to the quantified set of copy number features in the plurality of tumour samples from which the signatures of chromosomal instability have been obtained. The parameters of each of a set of Gaussian distributions may have been obtained using a Variational Bayes Gaussian mixture model. In particular, the parameters of Gaussian distributions (for example for features such as segment size and changepoint) may have been obtained by fitting Dirichlet-Process Gaussian mixture models using variational inference. The parameters of each of a set of Poisson distributions (for examples features such as breakpoint counts and lengths of oscillating chains) may have been obtained using a Finite Poisson mixture model. Obtaining a set of distributions may comprise: obtaining a raw set of distributions by fitting a mixture model to a feature distribution, and combining distributions that satisfy one or more similarity criteria to obtain a final set of distributions. The one or more similarity criteria may include the mean of a distribution being within a predetermined distance from another distribution. The predetermined distance may be based on the standard deviation of the other distribution. For example, a first distribution may be combined with a further distribution if the mean of the first distribution is within the standard deviation of the further distribution. Combining distributions may comprise defining a new distribution that combines a plurality of raw distributions. The mean of such a new distribution may be defined as the weighted mean of the plurality of raw distributions that are combined. The standard deviation of such a distribution may be obtained by sampling points from the distributions that are combined. Obtaining a raw set of distributions may comprise extracting all distributions resulting from fitting a mixture model that have weights above a threshold, such as e.g. 1%.

Determining exposure to one or more signatures of chromosomal instability based on the quantified features may comprise identifying the values of E that satisfy: PbC≈E×SbC where E is a vector of size n comprising coefficients E_{1, . . . , n} where Eᵢ is the exposure to signature i; PbC is a vector of size c, each element in the vector representing a summarised measure across the copy number profile associated with one of the copy number features; and SbC is a matrix of size c by n, each value representing the weight of a summarised measured C in a signature i. The signatures of chromosomal instability have been obtained by identifying the values of SbC and E that satisfy: PbC≈E×SbC where E is a matrix of size n by p, each element in the matrix representing the exposure to a signature in the copy number profile of one of the plurality of tumour samples; PbC is a matrix of size c by p, each element in the matrix representing a summarised measure associated with one of the copy number features, wherein the summarised measured is obtained for the copy number profile of one of the plurality of tumour samples; and SbC is a matrix of size c by n, each value representing the weight of a summarised measured C in a signature/. The values of E and SbC may be obtained by non-negative matrix factorisation. The parameter n may be the number of signatures identified using the process described above. The parameter c may be the total number of summarised measures associated with the set of copy number features. The parameter p may be the number of tumour samples used to obtain the signatures that satisfy the above equation. Each of c, n may be >1. P may be >1. As described above, the summarised measures may comprise the sum over all copy number events for which a feature has been quantified in the copy number profiles for the plurality of tumour samples, of the posterior probabilities of each feature value belonging to each of a set of predetermined distributions. The set of predetermined distributions may comprise c distributions, where c can be between 30 and 50, such as e.g. 43. The values in matrix/vector E may define the exposure/activity of the signatures defined in the vector/matrix SbC. The elements of the vector/matrix SbC may define the relative weights of the different summarised measures of copy number features in each of one or more signatures. The values of E and SbC may have been obtained by non-negative matrix factorisation. For example, any non-negative matrix factorisation algorithm known in the art may be used, such as e.g. as implemented in SignatureAnalyzer (Kim et al., 2016, Tan & Fevotte, 2013). The values of E and SbC may have been obtained by: performing non-negative matrix factorisation a plurality of times with random initialisation to obtain a plurality of sets of signatures, identifying a number of signatures as the mode of the distribution of the number of signatures obtained across the plurality of times, calculating the similarity between all signatures in the sets of signatures that have the identified number of signatures, clustering the signatures based on said similarity, and selecting a set of signatures based on the location of the signatures in the clusters thus obtained, such as e.g. by selecting the most optimal solution that has a signature in the highest possible number of clusters. Such an approach advantageously enables a computationally efficient and reproducible identification of a final set of signatures from a plurality of iterations of a non-exact optimisation process (due to the use of a finite set of NMF solutions, such as e.g. 100, 200 or 300 solutions and the deterministic process from the results of the NMF iterations). This is particularly advantageous when using a large number of samples to derive the signatures, which in turn underlines the ability of the methods of the invention to comprehensively capture patterns of CIN occurring in cancer. Optimality of the solutions may be determined based on the divergence between the term PbC and the term E×SbC for the set of signatures.

The summarised measures may comprise the sum over all copy number events for which a feature has been quantified of the posterior probabilities of each feature value belonging to each of a set of predetermined distributions, wherein the predetermined distributions are the distributions defined by the parameters in Table 6, or corresponding distributions obtained by fitting mixture models to summarised measures of the set of copy number features obtained for a plurality of tumour samples. The signatures may be those defined in Table 7 or corresponding signatures obtained by quantifying the set of copy number features in a plurality of tumour samples, and identifying one or more mutational signatures likely to result in the copy number profiles of the plurality of tumour samples. The plurality of tumour samples may comprise tumour samples from a plurality of types of tumours, and the one or more signatures may have been obtained by combining: a first set of signatures obtained by quantifying the set of copy number features in the plurality of tumour samples, and identifying one or more mutational signatures likely to result in the copy number profiles of the plurality of tumour samples; and one or more further sets of signatures obtained by quantifying the set of copy number features in a plurality of tumour samples from a respective tumour type for each further set of signatures, and identifying one or more mutational signatures likely to result in the copy number profiles of the plurality of tumour samples from the respective tumour type. The plurality of tumour types may comprise at least 10 tumour types, at least 20 tumour types, at least 30 tumour types, or all tumour types represented in the TCGA database, such as e.g. 33 tumour types. The present inventors have discovered that combining a first set of signatures obtained using a pan-cancer data set and additional sets of signatures obtained using cancer type-specific datasets advantageously enabled the identification of a combined set of signatures that captures more of the signal in the pan-cancer data set than would be possible by extracting signatures only at the pan-cancer level. For example, this process enabled the identification of signatures that were identified in cancer type specific data (e.g. CX9 discovered in OC, CX10 discovered in ESCA) and not when looking at the combined cancer data alone, but that are in fact present in many more cancer types than the one in which they were originally identified. The one or more signatures may have been obtained using copy number profiles of a plurality of tumour samples, each of which has a number of copy number alternation events above a predetermined threshold. For example, a threshold of 20 copy number alterations event may be used, particularly when looking at human genome wide copy number profiles. The plurality of sets of signatures may have been combined by removing signatures in the one or more further sets that have a similarity to any signature in the first set above a predetermined threshold and/or removing signatures in the one or more further sets that have a similarity to any signature in another of the one or more further sets above a predetermined threshold, and/or removing signatures in the one or more further sets that can be obtained by a linear combination of signatures in the first set. Similarity between signatures can be obtained as a cosine similarity. The cosine similarity is a measure of similarity between two non-zero vectors of an inner product space. It is equal to the cosine of the angle between the two vectors. It is also equal to the inner products of the two vectors, normalised to each have length 1. The cosine similarity between two signatures can be calculated as:

$$sim ( S 1 , S 2 ) = \frac{S 1 \cdot S 2}{‖ S 1 ‖ ‖ S 2 ‖}$$

where S1 and S2 are equally-sized vectors with nonnegative components being the respective signatures. Alternatively, the similarity between two signatures may be obtained as the angular distance or angular similarity between the two vectors representing the signatures. As another alternative, the similarity between two signatures may be obtained as the Euclidian distance between L₂ normalised version of the two vectors representing the signatures. As another alternative, the similarity between two signatures may be obtained as the correlation between the two vectors representing the signatures. A predetermined threshold for evaluating similarity between signatures may be chosen based on a simulated distribution of similarities associated with simulated signatures derived from the first and one or more further sets of signatures. For example, a predetermined threshold may be chosen as the 0.999 quantile (or the 0.90, 0.950, 0.980, 0.990, 0.995^{th} quantile) of a distribution of similarities between said simulated signatures. The simulated distribution of similarities may have been obtained from a set of signatures using a Dirichlet process, preferably maintaining the proportion of 0s and/or signature components summing to 1 for each signature. A predetermined threshold may be chosen as a cosine similarity between 0.7 and 0.8, such as e.g. 0.74. The plurality of sets of signature may have been combined without manual curation. In other words, the plurality of sets of signatures may have been combined by removing redundant signatures using objective criteria as described above, and no signature may be removed using subjective criteria (e.g. artefactual signatures). The present inventors have identified that the methods of the invention resulted in the identification of signatures that do not contain artefactual signatures and as such it is possible to collapse a set of signatures (such as e.g. a combination of signatures identified using a pan-cancer data set and a cancer type-specific dataset) using only objective criteria based on similarity/linear combinations of signatures.

The one or more signatures of chromosomal instability may comprise signatures associated with one or more processes causing chromosomal instability, and exposure to the one or more signatures is indicative of the presence of the respective process(es) in the sample. The method may comprise determining that a signature is present in the sample if the exposure to the signature in the sample is above a signature specific threshold. The signature-specific threshold may have been identified using a simulated background distribution of signature exposure for the respective signature. The one or more signatures may comprise one or more signatures selected from: one or more signatures associated with impaired DNA damage response during mitosis, one or more signatures associated with disruption of the spindle assembly checkpoint, one or more signatures associated with impaired homologous recombination, one or more signatures associated with tolerance of whole genome duplication, one or more signatures associated with impaired non-homologous end joining, one or more signatures associated with replication stress, one or more signature associated with impaired DNA damage sensing, and one or more signatures associated with cell cycle control impairment. The method may comprise determining whether the sample/tumour has one or more processes causing chromosomal instability based on the signature exposures. For example, a process causing chromosomal instability may be present if the exposure of a signature associated with said process indicates that the signature is present in the sample.

A signature-specific threshold for a signature may have been identified (or may be identified as part of a method described herein) using a simulated background distribution of signature exposure for the respective signature. A simulated background distribution of signature exposure may be obtained by adding noise (such as e.g. random noise, optionally limited to 10% of the original value) to the number of copy number alterations events and their associated copy number features from a plurality of tumour samples to obtain a plurality of simulated copy number profiles, and determining the exposure to the one or more signatures for the simulated copy number profiles. A signature specific threshold for a signature may have been identified using such a simulated background distribution of signature exposure for the respective signature by determining the distribution of exposure value in the simulated background distribution from samples with an exposure below a threshold (e.g. exposure of 0) prior to the addition of noise. For example, the 95^{th} percentile of such a distribution may be used as a signature-specific threshold. In particular, the one or more signatures of chromosomal instability may comprise one or more signatures selected from: one or more signatures associated with chromosome missegregation (such as e.g. signatures CX1, CX6 and/or CX14 in Table 7 or corresponding signatures), one or more signatures associated with chromosome missegregation via defective mitosis (such as e.g. signatures CX1, CX6 and/or CX14 in Table 7 or corresponding signatures), one or more signatures associated with impaired homologous recombination (such as e.g. signatures CX2, CX3 and/or CX5 in Table 7 or corresponding signatures), one or more signatures associated with impaired DNA damage sensing (such as e.g. signature CX3 in Table 7 or a corresponding signature), one or more signatures associated with replication stress (such as e.g. signatures CX3, CX5, CX8, CX9, CX10, CX11, and/or CX13 in Table 7 or corresponding signatures), one or more signatures associated with tolerance of whole genome duplication and/or PI3K/AKT-mediated tolerance of whole genome duplication (such as e.g. signature CX4 in Table 7 or a corresponding signature), one or more signatures associated with impaired NHEJ optionally with replication stress for example replication fork collapse (such as e.g. signature CX10 in Table 7 or a corresponding signature), and one or more signatures associated with disruption of the spindle assembly checkpoint (such as e.g. signature CX14 in Table 7 or a corresponding signature). Determining exposure to one or more signatures may comprise determining exposure to all signatures in Table 7 or corresponding signatures, such as e.g. signatures that have been obtained as described in any embodiment of the first aspect above or second aspect below. Thus, also described herein are methods of determining whether one or more processes causing chromosomal instability are present in a tumour sample, the method including the steps of: (a) obtaining a tumour copy number profile for the sample; (b) quantifying a set of copy number features of the copy number profile, wherein a copy number feature is a metric that characterises a copy number event in a copy number profile, and wherein the set of features does not comprise the absolute copy number of segments in the copy number profile; and (c) determining exposure to one or more signatures of chromosomal instability based on the quantified features, wherein the signatures of chromosomal instability have been obtained by quantifying the set of copy number features in a plurality of tumour samples, and identifying one or more mutational signatures likely to result in the copy number profiles of the plurality of tumour samples, and wherein the one or more signatures comprise signatures associated with the one or more processes causing chromosomal instabilities. Determining exposure to one or more signatures of chromosomal instability may comprise obtaining an estimate of exposure to each of the one or more signatures and normalising the exposures for each signature. The normalising may comprise scaling signature exposures using the parameters of a distribution of the exposure to the respective signatures in a cohort of samples.

The sample may have been obtained from a subject who has bene diagnosed as having cancer. The plurality of tumour samples may comprise tumour samples from one or more of a plurality of types of tumours. The cancer and/or the plurality of tumour types may be selected from: ovarian cancer, breast cancer, endometrial cancer, kidney cancer, lung cancer, pancreatic cancer, liver cancer, oesophagus cancer, stomach cancer, head and neck cancer, brain cancer, colon cancer, pancreatic cancer, prostate cancer, bladder cancer, cervical cancer, leukemia, lymphoma, testicular cancer, thyroid cancer, melanoma, adrenal cancer, bowel cancer, sarcoma, thymoma, neuroendocrine tumour, and bile duct cancer. The sample may be a tumour sample or a liquid biopsy sample. The method may further comprise one or more of obtaining the sample from a subject who has been diagnosed as having cancer, obtaining sequence data from the sample, determining a copy number profile from sequence data obtained from the sample, obtaining a matched germline sample, obtaining sequence data from a matched germline sample, and providing to a user one or more of: the exposure to the one or more signatures, a value derived therefrom, values for one or more copy number features, and a determination of whether one or more processes causing chromosomal instability are likely to be present in the sample. The method may further comprise obtaining the sample from a tumour of a subject. The method may further comprise obtaining sequence data from a sample from a tumour. The method may further comprise providing to a user one or more of: the exposure to the one or more signatures, a value derived therefrom (such as e.g. a probabilistic score), values for one or more copy number features, and a determination of whether one or more processes causing chromosomal instability are likely to be present in the sample. The method may further comprise obtaining a germline sample from the subject and/or obtaining sequence data from a germline sample from the subject. The tumour sample may be a sample comprising tumour cells or genetic material derived therefrom. The tumour sample may be a sample of cells or tissue that has been obtained directly from a tumour (e.g. a tumour biopsy). The tumour sample may be a sample comprising cells or genetic material derived from a tumour, such as e.g. a liquid biopsy sample comprising circulating tumour cells or circulating tumour DNA. Obtaining a copy number profile for a sample may comprise receiving a copy number profile from a database, a user interface, a computing device, etc. Obtaining a copy number profile for a sample may comprise determining a copy number profile from sequence data obtained from the tumour sample and optionally from a matched germline sample. The sequence data may have been obtained using next generation sequencing or a genomic array. The sequence data may have been obtained using a genotyping array, whole exome sequencing, whole genome sequencing, single cell sequencing or shallow whole genome sequencing. Depending on the type of data used, and in particular e.g. the resolution of the data, a different set of components and signatures may be identified. Thus, the exact distributions and signatures described herein are not essential to the invention and corresponding distributions and signature may be obtained (which may in particular include a higher number of distributions, particularly if the data used has higher resolution) using different data to extract the signatures. However, the process described herein is usable with any data that is capable of generating copy number profiles. Further, the particular distributions and signatures described herein are usable to analyse a copy number profile obtained with any such data, although they may not make full use of the resolution in the copy number profile if it has been derived from data with higher resolution than that used to obtain the signatures described herein.

Obtaining a tumour copy number profile for the sample may comprise obtaining absolute copy numbers at each of a plurality of genomic locations, such as e.g. genome bins (bins of any sizes may be used, such as e.g. 10, 20, 30, 50 or 100 kb, specifically 30 kb). Absolute copy number may be determined or may have been previously determined by obtaining a relative copy number at each of the plurality of genomic locations and determining an absolute copy number for the respective locations based on the relative copy number, the mean relative copy number of the sample, the tumour cell ploidy in the sample and the tumour purity of the sample. The tumour purity (% tumour cells in the sample) and tumour cell ploidy (e.g. average absolute copy number of the tumour cells in the sample) may be purity and ploidy estimates. Methods for estimated purity are known in the art. The tumour cell ploidy may be the average absolute copy number of the tumour cells in the sample. Purity and ploidy values may be jointly estimated by identifying the purity and ploidy that minimise the differences between estimated absolute copy numbers at the plurality of genomic locations and the closest integer to the respective absolute copy number. An absolute copy number for a genomic location may be calculated as

$${aCN}_{j} = \frac{1}{purity} \cdot ( \frac{{rCN}_{j}}{d} - 2 \times ( 1 - purity ) ) - 2 ,$$

wherein aCN is the absolute copy number at location j, rCN is the relative copy number at the location, purity is the tumour purity, and d is given by

$$d = \frac{r}{( ploidy \times purity + 2 \times ( 1 - purity ) )}$$

where r is the mean relative copy number of the sample, and ploidy is the average absolute copy number of the tumour cells in the sample.

According to a second aspect, there is provided a method of characterising the processes causing chromosomal instability occurring in a plurality of types of cancers, the method including the steps of:
(a) obtaining a tumour copy number profile for a plurality of tumour samples from each of the plurality of types of cancers;
(b) quantifying a set of copy number features of the copy number profiles, wherein a copy number feature is a metric that characterises a copy number event in a copy number profile, and wherein the set of features does not comprise the absolute copy number of segments in the copy number profile; and
(c) identifying one or more mutational signatures likely to result in the copy number profiles of the plurality of tumour samples.

The method may have any of the following optional features. The method of the present aspect may have any of the features described in relation to the first aspect. In particular, the copy number features and/or the step of quantifying the copy number features may have any of the features described in relation to copy number features and quantification thereof in any embodiment of the first aspect. The one or more mutational signatures and/or the step of identifying the one or more mutational signatures may have any of the features of any embodiment of the first aspect. The method may further comprise outputting one or more results of the method, for example to a user through a user interface, to a computing device, to a computer readable medium or memory. The one or more results may comprise information identifying the signatures. The information may comprise information identifying the copy number features used (such as e.g. parameters of a plurality of components/distributions of copy number features that have been identified in the plurality of tumour copy number profiles). The information may comprise the weights of each summarised copy number feature (component) in the signature. Thus, the present aspect also relates to a method for identifying signatures of chromosomal instability, for example for use in a method according to the first aspect. The method may further comprise identifying one or more processes causing chromosomal instability associated with at least one of the one or more signatures. Identifying one or more processes causing chromosomal instability associated with at a signature may comprise analysing the pattern of copy number alterations associated with the signature (for example using the summarised copy number features and weights thereof in the signature to identify prevalent patterns of copy number abnormalities). Identifying one or more processes causing chromosomal instability associated with at a signature may comprise identifying chromosomal instability related genes (such as e.g. cancer driver genes, genes involved in DNA repair, DNA replication, cell cycle and/or chromatin organisation) whose mutational status correlates with exposure to the signature. Identifying chromosomal instability related genes whose mutational status correlates with exposure to the signature may comprise determining the mutational status (e.g. presence of single nucleotide variants, deletion and/or amplification in the gene) of said genes in the plurality of tumour samples, and testing for a difference in exposure between samples with a mutation in the gene and sample without a mutation in the gene (for example using a statistical test for equality of means between two groups). The exposures for the signature across the plurality of tumour samples may be centred and scaled prior to testing for a difference in exposure.

According to a third aspect, there is provided a method of predicting whether a subject with cancer is likely to respond to a therapy that targets a particular gene, the method comprising characterising a DNA sample obtained from a tumour of the subject, using the method of any embodiment of the first aspect, as having a high or low exposure to one or more signatures of chromosomal instability associated with response to inhibition of the gene, wherein if the sample is characterised as having a high exposure to said signature, the subject is likely to respond to the therapy. A signature of chromosomal instability may be considered to be associated with response to inhibition of the gene if the exposure to the signature is significantly correlated with the effect (e.g. cellular proliferation, growth inhibition, toxicity, etc) of perturbation of the gene, for example by genetic perturbation (such as in a CRISPR essentiality screen, also referred to as CRISPR knockout screen, or in an RNAi essentiality screen) or by drug perturbation of the gene (such as in a drug response screen). Correlation between exposure to a signature and the effect of perturbation of a gene may be assessed using Kendall's tau correlation. A sample may be considered to have a high exposure to the signature if exposure to the signature is higher than the expected exposure for the signature in a set of samples that do not respond to the perturbation of the gene. Instead, or in addition to this, a sample may be considered to have a low exposure to the signature if exposure to the signature is lower than the expected exposure for the signature in a set of samples that respond to the perturbation of the gene. Alternatively, a sample may be considered to have a high exposure to the signature if exposure to the signature is above a signature-specific predetermined threshold, such as e.g. a signature-specific predetermined threshold as described above. The therapy may be a drug, such as a chemotherapy. The method may further comprise treating the subject with a therapy that targets the gene or recommending the subject for treatment with a therapy that targets the gene if the subject is predicted to be likely to respond to the therapy. The method may further comprise treating the subject with an alternative therapy that does not target the gene or recommending the subject for treatment with an alternative therapy that does not target the gene if the subject is predicted to be unlikely to respond to the therapy.

The therapy may inhibit CCND1 and the signature may be a signature associated with tolerance to whole genome duplication or PI3K/AKT-mediated tolerance of whole genome duplication (such as e.g. CX4 in Table 7 or a corresponding signature). The therapy may inhibit PARP1 and the signature may be a signature associated with impaired homologous recombination (such as e.g. CX5 in Table 7 or a corresponding signature). The therapy may inhibit a kinase in a mitogenic pathway (such as EGFR, JAK1, MET, PRKCA, PI3KCA) and the signature may be a signature associated with replication stress (optionally wherein the signature is further indicative of focal amplifications; such as e.g. CX9 in Table 7 or a corresponding signature). The therapy may inhibit CDK4 and the signature may be a signature associated with replication stress (optionally wherein the signature is further indicative of clustered amplifications, such as e.g. CX13 in Table 7 or a corresponding signature). The therapy may be a therapy selected from any of the drugs in Table 4 or a therapy that targets any of the targets in Table 4, and the signature may be the corresponding signature in Table 4, as defined in Table 7, or a corresponding signature. The therapy may be a therapy that targets a gene selected from the genes in Table 5, and the signature may be the corresponding signature in Table 5, as defined in Table 7, or a corresponding signature. The therapy may be a therapy that targets a gene selected from the genes in Table 8, and the signature may be the corresponding signature in Table 8, as defined in Table 7, or a corresponding signature.

A corresponding signature is a signature that has been identified using data from a plurality of tumour samples and the set of copy number features, and that has the same aetiology as the corresponding signature described herein and/or is associated with the same pattern of change (e.g. as described in Table 3) as the corresponding signature described herein and/or is the most similar to the corresponding signature described herein when comparing a newly derived set of corresponding signatures and the set of signatures described herein. Establishing the similarity between sets of signatures may be performed as described herein for example in Example 2. A signature of chromosomal instability may be considered to be associated with response to inhibition of the gene if the exposure to the signature is significantly correlated with the effect of perturbation of the gene. A sample may be considered to have a high exposure to the signature if exposure to the signature is higher than the expected exposure for the signature in a set of samples that do not respond to the perturbation of the gene.

According to a fourth aspect, there is provided a method of identifying a drug target for the treatment of a cancer, the method comprising characterising a plurality of DNA samples, using the method of any embodiment of the first aspect, wherein the plurality of DNA samples comprise samples obtained from a tumour or a tumour cell line in which the drug target has been the subject of inhibition and for which response to inhibition of the drug target has been quantified, and determining whether one or more signatures of chromosomal instability are associated with response to inhibition of the drug target, wherein the presence of a signature of chromosomal instability associated with response to inhibition of the drug target is indicative of the drug target being usable for the treatment of a cancer in which the signature is active.

A signature of chromosomal instability may be considered to be associated with response to inhibition of the drug target if the exposure to the signature is significantly correlated with the effect (e.g. cellular proliferation, growth inhibition, toxicity, AUC of drug response curve, etc) of perturbation of the drug target. Correlation between exposure to a signature and the effect of perturbation of a drug target may be assessed using Kendall's tau correlation. The drug target may be a gene. The perturbation of the drug target may have been obtained, for example by genetic perturbation (such as in a CRISPR essentiality screen, also referred to as CRISPR knockout screen, or in an RNAi essentiality screen). The plurality of DNA samples may comprise samples obtained from a tumour or a tumour cell line in which a plurality of drug targets have been the subject of inhibition and for which response to inhibition of the plurality of drug targets has been quantified. In such embodiments, identifying a drug target may comprise determining whether the one or more signatures of chromosomal instability are associated with response to inhibition of any of the drug targets. The method may further comprise identifying and optionally providing a drug that targets the drug target.

Also described herein is a method of providing a prognosis for a subject who has been diagnosed with a tumour of a particular type, the method comprising: characterising a DNA sample obtained from a tumour of the subject, using the method of any embodiment of the first aspect, as having a high or low exposure to one or more signatures of chromosomal instability associated with prognosis in the particular tumour type, wherein samples with a high or low exposure to the one or more signatures are associated with different prognosis. The particular type of tumour may be ovarian cancer. For example, samples with a high exposure to signature CX14 or signature CX5 (or corresponding signature) may be associated with a poorer prognosis than samples with a low exposure to said signature. As another example, samples with a high exposure to signature CX3, signature CX11 or signature CX16 (or corresponding signature) may be associated with a better prognosis than samples with a low exposure to said signature.

According to a fifth aspect, there is provided a method of predicting whether a subject with cancer is likely to respond to a platinum based therapy, the method comprising characterising a sample obtained from a tumour in the subject, e.g. using the method of any embodiment of the first aspect, as having a high or low exposure to a first signature (CX3) associated with impaired homologous recombination plus replication stress, impaired damage sensing and impaired nucleotide excision repair, wherein if the sample is characterised as having a high exposure to said signature, the subject is likely to respond to platinum-based therapy. Instead, or in addition to this, the method may comprise characterising a sample obtained from a tumour in the subject, using the method of any embodiment of the first aspect, as having a high or low exposure to a first signature (CX5) associated with impaired homologous recombination plus replication stress, wherein if the sample is characterised as having a high exposure to said signature, the subject is not likely to respond to platinum-based therapy. The first signature (CX3) associated with impaired homologous recombination plus replication stress, impaired damage sensing and impaired nucleotide excision repair may be signature CX3 provided in Table 7 or a corresponding signature. The first signature (CX5) associated with impaired homologous recombination plus replication stress, may be signature CX5 provided in Table 7 or a corresponding signature. While mutational signatures enriched in samples that are HR deficient have been proposed in the past, the present inventors have been able to characterise the landscape of HR deficiency in more detail, and have thus identified different categories of samples showing signs of HR deficiency but also other processes that underline CIN. This enabled them to develop a highly performant clinical classifier, which is able to make predictions that would not have been possible without this more nuanced characterisation of the CIN processes active in tumours. The sample may be considered to have a high exposure to the first signature if exposure to the first signature is higher than exposure to a second signature (CX2) associated with impaired homologous recombination alone. The sample may be considered to have a low exposure to the first signature if exposure to the first signature is lower than exposure to a second signature (CX2) associated with impaired homologous recombination alone. The second signature (CX2) associated with impaired homologous recombination alone may be signature CX2 provided in Table 7 or a corresponding signature. A sample may be considered to have a high exposure to the first signature if exposure to the first signature is higher than exposure to a second signature (CX2) associated with impaired homologous recombination alone. A sample may be considered to have a low exposure to the first signature if exposure to the first signature is lower than exposure to a second signature (CX2) associated with impaired homologous recombination alone. A sample may considered to have a high exposure to the first signature if exposure to the first signature is higher than exposure to the first signature in a control sample or set of samples. In particular, a sample may be considered to have a high exposure to the first signature if exposure to the first signature is higher than the expected exposure for the first signature in a cohort of patients that are resistant to platinum-based therapy. Similarly, a sample may be considered to have a low exposure to the first signature if exposure to the first signature is not higher than the expected exposure for the first signature in a cohort of patients that are resistant to platinum-based therapy. Alternatively, a sample may be considered to have a high exposure to the first signature if exposure to said signature is above a signature-specific predetermined threshold, such as e.g. a signature-specific predetermined threshold as described above (e.g. based on a background distribution of signature exposure for said signature). The cancer may be ovarian cancer (e.g. high grade serous ovarian cancer).

According to a related aspect, there is provided a method of predicting whether a subject with cancer is likely to respond to a platinum based therapy, the method comprising characterising a sample obtained from a tumour in the subject, e.g. using the method of any embodiment of the first aspect, as having a high or low exposure to a third signature (CX5) associated with impaired homologous recombination plus replication stress but not impaired damage sensing and impaired nucleotide excision repair, wherein if the sample is characterised as having a high exposure to said signature, the subject is not likely to respond to platinum-based therapy. The third signature (CX5) associated with impaired homologous recombination plus replication stress but not impaired damage sensing and impaired nucleotide excision repair may be signature CX5 provided in Table 7 or a corresponding signature. A sample may be considered to have a high exposure to the third signature if exposure to the third signature is higher than the expected exposure for the third signature in a cohort of patients that are sensitive to platinum-based therapy. Similarly, a sample may be considered to have a low exposure to the third signature if exposure to the third signature is not higher than the expected exposure for the third signature in a cohort of patients that are sensitive to platinum-based therapy. Alternatively, a sample may be considered to have a high exposure to the second signature if exposure to said signature is above a signature-specific predetermined threshold, such as e.g. a signature-specific predetermined threshold as described above (e.g. based on a background distribution of signature exposure for said signature). The first, second and/or third signature(s) may have been obtained using the method of any embodiment of the second aspect. The first, second and/or third signature exposure may have been normalised, for example by centring and scaling using predetermined parameters for the respective signatures. The predetermined parameters may have been obtained using a suitable cohort of patients/samples, such as a cohort of patients comprising platinum-sensitive and platinum-resistant patients.

According to a related aspect, there is provided a method of predicting whether a subject with cancer is likely to respond to a platinum based therapy, the method comprising characterising a sample obtained from a tumour in the subject, e.g. using the method of any embodiment of the first aspect, in terms of its exposure to a first signature (CX3) associated with impaired homologous recombination plus replication stress, impaired damage sensing and impaired nucleotide excision repair, a second signature (CX2) associated with impaired homologous recombination alone, and a third signature (CX5) associated with impaired homologous recombination plus replication stress but not impaired damage sensing and impaired nucleotide excision repair, and classifying the sample between at least a first class and a second class using a classifier that has been trained to classify samples between at least a first class and a second class based on their exposure to the first, second and third signatures, wherein samples in the first class are likely to respond to platinum-based therapy and samples in the second class are unlikely to respond to platinum-based therapy. The classifier may be a support vector machine.

The signatures may be copy number signatures, i.e. signatures derived from copy number profiles. Preferably, the signatures of chromosomal instability have been obtained using the methods of the first aspect. The method may further comprise administering a platinum-based therapy, to a subject that has been diagnosed as likely to respond to the platinum-based therapy. The method may comprise recommending a subject that has been diagnosed as likely to respond to the platinum-based therapy for treatment with the platinum-based therapy. The method may comprise administering an alternative therapy (e.g. another chemotherapy, radiotherapy, etc.) and/or recommending a subject for treatment with an alternative therapy, where the subject has been diagnosed as not likely to respond to the platinum-based therapy.

According to a further aspect, there is provided a method of selecting a subject having cancer for treatment with a platinum-based therapy, the method comprising characterising a sample obtained from a tumour in the subject as likely to respond to a platinum-based therapy according to any embodiment of the fifth aspect, and selecting the subject for treatment with a platinum-based therapy if the sample is characterised as likely to respond to platinum-based therapy.

According to a further aspect, there is provided an platinum-based therapy for use in a method of treatment of cancer in a subject from whom a DNA sample has been obtained and the DNA sample has been characterised by a method according to any embodiment of the fifth aspect as likely to respond to platinum-based therapy.

According to any of these aspects, the platinum-based therapy may be administered (or recommended for administration) in combination with one or more therapies, such as one or more chemotherapies, one or more courses of radiotherapy and/or one or more surgical interventions.

According to a further aspect, there is provided a platinum-based therapy for use in a method of treatment of cancer in a subject, the method comprising: (i) determining whether a DNA sample obtained from said subject is likely to respond to platinum-based therapy using a method according to any embodiment of the fifth aspect; and (ii) administering the platinum-based therapy to said subject if the DNA sample is determined to be likely to respond to platinum-based therapy.

According to a further aspect, there is provided a platinum-based therapy for use in a method of treatment of cancer in a subject, the method comprising: (i) determining whether a DNA sample obtained from said subject is likely to respond to platinum-based therapy using a method according to a method described herein; and (ii) administering the platinum-based therapy to said subject if the DNA sample is determined to be likely to respond to platinum-based therapy. The subject may have been diagnosed as having or being at risk of having ovarian cancer or oesophagal cancer.

According to a further aspect, there is provided a system comprising: a processor; and a computer readable medium comprising instructions that, when executed by the processor, cause the processor to perform the (computer-implemented) steps of the method of any preceding aspect.

According to a further aspect, there is provided a non-transitory computer readable medium or media comprising instructions that, when executed by at least one processor, cause the at least one processor to perform the method of any embodiment of any aspect described herein.

According to a further aspect, there is provided a computer program comprising code which, when the code is executed on a computer, causes the computer to perform the method of any embodiment of any aspect described herein.

According to a further aspect, there is provided a method of predicting whether a subject with cancer is likely to respond to a therapy that targets a particular gene, the method comprising: characterising a DNA sample obtained from a tumour of the subject as having a high or low exposure to one or more signatures of chromosomal instability associated with response to inhibition of the gene, wherein if the sample is characterised as having a high exposure to said signature, the subject is likely to respond to the therapy. In some embodiments, the one or more signatures may be as defined herein (for example a CX1, CX2, CX3, CX4, CX5, CX6, CX7, CX8, CX9, CX10, CX11, CX12, CX13, CX14, CX15, CX16 or CX17 signature as defined in Table 7). The signature may be obtained or obtainable by a method as defined in connection with the first aspect of the invention. The therapy may be a therapy selected from any of the drugs in Table 4 or a therapy that targets any of the targets in Table 4, and the signature may be the corresponding signature in Table 4, as defined in Table 7, or a corresponding signature. The therapy may be a therapy that targets a gene selected from the genes in Table 5, and the signature may be the corresponding signature in Table 5, as defined in Table 7, or a corresponding signature. The therapy may be a therapy that targets a gene selected from the genes in Table 8, and the signature may be the corresponding signature in Table 8, as defined in Table 7, or a corresponding signature. A corresponding signature is a signature that has been identified using data from a plurality of tumour samples and the set of copy number features, and that has the same aetiology as the corresponding signature described herein and/or is associated with the same pattern of change (e.g. as described in Table 3) as the corresponding signature described herein and/or is the most similar to the corresponding signature described herein when comparing a newly derived set of corresponding signatures and the set of signatures described herein. Establishing the similarity between sets of signatures may be performed as described herein for example in Example 2. A signature of chromosomal instability may be considered to be associated with response to inhibition of the gene if the exposure to the signature is significantly correlated with the effect of perturbation of the gene. A sample may be considered to have a high exposure to the signature if exposure to the signature is higher than the expected exposure for the signature in a set of samples that do not respond to the perturbation of the gene. In some embodiments in accordance with this aspect of the invention, the therapy inhibits CCND1 and the signature is a signature associated with tolerance to whole genome duplication or PI3K/AKT-mediated tolerance of whole genome duplication (such as e.g. CX4 in Table 7 or a corresponding signature), wherein the therapy inhibits PARP1 and the signature is a signature associated with impaired homologous recombination (such as e.g. CX5 in Table 7 or a corresponding signature), wherein the therapy inhibits a kinase in a mitogenic pathway (such as EGFR, JAK1, MET, PRKCA, PI3KCA) and the signature is a signature associated with replication stress (optionally wherein the signature is further indicative of focal amplifications; such as e.g. CX9 in Table 7 or a corresponding signature), wherein the therapy inhibits CDK4 and the signature is a signature associated with replication stress (optionally wherein the signature is further indicative of clustered amplifications, such as e.g. CX13 in Table 7 or a corresponding signature).

### BRIEF DESCRIPTION OF THE FIGURES

FIG. 1 is a flow diagram showing, in schematic form, a method of characterising a DNA sample according to the disclosure (A), and a method of characterising types of chromosomal instability in a plurality of samples according to the present disclosure (B).

FIG. 2 shows an embodiment of a system for characterising a DNA sample.

FIG. 3 is a flow diagram illustrating schematically a method of providing a prognosis, identifying a drug target, identifying a therapy or treating a subject according to an embodiment of the present disclosure.

FIG. 4 is a flow diagram illustrating schematically a method of identifying whether a patient is likely to respond to platinum-based therapy according to an embodiment of the present disclosure.

FIG. 5 shows the workflow of sample filtering and detectable chromosomal instability (dCIN) used in Example 1. a: REMARK diagram showing flow of samples through the study. b: For each copy number feature: a histogram of number of events per sample that could not be assigned to an ovarian copy number signature on the TCGA ovarian cohort. Red dotted line indicates the quantile 0.95. c: Scatterplot of cancer types comparing the estimate of detectable CIN described in Example 1 to estimates reported in the Mitelman database. d+e: Boxplots comparing our estimate of detectable CIN with aneuploidy score and four CNA-specific metrics. Boxes represent the interquartile range (IQR) with the median as a bolded line. The whiskers extend to the largest/smallest value no further than 1.5*IQR from the hinge. Outliers beyond the end of the whiskers are marked individually as points.

FIG. 6 shows an overview of copy number features and signature identification according to an example of a process described herein. a: Overview of 5 fundamental copy number features extracted from 6,335 samples with detectable CIN (dCIN). A feature capturing absolute copy number is not included. b: Mixture modelling to split the genome-wide feature distributions into smaller components by either Variational Bayes Gaussian mixture models or Finite Poisson mixture models. These components represent basic building blocks of each feature distribution. c: For all samples and features, calculating the probability of each CNA (no diploid segments) to belong to each mixture component (posterior probability) and summing the probabilities for each sample to obtain a 43-dimensional feature vector. d: The sum-of-posterior matrix for all 6,335 samples was split in two matrices by a Bayesian implementation of the non-negative matrix factorisation (NMF), resulting in a signature catalogue and an activity catalogue.

FIG. 7 shows a schematic of a signature compendium identification according to an example of a process described herein. a: From the complete input matrix 10 pan-cancer signatures were identified. b: For the 20 cancer types with over 100 samples each, 128 cancer-type enriched signatures (CTES) were identified. c: We removed all cancer-type enriched signatures that had a cosine similarity over 0.74 with any pan-cancer signature. d: We selected a representative signature (highest activity for the largest number of samples) from the groups of cancer-type enriched signatures that had a cosine similarity over 0.74. e: We performed non-negative least squares on each pair of pan-cancer specific signatures and cancer type enriched signature. For any combination which showed a reconstruction error below 0.1, the cancer-type enriched signature was removed. f: The sets of 10 pan-cancer and 7 CTES were joined to a compendium of 17 signatures. g: Using linear combination decomposition, the signature activities were calculated for the 6,335 TCGA samples.

FIG. 8 shows signature definitions in an exemplary compendium of signatures of the disclosure. Displayed on the left are the five features, their mixture components and component means. The heatmap on the right shows weights for each signature. Weights are normalised per row (component) for ease in comparison across signatures for each component.

FIG. 9 shows the prevalence of copy number signatures according to an embodiment of the disclosure. Signatures were labelled based on pan-cancer prevalence, with signature 1 having the highest frequency. The heatmap shows their frequency across 33 cancer types.

FIG. 10 shows Monte Carlo simulation results for determining signature-specific noise thresholds according to an embodiment of the disclosure. a: Each plot (1 per signature) shows the interquartile range of sample signature activities after the introduction of noise in the copy number features using Monte Carlo simulation. Samples are ordered by their observed signature activity (red line). b: Schematic showing how we fitted a Gaussian distribution to the simulated values of all samples with an observed signature activity of 0 (red line). The horizontal black line represents the quantile 0.95 of the fitted Gaussian and forms the basis of our signature specific noise threshold, where values below this line are not distinguishable from 0. c: Plot of the signature-specific thresholds for the 17 copy number signatures.

FIG. 11 shows data demonstrating the stability of signatures according to the disclosure across different copy number profiling technologies. This figure summarises the comparison of original copy number profiles and signatures, to copy number profiles and signatures derived using different copy number profiling technologies, across the same set of 478 tumours. The columns contain results for the different technologies and the rows contain results for comparison between copy number profiles (top), signature activities (middle) and signature definitions (bottom, limited to pan-cancer signatures). For each comparison we show results for a range of penalties for ASCAT's piecewise constant fitting or ASCAT.sc's circular binary segmentation.

FIG. 12 shows a workflow for determining signature aetiology and confidence rating for a signature compendium according to an embodiment of the disclosure. a: This flowchart shows how we take an association between a mutated gene and signature activity and derive a hypothesis for a putative aetiology. b: This flowchart represents the decision making process leading to the assignment of a 3-star rating confidence score. c: An example of the star rating process for CX3.

FIG. 13 shows a summary of associations between signatures according to an embodiment of the disclosure and other covariates. a: The main panel shows significant associations between copy number signatures and mutated genes. The panels below show gene annotations. b: The top four rows in the table show a summary of the copy number patterns represented by each signature. The remaining rows show highly significant associations between signatures and different covariates. Unless otherwise specified, only positive correlations are shown.

FIG. 14 shows associations of signature activities with five important biological covariates. a: Scatterplots of the Spearman correlation between signature activities and chromothriptic events (top left), kataegis events (top right), proportion of the genome altered by loss of heterozygosity (bottom left) and number of whole-chromosome CNAs (bottom right). Summary table is displayed in Extended Data FIG. 8b. b: Scatterplot of two statistical tests studying the relationship of signatures with whole-genome duplication: results of proportion tests for samples with zero activity (left) and results of Welsh's t-tests for samples with activities above zero (right).

FIG. 15 shows associations of signature activities with telomere length and telomere elongation machinery. a: Scatterplot of the slopes of robust linear models fitted to signature activities and telomere length. b: Scatterplots of the Spearman correlation between signature activities and Telomerase Signature Score. c: Boxplots comparing signature activities of samples split by the genomic amplification status of TERT and TERC, crucial parts of the telomerase elongation machinery. d-e: Scatterplots of the Spearman correlation between signature activities and TERC (d) or TERC (e) gene expression. f: Boxplots comparing signature activities of samples split by gene expression status of lncRNA TERRA. g: Boxplots comparing signature activities of samples split by the mutation status of ATRX and DAXX, associated with the activation of alternative telomere lengthening (ALT). For all boxplots: Boxes represent the interquartile range (IQR) with the median as a bolded line. The whiskers extend to the largest/smallest value no further than 1.5*IQR from the hinge. Outliers beyond the end of the whiskers are marked individually as points.

FIG. 16 shows impaired HR signatures according to an embodiment described herein and their associations. a: The boxplots summarise signature activities of different patient groups (rows) defined by their driver gene mutation status. Ovarian samples are coloured in dark green and breast in orange. The stars on the right hand side of each plot show where there was a significant difference between the WT BRCA 1/2 population and mutant (WT=wild type; LOH=loss of heterozygosity) b: The boxplots summarise the scaled signature activities of 5,466 TCGA samples split by low, medium and high cell cycle scores. The brackets and stars show where there was a significant increase from low to medium to high cell cycle groups. c: Volcano plots showing the results of a correlation between signature activity and expression of genes involved in NER. Each dot represents a gene, coloured dots show significant correlations. d: Spearman correlation coefficient (y-axis) of correlation between signature activities and seven common metrics of HRD listed at top). Individual coefficients are displayed for IHR signatures and the distribution of coefficients from remaining signatures are represented by boxplots.

FIG. 17 shows associations of signature activities described herein with tandem duplication events, scores and classes. a-b: Scatterplots of the Spearman correlation between signature activities and number of tandem duplications (a) and tandem duplication score (b). c: Heatmap of significant differences in signature activities of TCGA samples with no tandem duplications (non-TDP) and samples categorised into six tandem duplication classes tested by Welch's t-test.

FIG. 18 shows associations of signature activities described herein with rearrangement signatures. Heatmap of significant Spearman correlations between copy number signature activities and Rearrangement Signature exposures of the ICGC breast cancer cohort.

FIG. 19 shows associations of signature activities described herein with centrosome amplifications (CA20). a: Boxplots comparing centrosome amplification score (CA20) of samples split by dominant impaired HR signature. Statistical significance was tested by Welch's t-test. Boxes represent the interquartile range (IQR) with the median as a bolded line. The whiskers extend to the largest/smallest value no further than 1.5*IQR from the hinge. Outliers beyond the end of the whiskers are marked individually as points. b: Scatterplot of Kendall correlation between copy number signature activity and CA20 score.

FIG. 20 shows a comparison of 17 CIN signatures described herein with 7 ovarian copy number signatures previously described. a: Cosine similarities between the definitions of the 17 CIN signatures (x-axis) and ovarian copy number signatures (y-axis). b: Schematic of cosine similarity correction by maximum of background distribution for each signature pair. c: Corrected cosine similarities between signature definitions. d: Cosine similarities between the activities of the 17 CIN signatures (x-axis) and ovarian copy number signatures (y-axis) on the samples of the TCGA ovarian cancer cohort. e: Schematic of cosine similarity correction by maximum of background distribution for each signature pair. f: Corrected cosine similarities between signature definitions.

FIG. 21 shows associations of samples grouped by their dominant IHR signature activities as described herein with frequency of microhomologies of SVs. a: Boxplots comparing frequency of structural variants (SVs) with microhomologies split by the repair pathway that most likely created them. PCAWG Samples were split by dominant impaired HR signature. Statistical significance was tested by Welch's t-test. b: Zoomed in boxplot for single strand annealing (SSA) repair pathway. For boxplots: Boxes represent the interquartile range (IQR) with the median as a bolded line. The whiskers extend to the largest/smallest value no further than 1.5*IQR from the hinge. Outliers beyond the end of the whiskers are marked individually as points.

FIG. 22 shows data indicating that samples with more CNAs experience a mitotic catastrophe phenotype with major contributions by signatures CX2, CX3 and C5 described herein. a: Scatterplot of the number of structural variants and the number of copy number aberrations for the PCAWG cohort. Samples with low weights assigned in a robust linear model are samples where we predict an accumulation of CNAs through mitotic catastrophe (blue samples). b: Scatterplot of (a) with log 10 axis. c: Barplot comparing the proportion of high and low Cell cycle scores for samples with predicted mitotic catastrophe (see blue samples in panel a) and samples without (see green samples in panel a). Statistical test was Fisher's exact test. d: Boxplots comparing signature activities for samples with (blue) and without (green) inferred mitotic catastrophe. Comparisons made with a Welch's t-test. e: Boxplots comparing the frequency of estimated number of CNAs caused by impaired HR signatures for samples with (blue) and without (green) inferred mitotic catastrophe. Comparisons made with a Welch's t-test. f: Scatterplot comparing Cell cycle scores and proportion of estimated CNAs for samples with (blue) and without (green) inferred mitotic catastrophe. Lines are fitted linear models. g: Boxplots comparing the frequency of estimated number of CNAs caused by impaired HR signatures for samples with (blue) and without (green) inferred mitotic catastrophe and split by Cell cycle score category (low, medium, high). Comparisons made with a Welch's t-test. For boxplots: Boxes represent the interquartile range (IQR) with the median as a bolded line. The whiskers extend to the largest/smallest value no further than 1.5*IQR from the hinge. Outliers beyond the end of the whiskers are marked individually as points.

FIG. 23 shows a correlation of gene expression of known DNA repair pathway genes with the activities of impaired HR signatures described herein. Scatterplots of the Spearman correlations between signature activities of the three impaired HR signatures ordered by levels of perturbation (CX2, CX5, CX3) and gene expression of selected genes shown to be of importance to six DNA repair pathways. Positively associated genes are marked green and negatively associated genes red. The gene list is shown in Table 8.

FIG. 24 shows associations of signature activities described herein with frequency of microhomologies of SVs. a-c: Boxplots comparing frequency of structural variants (SVs) with microhomologies split by the repair pathway that most likely created them: non-homologous end joining (NHEJ) (a), polymerase theta-mediated end joining (TMEJ) (b) and single strand annealing (SSA) (c). PCAWG Samples were split by dominant impaired HR signature. Statistical significance was tested by Welch's t-test and coloured when there was a significant difference between depleted samples and samples with a dominant signature. Boxes represent the interquartile range (IQR) with the median as a bolded line. The whiskers extend to the largest/smallest value no further than 1.5*IQR from the hinge. Outliers beyond the end of the whiskers are marked individually as points.

FIG. 25 shows associations of signature activities described herein with cell cycle score. Boxplots comparing signature activity between groups of samples split by their Cell cycle score (CCS). Statistical significance was tested by Welch's t-test and coloured when there were significant differences between the low and high cell cycle group and the medium and high cell cycle group. Boxes represent the interquartile range (IQR) with the median as a bolded line. The whiskers extend to the largest/smallest value no further than 1.5*IQR from the hinge. Outliers beyond the end of the whiskers are marked individually as points.

FIG. 26 shows associations of signature activities described herein with extrachromosomal DNA (ecDNA). Barplots comparing the presence of ecDNA amplicons with the presence of the 17 CIN signatures for TCGA samples. Statistical significance was tested with Fisher's exact test. Log odds are displayed in the barplot on the right. If found significant, grey and black were switched to brighter colours: beige for no focal SCNA, and purple/red/green/yellow/blue for specific types of ecDNA amplicons.

FIG. 27 shows that the signatures described herein can be used as biomarkers for drug response and discovery of novel drug targets. a: Response biomarkers and novel drug targets were found by correlating signature activities with gene essentiality determined by CRISPR or RNAi screens, and with response to drug perturbations. b: Copy number signatures as biomarkers for predicting response to 44 therapies. c: Copy number signatures as biomarkers for drug target discovery. The listed targets were filtered for druggability according to their structure or by ligand-base approaches (n=104), and their previous known association with CIN (n=49).

FIG. 28 shows the prediction of platinum sensitivity using impaired homologous recombination signatures. a: Model of signature aetiologies. b: Hazard ratios for predicting overall survival in ovarian cancers (platinum sensitivity) for individual signatures. c: Clinical classifier built on CX3 and CX2 activities of ovarian samples with germline BRCA1 mutations. d: Performance of classifier from (c) applied to four cancer cohorts from the TCGA and PCAWG collections.

FIG. 29 shows associations of signature activities described herein with overall survival in TCGA-OV cancer. Cox proportional hazard model for the survival of the TCGA ovarian cancer cohort. Covariates are the activity of the 17 signatures, age, race and tumour stage of the 545 samples.

FIG. 30 shows the performance of classifiers for predicting platinum sensitivity as described herein and comparative classifiers. a: Kaplan-Meier curve showing the overall survival probabilities of TCGA ovarian cancer patients split into two groups using our CX3/CX2 classifier. b: Hazard ratios from a cox proportional hazards model trained on our CX3/CX2 classifier and other covariates predicting overall survival of TCGA ovarian cancer patients. c+d: Median survival and hazard ratios generated using predictions from three classifiers (our CX3/CX2 classifier, HRDetect and Myriad myChoice) applied to five cancer cohorts from the TCGA, PCAWG and ICGC projects. The number and proportion of patients predicted to be sensitive (with HRD) and resistant (without HRD) by each classifier are listed on the right.

FIG. 31 shows Kaplan-Meier curves and Cox proportional hazard model for germline BRCA1 samples of the TCGA-OV cohort. Top plot: Univariate survival modeling by a Kaplan-Meier estimate for germline BRCA1 mutant samples of the TCGA-OV cohort. Bottom plot: Multivariate survival modeling by a Cox proportional hazard model correcting for age and tumour stage. A: Clinical classification predicting sensitivity to platinum-based chemotherapy done by the CX2/CX3 classifier described herein. B: Clinical classification performed by HRDetect (no bottom plot as model did not converge). C: Clinical classification performed by Myriad myChoice.

FIG. 32 shows Kaplan-Meier curves and Cox proportional hazard model for all TCGA-OV samples. Top plot: Univariate survival modeling by a Kaplan-Meier estimate for the TCGA-OV cohort. Bottom plot: Multivariate survival modeling by a Cox proportional hazard model correcting for age and tumour stage. A: Clinical classification predicting sensitivity to platinum-based chemotherapy done by the CX2/CX3 classifier described herein. B: Clinical classification performed by HRDetect. C: Clinical classification performed by Myriad myChoice.

FIG. 33 shows Kaplan-Meier curves and Cox proportional hazard model for two PCAWG cohorts. Top plot: Univariate survival modeling by a Kaplan-Meier estimate for two PCAWG cohorts: Australian ovarian cancer cohort (OV-AU) and British oesophageal cancer cohort (ESAD-UK). Bottom plot: Multivariate survival modeling by a Cox proportional hazard model correcting for age and tumour stage. A: Clinical classification predicting sensitivity to platinum-based chemotherapy done by the CX2/CX3 classifier described herein. B: Clinical classification performed by HRDetect. C: Clinical classification predicting sensitivity to platinum-based chemotherapy done by our CX2/CX3 classifier. D: Clinical classification performed by HRDetect.

FIG. 34 shows Kaplan-Meier curves and Cox proportional hazard model for ICGC breast cancer cohort. Top plot: Univariate survival modeling by a Kaplan-Meier estimate for the ICGC breast cancer cohort. Bottom plot: Multivariate survival modeling by a Cox proportional hazard model correcting for age and tumour stage. A: Clinical classification predicting sensitivity to platinum-based chemotherapy done by our CX2/CX3 classifier. B: Clinical classification performed by HRDetect.

FIG. 35 shows Kaplan-Meier curves and Cox proportional hazard model for three alternative classifiers as described herein, utilising CX2, CX3 and CX5. Top plot: Univariate survival modeling by a Kaplan-Meier estimate for the TCGA ovarian cancer cohort. Bottom plot: Multivariate survival modeling by a Cox proportional hazard model correcting for age and tumour stage. A: Clinical classification predicting sensitivity to platinum-based chemotherapy done by a logistic regression model taking all three impaired HR signatures into account. B: Clinical classification predicting sensitivity to platinum-based chemotherapy done by support vector machine (SVM). C: Clinical classification predicting sensitivity to platinum-based chemotherapy done by a SVM on clustered samples.

FIG. 36 shows Estimated number of CNAs produced by each signature described herein. a: Heatmap showing estimated number of CNAs produced by the 17 signatures split by the 33 TCGA cancer types. b: Pie chart of the estimated number of CNAs produced by the 17 signatures. c: Lorenz curve of inequality showing the distribution of estimated number of CNAs produced by each signature across the 6,335 TCGA samples.

FIG. 37 shows a comparison between the use of copy number features as described herein to derive signatures, and the use of copy number features as described in Macintyre et al., 2018. A. Number of ovarian signatures decrease to six without copy number feature. Left: Scatterplots of four decision criteria used for settling on the final number of signatures. Right: Heatmap comparing the signature definitions between the OV7 (original signatures published in Macintyre et al. 2018) and the OV6 (redefined signatures without copy number feature). B. Comparing the signature exposures for the ovarian copy number signatures influenced by two different feature extraction methods: the updated feature extraction described herein (x-axis) and the original feature extraction method used in Macintyre et al. (y-axis). The data in A was obtained as described in Macintye et al. (2018), in relation to supplementary FIG. 3: the circle and solid lines represent the results from the BriTROC-1 samples run, whereas the triangles and dotted lines represent results from 1,000 randomly permuted BriTROC-1 matrices (these can be considered a null measure). Basis=signature-by-component matrix, coefficients=patient-by-signature matrix, consensus=connectivity matrix of patients clustered by their dominant signature across 1,000 runs. The best fit is the run that showed the lowest objective score across the 1,000 runs. The optimal value defines the point of stability in the cophenetic, dispersion and silhouette coefficients and is the maximum sparsity achievable above the null model for the basis matrix.

FIG. 38 shows an assessment of signature quantification and identification using whole-genome and whole-exome data. a/d: Heatmap showing goodness of copy number fits on 478 TCGA samples which were WGS (a) or WES (d) sequenced in the PCAWG project and show detectable levels of CIN. ble: Density plots of cosine similarities between SNP6-derived signature activities and WGS-derived (b) or WES-derived (e) activities. c/f: Heatmap showing cosine similarities between SNP6-derived signature definitions and WGS-derived (c) or WES-derived (f) signature definitions. (*): For settings marked with a star it was not possible to derive solutions for K=10, instead the optimal number of K was chosen (lower than K=10).

FIG. 39 shows an analysis of segment size distributions (<100 kb) across technology platforms and algorithms. a: Density plots of the number of copy number segments for 451 samples that were present for all segmentation algorithms. b: Histogram of segment sizes for segments smaller than 100 kB. c: Scatterplot of segments by their segment size (smaller than 100 KB) and their absolute copy number for the 451 TCGA/PCAWG samples.

FIG. 40 shows associations of signature activity and sample ploidy. Volcano plot of the t-Test statistic and −log (q-value) from a multivariate regression model testing the association between signatures and sample ploidy by correcting for CX4 activity and whole-genome duplication status. CX4 is therefore omitted.

FIG. 41 shows associations of signature activities with patient ethnicity. Boxplots comparing signature activity between groups of samples split by their ethnicity. Statistical significance was tested by Welch's t-test and coloured when there were significant differences between any of the three comparisons with the white patient population (q-value<0.001).

FIG. 42 shows an analysis of impaired homologous recombination signatures in different tumour types. Boxplots showing the signature activity of impaired HR signatures (CX2, CX5, CX3, ordered by increased severity) for TCGA samples with wild type BRCA 1/2 genes (also without loss of heterozygosity at either loci). Samples from ovarian cancer (OV with and without mutant BRCA1/2), positive triple-negative breast cancers (TNBC) and negative triple-negative breast cancers (non-TNBC) were highlighted. Differences in mean activity was tested with Welch's t-test and corrected for false discovery rate (q-value<0.05).

FIG. 43 shows the results of an analysis of sensitivity to platinum-based therapy using methods described herein. A. Results for each IHR signature after training a Cox proportional hazards model to predict progression free survival across 40 ovarian cancers treated with platinum-based chemotherapy. Hazard ratios, their 95% confidence interval and Wald test significance are reported. The dashed line indicates a hazard ratio of 1. B. Kaplan-Meier estimator showing the progression survival probabilities of ovarian cancer patients split into two groups using a classifier based on signatures CX3 and CX2 as described herein.

### DETAILED DESCRIPTION

In describing the present invention, the following terms will be employed, and are intended to be defined as indicated below.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

A “sample” as used herein may be a cell (including a circulating cell such as a circulating tumour cell) or tissue sample (e.g. a biopsy), a biological fluid, an extract (e.g. a protein or DNA extract obtained from the subject), from which genomic material can be obtained for genomic analysis, such as genomic sequencing (whole genome sequencing, whole exome sequencing, targeted (also referred to as “panel”) sequencing). In particular, the sample may be a tumour sample, a biological fluid sample containing DNA or cells, a blood sample (including plasma or serum sample), a urine sample, a cervical smear, an ascites fluid sample. It has been found that urine, ascites fluid and cervical smears contains cells, and so may provide a suitable sample for use in accordance with the present invention. Other sample types suitable for use in accordance with the present invention include fine needle aspirates, lymph nodes samples (e.g. aspirates or biopsies), surgical margins, bone marrow or other tissue from a tumour microenvironment, where traces of tumour DNA may be found or expected to be found. The sample may be one which has been freshly obtained from a subject or may be one which has been processed and/or stored prior to making a determination (e.g. frozen, fixed or subjected to one or more purification, enrichment or extractions steps). For example, the sample may be a formalin-fixed tumour sample. The sample may be derived from one or more of the above biological samples via a process of enrichment or amplification. For example, the sample may comprise a DNA library generated from the biological sample and may optionally be a barcoded or otherwise tagged DNA library. A plurality of samples may be taken from a single patient, e.g. serially during a course of treatment. Moreover, a plurality of samples may be taken from a plurality of patients. As such, a sample as described herein may refer to any type of sample comprising cells or genomic material derived therefrom, whether from a biological sample obtained from a subject, or from a sample obtained from e.g. a cell line. “The sample is preferably from a mammalian (such as e.g. a mammalian cell sample or a sample from a mammalian subject, including in particular a model animal such as mouse, rat, etc.), preferably from a human (such as e.g. a human cell sample or a sample from a human subject). Further, the sample may be transported ad/or stored, and collection may take place at a location remote from the genomic sequence data acquisition (e.g. sequencing) location, and/or the computer-implemented method steps may take place at a location remote from the sample collection location and/or remote from the genomic data acquisition (e.g. sequencing) location (e.g. the computer-implemented method steps may be performed by means of a networked computer, such as by means of a “cloud” provider).

“Patient” as used herein in accordance with any aspect of the present invention is intended to be equivalent to “subject” and specifically includes both healthy individuals and individuals having a disease or disorder (e.g. a proliferative disorder such as a cancer). Preferably, the patient is a human patient. In some cases, the patient is a human patient who has been diagnosed with, is suspected of having or has been classified as at risk of developing, a cancer. The cancer may be ovarian cancer, breast cancer, endometrial cancer (uterus/womb cancer), kidney cancer (renal cell), lung cancer (small cell, non-small cell and mesothelioma), central nervous system cancer including brain cancer (gliomas, astrocytomas, glioblastomas), melanoma (including choroid melanoma and skin cancers), merkel cell carcinoma, clear cell renal cell carcinoma (ccRCC), carcinoma of unknown primary (CUP), lymphoid cancer (such as e.g. lymphoma), gastrointestinal cancer (e.g. colorectal cancer, oesophagus cancer stomach cancer), small bowel cancers (duodenal and jejunal), leukemia, pancreatic cancer, hepatobiliary tumours, germ cell cancers, bone/soft tissue cancer, prostate cancer, head and neck cancers (such as e.g. adenoid cystic carcinoma, ACC), pancreatic cancer, cervical cancer (e.g. Cervical Squamous Cell Carcinoma and endocervical adenocarcinoma, CESC), liver cancer, bladder cancer (such as e.g. bladder carcinoma, BLCA), urinary tract cancer, neuroendocrine tumour (NET), thyroid cancer and sarcomas. For example, the cancer may be any cancer represented in The Cancer Genome Atlas (TCGA) such as LAML (Acute Myeloid Leukemia), ACC (Adrenocortical carcinoma), BLCA (Bladder Urothelial Carcinoma, LGG (Brain Lower Grade Glioma, BRCA (Breast invasive carcinoma), CESC (Cervical squamous cell carcinoma and endocervical adenocarcinoma), CHOL (Cholangiocarcinoma), LCML (Chronic Myelogenous Leukemia), COAD (Colon adenocarcinoma), ESCA (Esophageal carcinoma), GBM (Glioblastoma multiforme), HNSC (Head and Neck squamous cell carcinoma), KICH (Kidney Chromophobe), KIRC (Kidney renal clear cell carcinoma), KIRP (Kidney renal papillary cell carcinoma), LIHC (Liver hepatocellular carcinoma), LUAD (Lung adenocarcinoma), LUSC (Lung squamous cell carcinoma), DLBC (Lymphoid Neoplasm Diffuse Large B-cell Lymphoma), MESO (Mesothelioma), OV (Ovarian serous cystadenocarcinoma), PAAD (Pancreatic adenocarcinoma), PCPG (Pheochromocytoma and Paraganglioma), PRAD (Prostate adenocarcinoma), READ (Rectum adenocarcinoma), SARC (Sarcoma), SKCM (Skin Cutaneous Melanoma), STAD (Stomach adenocarcinoma), TGCT (Testicular Germ Cell Tumors), THYM (Thymoma), THCA (Thyroid carcinoma), UCS (Uterine Carcinosarcoma), UCEC (Uterine Corpus Endometrial Carcinoma), and UVM (Uveal Melanoma).

A “tumour sample” refers to a sample that contains tumour cells or genetic material derived therefrom. The tumour sample may be a cell or tissue sample (e.g. a biopsy) obtained directly from a tumour. A tumour sample may be a sample that comprises tumour cell or genetic material derived therefrom, that has not be obtained directly from a tumour. For example, a tumour sample may be a sample comprising circulating tumour cells or circulating tumour DNA. Thus, a tumour sample may also be a biological fluid (e.g. a liquid biopsy such as a blood, urine, or cerebrospinal fluid biopsy). A sample comprising a mixture of tumour cells and other cells (or material genetic derived therefrom) may be subject to one or more processing steps, whether prior to or subsequent to the acquisition of sequence data, in order to identify sequence data that is representative of the genetic material from the tumour. For example, a sample comprising cells may be subject to one or more cell purification steps which selectively enrich the sample for tumour cells. As another example, a sample of genetic material may be subject to one or more capture and/or size selection steps to selectively enrich the sample for tumour-derived genetic material. Protocols for doing this are known in the art. As another example, sequence data may be subject to one or more filtering steps (e.g. based on fragment length) to enrich the data for information that relates to tumour-derived genetic material. Protocols for doing this are known in the art. In embodiments, the sample is a sample comprising tumour cells. Preferably, such a sample has a tumour purity (where tumour purity can be quantified as the proportion of cells in the sample that are tumour cells) of at least 30%, at least 35%, at least 40%, at least 45%, or at least 50%. Advantageously, the sample has a tumour purity of at least 40%. Without wishing to be bound by theory, it is believed that the copy number profiles generated from samples that have lower tumour purity may be less suitable for the purpose of the present invention as the signal corresponding to the tumour genome may be lost amongst the signal from the genomes of other cells.

A “normal sample” (also referred to as “germline sample”) refers to a sample that contains non-tumour or non-modified cells or genetic material derived therefrom. A normal sample may be matched to a particular tumour or modified sample in the sense that it is obtained from the same biological source (subject or cell line) as the tumour or modified sample. A normal sample may be a cell or tissue sample obtained from a subject, or a sample of biological fluid. A normal sample may be, e.g. a blood sample. A sample comprising a mixture of normal cells (or material genetic derived therefrom) may be subject to one or more processing steps, whether prior to or subsequent to the acquisition of sequence data, in order to identify sequence data that is representative of the genetic material from the normal cells (as already described above). For example, a sample comprising normal and tumour-derived cells (e.g. a blood sample comprising circulating tumour cells or a blood sample comprising cells froma haematological tumour) can be subject to one or more purification steps which selectively enrich the sample for normal cells. A normal sample may be used in the context of the present invention as a control to analyse a tumour sample. For example, a tumour sample and a (typically matched) normal sample may be analysed together in order to obtain a copy number profile for the tumour. Methods to obtain tumour copy number profile from a pair of normal and tumour samples are known in the art. For example, these include ASCAT [Van Loo et al. 2010] and Sequenza [Favero et al. 2015]. Such methods may provide a tumour copy number profile as well as an estimate of the purity (proportion of tumour cells) of the tumour sample. The purity estimate may be used to exclude tumour samples that have low purity and hence could lead to low quality copy number estimates.

The term “sequence data” refers to information that is indicative of the presence and/or amount of genomic material in a sample that has a particular sequence. Such information may be obtained using sequencing technologies, such as e.g. next generation sequencing (NGS, such as e.g. whole exome sequencing (WES), whole genome sequencing (WGS), or sequencing of captured genomic loci (targeted or panel sequencing)), or using array technologies, such as e.g. SNP arrays, or other molecular counting assays. When NGS technologies are used, the sequence data may comprise a count of the number of sequencing reads that have a particular sequence. When non-digital technologies are used such as array technology, the sequence data may comprise a signal (e.g. an intensity value) that is indicative of the number of sequences in the sample that have a particular sequence, for example by comparison to an appropriate control. Sequence data may be mapped to a reference sequence, for example a reference genome, using methods known in the art (such as e.g. Bowtie (Langmead et al., 2009)). Thus, counts of sequencing reads or equivalent non-digital signals may be associated with a particular genomic location. Further, a genomic location may contain a mutation, in which case counts of sequencing reads or equivalent non-digital signals may be associated with each of the possible variants (also referred to as “alleles”) at the particular genomic location. The process of identifying the presence of a mutation at a particular location in a sample is referred to as “variant calling”, and can be performed using methods known in the art (such as e.g. the GATK HaplotypeCaller, https://gatk.broadinstitute.org/hc/en-us/articles/360037225632-HaplotypeCaller). For example, sequence data may comprise a count of the number of reads (or an equivalent non-digital signal) which match a germline (also sometimes referred to as “reference”) allele at a particular genomic location, and a count of the number of reads (or an equivalent non-digital signal) which match a mutated (also sometimes referred to as “alternate”) allele at the genomic location.

A composition as described herein may be a pharmaceutical composition which additionally comprises a pharmaceutically acceptable carrier, diluent or excipient. The pharmaceutical composition may optionally comprise one or more further pharmaceutically active polypeptides and/or compounds. Such a formulation may, for example, be in a form suitable for intravenous infusion.

As used herein “treatment” refers to reducing, alleviating or eliminating one or more symptoms of the disease which is being treated, relative to the symptoms prior to treatment.

The systems and methods described herein may be implemented in a computer system, in addition to the structural components and user interactions described. “Computer-implemented method” where used herein is to be taken as meaning a method whose implementation involves the use of a computer, computer network or other programmable apparatus, wherein one or more features of the method are realised wholly or partly by means of a computer program. As used herein, the term “computer system” includes the hardware, software and data storage devices for embodying a system or carrying out a method according to the above described embodiments. For example, a computer system may comprise a processing unit (such as a central processing unit, CPU, and/or a graphics processing unit, GPU), input means, output means and data storage, which may be embodied as one or more connected computing devices. Preferably the computer system has a display or comprises a computing device that has a display to provide a visual output display. The data storage may comprise RAM, disk drives or other computer readable media. The computer system may include a plurality of computing devices connected by a network and able to communicate with each other over that network. It is explicitly envisaged that computer system may consist of or comprise a cloud computer. The methods described herein may be provided as computer programs or as computer program products or computer readable media carrying a computer program which is arranged, when run on a computer, to perform the method(s) described herein. As used herein, the term “computer readable media” includes, without limitation, any non-transitory medium or media which can be read and accessed directly by a computer or computer system. The media can include, but are not limited to, magnetic storage media such as floppy discs, hard disc storage media and magnetic tape; optical storage media such as optical discs or CD-ROMs; electrical storage media such as memory, including RAM, ROM and flash memory; and hybrids and combinations of the above such as magnetic/optical storage media.

The present invention relates broadly to the characterisation of DNA samples, particularly tumour samples, in terms of their copy number profiles.

A “copy number profile” refers to the quantification of the number of copies for each of a plurality of portions of a genomic sequence. In the context of the present disclosure, a copy number profile is preferably a genome-wide copy number profile. A copy number profile is typically obtained by sequencing a sample of genomic DNA (or a DNA library derived therefrom, as explained above, including a sample of DNA derived from genomic DNA by fragmentation, such as e.g. cell free DNA), and quantifying the number of copies per portion (e.g. per bin, where a bin can be e.g. a 30 kb region) of the genomic sequence, as known in the art. A “tumour copy number profile” refers to a copy number profile that is associated with a tumour genome. A tumour copy number profile may be obtained by sequencing a sample of genomic DNA (or a DNA library derived therefrom, as explained above, or a sample of DNA derived from genomic DNA by fragmentation) that is derived primarily from tumour cells or assumed to be derived primarily from tumour cells. As the skilled person understands, such samples can be contaminated with non-tumour DNA, which contamination can be minimised through processing of the sample or of the sequencing data, as known in the art. Preferably, a tumour copy number profile is a copy number profile that has been obtained by sequencing a sample of genomic DNA derived from tumour cells. In other words, a tumour copy number profile is preferably obtained using a sample comprising tumour cells.

A “segment” in a copy number profile refers to a portion of a sequence represented in a copy number profile which is associated with a consistent absolute copy number. The consistent copy number is different from that associated with the sequence directly upstream (if such a sequence is present and associated with a copy number estimate) and the sequence directly downstream (if such a sequence is present and associated with a copy number) of said portion. In other words, a segment refers to a portion of sequence that has a copy number associated with it, where the copy number associated with the segment differs from the copy number associated with its immediate neighbouring segment(s). The copy number associated with a segment may differ from the copy number associated with its immediate neighbouring segment(s) because the segment(s) that surround the segment are associated with a different copy number, because the segment(s) that surround the segment are not associated with a copy number (e.g. because data for the segment(s) is missing, or of insufficient quality), or a combination of both (e.g. a segment may be surrounded by a segment that is associated with a different copy number on one side, and a segment that is not associated with a copy number on the other side). In other words, segments refer to the longest continuous portion of a copy number profile that are each associated with a single copy number. A segment may be associated with a set of coordinates, e.g. genomic coordinates, which define the boundaries of the segment. Each boundary may be associated with a copy number changepoint (also referred to as “changepoint”). In embodiments, the copy number profiles comprise at most 350 segments (copy number events), at most 300 segments, or at most 250 segments. Preferably, the copy number profiles comprise at most 250 segments (copy number events). These numbers may be particularly useful when looking at human genome wide copy number profiles. Without wishing to be bound by theory, it is believed that higher numbers of segments may be indicative of unwanted DNA degradation (such as e.g. formalin-mediated DNA degradation). The copy number of a segment may in practice be a copy number estimate obtained using a method for determining tumour copy number profiles, such as e.g. ASCAT [Van loo et al., 2011] or Sequenza [Favero et al., 2015]. A “copy number event” refers to an instance in a copy number profile that is associated with one or more segments, and for which one or more copy number features can be quantified. For example, a segment may constitute an event and one or more features can be quantified for the segment such as its length, absolute copy number can be quantified, difference in absolute copy number with a neighbouring segment. A set of segments may constitute a copy number event and one or more features can be quantified for the set such as the number of breakpoints relative to the total length of the set of segments, and the number of contiguous segments oscillating between two copy number states.

The term “copy number (CN) features” refers to properties of copy number events observable in a copy number profile. Copy number features may include: segment size (also referred to as “segment length” typically expressed in number of bases), breakpoint count per×MB (the number of changepoints appearing in a sliding windows across the copy number profile, where the window is preferably 10 MB and the copy number profile is preferably genome-wide), change-point copy number (the absolute difference in copy number between a segment and an adjacent/neighbouring segment in the copy number profile, which may be defined relative to the upstream or downstream neighbouring segment), breakpoint count per chromosome arm (the number of changepoints occurring per chromosome arm), and number of segments with oscillating copy number (sometimes referred to as “length of segments with oscillating copy number”; number of continuous segments alternating between two copy number states, rounded to the nearest integer copy-number state; also referred to as length of chain of oscillating copy number states). The number features used according to the present invention do not include the segment copy number (the observed absolute copy number state of each segment, also referred to herein as “copy number” or “absolute copy number”). CN features can be observed on a genome-wide basis for a sample or collection of samples. In the context of the present disclosure, the term “genome-wide” refers to the assessment of a feature over a copy number profile that represents a substantial portion of a genome. For example, a substantial portion of a genome may comprise or consist of a chromosome, a plurality of chromosomes, or a portion of a genome determined by the parameters of the sequencing process used such as e.g. sequencing depth. Indeed, as the skilled person understands, even whole genome sequencing protocols may fail to accurately capture every sequence in a genome, especially at lower sequencing depths.

The genome wide characteristics of a copy number feature can be assessed by quantifying the copy number feature for each copy number event in a copy number profile, and obtaining one or more summarised measure for the copy number profile. One or more summarised measure may be obtained for each copy number feature. Alternatively, one or more summarised measure may be obtained which captures the contribution of multiple features. One such measure is exposure to a signature representing the genome-wide imprint of distinct putative mutational processes (where a “mutational process” as used herein refers to any process that can cause chromosomal instability), also referred to herein as “copy number signature” or simply “signature”. As described in Alexandrov et al. in relation to single base substitutions (Cell Rep. 2013 Jan. 31; 3 (1): 246-59. doi: 10.1016/j.celrep.2012.12.008.), exposure to a mutational signature represents the number of mutations attributed to that signature in a particular genome. The signature of a mutational process is the probabilities of a mutational process causing each of the possible mutation types in a mutation catalogue (where mutation types are defined in Alexandrov et al. as C:G>A:T, C:G>G:C, C:G>T:A, T:A>A:T, T:A>C:G, and T:A>G:C). In Macintyre et al. (Nat Genet. 2018 September; 50(9): 1262-1270), the present inventors extended the concept of mutational signatures to copy number events instead of single base substitutions. The mutation types in the mutational catalogue were defined as individual components of mixture models fitted to the distribution of values obtained for each of 6 copy number features (including the segment copy number) across a set genome-wide copy number profiles from a HGSOC cohort. Copy number signatures (i.e. signatures of copy number alteration processes) were obtained using these, which capture the probability of copy number alteration processes causing copy number events that are distributed according to each of the copy number feature components. Thus, the term “exposure” (also referred to as “signature activity” or “activity”) in this context captures the strength of evidence for the presence of copy number alteration events attributable to the signature. In the present work, the inventors built on this work and identified that a compact set of features that did not include a feature representing the copy number of a segment advantageously avoided redundancy if signatures for the same aetiology appear across different ploidy background. This therefore resulted in more robust, and more biologically relevant signatures identified using these copy number features. Further, the inventors identified and demonstrated that this approach could be used to identify signatures that are recurrent across all cancer types investigated, thus providing a robust pan-caner framework for the characterisation of chromosomal instability in cancer.

Methods for determining the exposure to a signature are known in the art (see e.g. Alexandrov et al., 2020; Macintyre et al., 2018). In particular, the determination of the exposure to one or more mutational signatures may be performed by identifying the matrix E that satisfies C≈PE where C is a mutational catalogue for one or more samples for which exposure is to be determined (also referred to as patient-by-component, PbC matrix), P is a signature matrix comprising the one or more mutational signatures for which exposure is to be determined (also referred to as signature-by-component, SbC matrix), and E is an exposure matrix (also referred to as patient-by-signature, PbS). For example, exposure to a signature performing matrix decomposition to identify the value (or vector of values) that satisfies the equation: PbC=PbS×SbC (or, in practice PbC=PbS×SbC+ε, where ε is a residual term to be minimised), where SbC is the row of the signature-by-component matrix that corresponds to a particular signature of a set of signatures, and PbS is the patient-by-signature value (or vector of values). Exposure to multiple copy number signatures can be calculated using the corresponding rows of the signature-by-component matrix. In embodiments, exposure (E) to a copy number signature i as described herein (from a set of n signatures, where n can be e.g. 17, where the signatures can include any or all of the signatures disclosed herein or corresponding signatures) is the value Eᵢ that satisfies the equation:

$$PbC \approx E \times SbC \qquad ( Equation 1 )$$

where: E is a vector of size n comprising coefficients E_{1, . . . , n} where Eᵢ is the exposure to signature i; PbC is a vector of size c, each value representing the sum-of-posterior probabilities of each copy number event in the copy number profile belonging to a component C, where each component C is a distribution of values for a copy number feature; and SbC is a matrix of size c by n, each value representing the weight of a component C in a copy number signature i as described herein.

### Identification of Copy Number Signatures Present in a Sample

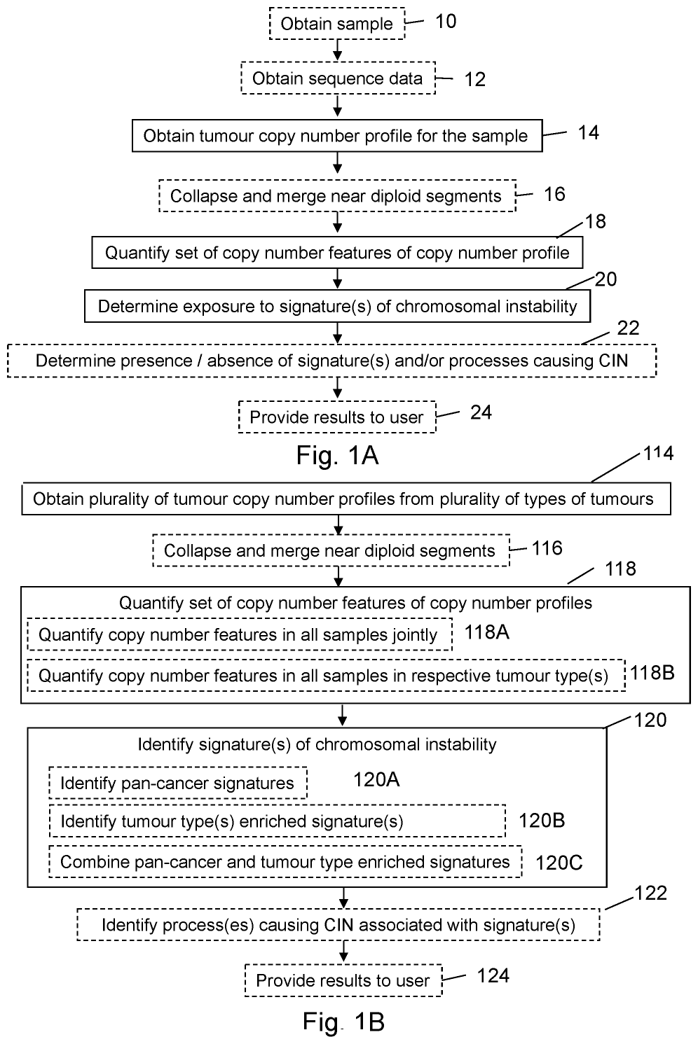

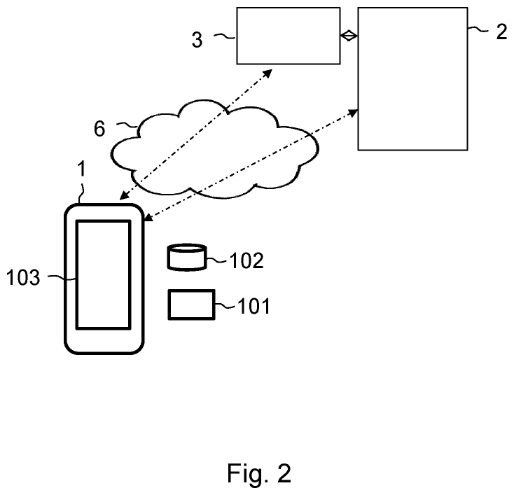

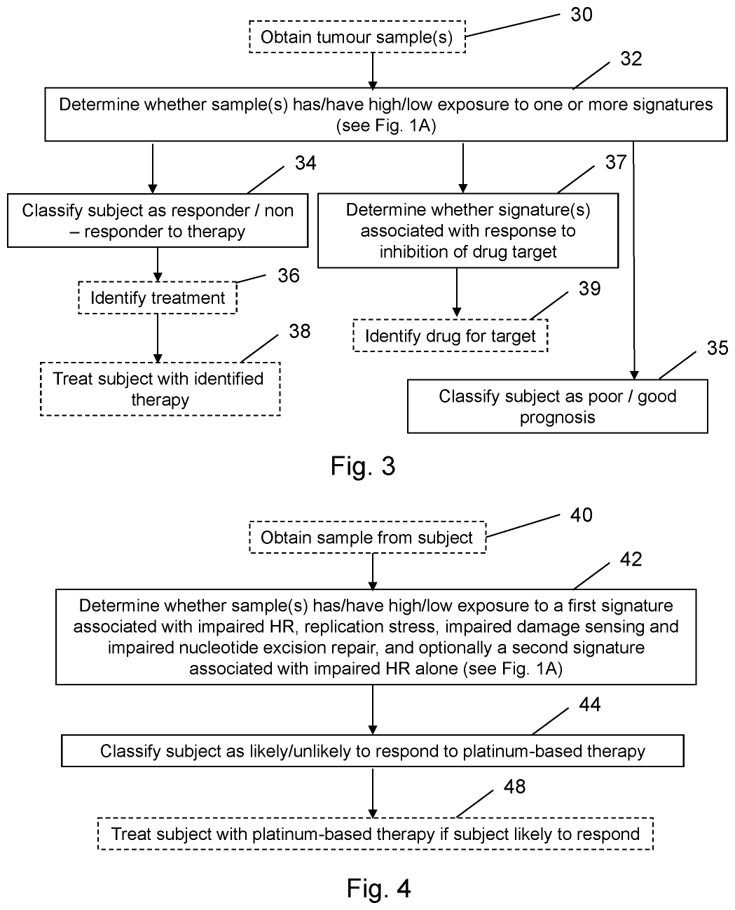

FIG. 1A is a flow diagram showing, in schematic form, a method of characterising a DNA sample according to the disclosure. At optional step 10, a DNA sample is obtained from a tumour of a subject. Optionally, a matched normal sample may also be obtained from the subject. At optional step 12, sequence data is obtained from the tumour (and optionally the matched normal) DNA sample(s). At step 14, a tumour copy number profile for the sample is obtained, for example by analysing the sequence data obtained at step 12 or by receiving the copy number profile from a user, database etc. At optional step 16, near diploid segments in the copy number profile may be collapsed and merged. At step 18, a predetermined set of copy number features is quantified across the copy number profile. The predetermined set of copy number features depends on the signatures for which exposure is calculated. According to the present disclosure, this does not include absolute copy number, but may include one or more (or all) of: segment size, copy number change-point, breakpoint count per predetermined length of sequence and/or per chromosome arm, and number of segments with oscillating copy number, optionally wherein the set of copy number features includes all of the following features: segment size, breakpoint count per predetermined length of sequence, copy number change-point, breakpoint count per chromosome arm, and number of segments with oscillating copy number. At step 20, exposure to one or more predetermined signatures of chromosomal instability (CIN) is determined, wherein the signature have been obtained by quantifying the same features for a plurality of tumour copy number profiles and identifying the combination of signatures that best explain the patterns of copy number features in these tumour copy number profiles (e.g. using NMF, non-negative matrix factorisation), for example as described in relation to FIG. 1B. At step 22, the presence or absence of the one or more signatures may optionally be determined, for example using a signature specific threshold on exposure to call a signature present or absent. Further, the presence or absence of one or more processes causing CIN may be determined using the exposures obtained at step 20, for example by determining the presence of a process causing CIN if a signature associated with said process is present in the sample. At step 24, one or more results of this analysis may optionally be provided to a user through a user interface.

FIG. 1B is a flow diagram showing, in schematic form, a method of characterising types of chromosomal instability in a plurality of samples according to the present disclosure. The method may be used to identify signatures of chromosomal instability, for example for use in a method as described in relation to FIG. 1A. A step 114, a plurality of tumour copy number profiles are obtained. These may be obtained from a database, user, etc., or may be obtained by analysing sequence data for a plurality of tumour samples obtained from a user, database etc. The plurality of tumour copy number profiles preferably originate from a plurality of types of tumours. At optional step 116, near diploid segments in the copy number profile may be collapsed and merged. At step 118, a predetermined set of copy number features is quantified across the copy number profile. According to the present disclosure, this does not include absolute copy number, but may include one or more (or all) of: segment size, copy number change-point, breakpoint count per predetermined length of sequence and/or per chromosome arm, and number of segments with oscillating copy number, optionally wherein the set of copy number features includes all of the following features: segment size, breakpoint count per predetermined length of sequence, copy number change-point, breakpoint count per chromosome arm, and number of segments with oscillating copy number. The quantification of copy number features may be performed separately for all tumour copy number profiles at step 118A, and for each of a respective plurality of sets of copy number profiles from respective tumour types at step 118B. The step of quantifying a set of copy number features may comprise identifying a plurality of distributions of said copy number features across the plurality of samples that are jointly analysed, for example using mixture modelling. At step 120, one or more signatures of chromosomal instability are identified. This may comprise step 120A of identifying pan-cancer signatures using the information from step 118A, and step 120B of identifying tumour type enriched signatures using the information from step 118B. This may further comprise combining the signatures from steps 120A and 120B. For example, signatures from step 120B that have a similarity to any signature in the set from step 120A above a predetermined threshold may be removed. Instead or in addition to this, signatures from step 120B that have a similarity to any signature in another of the one or more sets of tumour type enriched signatures above a predetermined threshold may be removed. Instead or in addition to this, signatures in the one or more sets of step 120B that can be obtained by a linear combination of signatures in the set from step 120A may be removed. At optional step 122, one or more processes causing CIN that are associated with the signatures identified in step 120 may be identified. At step 124, one or more results of this analysis may optionally be provided to a user through a user interface.

### Systems

FIG. 2 shows an embodiment of a system for characterising a DNA sample and/or for providing a prognosis or treatment recommendation, according to the present disclosure. The system comprises a computing device 1, which comprises a processor 101 and computer readable memory 102. In the embodiment shown, the computing device 1 also comprises a user interface 103, which is illustrated as a screen but may include any other means of conveying information to a user such as e.g. through audible or visual signals. The computing device 1 is communicably connected, such as e.g. through a network, to sequence data acquisition means 3, such as a sequencing machine, and/or to one or more databases 2 storing sequence data. The one or more databases 2 may further store one or more of: mutational signatures information, training data, parameters (such as e.g. parameters of a machine learning model used to predict whether a tumour is HR-deficient, e.g. weights of a logistic regression model, architecture and parameters of a decision tree model, etc.), clinical and/or sample related information, etc. The computing device may be a smartphone, tablet, personal computer or other computing device. The computing device is configured to implement a method for characterising a DNA sample, as described herein. In alternative embodiments, the computing device 1 is configured to communicate with a remote computing device (not shown), which is itself configured to implement a method of characterising a DNA sample, as described herein. In such cases, the remote computing device may also be configured to send the result of the method of characterising a DNA sample to the computing device. Communication between the computing device 1 and the remote computing device may be through a wired or wireless connection, and may occur over a local or public network 6 such as e.g. over the public internet. The sequence data acquisition means may be in wired connection with the computing device 1, or may be able to communicate through a wireless connection, such as e.g. through WiFi and/or over the public internet, as illustrated. The connection between the computing device 1 and the sequence data acquisition means 3 may be direct or indirect (such as e.g. through a remote computer). The sequence data acquisition means 3 are configured to acquire sequence data from nucleic acid samples, for example genomic DNA samples extracted from cells and/or tissue samples. In some embodiments, the sample may have been subject to one or more preprocessing steps such as DNA purification, fragmentation, labelling, library preparation, target sequence capture (such as e.g. exon capture and/or panel sequence capture). Preferably, the sample has not been subject to amplification, or when it has been subject to amplification this was done in the presence of amplification bias controlling means such as e.g. using unique molecular identifiers. Any sample preparation process that is suitable for use in the determination of a copy number alteration profile may be used within the context of the present invention. The sequence data acquisition means may be a next generation sequencer or an array reader.

### Uses of Copy Number Signatures

The characterisation of a DNA sample from a tumour in terms of signatures of chromosomal instability active in the sample can be used to identify a treatment for a subject with cancer, and to identify a drug target for the treatment of cancer. Thus, the invention also provides a method of treating cancer in a subject, wherein the method comprises administering or recommending a subject for administration of a particular therapy, depending on the signature(s) of chromosomal instability that have been found to be active in the sample. FIG. 3 illustrates a method of providing a prognosis, identifying a drug target, identifying a therapy or treating a subject according to an embodiment of the present disclosure. The method may comprise optional step 30 of obtaining a DNA sample from a tumour of a subject, or from a plurality of tumour samples. Optionally, a matched normal sample may also be obtained from the subject. This may be used to provide a tumour copy number profile, as known in the art (see e.g Van Loo et al., 2010). The step of obtaining a sample from a subject may comprise physically obtaining the sample from the subject. Alternatively, the sample may have been previously obtained and no interaction with the subject may be required. In other words, obtaining a DNA sample may comprise receiving a previously acquired DNA sample.

Optionally, sequence data may be obtained from the tumour (and optionally the matched normal) DNA sample(s). The step of obtaining sequence data from a DNA sample may comprise sequencing the DNA sample or analysing the sample using a genomic array.

Alternatively, sequence data may have been previously obtained. Thus, obtaining sequence data may comprise receiving the data from one or more databases, or from a user through a user interface. At step 32, the sample(s) is/are characterised using methods described herein such as e.g. by reference to FIG. 1A. Based on this determination, a subject may be classified at step 34 as being likely to respond or unlikely to respond to a particular course of treatment, where responder/non-responder status is known to be associated with exposure to one or more signatures. The particular course of treatment may target a particular gene, A signature of chromosomal instability may be known to be associated with response to inhibition of a particular gene if the exposure to the signature is significantly correlated with the effect (e.g. cellular proliferation, growth inhibition, toxicity, etc) of perturbation of the gene, for example by genetic perturbation (such as in a CRISPR essentiality screen, also referred to as CRISPR knockout screen, or in an RNAi essentiality screen) or by drug perturbation of the gene (such as in a drug response screen). At optional step 36, a particular course of treatment (which may comprise one or more different individual therapies) may be identified based on the results of step 34. For example, a subject that has been identified at step 34 as unlikely to respond to the particular course of therapy may be identified as likely to benefit from a therapy that is different from the particular course of therapy. Alternatively, a subject that has been identified at step 34 as likely to respond to the particular course of therapy may be identified as likely to benefit from a therapy that includes the particular course of therapy.

At optional step 38, the subject may be treated with the therapy identified at step 40. Alternatively, the methods described herein may be used to provide a prognosis for a subject. Thus, based on the determination made at step 32, a subject may be classified at step 35 as having a good or a poor prognosis, where prognosis is known to be associated with exposure to one or more signatures. For example, a signature may be known to be associated with prognosis if samples with a high or low exposure to the one or more signatures are associated with different prognosis in a cohort of patients. In other words, a signature may be known to be associated with prognosis if samples with a good prognosis in a cohort of patients have a significantly different expected exposure to the signature than samples with a poor prognosis.

The prognosis may be specific to a particular tumour type, such as e.g. ovarian cancer. Alternatively, the methods described herein may be used to identify a drug target for the treatment of cancer. This may comprise obtaining tumour copy number profiles for a plurality of samples and characterising them at step 32 using the methods described herein. The plurality of DNA samples in this case comprise samples obtained from a tumour or a tumour cell line in which the drug target has been the subject of inhibition and for which response to inhibition of the drug target has been quantified. This may further comprise at step 37 determining whether one or more signatures of chromosomal instability are associated with response to inhibition of the drug target, wherein the presence of a signature of chromosomal instability associated with response to inhibition of the drug target is indicative of the drug target being usable for the treatment of a cancer in which the signature is active. This may further comprise optional step 39 of identifying a drug that targets the drug target, for example through a drug database or using any drug design method known in the art.

Any treatment described herein may be used alone or in combination with another treatment. For example, any treatment with a drug may be used in combination with one or more chemotherapies, one or more course of radiation therapy, and/or one or more surgical interventions. In particular, any treatment described herein may be used in combination with a treatment for which the subject has been identified as likely to be responsive.

For example, signatures of chromosomal instability described herein have been shown to be predictive of whether a patient with cancer is likely to respond to platinum-based therapy. FIG. 4 is a flow diagram illustrating schematically a method of identifying whether a patient is likely to respond to platinum-based therapy according to an embodiment of the present disclosure. The method may comprise optional step 40 of obtaining a DNA sample from a tumour of a subject. Optionally, a matched normal sample may also be obtained from the subject. The step of obtaining a sample from a subject may comprise physically obtaining the sample from the subject. Alternatively, the sample may have been previously obtained and no interaction with the subject may be required. In other words, obtaining a sample may comprise receiving a previously acquired DNA sample. Optionally, sequence data may be obtained from the tumour (and optionally the matched normal) DNA sample(s). The step of obtaining sequence data from a DNA sample may comprise sequencing the DNA sample or analysing the sample using a genomic array. Alternatively, sequence data may have been previously obtained. Thus, obtaining sequence data may comprise receiving the data from one or more databases, or from a user through a user interface. At step 42, the sample is characterised as having a high or low exposure to a signature of chromosomal instability that is associated with impaired homologous recombination (HR), replication stress, impaired damage sensing and impaired nucleotide excision repair. Optionally, the sample may also be characterised as having a high or low exposure to a signature of chromosomal instability that is associated with impaired homologous recombination (HR) alone. This may be performed using methods described herein such as e.g. by reference to FIG. 1A to determine exposure to the first and optionally second signature. Based on this determination, a subject may be classified at step 44 as being likely to respond or unlikely to respond to platinum-based therapy. In particular, if the sample is characterised as having a high exposure to the first signature, the subject may be classified as likely to respond to platinum-based therapy. A sample may be considered to have a high exposure to the first signature if exposure to the first signature is higher than exposure to the second signature. Optionally, exposure to a third signature associated with impaired homologous recombination plus replication stress but not impaired damage sensing and impaired nucleotide excision repair may also be quantified at step 42. In such embodiments, step 44 may comprise classifying the sample between at least a first class and a second class using a classifier that has been trained to classify samples between at least a first class and a second class based on the exposure to the first, second and third signatures, wherein samples in the first class are likely to respond to platinum-based therapy and samples in the second class are unlikely to respond to platinum-based therapy. At optional step 48, the subject may be treated or selected for treatment with platinum-based therapy if the subject was classified as likely to respond to platinum-based therapy. Alternatively, the subject may be treated or selected for treatment with an alternative therapy if the subject was classified as unlikely to respond to platinum-based therapy.

Additionally, the presence of some processes causing chromosomal instability in a tumour have been shown to be associated with different prognosis in cancer. Thus, also described herein are methods of providing a prognosis for a subject that has been diagnosed as having a cancer, the method comprising determining the activity of one or more signatures as described herein in a tumour from the subject. The method may further comprise classifying the subject between a group that has good prognosis, and a group that has poor prognosis.

For example, the method may comprise determining whether a sample from a tumour of the subject has a high or low exposure to one or more signatures that have been identified to be associated with prognosis (as explained above). A subject may then be classified in the group that has poor prognosis if the sample is determined to have a high exposure to the signature(s), and in a group that has good prognosis otherwise. Alternatively, a subject may be classified in the group that has poor prognosis if the exposure(s) to the signature or a score derived therefrom is above a threshold, and in the group that has good prognosis otherwise.

Whether a prognosis is considered good or poor may vary between cancers and stage of disease. In general terms a good prognosis is one where the overall survival (OS), disease free survival (DFS) and/or progression-free survival (PFS) is longer than that of a comparative group or value, such as e.g. the average for that stage and cancer type. A prognosis may be considered poor if OS, DFS and/or PFS is lower than that of a comparative group or value, such as e.g. the average for that stage and type of cancer. Thus, in general terms, a “good prognosis” is one where survival (OS, DFS and/or PFS) and/or disease stage of an individual patient can be favourably compared to what is expected in a population of patients within a comparable disease setting. Similarly, a “poor prognosis” is one where survival (OS, DFS and/or PFS) of an individual patient is lower (or disease stage worse) than what is expected in a population of patients within a comparable disease setting.

The subject is preferably a human patient.

The following is presented by way of example and is not to be construed as a limitation to the scope of the claims.

### EXAMPLES

### Example 1—Robust Identification of Pan-Cancer Copy Number Signatures

In this example, the inventors present a robust analysis framework for chromosomal instability in human cancers. The approach substantially extends previous work in ovarian cancer (Macintyre et al., 2018) and related work in sarcoma (Steele et al., 2019) using a pan-cancer analysis of 7,880 high-quality samples across the 33 cancer types in the TCGA collection. A compendium of 17 copy number signatures characterises different types of CIN and their aetiologies are supported by a wide array of independent data sources, as demonstrated in Example 2. In Example 3, the inventors apply the signatures to predict drug response and to identify new drug targets. In Example 4, the inventors show how the new framework refines the understanding of impaired homologous recombination, one of the most clinically relevant types of CIN.

### Methods

Data sources. Table 1 lists the data used throughout these examples and their respective source. All data are publicly accessible, except for the TCGA, PCAWG and ICGC raw data (SNp 6.0 arrays, WGS, WES). Access to the TCGA data (SNP 6.0 arrays, WES) and the TCGA-part of the PCAWG data (WGS) can be obtained by a Data Access Request through the Database of Genotypes and Phenotypes (dbGaP, http://www.cancergenomicscloud.org/controlled-access-data). For the ICGC data (SNP 6.0) and non-TCGA PCAWG data (WGS), an application to the ICGC DACO (https://daco.icgc.org/) has to be made. The COSMIC database can be accessed with a free account without an application (https://cancer.sanger.ac.uk/cosmic).

TABLE 1
Sources of publicly available data.

| Data | Source |
|---|---|
| TCGA SNP6 Affymetrix Arrays (CNA) | Published here1 but accessed through |
| | TCGA Research Network: |
| | https://www.cancer.gov/tcga |
| TCGA Gene expression | Published here1 but accessed through |
| | COSMIC2: |
| | https://cancer.sanger.ac.uk/cosmic/ |
| PCAWG Copy number calls | Gerstung et al., 2020, Denstro et al. 2020 |
| DepMap CRISPR screen | Meyers et al., 2017 |
| Cell Lines from the CCLE project | Ghandi et al., 2019 |
| PRISM drug repurposing screen | Corsello et al., 2019 |
| DEMETER2 RNAi screen | McFarland et al., 2018 |
| Germline and Somatic BRCA1/2 status for | Wang et al., 2017, Maxwell et al., 2017 |
| TCGA-OV and TCGA-BRCA patients | |
| Promoter hypermethylation of RD51C and | Cancer Genome Atlas Research Network, |
| BRCA1 in TCGA-OV samples | 2011, Sample IDs obtained from |
| | corresponding author |
| TCGA clinical data (incl. survival data) | TCGA Research Network: |
| | https://www.cancer.gov/tcga |
| PCAWG clinical data (incl. survival data) | Published in ICGC/TCGA Pan-Cancer |
| | Analysis of Whole Genomes Consortium, |
| | 2020 but hosted on synapse.org ID |
| | syn10389158 |
| ICGC 560 breast cancer samples plus | Nik-Zainal et al., 2020 |
| clinical data | |
| extrachromosomal DNA for TCGA | Kim et al., 2020 |
| Tandem duplication classification for TCGA | Menghi et al., 2018 |
| HRDetect on 560 breast cancers | 1Davieset al., 2017 |
| HRDetect on PCAWG | Degasperi et al., 2020 |
| HRD score, telomeric imbalance score, | Knijnenburg et al., 2018, Marquard et al., |
| large-scale state transitions score, loss of | 2015 |
| heterozygosity score | |
| Cell cycle score | Lundberg et al., 2020 |
| Structural variants, including | Li et al., 2020 |
| microhomologies | |
| Somatic exon SNCs in TCGA | Ellrott et al., 2018 |
| Exposures to SBS, DBS and ID signatures | Explained on COSMIC website (Tate et al., |
| | 2019) but hosted |
| | on synapse.org ID syn12009743 |
| Whole genome duplication status | Dentro et al., 2020 |
| Chromotrhipsis and Kataegis | ICGC/TCGA Pan-Cancer Analysis of Whole |
| | Genomes Consortium, 2020 |
| Telomere length, TSS, TERT/TERC, | Barthel et al., 2017 |
| TERRA, Alternative Telomere Lengthening | |
| (ATRX/DAXX) | |
| Driver gene classification | Bailey et al., 2018, Jassal et al, 2020 |
| Bayes NMF (used for signature derivation) | Kim et al., 2016, Tan & Fevotte, 2013 |
| Other detectable CIN measures | Weaver & Celveland, 2006, Taylor et al., |
| | 2018, Davoli et al., 2017 |
| SNP loci from the 1,000 Genome Project | Published in Frankish et al., 2019 but |
| (phase 3) falling into exonic regions | downloaded from |
| (GENCODE v19 exome) | GDC: |
| | https://api.gdc.cancer.gov/data/7f0d3ab9- |
| | 8b |
| | ef-4e3b-928a-6090caae885b |

Inferring absolute copy numbers. Sample-specific CEL files from TCGA were processed with Affymetrix Power Tools (APT; v2.11.2; standard options) and ASCAT (Van Loo et al., 2010, R package, v2.4). By default ASCAT produces an absolute, genome-wide copy number segmentation where segments are rounded to the nearest integer copy number state and merged. However, the copy number signature framework described herein used unrounded copy number segments. Therefore, the inventors determined an optimal segmentation penalty to avoid over- or under-segmentation. They compared ASCAT generated breakpoints with multiple segmentation penalties (25, 35, 50, 70, 100, 140, 200) to the PCAWG consensus copy number calls for 714 samples from 22 cancer types which were generated using whole-genome sequencing. They found that a penalty of 70 offered the highest degree of overlap. They ran ASCAT on 12,240 samples of which 12,141 produced usable solutions. To remove any noise around diploid copy states, all segments around 2 (1.9<x<2.1) were collapsed to exactly 2 and adjacent segments were merged. This merging was also performed for deleted segments (copy number equals 0).

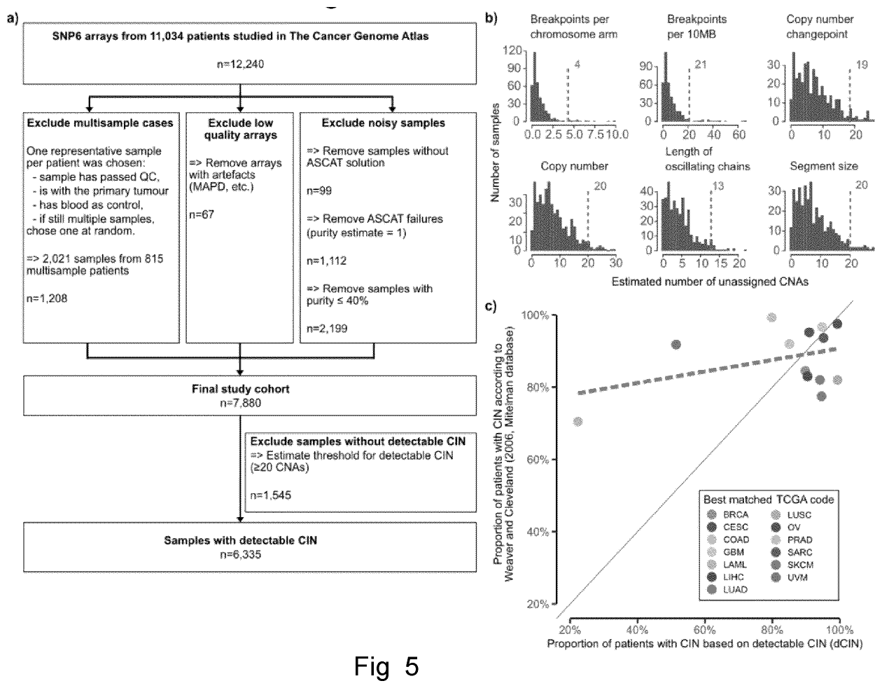

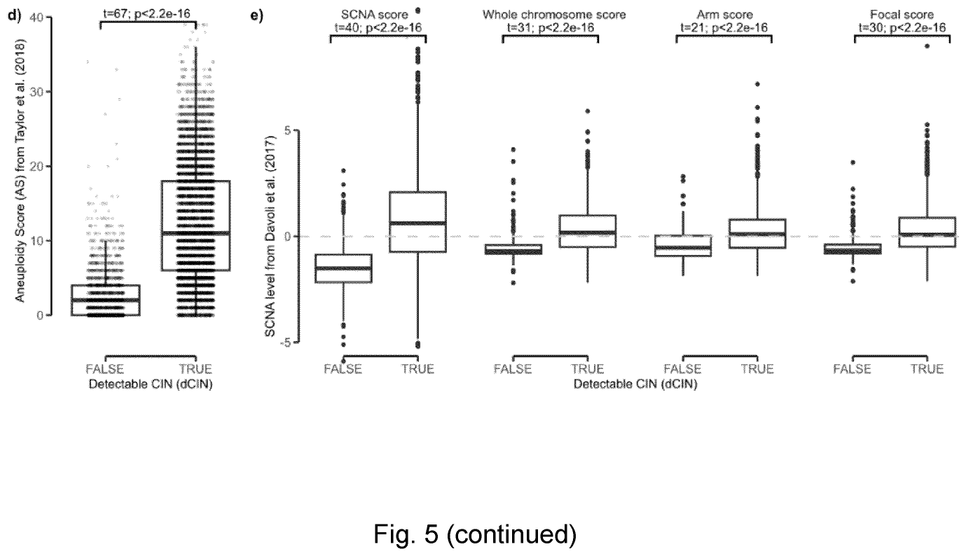

Identifying high-quality copy number profiles. For 815 multisample patients with 2,021 samples, the inventors identified a representative sample based on the following criteria: sample passed quality control; from the primary tumour; had blood as normal control; if there were still multiple options at that point, one was chosen at random. This reduced the number of available copy number profiles to 11,032. The following four quality controls were applied to the absolute copy number estimates: 1) purity>0.4 and purity<1: samples with a purity lower than 40% were removed to avoid low quality absolute copy number estimates. A purity equal to 1 generally indicates that ASCAT has identified a copy number profile from a normal tissue and therefore only samples with purity<1 were considered. 2) tumour or normal MAPD<0.75: The median of absolute pairwise differences between adjacent probes (MAPD) is a per-microarray estimate of the variance, similar to the standard deviation or the interquartile range 33. Filtering for the MAPD removed noisy samples. 3) tumour and normal MAPD<0.4 and fraction homozygous segments <0.1: Occasionally tumour samples are matched with the wrong germline sample. This results in a high fraction of homozygous segments. The above thresholds removed these samples. 4) differences between log R and BAF segments <250: We identified and removed 67 samples from 17 cancer types with extreme noise in log R values, which manifested as a large-scale wave pattern which could not be captured by the median of absolute pairwise differences between adjacent probes (MAPD). The corresponding B-allele frequencies (BAF) values did not show this wave pattern and generally appeared consistent. These samples were identified by having more than 250 segments difference between their log R and BAF segmentations. All filters combined resulted in 7,880 high-quality copy number profiles across 33 cancer types (FIG. 5a).

Computing detectable chromosomal instability (dCIN). For signature identification, we aimed to use only samples with sufficient evidence of chromosomal instability (CIN). To that end, we identified and established a pragmatic threshold for the detectability of CIN (dCIN) from copy number profiles. We called ovarian copy number signatures on 539 high-grade serous ovarian cancer samples to estimate the number of undetected CNAs in order to establish an empirical threshold for detectable CIN. The first step of calling ovarian copy number signatures is to extract six fundamental features from all copy number segments: segment size, absolute copy number, copy number change to the left neighbouring segment, breakpoints per 10 megabases (Mb), breakpoints per chromosome arm, and length of oscillating chains. Based on previously established mixture models (Macintyre et al., 2018) for each of these six feature distributions, posterior probabilities for the extracted feature values were calculated. The output of this process is a matrix with 539 samples in its rows and the 36 mixture components as columns. The entries are the sum-of-posteriors of the previously determined posterior probabilities. Using YAPSA (Huenschmann et al., 2019, R package, v1.12.0) and the signature definition matrix from Macintyre et al., we calculated signature activities for each of the 539 samples. Not all the signal from the input matrix can be assigned to the different signatures. Using 5% as a threshold for assessing the activity of a signature in a sample, we set signature activities above 5% to 0 and multiplied the activity matrix by the signature definition matrix to derive a matrix with undetected signal. For each sample and feature, we summed the remaining probabilities to estimate the number of undetected CNAs. For each feature, we defined the quantile 0.95 of the distribution of undetected CNAs. From this distribution of quantiles 0.95, we took the 3rd quartile (which is less volatile and more robust than the maximum) as the empirical threshold of the detectability of CIN which is 20 CNAs (FIG. 5b).

Comparison to previous dCIN measures. Previous studies to quantify the proportion of cancer samples with CIN used aneuploidy as a surrogate measure with pan-cancer estimates ranging between 60% and 95% depending on the method (karyograms see Cimini et al., 2008; genomical case studies-sLens & Medema, 2019, Mitelman er al.; computational methods-Carter et al., 2012; FISH-Cisyk et al., 2015; reviewed Ben-David & Amon, 2020). Multiple studies found that around 90% of solid tumours are aneuploid (Weaver & Cleveland, 2006; Taylor et al., 2018, Cimini et al., 2008, Lens & Medema, 2019, Ben-David & Amon, 2020, Bakhoum et al. 2018, Duijf et al., 2013). This number goes back to a snapshot of the Mitelman database of chromosome aberrations and gene fusions, which analysed 17 solid and 9 hematopoietic cancers of which 13 could be matched to organs included in the TCGA project (FIG. 5c). Apart from LAML (acute myeloid leukemia) and UVM (uveal melanoma), all cancer types agreed with minor discrepancies of up to 20%. The larger discrepancies for UVM can be explained as it was mapped onto retinoblastoma, two fairly different diseases (Harbour et al, 2006). For LAML, the majority of samples had a near diploid number of chromosomes (<68). Given that LAML samples have quiet copy number landscapes (Zack et al., 2013), it is most likely the case that the dCIN threshold with 20 CNAs lies just above the average changes in LAML. We compared our dCIN measure to two other measures previously published in the literature: the aneuploidy score from Taylor et al. and SCNA levels from Davoli et al0. Taylor and colleagues analysed 10,522 SNP arrays from the TCGA and found 88% aneuploid samples. Davoli et al. did not summarise a single percentage of how many cancers have aneuploidy, instead they supplied a single somatic CNA (SCNA) score plus three more specific scores for whole chromosomes, arms and focal changes. We divided the samples into samples with detectable CIN and undetectable CIN and compared the measures from Davoli and Taylor. All comparisons were highly significant. As before, the dCIN threshold missed some samples with mostly quiet profiles largely containing whole chromosome CNAs and arm level changes. It is unclear whether such quiet genomes were produced by a mutational process or simply originated from random mitotic errors and unrepaired double-strand breaks.

Deriving pan-cancer chromosomal instability signatures-Choice of features. Research into genomic and chromosomal instability revealed a long list of known mutational processes resulting in unique copy number patterns. Table 2 lists well-studied copy number patterns and describes which specific copy number feature is able to capture their unique characteristics. These observations motivate our selection of five fundamental features to model known patterns and potentially distinguish their mutational processes.

TABLE 2
Copy number features capture known copy number patterns.

| | Seg. | | | | |
|---|---|---|---|---|---|
| Pattern | size | CNC | BP10 | BPARM | OSC |
| Amplification | x | x | X | | |
| Aneuploidy | x | X | | | |
| Breakage-fusion-bridge | | x | | X | |
| Chromothripsis | | x | x | x | X |
| Complex genomic | x | x | x | x | x |
| rearrangement | | | | | |
| Deletions | | X | | | |
| Extrachromosomal DNA | x | X | | | |
| (ecDNA) | | | | | |
| Homologous recombination | x | x | x | x | x |
| deficiency | | | | | |
| Loss of heterozygosity | x | x | | X | |
| Micronuclei | x | X | | | |
| Tandem duplications | x | x | | x | X |
| Whole genome duplication | | x | | | |
| (WGD) | | | | | |

Seg. size = Segment size, CNC = Copy number changepoint, BP10 = Breakpoints per 10 Mb, BPARM = Breakpoints per chromosome arm, OSC = Length of oscillating copy number states.

Deriving pan-cancer chromosomal instability signatures-breakpoint density. We included two features capturing potential clusters of CNAs: breakpoints per 10 Mb and breakpoints per chromosome arm. These two features are needed to accurately model events of different sizes: one for small and medium sized clusters with the largest possible window size and one feature for larger clusters. In more detail: Clusters of CNAs can come in various sizes, e.g. short tandem duplications with less than 10 kb in length each, long tandem duplications with lengths of over 100 kb each, and up to large-scale transitions and chromothriptic events which can span multiple dozens of mega bases. Their respective cluster sizes can be in the tens of kb up to whole chromosome arms. However, the smallest chromosome arm on a SNP 6.0 array is 12.8 Mb in length. Having larger window sizes than 10 Mb would therefore lead to a skewed breakpoint density on smaller chromosome arms. A smaller window size might be unfeasible to capture medium sized clusters, which would require introducing a third feature capturing clusters of CNAs.

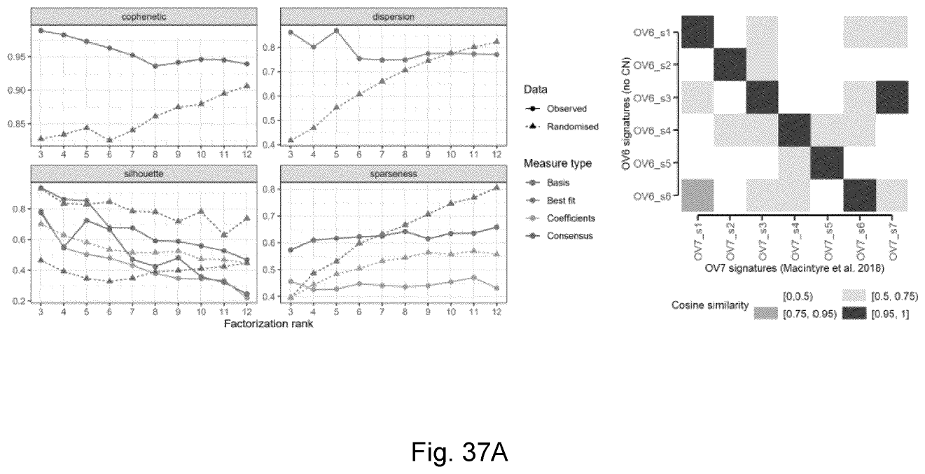

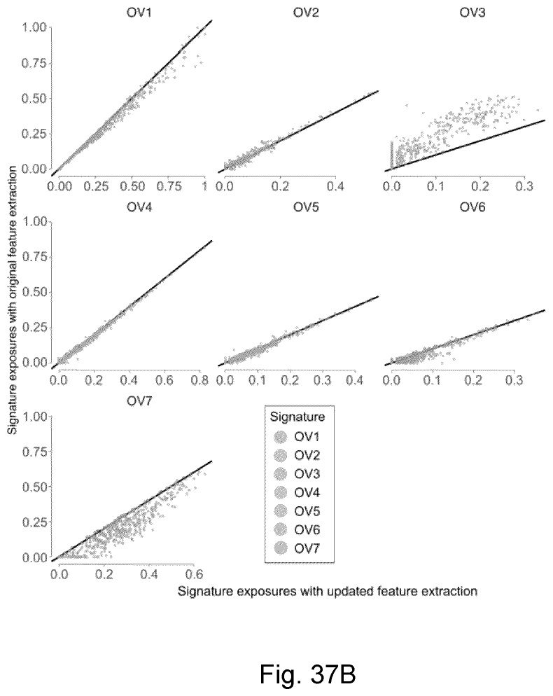

Deriving pan-cancer chromosomal instability signatures-exclusion of absolute copy number as a feature. In a major deviation from previous work in Macintyre et al., we did not use the absolute copy number of a segment as a feature, because we found that it can artificially split a signature by ploidy status despite representing the same mutational process. This effect is very clear in the original ovarian copy number signatures, where two (signatures 3 and 7) defined the same process for different ploidy levels. When we rederived the ovarian signatures without the copy number feature we found six signatures (rather than the published seven), with signatures 3 and 7 collapsed to one (FIG. 37A). The data on FIG. 37A shows that the optimal number of ovarian cancer signatures without the copy number feature is 6, one less than published in Macintyre et al., according to 4 different criteria (dispersion, silhouette width, sparseness and cophonetic correlation-which were used in Macintyre et al to select an optimal signature number). The sparseness criterion is believed to be on the most informative criterion—in FIG. 73A, lower right panel, the number of signatures before the two upper lines cross is a strong indicator for the optimal number of signatures (which is 6 in this case). In more detail: The absolute copy number of a CNA can be the result of a complex process. As multiple copy number changes can happen at the same genomic location, the resulting absolute copy number of that segment can vary wildly depending on the order of events. For example, a single copy loss on a whole-genome duplicated background results in a copy number state of 3 but a deletion in a non-WGD background would result in a copy number state of 1 and therefore be encoded by two different signatures, even though they might be caused by the same mutational process that results in deletion patterns. This effect is present in the ovarian copy number signatures in Macintyre et al., where signature 3 (BRCA1/2-related homologous recombination deficiency) acts on a diploid genome and signature 7 (non-BRCA 1/2-related homologous recombination deficiency) on a tetraploid genome. The rest of the signature definitions are very similar. However, a few studies looked at BRCA 1/2-related homologous recombination deficient cancer samples and tetraploid genome status. Jonsdottir et al. found that BRCA2-linked homologous recombination deficient tumours were three times more likely to have a tetraploid genome. Two studies found no significant difference between HRD in tetraploid and diploid genomes (Pradhan et al., 2010). However, a study on a small cohort of basal-like breast cancers found that BRCA1 germline mutations happen more often in diploid (12/24 samples, 50%) than in tetraploid cancers (11/41 samples, 27%) (Popova et al., 2012).

Deriving pan-cancer chromosomal instability signatures-exclusion of loss of heterozygosity as a feature. We are not using a feature to describe LOH, because cheap genomic technologies like shallow WGS, which might be very useful in the clinic, do not have allele-specific information. In more detail: Loss of heterozygosity (LOH) is the loss of one allele and is a common genetic event during cancer evolution and might pose a novel class of cancer vulnerabilities by removing genetic redundancy in cancer cells. Most LOH events are so-called copy-loss events and can be detected through a change in copy number. In our data set, the median length of LOH events across the TCGA was 3.6 million bp, which suggests that the majority of events can be detected by the SNP6 technology. Implementing a specific LOH feature, i.e. the proportion of the major allele over both alleles, would add the ability to detect copy-neutral LOH and probably add resolution to further differentiate on copy-loss LOH events. But this would require the use of a high-throughput method allowing for the differentiation of the signal between alleles, such as WGS or SNP 6.0 arrays. However, with the advent of shallow WGS and single-cell sequencing in the scientific mainstream which do not yet allow allele-specific resolution 34, 63-65, a dedicated LOH feature might lead to constraints in the use of the future signatures.

Implementation changes to Macintyre et al., 2018. For the extraction of features from copy number profiles, we introduced three changes from the original implementation described in Macintyre et al.: we smoothed normal segments by collapsing and merging near diploid segments, we only used CNAs (non-diploid segments) for the segment size and changepoint feature and we removed the absolute copy number feature. In more detail: We collapsed and merged near diploid segments to a diploid state (1.9<x<2.1) to avoid signal from segments that were most likely normal diploid segments. We then ignored normal segments when extracting the segment size and changepoint features to avoid inflating the distributions. For the changepoint distribution, we skipped the first segment of a chromosome if it was a normal segment or subtracted 2 from the absolute copy number if it was a copy number aberration.

The major difference brought about by these changes was a shift between signatures 3 and 7 (FIG. 37B), explained by the fact that signature 3 is acting on a diploid genome and 7 on a tetraploid genome, suggesting that both signatures represented the same mutational process artificially split up by the copy number feature. This finding also confirmed that normal, diploid segments influenced signature activity even on strongly rearranged genomes and therefore should be removed from a pan-cancer setting where the majority of samples had quieter, less rearranged genomes. We derived genome-wide distributions of five fundamental copy number features. Using all copy number profiles, we computed distributions of segment size, copy number change to the left neighbouring event (called changepoint), breakpoints per 10 Mb, breakpoints per chromosome arm, and the number of neighbouring segments that oscillated between two copy number states. We ignored normal segments when extracting the segment size and changepoint features to avoid inflating these distributions in genomes with moderate CIN. For the changepoint distribution, we skipped the first segment of a chromosome if it was a normal segment or subtracted 2 from the absolute copy number if it was a copy number aberration. Removing diploid segments influenced signature exposures slightly. It is of interest to know how this change affects the relationship between samples which had highly similar signature exposures under the original framework including diploid segments. First, we studied sample TCGA-09-1673 which had the most CNAs (700) and segments (720) in this cohort. The third most similar sample according to the cosine similarity between ovarian signature exposures (cosine similarity 0.936) was sample TCGA-24-2026 which had a more moderate CIN landscape with 354 segments and 353 CNAs. After removing diploid segments, the cosine similarity improved slightly to 0.941. Despite their different CIN landscape, both samples remained similar after removing diploid samples. Looking at the overall change in cosine similarities between signature activities before and after removing diploid segments shows that overall similarities increased slightly: 95% of cosine similarities changed between 0.27 and −0.06 with a mean of 0.05 and a median of 0.029. Diploid segments can either be normal segments or the result of copy-neutral LOH. Under the assumption that most diploid segments were normal segments, this improvement in cosine similarities is explained by removing the artificial inflation of segments, ultimately influencing all six copy number features and thus signature activities. This increase in cosine similarity can therefore be seen as a refinement in signature activities.

Feature distributions and mixture modelling. We used mixture models for each feature distribution to denoise the data and to identify a core set of underlying copy number features. For the segment size and changepoint feature distributions, being (quasi)-continuous data, we fitted Dirichlet-Process Gaussian mixture models using variational inference (code implementation followed Blei and Jordan, 128 initial components and we ran 10,000 iterations for segment size distribution, and 5,000 iterations for changepoint distribution), resulting in 30 and 22 mixture components, respectively, with weights of more than 1%. Mixture components were merged if a mean was within or near the first standard deviation of another mixture component. The mean of the newly merged component was calculated by the weighted mean of the involved components. Standard deviation was estimated by sampling 100,000 points from the inferred distributions. This reduced the number of mixture components from 30 down to 22 for the segment size mixture model and from 22 down to 10 for the changepoint mixture model. For the count distributed breakpoints per 10 MB, breakpoints per chromosome arm and lengths of oscillating chains, we used Poisson mixture models from flexmix (Grun & Leisch, 2008, R package, v2.3-15) and used the Bayesian Information Criterion (BIC) to decide on the number of components. We derived 3 components for the breakpoints per 10 Mb distribution, 5 for the breakpoints per chromosome arm, and 3 for the length of oscillating chains. In total, we derived 43 mixture components. Once derived, we calculated the probability for each feature value to belong to each component. For a given sample, the probabilities of each mixture component were then summed together, resulting in a 1×43 dimensional vector of sum-of-posterior probabilities. Deriving the sum-of-posterior vector for all 6,335 samples resulted in a 6,335 by 43 sum-of-posterior matrix, referred to as input matrix from here on.

Deriving pan-cancer signatures. We used non-negative matrix factorisation (NMF) on the input matrix. The 43 components contain copy number events generated by all types of CIN and NMF facilitates them identification of separate types of CIN. We used SignatureAnalyzer (Kim et al., 2016, Tan & Fevotte, 2013, Python 3.6.7, cloned on 15/03/19) and L1 regularisation on both output matrices to perform non-negative matrix factorisation (NMF) on the input matrix. We ran the NMF 200 times with random initialisation to navigate the non-convex optimisation function and compared the overall Kulback-Leibler (KL) divergence between the input matrix and the reconstructed input matrix from the output matrices. A lower KL divergence indicates a better solution. The number of signatures, K, was defined as the mode of the distribution of K's over the 200 runs, which was equal to 10 for all 6,335 samples pan-cancer. Of the 200 runs, 95 solutions had a K of 10. To pick a representative solution, we first calculated the cosine similarity between the signatures in the 95 sets and visualised the results as a graph.

The graph had 950 nodes, each one representing a signature, and edges connecting nodes if the cosine similarity between these two signatures was above 0.85, a threshold established in the literature (Alexandrov et al., 2020). This graph showed 12 independent clusters with sporadic outliers attached to them, as well as a few single independent nodes. In a second step, we ordered the 95 solutions by their KL divergence and, starting with the highest-ranked solutions, evaluated their spread in the graph. We chose the 9th optimal solution as it had a signature in 10 of the 12 clusters. The previous 8 solutions all had at least two signatures in the same cluster, indicating almost identical signatures. In terms of the Kullback-Leibler divergence, the solution we picked is about 1% worse than the most optimal solution we derived.

Deriving cancer-type enriched signatures. Next, we aimed at deriving cancer-type enriched signatures to complement the pan-cancer set of 10 signatures. We hypothesised that there might be signatures present in a certain cancer type but that their signal may be drowned out during pan-cancer NMF. Therefore, for 20 of the 33 cancer types with over 100 samples with detectable CIN, we derived cancer-type enriched signatures following the above described method. The number of samples was similar to those found in previous publications on copy number signatures (Macintyre et al., 2018, Steele et al., 2019). K ranged from three in testicular germ cell tumours to eight in five cancers. For 11 cancer types the mathematically optimal set of signatures was also the representative of other solutions. For the other 9 cancer types we picked a representative solution over the mathematical solution. In total we derived 128 cancer-type enriched signatures.

Merging to a signature compendium. For merging the pan-cancer and the cancer-type enriched signatures to create a compendium of CIN signatures, we compared signatures by cosine similarity. To decide which signatures to merge we derived a threshold using simulation. Based on the set of 138 pan-cancer and cancer-type enriched signatures (138×43 matrix), we simulated 1,000 sets of signatures using a Dirichlet process from MCMCpack (Martin & Park, 2011, R package, v1.4-7), maintaining the proportion of 0s and signature components summing to 1 for each signature. Descriptive analyses (heatmaps) were then used to display (i) the distribution of cosine similarities between the signatures, (ii) the simulated signatures, and (iii) the distribution of cosine similarities between the original signatures and the simulated signatures. These analyses showed that the simulated signatures were similar to the original ones. Therefore, we used the quantile 0.999 of the distribution of simulated cosine similarities, which was equal to 0.74, as a cosine similarity threshold. In order to merge the pan-cancer and cancer-type enriched signatures, we employed a number of strategies to avoid redundancies amongst the signatures: (1) remove cancer-type enriched signatures which were similar to pan-cancer signatures. To do this, we remove all cancer-type enriched signatures that had a cosine similarity over 0.74 with any pan-cancer signature. (2) remove cancer-type enriched signatures which were too similar to each other. To do this we selected a representative signature (highest activity for the largest number of samples) from the groups of cancer-type enriched signatures that had a cosine similarity over 0.74. (3) remove cancer-type enriched signatures which constitute a combination of pan-cancer signatures. To do this we performed non-negative least squares on each pair of pan-cancer specific signatures and cancer-type enriched signatures. For any combination which showed a reconstruction error below 0.1, the cancer type enriched signature was removed. The combination of three strategies reduced the 128 cancer-type enriched signatures to 7. No combination of pan-cancer signatures resulted in a cancer-type enriched signature, and vice-versa, therefore the 10 pan-cancer signatures were combined with the 7 cancer-type enriched signatures to yield a set of 17 signatures of CIN in human cancers. These signatures were then named by their rank determined by the number of samples with non-zero signature activity. Once defined, the final signature activities for all 6,335 samples with detectable chromosomal instability were computed using the linear combination decomposition function from YAPSA (R package, v1.12.0) on the original input matrix.

Simulation of signature activities. We tested how stable the signature activities are by performing a Monte Carlo simulation where we added noise to the number of CNAs and their characteristics and then measured how strong the signature activities changed. For each value of the segment length and the copy number changepoint feature, we added or subtracted up to 10% of their value which we drew from a Gaussian distribution with a mean of that value and a standard deviation of one-twentieth of the mean. If a drawn value was larger than 10% we discarded that value and drew again. This procedure had the effect that longer or higher copy number segments were modified stronger than smaller or lower copy number segments. In addition, we modified the breakpoints per chromosome arm, breakpoints per 10 Mb and length of oscillating copy number chain features by drawing the same number of values from a sample's feature distribution with replacement. In order to avoid a strong deviation from the original sample feature distribution, we applied a maximum change of +/−10% to the number of CNAs. If a draw resulted in a higher change in CNAs, then we redrew values until the number of CNAs was below 10% change from the original value. One run of the simulation included a change of all five feature distributions for all 6,335 samples with a subsequent computation of the signature activities. In total, we ran the simulation 1,000 times. We plotted the interquartile range (IQR) for all samples and all 17 signatures (FIG. 10).

Definition of signature-specific thresholds. To ensure robustness of the signature activities and to enable trust in small signature activities, we sought to identify signature-specific thresholds. Based on the 1,000 simulations, we used the non-zero simulated values from samples with true zero activity. We fitted a single Gaussian distribution to the non-zero values with the function Mclust from mclust (Scrucca et al., 2016, R package, v5.4.6) and took the quantile 0.95 as a threshold (FIG. 10). To calculate pan-cancer prevalence we counted in how many samples each signature activity was above its specific threshold. This approach gives identical results for pooled data or merging prevalence per cancer type.

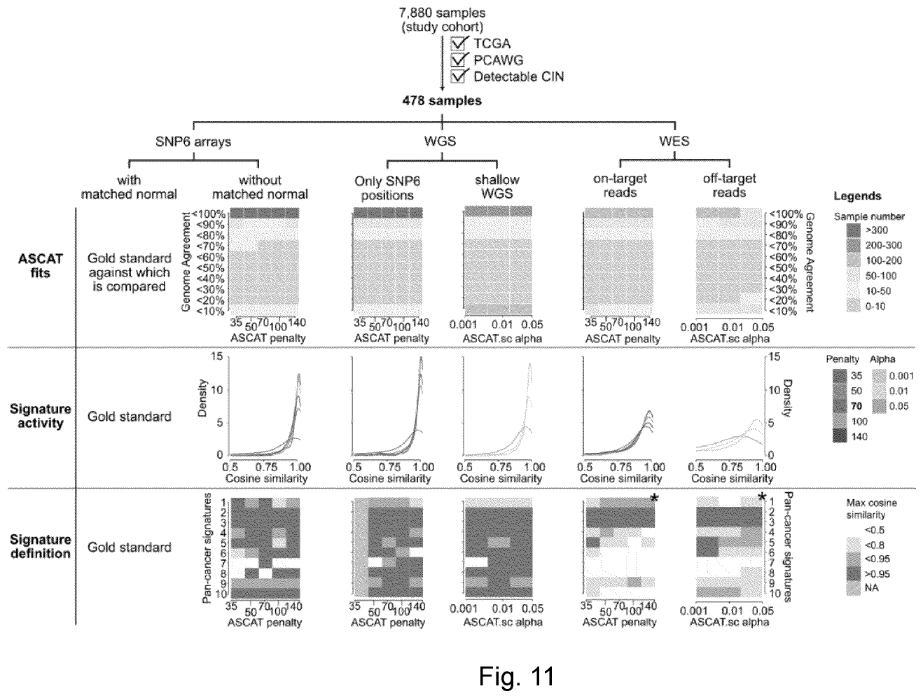

Testing robustness of signatures across sequencing technologies. In order to test the robustness of the signatures across sequencing technologies, we used 478 TCGA samples with detectable levels of CIN that were also part of the PCAWG project and derived signature activities and definitions in five settings: 1. SNP 6.0 without matched normal, 2. WGS downsampled to SNP 6.0-positions, 3. WGS downsampled to shallow WGS, 4. WES on-target reads, 5. WES off-target reads. For each setting, we fitted absolute copy number profiles (step 1—see “Absolute copy number profiling below”), extracted signature activities for the 17 compendium signatures and derived 10 signatures (step 2-see “Extract signature activities and definitions”) and compared them to the 10 pan-cancer signatures (FIG. 11, step 3-see “Comparison to gold standard” below). The data set was not big enough for cancer-type specific analyses. For absolute copy number profiling of 1, 2 and 4 we used the same ASCAT (R package, v2.4) as described in section “Inferring absolute copy number from SNP 6.0 arrays” (pages 5-6) available at https://github.com/VanLoo-lab/ascat. For the two approaches using binned reads (3 and 5), and for deriving the copy-number profiles of cell lines, we used a new method based on ASCAT, called ASCAT.sc (R package, v0.1, manuscript in preparation) which is currently available here: https://github.com/VanLoo-lab/ASCAT.sc.

Absolute copy number profiling-SNP 6.0 without matched normal. For many bulk tumour SNP 6.0 profiles, the non-tumour contaminant pulls the B-allele frequency (BAF) of heterozygous SNPs close to 0.5, such that 0<BAFₕₑₜ<1 and it can be distinguished from the BAF of homozygous SNPs, i.e. BAFₕₒₘ=1 or BAFₕₒₘ=0. Using this rationale, ASCAT proposes a built-in function to infer which SNPs are heterozygous from the tumour profile only. The parameters of this function are based on the noise profile of the platform and the array design, as manually defined after visual inspection of multiple profiles from the same platform. In this case for Genome-Wide Human SNP Array 6.0, the function expects a range of BAF values for homozygous SNPs (BAFₕₒₘ<0.04 or BAFₕₒₘ>0.96) and a minimum fraction of noisy SNPs (>4% expected with BAFₕₒₘ<BAF_{noisy}<BAFₕₑₜ), as well as a platform-specific maximum fraction of heterozygous SNPs (25%) and minimum fraction of homozygous SNPs (67%). To identify heterozygous SNPs, the BAF of each SNP is first flipped around 0.5 such that all BAF values are within [0, 0.5], i.e. if BAF>0.5, BAF_{flipped}=1-BAF. Then the fractions of homozygous fₕₒₘ, noisy fₙₒᵢₛₑ, and heterozygous fₕₑₜ SNPs are determined from the platform-specific parameters and the sample's BAF values: from the total number of SNPs N, the fₕₒₘ×N SNPs with a BAF closest to 0 are labeled homozygous; from the remaining SNPs, fₕₑₜ×N SNPs with the lowest distance between their BAF and a local median of the BAF within a window of 301 (of the non-homozygous) SNPs centred around the SNP positions are labeled heterozygous; the remaining SNPs are labeled noisy and left out from downstream analyses. While this methodology allows to identify heterozygous SNPs in most unbalanced segments, in tumours for which purity is close to 100%, the BAF of heterozygous SNPs in regions that are largely unbalanced, and especially with loss of heterozygosity (LOH), is indistinguishable from the BAF of homozygous SNPs, i.e. BAF=1 or BAF=0. Thus, highly imbalanced segments in pure samples are devoid of identifiable heterozygous SNPs. Moreover, because ASCATs segmentation step only considers heterozygous SNPs, this leaves large blind spots for the identification of copy-number segments in those samples, which would significantly influence our signature framework. To counter this, we have added a step after the identification of heterozygous SNPs, which rescues SNP positions in segments of BAF>0.75 and where the density of heterozygous SNPs is much lower than the average in non-LOH regions (<20%). We rescue as many SNP positions from the SNPs labeled as homozygous, as to reach a 20% density of heterozygous SNPs within the segment. To avoid inducing spurious segments in the BAF of rescued SNPs, we do not rescue SNPs at random but rather the SNPs with BAF values closest to BAF=0.5. We then run ASCAT on the union of the SNPs identified as heterozygous in the first step and those rescued in segments depleted from heterozygous SNPs.

Absolute copy number profiling—WGS downsampled to SNP 6.0 positions. From PCAWG WGS (hg19) data, we considered allele counts from alleleCounter (v4.1.0, default parameters with the “--dense-snps” option; https://github.com/cancerit/alleleCount) at SNP 6.0 loci. For each sample, loci with at least 1 count in the tumour and 10 counts in the matched normal were used to derive log R and BAF. Log R was then corrected for GC content and replication, implemented following the methodology in the Battenberg R package (https://github.com/Wedge-lab/battenberg). ASCAT was then used with different penalty values of interest and a gamma value set to 1.

Absolute copy number profiling—Bin size estimation allowing for downsampling to shallow WGS. As genome binning, or lack thereof, differs between SNP 6.0 array copy number calling and shallow WGS approaches, we sought to identify a genomic bin size which most accurately recapitulates the segment feature distribution seen in SNP 6.0. Segment size was selected as an appropriate measure of the similarity in sample segmentation to compare between differing genomic technologies. A set of 40 ovarian cancer WGS tumour samples from the PCAWG study were used to verify segment size agreement due to a high variability in segment lengths and high segment counts per sample. WGS samples were downsampled to between 3-15M reads, with consideration for ploidy, purity, bin size. Segment size distributions were calculated for five genomic bin sizes (5 kb, 15 kb, 30 kb, 50 kb, and 100 kb) and compared with segment size distributions generated from matched SNP 6.0 array segmentation as described in section “Inferring absolute copy number for SNP 6.0 arrays”. Segment size distributions were tested against the reference set using a Mann-Whitney U test after removing segments longer than 10 Mb. We determined and selected a bin size of 30 kb because its the segment size distribution resulted in a non-significant change (or least significant change) and therefore most accurately replicated the segmentation profiles in matched SNP 6.0 array data (30 kb p-value=0.11, all other bin sizes p-value <0.001). We downsampled WGS samples to appropriate read counts, based on the bin size determined prior, sample ploidy, and sample purity and performed absolute copy number fitting as described in section “Shallow WGS: Absolute copy number fitting.

Absolute copy number profiling—Shallow WGS: Absolute copy number fitting. To infer copy numbers in shallow coverage WGS, we defined 30 kb non-overlapping windows along the genome, excluding centromeres and regions of undefined sequence, i.e. “N”, in the reference genome hg19 (see section above “Bin size estimation allowing for downsampling to shallow WGS”). Then, we empirically identified low-quality bins akin to Scheinin et al., which can be bins of low mappability or high variability of sequence repeats across individuals. This is done as follows: for 10 normal diploid blood samples from the PCAWG project with an estimated fraction of tumour contaminant at 0%, we count the number of reads in each bin and derive the log R across the bins

$$log ( R ) = {log}_{2} \frac{{N}_{bin} + 1}{\overline{{N}_{b ι n}}}$$

where N_{bin} is the number of non-duplicated reads with mapping quality >=30 in the while, and N̅_̅{̅b̅i̅n̅}̅ is the average number of such reads across all the bins. We then corrected the log R for GC content within the bin using the function loess (R core package, v.3.6.1), with default parameters. We then ranked the log R values by their counts in each sample and derive a symmetric rank-based score to identify outliers with low or high read counts:

$$s = log ( \frac{r}{N + 1 - 5} )$$

where is the rank of each bin's log R and N is the number of bins. We then summed the scores across samples and took the absolute value. After visual inspection of the profiles, a conservative fifteen percent of the bins with the highest final score were labelled as bad bins and removed from downstream analyses. This was done separately for autosomes and chromosome X. We then process each tumour sample individually: we counted reads in the remaining 30 kb bins and derived the log R as described. The log R was then corrected for the GC fraction of the bins using loess, and segmented using circular binary segmentation (CBS, Olshen et al., 2004) using the function segment (Seshan & Olsen, 2019, R package DNAcopy, v1.64.0) with default parameters. We assessed the influence of the alpha parameter (0.001, 0.01, 0.05), which is the p-value threshold used to identify segments with different mean values. After the profile was segmented, we searched the space of combinations of realistic purity and ploidy values (0.01<purity<1, 1.5<average tissue ploidy<5, and tumour ploidy<5) to fit the log R to integer values, solving the log R equation for each segment:

$$log ( R ) = {log}_{2} \frac{{n}_{T} ρ + 2 ( 1 - ρ )}{ψ}$$

where n_{T} is the total number of copies in the tumour, ρ is the purity and ψ is the average tissue ploidy. We picked the purity and ploidy combination that minimized the distance between nr and integer values and solved for each segment to obtain a non-rounded total number of copies.

Absolute copy number profiling—WES: Absolute copy number fitting of on-target reads. From TCGA WES (hg19) data, we considered allele counts from alleleCounter (v4.0.0, default parameters with the “--dense-snps” option; https://github.com/cancerit/alleleCount) at SNP loci from the 1,000 Genome Project (Auton et al., 2015, phase 3) falling into exonic regions (GENCODE v19 exome Frankish et al., 2019, see Table 1 for details); extended by 1,000 bp upstream and downstream). For each sample, loci with at least 1 count in the tumour and 20 counts in the matched normal were used to derive log R and BAF. Log R was then corrected for GC content and replication timing, following the methodology implemented in Battenberg (Wedge et al., 2021R package, v.2.2.9). ASCAT was then used with different penalty values of interest (35, 50, 70, 100, 140) and a gamma value set to 1. The following rules were used to discard samples: No ASCAT solution; Purity>0.99 and 1.99≤ploidy≤2.01; Tumour MAPD>0.75; Tumour MAPD>0.4 and fraction homozygous segments ≥0.1; Differences between log R and BAF segments >250; Number of considered SNPs ≤75,000. To pick a single representative sample for multi-sample cases, we considered the following priority: matched blood over matched adjacent normal tissue and most recent analysis date (if available). If multiple samples still fitted these criteria, a sample was randomly selected.

Absolute copy number profiling—WES: Absolute copy number fitting of off-target reads. We calculated and estimated downsampling read counts using the same methodology and bin size estimates calculated for down sampling of WGS to shallow WGS (see section “Bin size estimation allowing for downsampling to shallow WGS”). To derive the copy-number profiles, we first define an on-target region from the bed definition of the targets in TCGA, adding 1000 base pairs either side of the targets. We then bin the genome into 30 kb bins but excluding the on-target regions, i.e. the bins are not necessarily made of contiguous sequences and their genomic width is usually larger than 30 kb. We remove bins that overlap with “bad bins” as defined earlier, we derive the log R in each bin, segment it with CBS, and apply the ASCAT log R-only methodology as described above in section “Shallow WGS: Absolute copy number fitting”.

Extract signature activities and definitions. Extraction of the feature values from the copy number profiles were performed as described above in section “Feature distributions and mixture modelling”. For identifying the signature activities, we used the linear combination decomposition function from YAPSA35 (R package, v1.12.0) and the 17 CIN signatures. For identifying new signatures and comparing the definitions to the original 10 pan-cancer signatures, we used the same procedure as described above (“Deriving pan-cancer signatures”) with the notable change that we used only solutions with K, the number of the original pan-cancer signatures, equal to 10. As no solution with K=10 were found for the two WES settings and the SNP6-positions only WGS setting, we chose the optimal K (K=7) for the two WES settings and greyed out the figure for the SNP6-positions only WGS setting.

Comparison to gold standard (SNP6 with matched normal). All signature activities were significantly different from a random background distribution of signature activities. For this background distribution, we simulated a 1000 sets of random signature activities for the 478 samples similar to the method described above in section “Merging to a signature compendium”. The expected random cosine similarity was around 0.05 (average random cosine similarity across all samples, peaking at 0.05). The signature definitions were compared by cosine similarity where we chose a cutoff of 0.95 to define equality. Every one of the 10 original pan-cancer signatures (FIG. 11, bottom row, y-axis) was found in at least one other setting, showing that the signal can be captured by a small subset of samples from other sequencing settings.

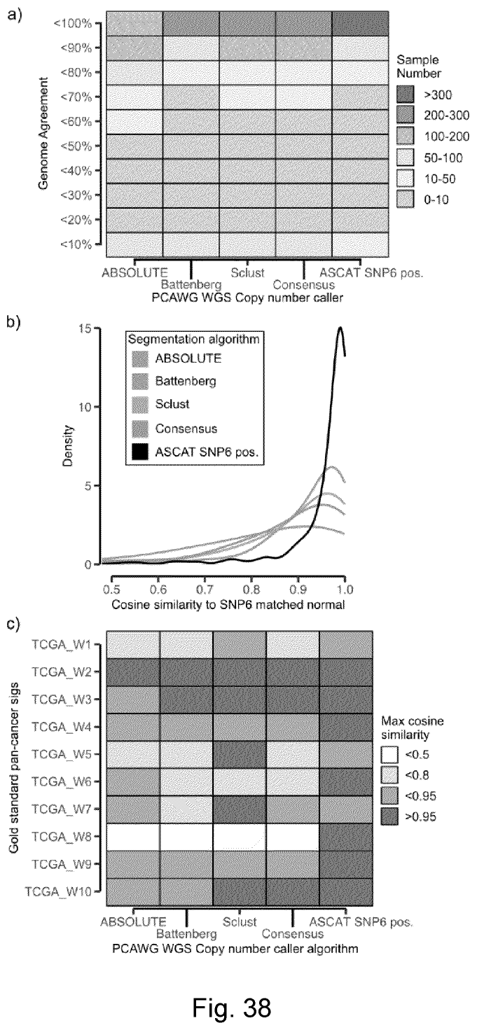

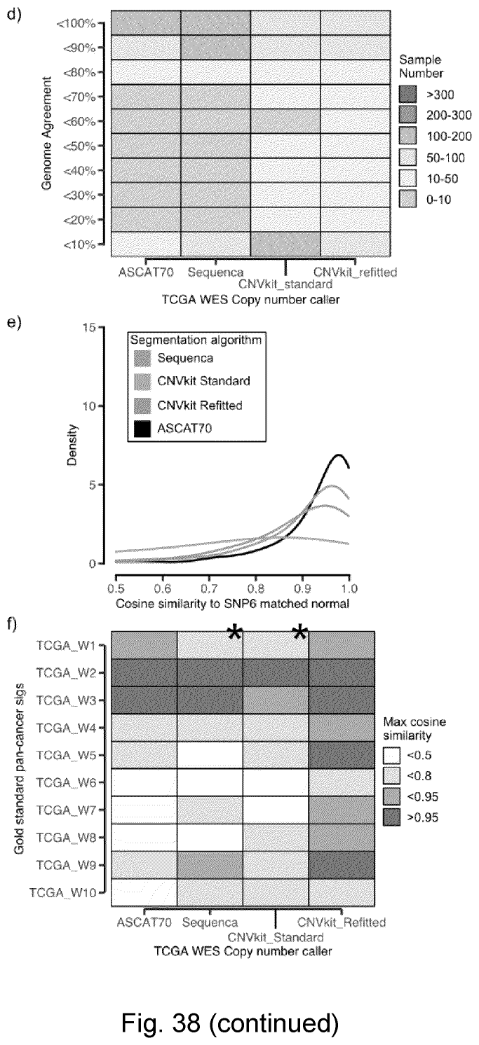

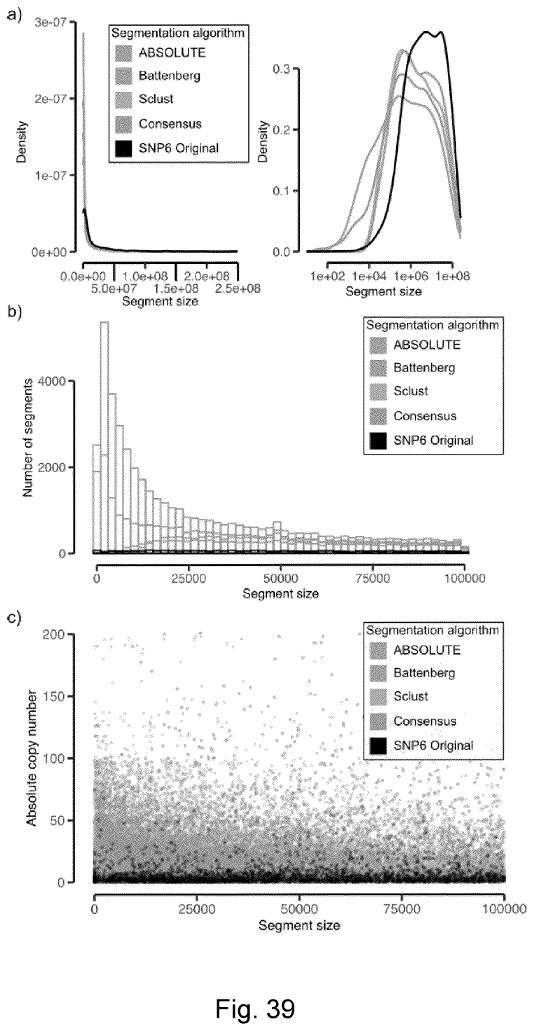

Extension to WGS and WES data. We developed our signature approach using TCGA SNP6 data because this represents the largest pan-cancer collection of high-resolution copy number profiled tumours to date. Nevertheless, the resolution of copy number calling from SNP6 data is lower than whole-genome sequence data, which is a limitation of the particular implementation in this example. In the cross-technology platform comparison, we normalised all data to match the resolution of SNP6 arrays and showed that our signature methodology is robust across technology platforms at this resolution (FIG. 11). If the resolution of copy number calling is increased (e.g. by using base-pair resolution whole-genome sequencing derived copy number), the underlying feature distributions change (FIG. 39). This change can have two interpretations. Firstly, it may be possible to identify new signatures that were not detected at SNP6 resolution. As more pan-cancer WGS data becomes available, it may therefore be possible to redefine our signatures at higher resolution (if needed). Secondly, the SNP6 resolution signatures may not be fit for purpose applied to high resolution data. In order to test this, we applied our SNP6 signatures to higher resolution WGS and to WES data processed by multiple different copy number callers (FIG. 38). As expected, we see a drop in similarity scores for the activities and signature definitions. In some cases (such as ABSOLUTE on WGS), these reductions are minor and it could be possible to use our signatures “out-of-the-box” on these data. In other cases, the signatures may not perform as well “out-of-the-box” but simple precautions can be taken to ensure that the signatures perform as well as possible. In particular, the following recommendations may be useful when applying the pan-cancer copy number signatures to new data: (i) SNP6 arrays: recommended use with and without matched normals using ASCAT with a penalty of 70; (ii) WGS: recommended use with ASCAT focusing on SNP6 positions or ABSOLUTE on full WGS profiles; (iii) Shallow WGS: recommended use with ASCAT.sc and a penalty of 0.001; (iv) WES: for on-target reads, either ASCAT (penalty 70) or Sequenza [Favero et al., 2015] can be used; for off-target reads, ASCAT.sc with penalty 0.001 is recommended.

CIN activity cannot be simply measured by the number of CNAs. Modelling the effects of different types of chromosomal instability presents a unique challenge compared to SNV and SV signatures. This challenge stems from the fact that for CNA analyses we need to distinguish between a genome-changing event and the number of CNAs it produces. For some types of chromosomal instability, a single event causes a single copy number change (e.g. whole chromosome missegregation), while for other types a single event can cause many changes (e.g. chromothripsis). Therefore, to adequately model the activity of different types of CIN, the number of CNAs needs to be decoupled from the number of “events”. This is different from SNV signatures, where the number of SNVs is assumed to be a good measure of the activity of a mutational process. Developers of SV signatures faced a similar problem and Li et al. (2020) addressed it by performing extensive clustering of SVs into mutually exclusive “event” types, ranging from events that encompassed a single deletion of a certain size, to events that involved large numbers of SVs as part of chromoplexy chains. This facilitated the counting of these events in a tumour and the application of NMF, similar to what had been previously done for SNVs. The approach of Li et al thus allowed SV signatures to be interpreted in a similar manner to SNV signatures. Copy number data, however, presents unique challenges, precluding the use of a similar approach. CNAs cannot be easily clustered into single “events” as is the case for SVs, as they lack the information required to relate one CNA to another. Further confounding this problem is the fact that copy number changes are quantified with respect to reference genome coordinates. For example, the loss of a “chimeric” chromosome (an already rearranged chromosome containing parts of many different chromosomes), is a single “event” in the tumour cell. However, mapped to a reference genome, this can manifest as many copy number changes across multiple chromosomes. Therefore, it is not possible to generate a vector of “event” counts and thus approaches similar to those used in the SNV and SV setting cannot be applied. The section “Estimation of CNAs produced by a signature” in the Methods of Example 2 shows how to provide an estimate of how many copy number changes are represented by each signature in a tumour.

Correlations and substructure in the feature embeddings. Modelling copy number signatures demands a completely new and different approach. As originally published in Macintyre et al. (2018) and substantially enhanced here, we used the published literature to define a set of copy number related features representing known outcomes of different types of CIN including chromothripsis, tandem duplication, whole-genome duplication and focal amplification. This formed the basis of our set of 5 features. Embedding CNAs observed in a tumour into this feature space, means single CNAs can add weight to multiple features. This has the significant advantage of allowing “events” to be encoded that correspond to low or high numbers of CNAs, and thus overcomes the significant challenges outlined above of working with copy number data. However, this approach does have some potential caveats when interpreting the signature definitions. First of all, our feature encoding introduces substructure in the input matrix that is different to that of a typical SNV count matrix. However, this structure does not violate any assumptions of NMF (the only assumption of NMF is that the matrix does not contain negative numbers), so NMF can still be applied. When faced with deciding on which approach to use for matrix decomposition, we identified two possibilities: 1) a bespoke approach, similar to the Hierarchical Dirichlet model with latent variables that was used in Li et al. (2020), where we specifically take into account the substructure of our feature encoding; or 2) the widely used NMF approach, which is agnostic to the substructure. The advantage of the HDP-like approach is that we can impose prior knowledge that certain features should behave in certain, coordinated ways. However, as the feature encoding itself already strongly imposes prior knowledge, this additional constraint may suppress any ability to perform data driven discovery and could be overly biased by our prior knowledge. The advantage of using NMF is that it is robust and widely used across many scientific domains, including face recognition, astronomical image analysis and text mining. It has well defined behaviour and provides a data driven approach to signature identification across our feature space. In this work we aimed for a balance between using prior knowledge and data driven discovery: we encoded prior knowledge in the selection of copy number features (which were designed to capture known patterns of CIN) but used a widely used, well-tested, and robust “off-the-shelf” method for deconvolution. However, it is important to note that NMF may result in signature weights that do not reflect the expectations of our prior knowledge. A significant strength of our approach is that it is easy to identify potentially erroneous signature definitions as these have strong weights on a single component. In this case, the single defining weight is so strong that other related weights (which we might expect to see) get shrunk to 0 (this is part of the NMF regularisation to ensure a unique solution). Fortunately, CX13 is the only signature that exhibits such behaviour. Our low to medium amplification signatures (C×8 and C×9) have weights on high change-points but also have weights on the expected segment sizes (both short and long). However, in the case of CX13, the high changepoint component is such a strong defining feature that no weight is given to segment size components. While this may yield a slightly unexpected signature definition, this does not necessarily imply that this signature is not robust in representing the process generating high-level focal amplification. Indeed, post hoc analysis of segments with the defining change point value (at least 10 copies) yields a median segment size of 790 kB, with an interquartile range between 280 kb and 2.7 Mb which is consistent with estimates for ecDNA sizes of 1-3 MB. This effect may be more subtly manifested across all signatures. The replication of all pan-cancer signatures during our cancer-type enriched signature analysis suggests that our signatures are robust readouts of biological signals.

Testing for an ecDNA-focused signature. In the CIN signature compendium, the maximum mixture component for the copy number changepoint feature was located at around 10 copies, in stark contrast to the ovarian copy number signature components (Macintyre et al., 2018) where the maximum was at 30. This component at 30 copies change was crucial for defining ovarian signature 6. Given that focal amplifications such as extrachromosomal DNA (ecDNA) are present in a wide range of cancer type but still show cancer-type specific enrichment, particularly in ovarian cancer (Kim et al., 2020, Gu et al., 2020), we were wondering whether we would miss a signature focusing on extremely high amplifications. We manually added the mixture component around 30 copies to the components of the changepoint feature and rederived pan-cancer signatures. This additional component did not create an additional signature focusing on ultra-high amplification. Instead the additional mixture component was added to the signature already containing the strongest weight on the changepoint component around 10 copies.

All examples used the reference genome GRCh37/hg19. Unless stated otherwise, statistical software R (v3.6.1) was used.

### Results

The inventors derived 7,880 high-quality absolute copy number profiles across 33 tumour types using ASCAT (Van Loo et al., 2010) on the SNP array data of TCGA (FIG. 5a). Using the framework described in Macintyre et al., 2018 for quantifying signatures of CIN in ovarian cancer, they determined that 6,335 of the 7,880 samples (80%) had detectable CIN and would be suitable for pan-cancer detection of CIN signatures (FIG. 5b). This estimate is consistent with previous pan-cancer estimates of CIN (FIG. 5c-e).

Using these 6,335 genome-wide copy number profiles, they computed distributions of five fundamental copy number features previously demonstrated to encode patterns of copy number change representing different underlying causes of CIN (Macintyre et al., 2018). These features are the copy number change between a segment and neighbouring segment, segment length, breakpoint count per 10 megabases, breakpoint count per chromosome arm and length of chains of oscillating copy number states (Methods). In a major departure from the previous work, the inventors did not include a feature representing the copy number of a segment as they discovered that this would advantageously avoid redundancy if signatures for the same aetiology appear across different ploidy backgrounds.

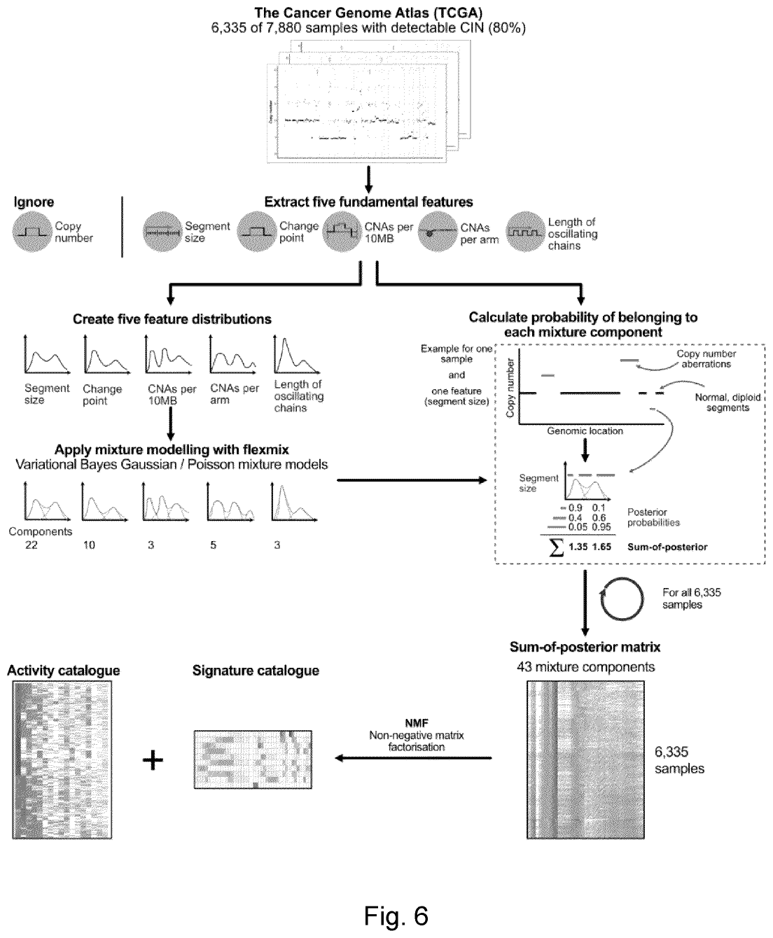

Then, the inventors applied mixture modelling to define distinct components for each cohort-wide feature distribution, which conceptually represent the basic building blocks for defining CIN processes, and identified a total of 43 mixture components across the 5 features (Methods, FIG. 6). Probabilistically assigning the CNAs of each copy number profile to these mixture components resulted in a 6,335×43 matrix, which was used in a Bayesian implementation of non-negative matrix factorisation (Kim et al., 2016; Tan & Fevotte, 2013) for CIN signature identification.

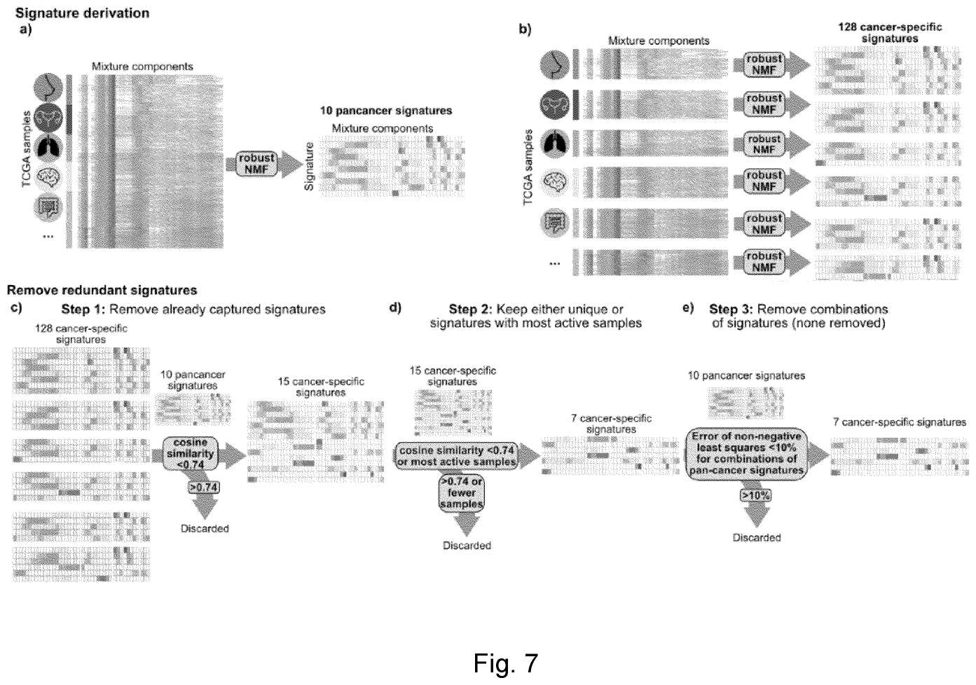

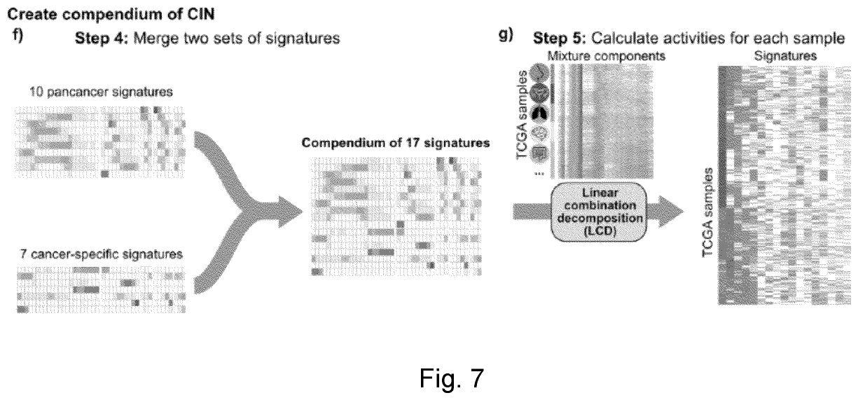

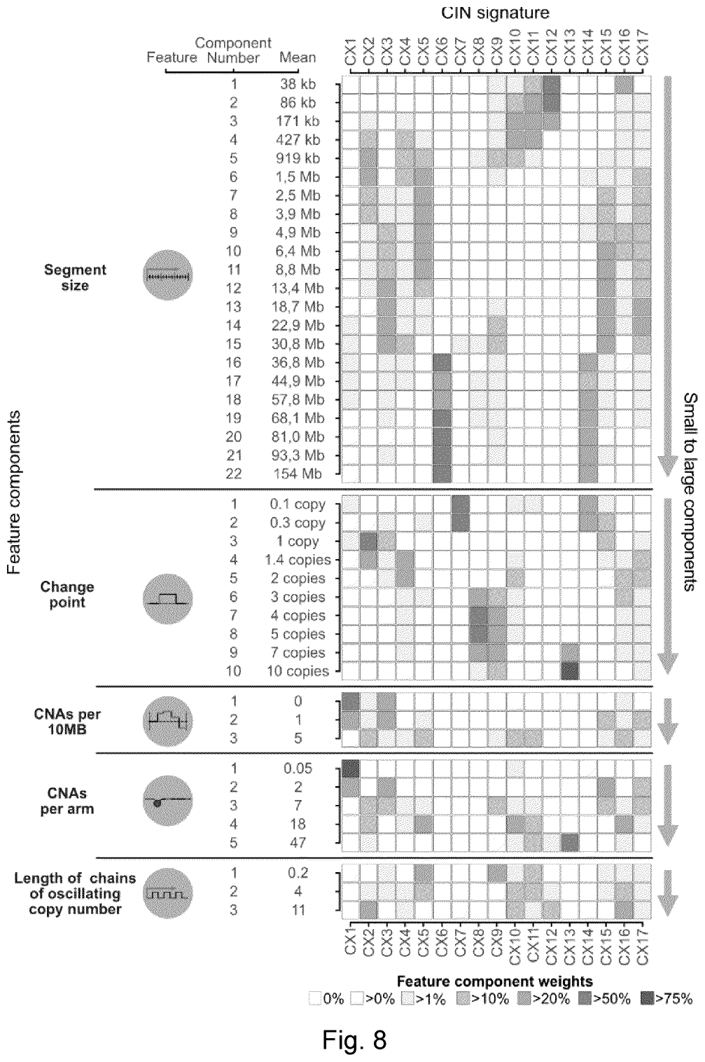

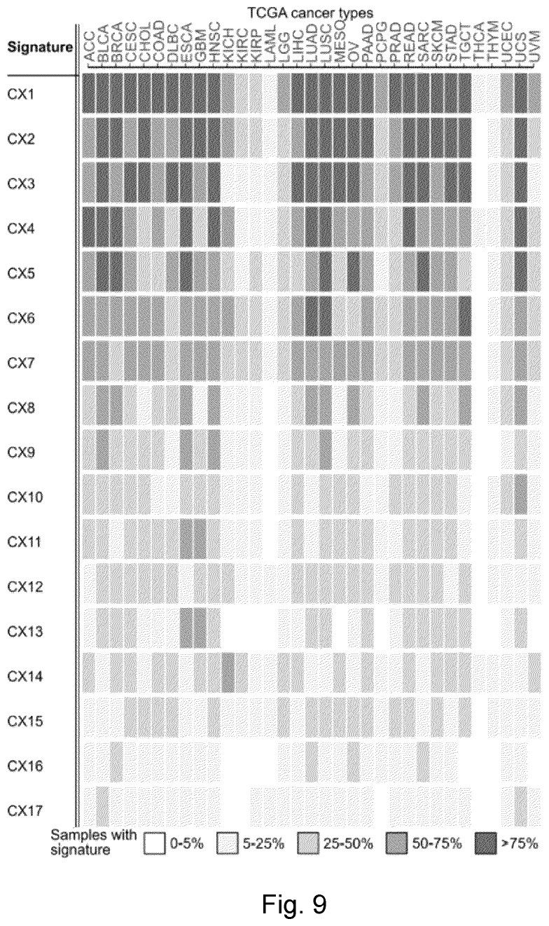

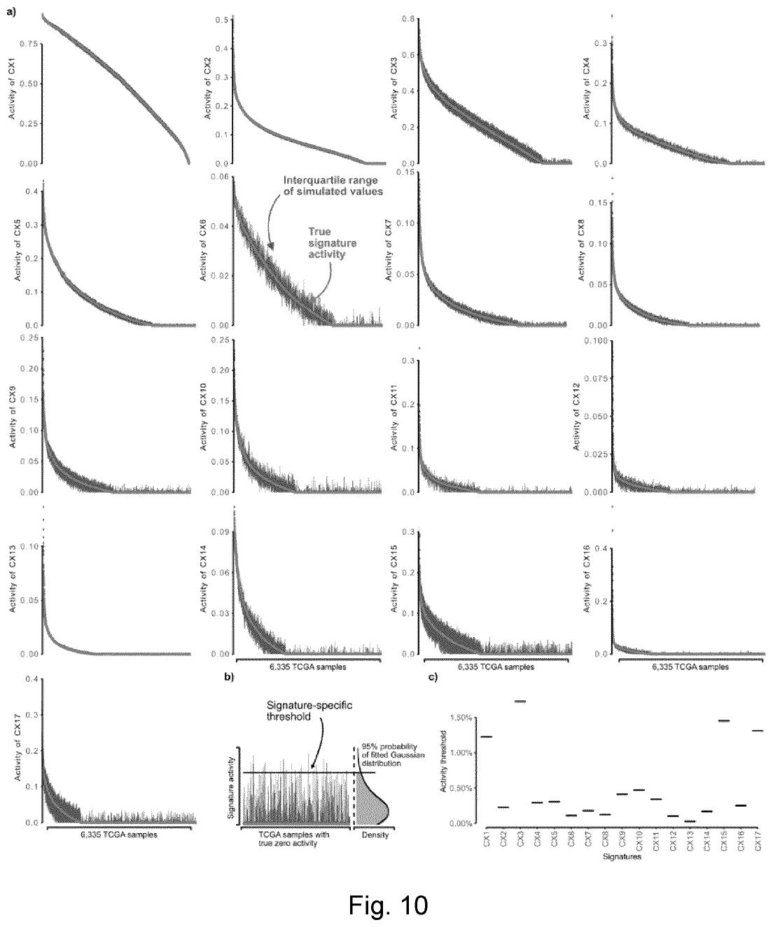

The inventors investigated this data set in two different ways (FIG. 7). They first used the complete matrix and found 10 pan-cancer copy number signatures. Then, by using individual cancer types with at least 100 samples, they found additional 128 signatures, 7 of which were distinct from the pan-cancer signatures based on analysis of pairwise cosine similarities and multivariate regression (Methods). All 7 single-cancer derived signatures were active in more than one cancer type, showing that the process identified signatures enriched in a subset of cancers but diluted in a pan-cancer analysis. Merging both sets of signatures and computing their activities using linear combination decomposition yielded a pan-cancer compendium of 17 copy number signatures (FIG. 7f-g, FIG. 8) and their activities across the 33 cancer types (Table 2, FIG. 9).

The inventors tested the robustness of their signatures in several ways. First, they used a Monte Carlo simulation study to ensure that low signature activities are not just due to noise and derived signature-specific activity thresholds (Methods, FIG. 10). Second, they tested the robustness of their approach across high-throughput technologies using 478 samples profiled both by TCGA (SNP6 and whole exome sequencing, WES) and PCAWG (whole genome sequencing, WGS) (FIG. 11). They compared five platforms: SNP6 without matched normal, WGS downsampled to SNP6 positions, WGS down-sampled to shallow WGS, on-target WES, and off-target WES. This analysis accurately replicated the pan-cancer signatures (cosine similarity threshold 0.95) and found significant similarities in signatures activities across all platforms.

### Example 2—Identification of Signature Aetiologies

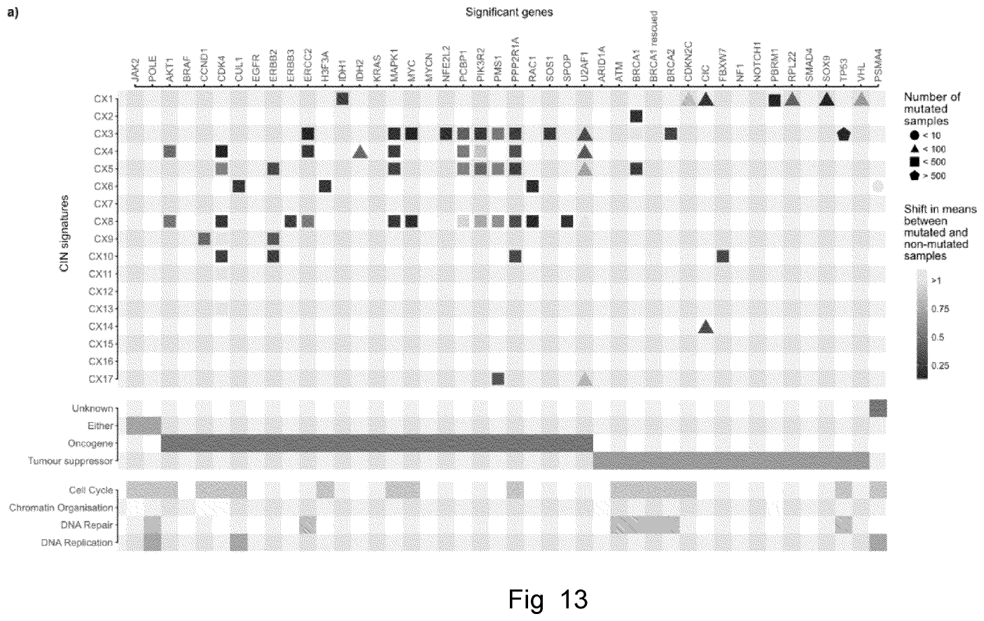

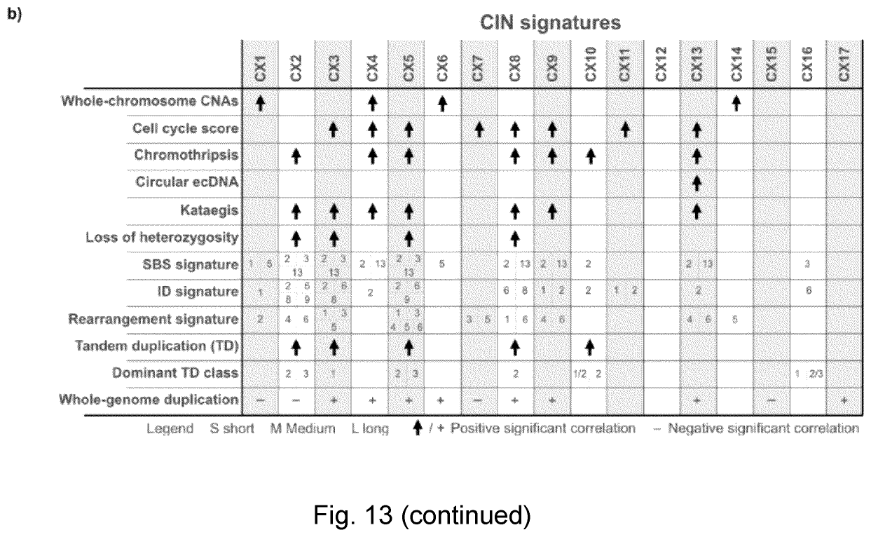

In this example, the inventors investigated the putative causes underlying the signatures identified in Example 1. In order to do this, the inventors decided to derive an initial hypothesis based on the signature definitions (i.e. the patterns of copy number change encoded by the signature, FIG. 8) and signature associations with known cancer driver mutations (FIG. 13a). The explicit assumption made was that the driver gene associations acted as markers for putative pathways involved in the aetiologies and assumed a common pathway deregulation for samples without a driver gene mutation.

### Methods

See Example 1. The paragraphs below first describe how the inventors compiled the list of genes of interests in order to link signature activities to mutated genes, then how they identified the signature aetiologies and then how they associated the signatures with orthogonal data.

Selection of 1,146 genes of interest. We performed mutation analysis on 1,146 putative CIN related genes compiled from known pan-cancer driver genes (from the TCGA, Bailey et al., 2018), and genes reported as involved in DNA repair, DNA replication, the cell cycle, and chromatin organisation by the Reactome database (Date of access: Mar. 3, 2020; Jassal et al., 2020, Fabregat et al., 2018). The Reactome query resulted in a list of 1,330 proteins known to be involved in DNA repair, DNA replication, the cell cycle, and chromatin organisation. Reactome supplied three types of identifiers: UniProt, ensembl gene identifier, and EMBL identifier. Using the three identifiers, we mapped the proteins to 1,443 ensembl gene entries (ensembl96 on a GRCh37 reference genome). Ignoring the UniProt identifier and removing duplicated entries, the total went down to 1,036 gene names with 1,136 ensembl identifiers. We added to this list 200 genes reported by Bailey et al. as pan-cancer drivers. We transferred the names to ensembl96 on a GRCh37 reference genome and merged them with the Reactome-derived list of genes. The resulting list had 1,296 ensembl gene identifiers for 1,179 gene names. An additional functional descriptor supplied by Bailey et al. was taken into account for the pan-cancer driver genes: “oncogene” and “tumour-suppressor gene”. If none existed, we assigned the descriptor “ambivalent”. In total, 58 oncogenes, 112 tumour-suppressors, and 30 ambivalent genes were labelled as such. For all 1,179 genes, we identified their transcriptional start sites (TSS) on a GRCh37 reference genome. Since most genes have multiple TSS, the canonical or strongest proven TSS was obtained by the following decision tree: 1) If only one TSS was present for a gene, then take this one independent of quality; 2) if a GENCODE basic flag was present, take only these, 3) if multiple TSS were present, choose one at random. This decision tree worked for all genes, resulting in 1,179 TSS. After filtering for TSS sites on canonical chromosomes (1-22, X, Y), 1,146 genes were present containing 57 oncogenes, 108 tumour-suppressors, and 30 ambivalent genes. We used somatic, exonic single nucleotide mutation (SNVs) calls from the TCGA (22). Gene names, if necessary, were replaced by ensembl96 (Hunt et al., 2018, Yates et al., 2020) gene names using biomaRt (Durinck et al., 2009 and 2005; R package, v2.42.1) to reach consensus names across multiple data sources. The only gene from our list of 1,146 key genes that was not present was TERC, which is a long non-coding RNAs. We filtered the set of SNVS mutations for synonymous and ambivalent SNVs defined by having received the following variant classification: Silent, Intron, 3′Flank, 3′UTR, 5′Flank, 5′UTR. In addition, we only kept SNVs that passed the TCGA filter criteria. Samples with known hypermutation status have been removed in accordance with previous publications (Bailey et al., 2018). The set of SNVs did not include germline mutations which had been removed by the NCI Genomics Data Commons (GDC) because of potential issues around patient identification. Germline mutations for BRCA1 and BRCA2, as well as promoter methylations for BRCA1 and RAD51C, were obtained from other TCGA publications (see next section). For evaluating the copy number state of the 1,146 key genes, we used the same copy number profiles as we did for deriving the CIN signatures. The copy number at the transcriptional start site (TSS) was taken as a surrogate copy number for the whole gene. For each TSS in every sample, we evaluated whether a loss-of-heterozygosity (LOH), and a deletion (DEL) or amplification (AMP) was present. LOH was defined as a state where the b-allele (the allele with the minor copy number) had a copy number of less than 0.4. For deletions the same threshold of 0.4 was applied. Amplifications were called in cases with copy numbers over 5 copies. Both deletions and amplifications were checked with RNA expression data which were obtained from the Catalogue of Somatic Mutations in Cancer (COSMIC) website (2). Again names of the genes were updated to ensemble (96) as described above. Only if an overexpression in the case of oncogenes or under-expression in all other cases matched with an amplification, respectively deletion, was this status kept and moved forward in the analysis.

Identification of samples with germline mutations or promoter hypermethylation in BRCA1, BRCA2 and RAD51C. We obtained BRCA 1/2 mutation status from two studies: Maxwell et al. 2017 identified 100 BRCA and OV samples with a germline BRCA1/2 mutant (37 breast cancer, 63 ovarian); and Wang et al. 2017 identified 99 ovarian samples, 41 germline and 58 somatic samples. 149 of these 161 showed detectable levels of CIN. Of those, 139 samples also harboured a loss of heterozygosity (LOH) of either gene. LOH was defined as a state where the b-allele (the allele with the minor copy number) had a copy number of less than 0.4. Thirteen TCGA ovarian samples in which RAD51C was epigenetically silenced by promoter hypermethylation were identified by the TCGA Research Network (Cancer Genome Atlas Research Network, 2011), who measured methylation at CpG dinucleotides by using an Illumina GoldenGate methylation assay following the protocols from the TCGA Research Network (Cancer Genome Atlas Research Network, 2008). Testing for differences in signature activity. Samples were classified as mutant using the following criteria: for oncogenes, only amplifications and point mutations that did not result in a frame shift were considered mutant; for tumour-suppressor genes, we used deletions and all types of point mutations. Amplifications and deletions were only used when they were corroborated by up- or downregulated gene expression. For BRCA1 we added two extra comparisons where we evaluated whether a potential mutation was rescued or not. Rescue was defined by a mutation in either TB53BP1, RIF1, or MAD2L2 (Boersma et al., 2015, Isono et al., 2017). Not rescued was defined by having no mutation in any of these three genes. As we had prior knowledge about germline mutation and promoter methylation for BRCA1, BRCA2, and RAD51C, we treated these samples as mutated. To test for differences in signature activity between samples with driver mutations and without a mutation in one of the 1, 146 key genes, we scaled and centered the activities per signature (Z-scores). We split the samples into two groups, one group with a mutation and one without. Then we used Welch's t-test to perform a test of equality of means between the two groups. We corrected the resulting 19,006 p-values for the false discovery rate (Benjamini & Hochberg, 1995). Using an alpha of 0.05, we identified 788 genes with at least one significant correlation with the 17 signatures. Of those, 74 genes showed positive associations generating 182 significant correlations with the 17 signatures. We only regarded positive associations in order to avoid compositional data effects.

Selection of high-confidence genes. To ensure that we only regard genes with high confidence when proposing a putative cause, we further filtered the 74 genes down to 32 by applying two additional criteria: q-value <0.005 and effect size >+0.4. The following 32 genes were identified as high-confidence genes: AKT1, BRCA1, BRCA2, CCND1, CDK4, CDKN2C, CIC, CUL1, ERBB2, ERBB3, ERCC2, FBXW7, H3F3A, IDH1, IDH2, MAPK1, MYC, NFE2L2, RAC1, RPL22, PBRM1, PCBP1, PIK3R2, PMS1, PPP2R1A, PSMA4, SPOP, SOS1, SOX9, TP53, U2AF1, VHL. We studied the literature around those genes and collated information on how they are known to influence genomic stability.

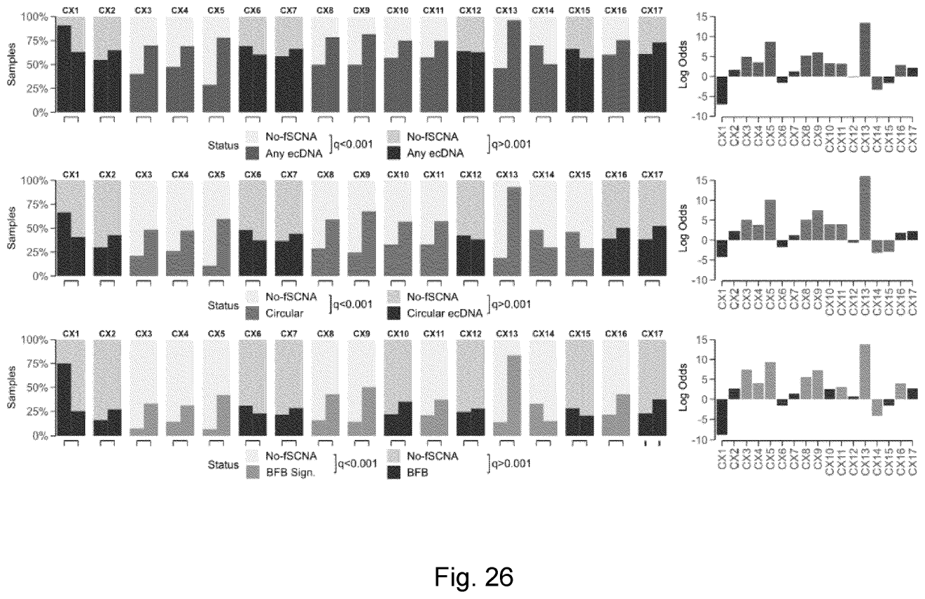

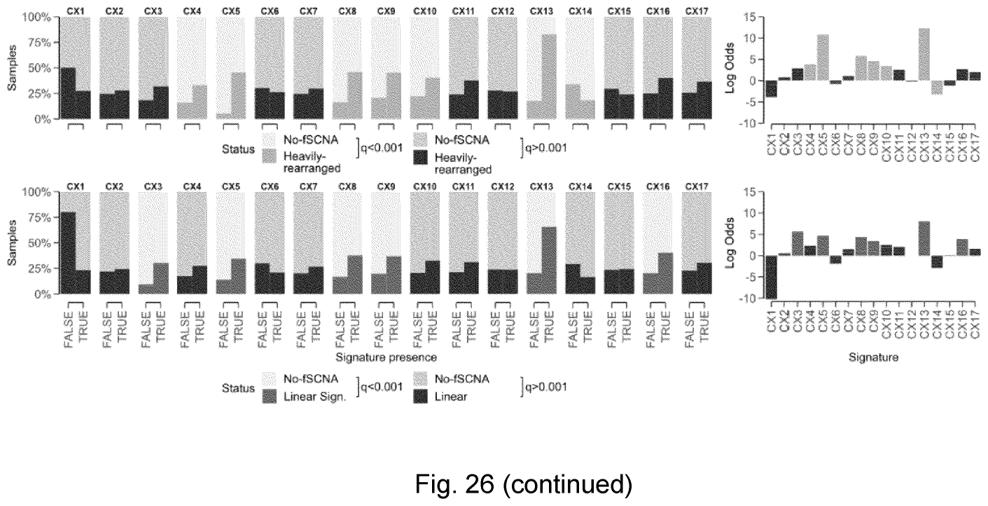

Enrichment in extrachromosomal DNA amplicons. For the three signatures (CX8, CX9, CX13) that showed a pattern of amplification (changepoint above 3 copies, FIG. 8), we studied their relationship with extrachromosomal DNA amplicons. We hypothesised that genes amplified via ecDNA amplicons could be a consequence of the mutational process producing those amplicons and can therefore be not causally related to the mutational process. First, we tested for an enrichment of different ecDNA categories (Kim et al., 2020) using Fisher's exact test in samples with signature activity versus samples with no signature activity (FIG. 26). In detail, we used their Supplementary Table 3 (Kim et al., 2020) containing a list of TCGA sample hierarchically categorised by the presence of amplicons falling into four ecDNA categories (circular-breakage-fusion-bridge→heavily-rearranged→linear→no focal somatic CAN [No-fSCNA]). That means, if a sample had evidence of circular ecDNA, then it fell into the “circular” category independent of how many other categories or amplicons were present. Likewise, only if a sample had no circular ecDNA amplicons, it could have been classified as any other category. We added a fifth category, the presence of any kind of ecDNA which we called “any ecDNA”. We did test all categories and displayed the results but given the hierarchical categorisation, we only had the sample size to derive meaningful tests for the circular and any ecDNA category (FIG. 26). CX13 had by far the strongest association with “any ecDNA” and “circular ecDNA” categories. Given that this signature also had the highest changepoints (>7 and 10 copies), we associated it with ecDNA (FIG. 13b). As oncogenes are prone to be amplified by extrachromosomal DNA amplicons (Kim et al., 2020, Kumar et al., 2020), we tested for a shift in signature activities for high-confidence genes amplified in ecDNA amplicons. If found significant, then we assumed the correlation between signature activity and mutation not to be causal and we did not regard this gene for identifying a putative cause.

In detail, we used their Supplementary Table 1 (Kim et al., 2020) to identify whether high-confidence genes were part of ecDNA amplicons. For each of the high-confidence genes of the three amplification signatures, using a Welsh's t-test we tested whether samples with amplicons for that respective gene had a significantly higher signature activity than samples without amplicons. This led to an additional filtering step, resulting in the removal of association of seven genes with the three amplification signatures. For CX8 we found that amplicons including the genes SPOP, MYC, RAC1 lead to a significantly higher signature activity. For CX9 we excluded CDK4, CCND1, ERBB2 and for CX13 we excluded CDK4.

Proposing an aetiology and assigning a confidence rating. The two pieces of evidence we used for proposing aetiologies (i.e. putative causes and mechanisms) underpinning the signatures were the high-confidence genes and the pattern of change (signature definition).

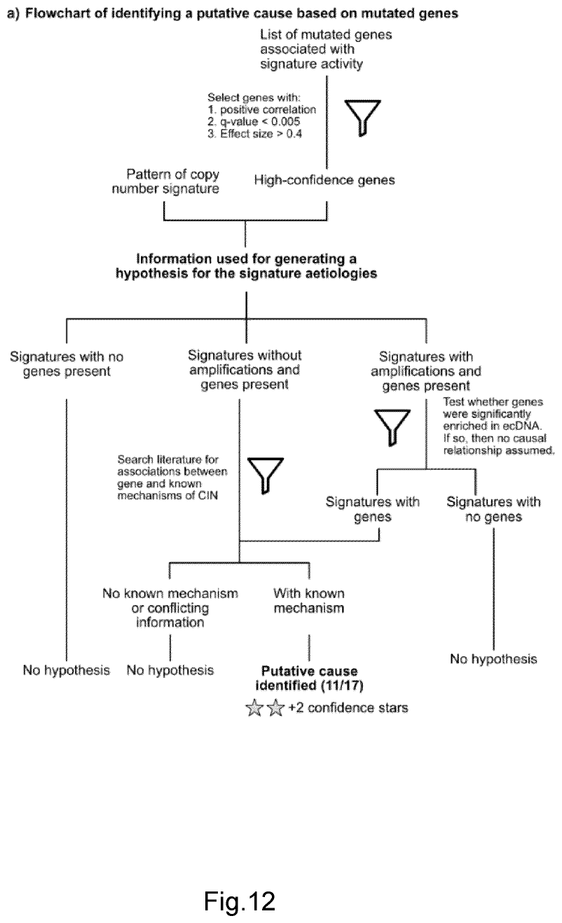

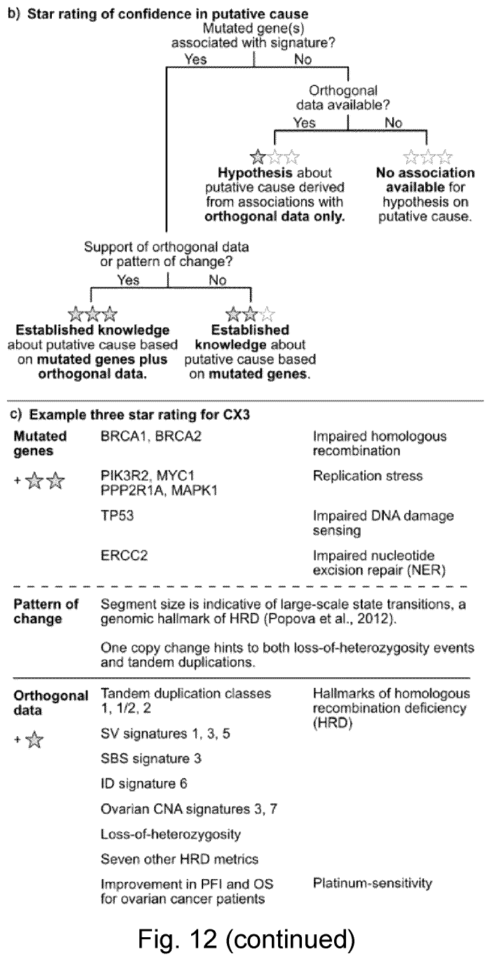

In order to show our confidence in the evidence supporting a putative cause, we devised a simple confidence rating from zero to three (FIG. 12). If high-confidence genes linked a signature to a known type of CIN, we assigned two confidence points. If there were additionally orthogonal data (i.e. SBS signature or tandem duplication association) supporting the putative cause, we added a third confidence point. Three confidence points were given to eight signatures (CX1-CX6, CX8, CX11). Two confidence points were given to three signatures (CX10, CX14, CX17) with at least one high-confidence gene linked to a known type of CIN but no additional orthogonal data were available. One confidence point was given to three signatures (CX9, CX13, CX16) where there were no high-confidence genes available but some additional orthogonal associations allowed us to propose a speculative aetiology. Zero confidence points were given to three signatures (CX7, CX12, CX15) where no gene and no orthogonal data were associated with the signature.

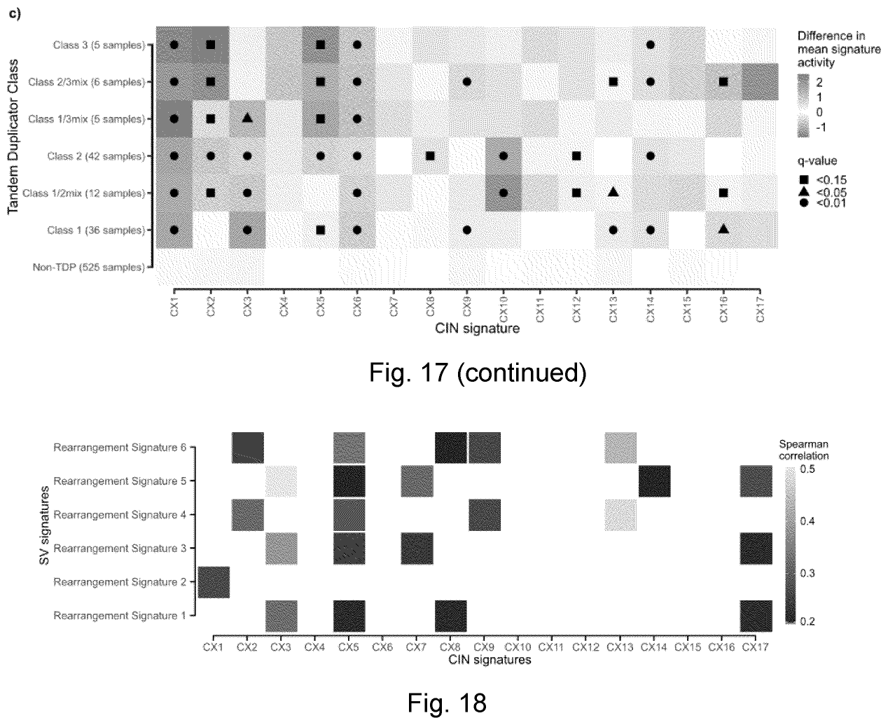

Focused analysis of CCNE1 and CDK12. There is ample evidence for the importance of CCNE1 and CDK12 mutations in cancer and chromosomal instability. CDK12 showed strong associations with CIN signatures CX2 and CX5 but the strong p-value correction (for 19,000 multiple tests) scaled their q-values to 0.06, above our q-value threshold of 0.005. However, those trends support the notion that CX2 and CX5 are associated with class 2 and 3 tandem duplications (FIG. 17). CCNE1 was not identified as a driver gene in the TCGA landmark study (Bailey et al., 2018), and therefore did not form part of the above gene enrichment analysis. We therefore performed a separate analysis for differences in signature activity in CCNE1 mutants (222 samples) and non-mutants (5003 samples). The mean signature activity for the mutants, the non-mutants, and the adjusted p-value associated with the difference in activities were: CX1: −0.8948/0.02564/3.31E-34; CX2: 0.5389; −0.02311 0.562 8.38E-06; CX3: 0.7946/−0.03026/2.29E-29; CX4: 0.775/0.01175/3.13E-08; CX5: 0.8564/0.003766/9.38E-16; CX6: −0.1438/0.04042/0.03818669; CX7: 0.08714/0.01124/0.79330761; CX8: 0.7787/0.01299/4.06E-10; CX9: 0.7212/0.01558/4.54E-10; CX10: 0.6581/0.005778/8.48E-06; CX11: 0.04352/0.01781/0.93504243; CX12: −0.01747/−0.01413/0.99471727; CX13: 0.2693/0.02006/0.00036043; CX14: −0.2866/9.25E-05/3.23E-11; CX15: −0.2824/0.002981/5.41E-12; CX16: 0.04237/−0.01184/0.45347171; CX17: 0.3179/0.008652/0.01508555.

Thus, we found that, classified as pan-cancer oncogene, CCNE1 is significantly associated with eight signatures (CX2, CX3, CX4, CX5, CX8, CX9, CX10-signatures with an effect size >0.4 and an adjusted p-value<0.005).

Correcting for cancer type when testing for driver gene associations. The above approach for testing associations between genes of interest and signature activities may be confounded by cancer type (see “Linking signature activities to mutated genes”). One example is VHL mutation, which is nearly exclusively limited to renal cancer. However, having a single tumour type enriched for a specific gene mutation and type of chromosomal instability does not mean that the association is not putatively causal (there is good support in the literature for VHL's role in regulating mitosis). Therefore, when performing this type of analysis, we faced a conundrum: an analysis without correction potentially reports confounded results, and an analysis with correction potentially excludes strong and real biology (e.g. BRCA2 is mutated in only a small number of tumour types and therefore may not pass correction). To deal with this, we opted to combine both approaches. We used an uncorrected approach to propose putative aetiologies, then used a corrected approach to see if these aetiologies continued to have support. The corrected approach tested the association between activity and mutations with a multivariate regression model correcting for cancer type. The original list of genes associated with signatures is provided below.

CX1: CDKN2C—Cell cycle checkpoint failure—Inhibitor of CDK4, controls G1 progression; VHL—supports the aetiology—Mitotic errors—Mitotic fidelity and spindle orientation; RPL22—Cell cycle checkpoint failure—Ribosomal protein activating TP53; IDH1—Proliferation—Amplification or deletions associated with increased proliferation; CIC—supports the aetiology Mitotic errors RTK/MAPK inhibition. Loss promotes mitotic errors, especially chromosome segregation effects; PBRM1—supports the aetiology—Mitotic errors—Loss may give rise to spindle checkpoint expression phenotype. Also implicated in sister chromatid and centromeric cohesion. Alternative name is BAF180 which is part of the SWI/SNF complex BAF/PBAF; SOX9—No clear role-Loss-of-function related to promotion of metastasis.

CX2: BRCA1—supports the aetiology—Impaired HR—Major role in HR and DNA replication fidelity.

CX3: BRCA1 (not rescued)—supports the aetiology-Impaired HR-Major role in HR and DNA replication fidelity. Samples with additional mutations in TP53BP1, RIF1 or MAD2L2 (also known as REV7) were ignores as they were shown to rescue BRCA1 loss; BRCA1-supports the aetiology-Impaired HR-Major role in HR and DNA replication fidelity; PMS1—Compensatory MMR, Downregulation of HR-Integral part of mismatch repair (MMR).

Upregulated it could compensate for higher SNVs due to impaired HR. MMR is also known to downregulate HR pathway. It has been suggested that MMR is involved in double-strand break repair, especially single-strand annealing (SSA); PCBP1—Alleviating replication stress-Regulates POLH which is important for translesion DNA synthesis (TLS). TLS is involved in resolving stalled forks during DNA replication. Also regulates multiple genes involved in DNA repair. Upregulation is likely to achieve a compensatory dosage effect of repair pathways; U2AF1—supports the aetiology—Replication stress (R loops)-Splicing factor contributing to cancer progression. Mutants involved in increased R loops and replication stress; BRCA2—supports the aetiology—Impaired HR—Major role in HR and DNA replication fidelity; PPP2R1A—supports the aetiology—Replication stress (Fork collapse)—Subunit of PP2. Activation of PP2 has been shown to interrupt DNA replication, resulting in fork collapses and HR activity; SOS1—supports the aetiology—Cell cycle progression-Produces protein involved in activation of Ras proteins; PIK3R2—supports the aetiology—Cell cycle progression—Regulatory subunit of PI3K, which recruits AKT and PDK1, involved in cell growth, survival; MAPK1—supports the aetiology-Cell cycle progression, Replication stress (Asymmetric forks, hyperreplication)—Essential component for progressing cell cylce, inducing mitosis, regulation of transcription etc. Causes Ras—induced CIN which is linked to replication stress by asymmetric forks and re-replication (DNA hyperreplication); NFE2L2—DNA damage response-Produces NRF2. Transcription factor. Involved in antioxidant response. Also activator of ATR. Involved in cellular response to double-strand breaks; TP53—supports the aetiology-Cell cycle progression, DNA damage response, Replication stress (Fork stalling)—Regulating the G1/S and G2/M checkpoints. Defective p53 leads to impaired HR and failed recoveries from replication fork stalling; MYC-supports the aetiology-Cell cycle progression, Replication stress (pot. fork stalling)—Known oncogene. Overexpression shown to influence cell cycle progression (G1/S transition), increase of genomic instability and replication stress. It is responsible for inducing DNA replication origins and bubbles. It is assumed fork stalling due to mechanical interaction of close replication bubbles; ERCC2—supports the aetiology—Impaired NER—Encodes a DNA helicase involved in nucleotide excision repair (NER). Many mutations are missense mutations. Most missense studied are known to inactivate NER. Mutations known to drive cisplatin sensitivity.

CX4: PIK3R2—supports the aetiology—Cell cycle progression-Regulatory subunit of PI3K, which recruits AKT and PDK1, involved in cell growth, survival; PCBP1—Alleviating replication stress—Regulates POLH which is important for translesion DNA synthesis (TLS). TLS is involved in resolving stalled forks during DNA replication. Also regulates multiple genes involved in DNA repair. Upregulation is likely to achieve a compensatory dosage effect of repair pathways; AKT1—supports the aetiology—Cell cycle progression, Mitotic errors-Known oncogene. Part of the AKT/PI3K pathways, regulating wide variety of functions. Induces asymmetric cell divisions; IDH2—No clear role-Proteins are used i.a. in metabolism. Mutants are known to suppress HR and induce sensitivity to PARP inhibitors; U2AF1—Replication stress (R loops)-Splicing factor contributing to cancer progression. Mutants involved in increased R loops and replication stress; PPP2R1A—Replication stress (Fork collapse)—Subunit of PP2. Activation of PP2 has been shown to interrupt DNA replication, resulting in fork collapses and HR activity; MAPK1—supports the aetiology—Cell cycle progression, Replication stress (Asymmetric forks, hyper-replication)—Essential component for progressing cell cycle, inducing mitosis, regulation of transcription etc. Causes Ras-induced CIN which is linked to replication stress by asymmetric forks and re-replication (DNA hyper-replication); ERCC2—Impaired NER—Encodes a DNA helicase involved in nucleotide excision repair (NER). Many mutations are missense mutations. Most missense studied are known to inactivate NER. Mutations known to drive cisplatin sensitivity; CDK4—supports the aetiology—Cell cycle progression, Mitotic errors-Regulation of cell cycle. Important for centrosome separation.

CX5 U2AF1—supports the aetiology—Replication stress (R loops)—Splicing factor contributing to cancer progression. Mutants involved in increased R loops and replication stress; CDK4—supports the aetiology-Cell cycle progression, Mitotic errors-Regulation of cell cycle. Important for centrosome separation; PCBP1—supports the aetiology-Alleviating replication stress-Regulates POLH which is important for translesion DNA synthesis (TLS). TLS is involved in resolving stalled forks during DNA replication. Also regulates multiple genes involved in DNA repair. Upregulation is likely to achieve a compensatory dosage effect of repair pathways; PMS1—supports the aetiology-Compensatory MMR, Downregulation of HR—Integral part of mismatch repair (MMR). Upregulated it could compensate for higher SNVs due to impaired HR. MMR is also known to downregulate HR pathway. It has been suggested that MMR is involved in double-strand break repair, especially single-strand annealing (SSA); MAPK1—supports the aetiology—Cell cycle progression, Replication stress (Asymmetric forks, hyperreplication)—Essential component for progressing cell cylce, inducing mitosis, regulation of transcription etc. Causes Ras-induced CIN which is linked to replication stress by asymmetric forks and re-replication (DNA hyperreplication); PPP2R1A—supports the aetiology-Replication stress (Fork collapse)-Subunit of PP2. Activation of PP2 has been shown to interrupt DNA replication, resulting in fork collapses and HR activity; BRCA1—supports the aetiology—Impaired HR-Major role in HR and DNA replication fidelity.

CX6: PSMA4—supports the aetiology-Mitotic errors (Spindle assembly checkpoint), APC and SCF Regulation—Part of the 20S proteasome, forming the 26S proteasome which activates the anaphase promoting complex (APC/C) and is implicated in the Spindle Assembly checkpoint (SAC) machinery. SCF protein complex is regulated by APC and indirectly by 26S proteasome; CUL1—supports the aetiology-Cell cycle progression, DNA damage response (NER), SCF Regulation-Part of the SCF ubiquitin ligase and promotes G1/S transition. CUL1 is known to upregulate mTORC1 which leads to cell growth. Cells can undergo tetraploidization involning activation of mTOR. Also implicated in activation of nucleotide excision repair (NER); H3F3A—supports the aetiology—Chromatin remodelling, Integrity of DNA, Mitotic errors-Core component of Histone 3.3 regulating transcription, DNA repair, DNA replication, and chromosomal stability. Histone 3.3 depletion causes dysfunction of heterochromatin structures at telomeres, centromeres, and pericentromeric regions of chromosomes, leading to mitotic defect; RAC1—supports the aetiology—Cell cycle progression, SCF Regulation, Centrosome amplification-Activates aldolase A and ERK pathways. Associated with poor outcome. Overexpressed RAC1 activates MAPK in cell lines. Regulated by SCF protein complex. Overexpression known to correlate with centrosome amplification. Prominent role of RAC1 in upregulating R5P synthesis and nucleoside metabolism, which is involved in DNA damage repair.

CX8: U2AF1—supports the aetiology—Replication stress (R loops)—Splicing factor contributing to cancer progression. Mutants involved in increased R loops and replication stress; PCBP1—supports the aetiology—Alleviating replication stress—Regulates POLH which is important for translesion DNA synthesis (TLS). TLS is involved in resolving stalled forks during DNA replication. Also regulates multiple genes involved in DNA repair. Upregulation is likely to achieve a compensatory dosage effect of repair pathways; PIK3R2—supports the aetiology—Cell cycle progression-Regulatory subunit of PI3K, which recruits AKT and PDK1, involved in cell growth, survival; PMS1—Compensatory MMR Downregulation of HR-Integral part of mismatch repair (MMR). Upregulated it could compensate for higher SNVs due to impaired HR. MMR is also known to downregulate HR pathway. It has been suggested that MMR is involved in double-strand break repair, especially single-strand annealing (SSA); ERCC2—Impaired NER-Encodes a DNA helicase involved in nucleotide excision repair (NER). Many mutations are missense mutations. Most missense studied are known to inactivate NER. Mutations known to drive cisplatin sensitivity; AKT1—supports the aetiology—Cell cycle progression, Mitotic errors-Known oncogene. Part of the AKT/PI3K pathways, regulating wide variety of functions. Induces asymmetric cell divisions; PPP2R1A—supports the aetiology-Replication stress (Fork collapse)—Subunit of PP2. Activation of PP2 has been shown to interrupt DNA replication, resulting in fork collapses and HR activity; ERBB3—supports the aetiology—Cell cycle progression—Dimerises with ERBB2 and can activate both MAPK and AKT/PI3K pathways; MAPK1-supports the aetiology-Cell cycle progression, Replication stress, (Asymmetric forks, hyper-replication)-Essential component for progressing cell cylce, inducing mitosis, regulation of transcription etc. Causes Ras-induced CIN which is linked to replication stress by asymmetric forks and re-replication (DNA hyper-replication); CDK4—supports the aetiology-Cell cycle progression, Mitotic errors—Regulation of cell cycle. Important for centrosome separation; SPOP—Not taken into account due to their significant enrichment in ecDNA amplicons which we believe results in a spurious correlation with amplification-signatures; MYC—Not taken into account due to their significant enrichment in ecDNA amplicons which we believe results in a spurious correlation with amplification-signatures; RAC1—Not taken into account due to their significant enrichment in ecDNA amplicons which we believe results in a spurious correlation with amplification-signatures.

CX9: CDK4—Not taken into account due to their significant enrichment in ecDNA amplicons which we believe results in a spurious correlation with amplification-signatures; CCND1—Not taken into account due to their significant enrichment in ecDNA amplicons which we believe results in a spurious correlation with amplification-signatures; ERBB2—Not taken into account due to their significant enrichment in ecDNA amplicons which we believe results in a spurious correlation with amplification-signatures.

CX10: PPP2R1A—supports the aetiology-Replication stress (Fork collapse)—Subunit of PP2. Activation of PP2 has been shown to interrupt DNA replication, resulting in fork collapses and HR activity; FBXW7—supports the aetiology—Impaired NHEJ—Known tumour-suppressor. Part of the ubiquitin protein ligase complex SCF. Has been shown to facilitates NHEJ; CDK4—supports the aetiology—Cell cycle progression, Mitotic errors-Regulation of cell cycle. Important for centrosome separation; ERBB2—Cell cycle progression-ERBB2, or HER2, is involved in cell-cycle progression and mitosis. ERBB2 signalling is shown to initiate CIN and defective cell-cycle control in certain breast cancer models.

CX11: CDK4—supports the aetiology—Cell cycle progression, Mitotic errors—Regulation of cell cycle. Important for centrosome separation.

CX13: CDK4—Not taken into account due to their significant enrichment in ecDNA amplicons which we believe results in a spurious correlation with amplification-signatures.

CX14: CIC—supports the aetiology-Mitotic errors RTK/MAPK inhibition. Loss promotes mitotic errors, especially chromosome segregation effects.

CX17: U2AF1—supports the aetiology-Replication stress (R loops)—Splicing factor contributing to cancer progression. Mutants involved in increased R loops and replication stress; PMS1—Compensatory MMR, Downregulation of HR-Integral part of mismatch repair (MMR). Upregulated it could compensate for higher SNVs due to impaired HR. MMR is also known to downregulate HR pathway. It has been suggested that MMR is involved in double-strand break repair, especially single-strand annealing (SSA).

Nearly all genes in the uncorrected analysis (n=24/32) appeared in the corrected list. The following gene/signature associations appeared in the original list, but did not appear in the corrected list: CX1: IDH1, CDKN2C, SOX9, PBRM1, VHL; CX3: NFE2L2, PIK3R2, PPP2R1A; CX10: PPP2R1A. These associations have the potential to be confounded and therefore aetiologies based on these need to be treated with caution. Of these, the only ones that pose significant concern are VHL and PBRM1, as these are used to link CX1 with mitotic errors. However, this concern is mitigated by the fact that in the corrected list, tumours with inactivating mutations in CDH1, a key regulator of mitosis, are significantly higher in CX1 activity, supporting the putative aetiology of mitotic errors. These results demonstrate that our proposed aetiologies, which use driver gene mutation associations, are robust to potential confounding effects of tumour type.

Supporting aetiologies by orthogonal data. In order to corroborate the putative causes we used an array of orthogonal data: two additional patient cohorts and their clinical metadata, (˜1,900 patients from the PCAWG project, ˜400 patients from the ICGC project); 5 types of mutational signatures, (SBS, indel, DBS, ovarian copy number, rearrangement); 14 molecular features (somatic point mutations, gene expression, cell cycle score, aneuploidy score, whole-chromosome CNAs, SCNA levels, tandem duplications, loss of heterozygosity (LOH), chromothripsis, kataegis, whole-genome duplication status, telomere length and elongation machinery activity, ecDNA, centrosome amplifications); and 11 DNA repair specific features (germline BRCA1 and BRCA2 mutations, BRCA1 and RAD51C hypermethylation data, HRDetect response, Myriad myChoice score, TP53 inactivation score, telomeric imbalances score, large-scale state transition score, LOH score, DNA repair proficiency score, protein expression score for 23 DNA-damage repair genes, PCAWG structural variant with associated microhomologies). The sources of the data are listed in Table 1. These data were processed in the following way.

Chromothripsis. The detection of chromothripsis events was performed for the PCAWG samples 12. We only used events that received “Chromothripsis” as a final call. We counted the number of chromothriptic events for the 410 samples with signature activities and tested the Spearman correlation to the signature activities of the 17 CIN signatures. Multiple testing correction was performed according to Benjamini and Hochberg.

Kataegis. The detection of kataegis events was performed for the PCAWG samples (ICGC/TCGA Pan-Cancer Analysis of Whole GenomesConsortium, 2020). For 498 samples we had with signature activities. We reduced the list of detected kataegis events to those with a significant adjusted p-value. We counted the number of kataegic events per sample and tested the Spearman correlation to the activities of the 17 signatures of CIN. Multiple testing correction was performed according to Benjamini and Hochberg.

Loss of heterozygosity (LOH). Loss of heterozygosity (LOH) segments were obtained from our copy number profiles. LOH was defined as a state where the b-allele (the allele with the minor copy number) had a copy number of less than 0.4. We calculated the proportion of the genome affected by LOH by using the hg19 chromosome sizes. For each signature, we tested the Spearman correlation between this proportion of LOH and signature activity. All p-values have been corrected for multiple testing using the Benjamini-Hochberg method.

SBS, DBS and ID signatures. Signature exposures to single base substitutions (SBS), doublet base substitutions (DBS) and small insertion and deletion (ID) signatures were created in Alexandrov et al. 2020 and were downloaded from Synapse.org. We used signature exposures for both Signature Profiler (SP) and Signature Analyzer (SA). Spearman correlations between SBS, DBS and ID signatures, and the CIN signature activities were performed for the 1900 PCAWG samples. P-values were corrected for multiple testing using the Benjamini-Hochberg method for each type of signature (SBS, DBS, ID) and software (SP, SA).

Potential correlation with age for CX1. CX1 correlated positively with the “clock-like” SBS1 signature suggesting these errors might also be mediated via a natural ageing process. Therefore, we tested the correlation between CX1 and the age of patients by using a linear as well as a robust linear model correcting for cancer type (R package MASS96, v7.3_51.5; R core package, v.3.6.1). Pan-cancer, we found no significant relationship (linear model: p-value for CX1 and age 0.81; robust linear model: p-value 0.88). This result is in line with previous publications which found very few CNAs genome-wide in normal tissue, suggesting negative selection. However, we performed the same analysis for each cancer type according to the method of Alexandrov et al. (2020) and found that for ovarian and lung squamous cell cancer, there was a positive correlation between age and number of estimated CNAs for CX1 (OV: F-statistic=5.9, q-value=0.048; LUSC: F-statistic=6.8, q-value=0.044). Given that ovarian cancer has one of the longest latencies (over 20 years), it might be that extreme latency periods lead to an accumulation of mitotic errors.

Whole-chromosome CNAs. The smallest canonical human chromosome in the hg19 reference genome is chromosome 22 with 48 Mb. However, SNP 6.0 arrays do not fully cover the whole range of chromosomes. The resulting actual coverage of chromosomes shrunk to 35.2 Mb for chromosome 22 and 37.4 for chromosome 21. We counted a segment as a whole chromosome CNAs if it was larger than 95% of the whole chromosome length covered by SNP6 arrays and had a copy number value greater or smaller than 2. For each signature, we tested the Spearman correlation between counts of whole chromosome CNAs and signature activity. P-values were corrected for multiple testing using the Benjamini-Hochberg method.

Whole-genome duplication. To test whether the whole-genome duplication status associated positively, negatively, or independently with the activity of a signature, we performed two tests for each signature: a proportion test at zero activity for the proportion of WGD samples compared to the proportion of WGD samples across the 6,335 samples, and a Welsh's t-test for samples with activities higher than zero. Only if a signature showed both significantly depleted levels at zero and a significant positive shift in non-zero activities, we positively associated that signature with WGD. If a signature had both an enriched proportion of WGD samples at zero and a significant negative shift in non-zero activities, then we negatively associated that signature with WGD. Otherwise, we classified the signature as independent. P-values were corrected for multiple testing using the Benjamini-Hochberg method.

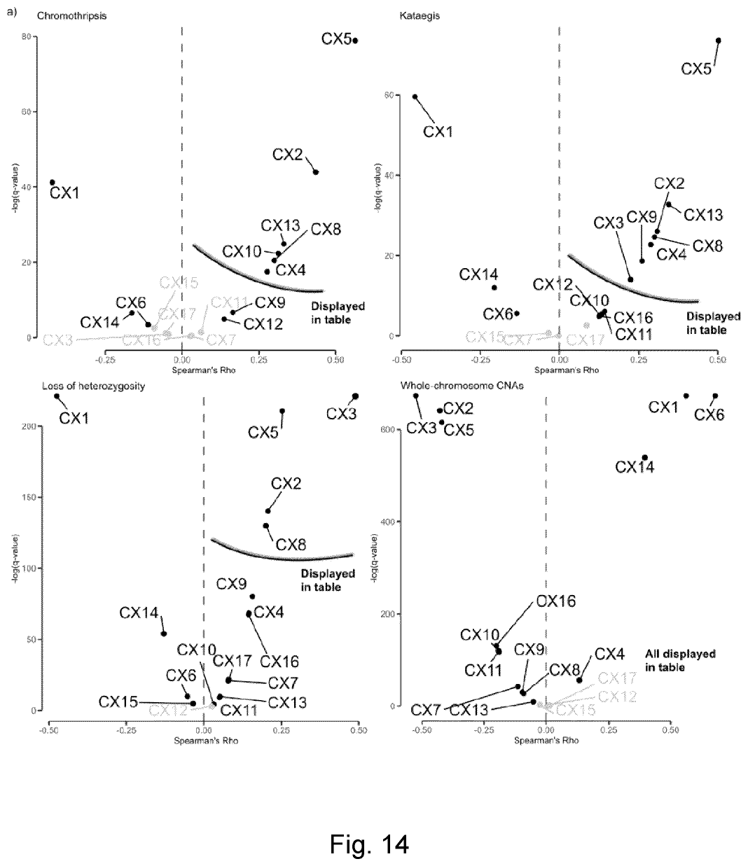

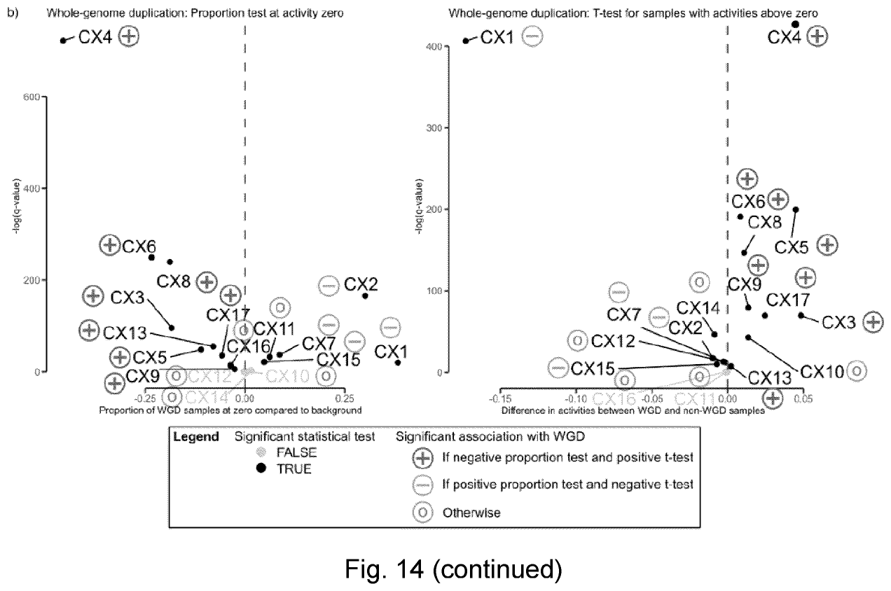

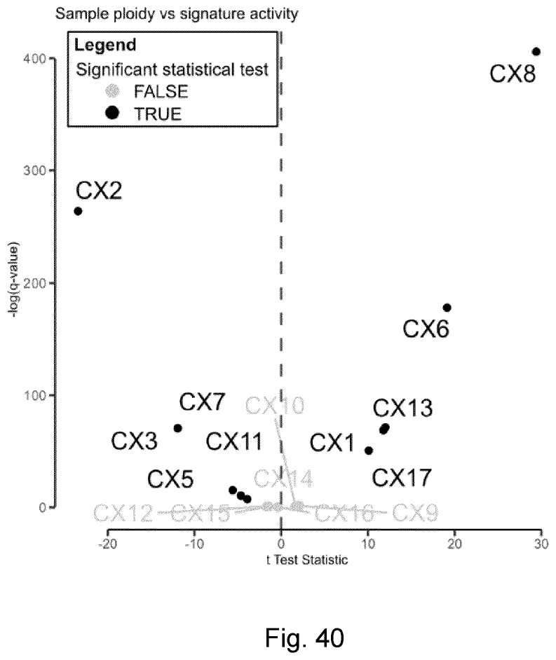

Identification of signatures related to ploidy changes. There are generally two ways a tumour cell can change its ploidy status (where ploidy is measured as the average copy number state observed across the genome): 1) whole-genome duplication which increases the ploidy status by two; or 2) an accumulation of copy number gains which increases the overall ploidy status (similarly for accumulation of losses which reduce the ploidy status). Number 1) is represented by CX4 (FIG. 14). Number 2) is not directly read out by our signatures. Therefore, we tested if any signature might be linked to a general shift in ploidy status. By using the function Im74 (R core package, v.3.6.1), we performed a multivariate regression analysis correlating signature activity with tumour ploidy and correcting for CX4 activity and WGD status (FIG. 40). This analysis shows that CX6 and CX8 seem to be linked to an overall increase in ploidy state, whereas CX2 to a decrease. This is consistent with the proposed aetiologies.

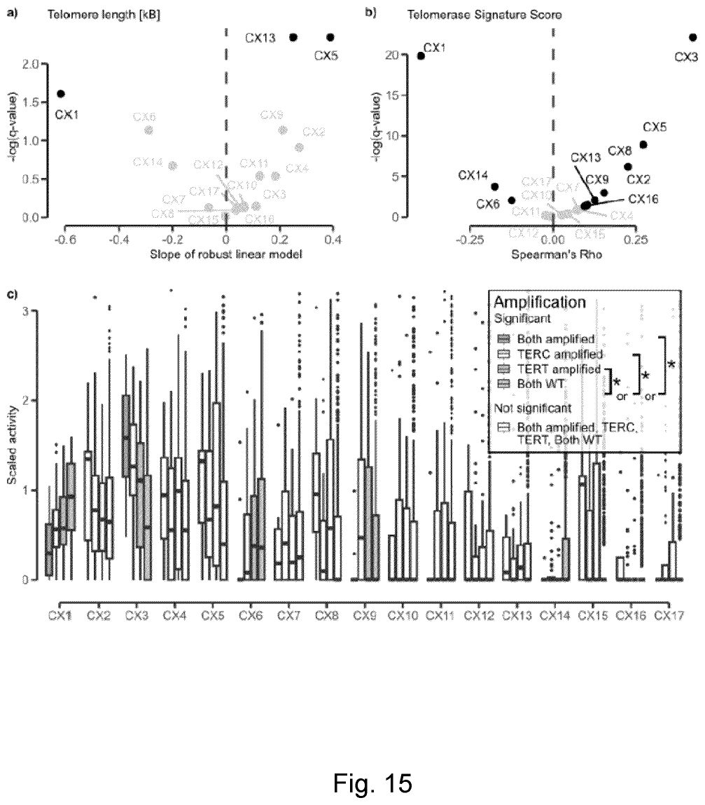

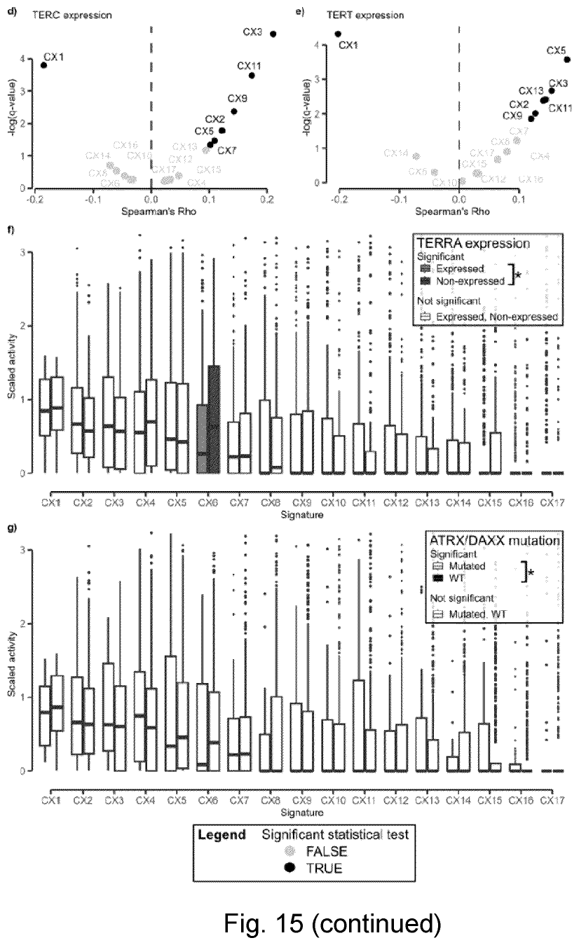

Telomerase length and activity of telomere elongation machinery. Telomere length data were obtained from Barthel et al. 2017. The authors estimated telomere length with TelSeq89 for TCGA samples. We removed estimates for samples based on shallow whole-genome sequencing (sWGS) and whole-exome sequencing (WXS) because we could not evaluate how well telomere length estimates were from off-target reads. A supplementary figure in Barthel et al. shows significant differences between sequencing technologies. We therefore focused on estimates derived from whole-genome sequenced samples (WGS). The supplied telomere length ratios were converted to lengths in base pairs. Tumour telomere lengths were subtracted by control telomere lengths. Positive values indicate longer telomere lengths in tumours. For each signature, a robust linear regression model (R package MASS Venables & Ripley 2002, v7.3_51.5) was fitted to the telomere lengths and signature activities. An F-test to test whether the slope of the robust linear model was different from zero was performed with sfsmisc (Maechler, 2020 R package, v1.1-7). The resulting 17 p-values were corrected for the false discovery rate according to Benjamini and Hochberg (FIG. 15a).

Telomerase signature scores (TSS), a gene expression score to assess the activity of the telomere elongation machinery, were obtained from Barthel et al., For each signature, we tested the Spearman correlation between TSS and signature activity. P-values have been corrected for multiple testing using the Benjamini-Hochberg method (FIG. 15b). Amplification status for two important genes of the telomere elongation machinery, TERT and TERC, were obtained from Bartel et al., We categorised the samples into four groups: both wild type, TERT amplified only, TERC amplified only, both amplified. For each of the three non-wild type groups, we tested a difference in signature activity to the wild type group with a Welsh's t-test. P-values have been corrected for multiple testing using the Benjamini-Hochberg method (FIG. 15c). Gene expression values for TERT and TERC, were obtained from Bartel et al., For each signature, we tested the Spearman correlation to both TERT and TERC gene expression. P-values have been corrected for multiple testing using the Benjamini-Hochberg method (FIG. 15d-e). The expression status of TERRA, a long non-coding RNA transcribed from telomeres, was also obtained from Bartel et al. Using Welsh's t-test for the binary expression status and signature activities for the 17 CIN signatures, we found a significant depletion of TERRA expression for CX6. TERRA is known to inhibit the elongation of telomeres but is also known to limit genomic instability by protecting telomeres hinting at a more complex picture (Luke & Lingner, 2009, Oliva-Rico Herrera, 2017). P-values have been corrected for multiple testing using the Benjamini-Hochberg method (FIG. 14f). In addition to the telomere elongation machinery, cells utilise a second pathway for telomere elongation called alternative telomere lengthening (ATL) with the chromatin remodeler ATRX and DAXX being key players. A deactivating mutation in those genes is generally associated with an activation of the ATL pathway (Heaphy et al., 2011, Dyer et al., 2017). The mutation status of ATRX and DAXX was obtained from Barthel et al. Welsh's t-tests with the signature activities found no significant difference. P-values have been corrected for multiple testing using the Benjamini-Hochberg method (FIG. 15g).

Tandem duplications. Number of tandem duplication events, tandem duplication score and Tandem Duplicator Class categorisation were sourced from Menghi et al. For each signature, we tested the Spearman correlation between the number of tandem duplication events, the tandem duplication score and signature activity. P-values were corrected for multiple testing using the Benjamini-Hochberg method (FIG. 17a-b). The Tandem Duplicator Class split the samples into a non-TDP group and six different TDP classes (1, 2, 3, ⅓, ½, ⅔). The activities of the non-TDP group were compared to the activities of each of the TDP classes using Welsh's t-test. P-values have been corrected for multiple testing using the Benjamini-Hochberg method (FIG. 17c).

Cell cycle score (CCS). The Cell cycle scores (CCS), an additive gene expression score implying the speed of cancer cells going through the cell cycle, for 5466 samples were obtained from Lundberg et al., We used the same 3 categories as the authors: low, medium (intermediary), high. The signature activities of the high CCS group were compared to the activities of the low and medium group using Welsh's t-test. P-values have been corrected for multiple testing using the Benjamini-Hochberg method (FIG. 25). We only associated a signature with an increased cell cycle if both the low to medium and the medium to high group comparison were significant.

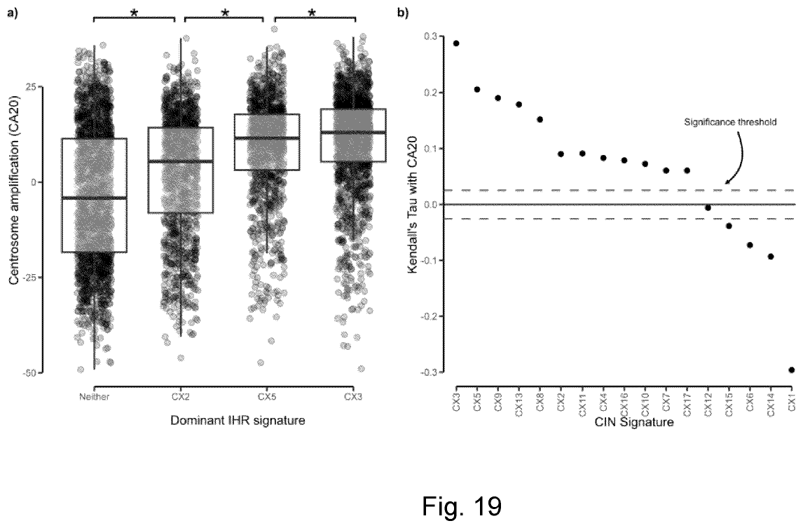

Centrosome amplification (CA20). The CA20 score, an additive gene expression score of 20 genes experimentally proven to be involved with centrosome amplification, for 5437 samples were obtained from Almeida et al. For each signature, we tested the Spearman correlation between the CA20 score and signature activity. P-values were corrected for multiple testing using the Benjamini-Hochberg method (FIG. 19b). We also split the 5437 samples into four groups depending on their dominant impaired HR group. A IHR signature (CX2, CX3, CX5) was dominant in a sample when the scaled z-score was higher than 1.25 and higher than the other IHR signatures. Samples with no dominant IHR signature were put in a “Neither” category. Welsh's t-test confirmed an increasing CA20 score from the Neither group to the CX2-dominant, CX5-dominant and CX3-dominant group. P-values were corrected for multiple testing using the Benjamini-Hochberg method (FIG. 19a).

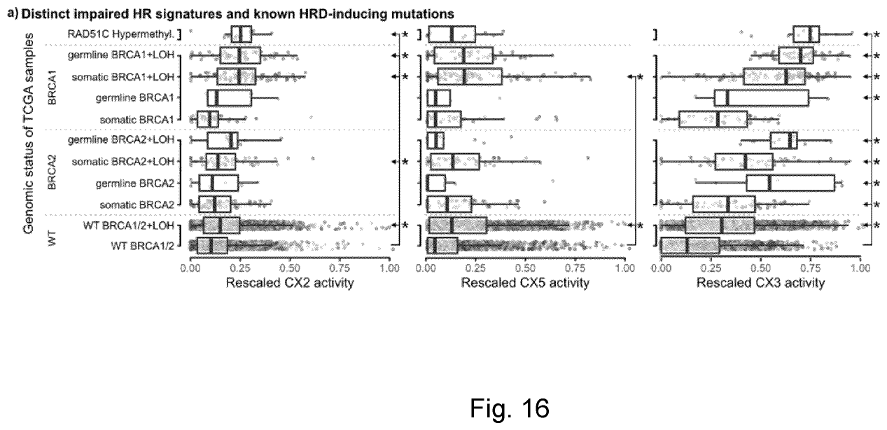

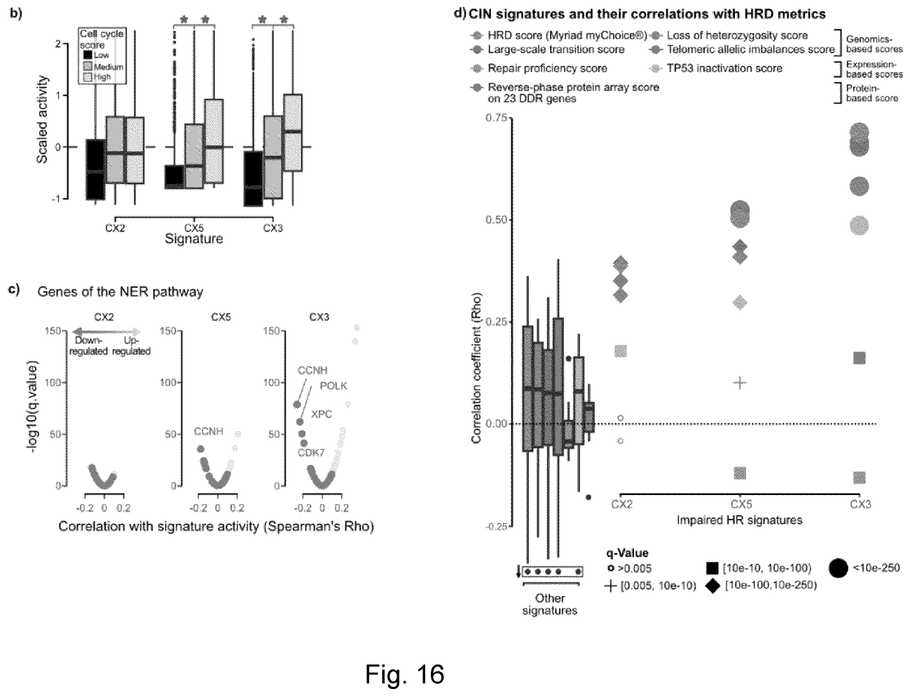

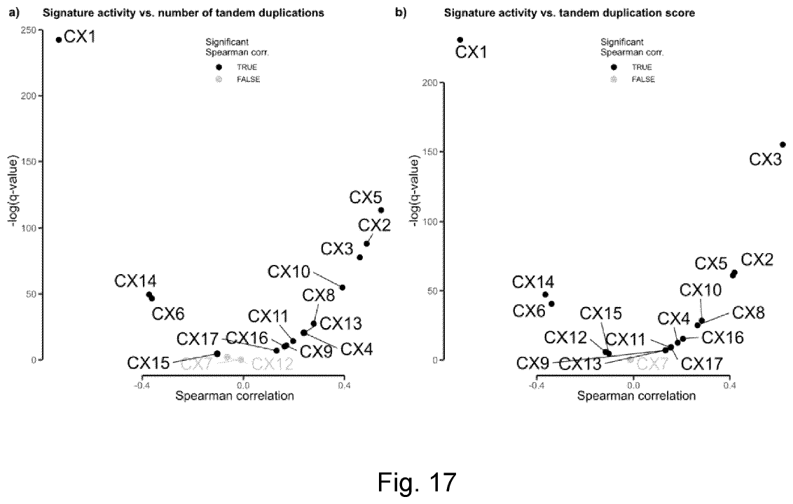

Seven HRD metrics. Sourced from Knijnenburg et al., pre-processed by Jordan Griffin (Gerke lab) and published in this Github repository (https://github.com/GerkeLab/TCGAhrd), we used the following seven metrics known to correlate with homologous recombination deficiency (HRD): Myriad myChoice score (also called HRD score-Telli et al., 2016), TP53 inactivation score (Knijnenburg et al., 2018, More information in this github repository: https://github.com/greenelab/pancancer), telomeric imbalances score (Birkbak et al., 2012), large-scale state transition score (Popova et al., 2012), LOH score (Abkevich e al., 2012), DNA repair proficiency score (RPS, Pitroda et al., 2014), protein expression score for 23 DNA-damage repair genes (Knijnenburg et al., 2018). For each signature, we tested the Spearman correlation between the seven metrics and signature activity. P-values for each metric were corrected for multiple testing using the Benjamini-Hochberg method (FIG. 16b).

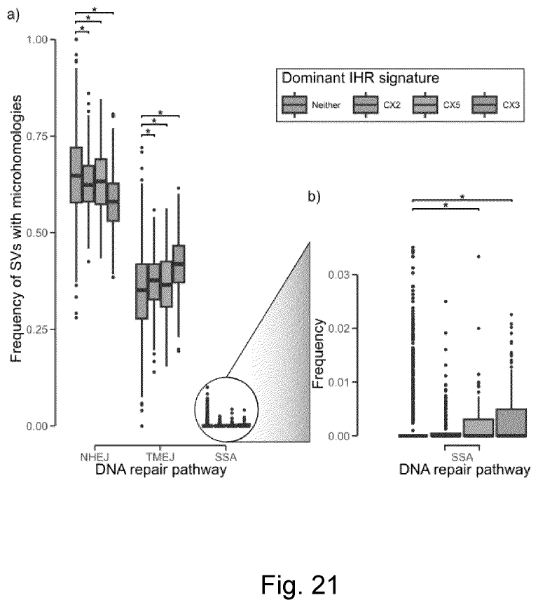

Microhomologies of PCAWG structural variants. From the structural variant (SV) data of the PCAWG project (ICGC/TCGA Pan-Cancer Analysis of Whole Genomes Consortium, 2020), we extracted the length of microhomologies. Two columns were used: Homlen (length of homology) and homseq (sequence of homology). If the homlen column had a NA entry and the homseq column showed “ ” [sic], we interpreted this as a length of zero. Structural variants with NAs in both columns were ignored. SVs with 0-1 bp microhomologies were assigned to the nonhomologous end joining (NHEJ) pathway, SVs with 2-20 bp to the theta-mediated end-joining (TMEJ) and SVs with longer stretches of microhomologies were assigned to the single strand annealing (SSA) pathway (Stok et al., 2021, Schimmel et al., 2019). We performed two analyses: One with a focus on samples with a dominant impaired HR signature activity (FIG. 21) and a broad comparison of signature activity and DNA repair pathway (FIG. 24). For the former analysis, we split the 1643 PCAWG samples into four groups depending on their dominant impaired HR group. A IHR signature (CX2, CX3, CX5) was dominant in a sample when the scaled z-score was higher than 1.25 and higher than the other IHR signatures. Samples with no dominant IHR signature were put in a “Neither” category. Welsh's t-test confirmed a decrease in NHEJ contribution going from the Neither group to the CX2- and CX5-dominant and again to the CX3-dominant group. P-values were corrected for multiple testing using the Benjamini-Hochberg method (FIG. 21a). SSA showed an increase from the Neither group to CX5- and CX3-dominant groups (FIG. 21b). For the latter analysis, we split samples into three groups based on their scaled signature activity: depleted (<0), intermediary (0-1.25) and dominant (>1.25). For each signature and repair pathway (NHEJ, TMEJ, SSA) Welsh's t-tests were performed between the depleted and the dominant sample groups. P-values were corrected for multiple testing using the Benjamini-Hochberg method (FIG. 24).

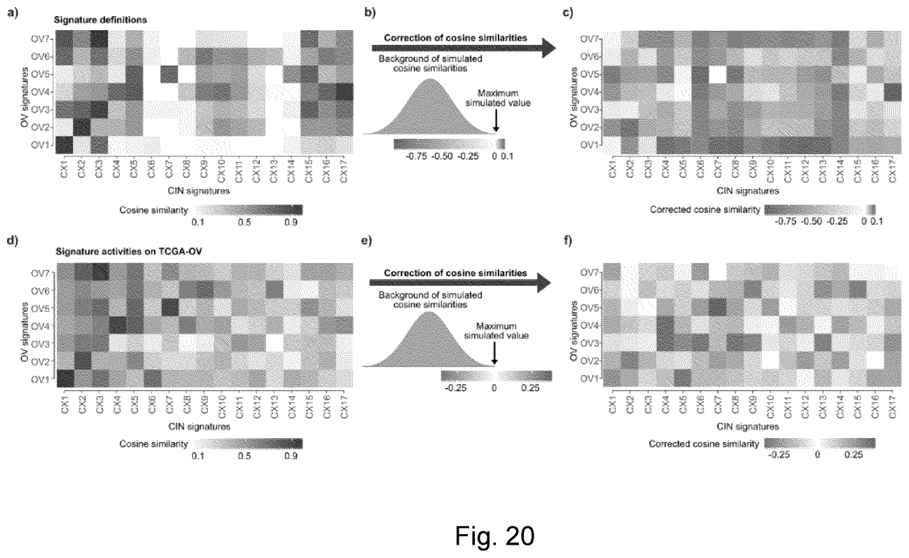

Ovarian copy number signatures. We compared the 17 CIN signatures to the previously identified 7 ovarian copy number signatures (Macintyre et al., 2018) by computing the cosine similarities of the signature definitions and signature activities called on the 545 TCGA-OV samples (FIG. 20). We excluded the copy number feature of the ovarian copy number signatures since there was no equivalent feature in the CIN signatures. We mapped the CIN signature feature components onto the ovarian signature components by taking the means of the 43 mixture components and calculating the probabilities of belonging to the mixture components of their respective feature. Using this conversion matrix, we brought the 17×43 CIN signatures to the feature space of the ovarian copy number signatures which has 17×36 dimensions. Each CIN signature was compared to every ovarian copy number signature by using cosine similarity. To test the significance we used a cosine permutation test with 10,000 permutations with the R function cosinePerm from PharmacoGx (Smirnov et al., 2016, R package, v1.17.1). Taking the maximum from the cosine similarity simulations, we corrected the observed cosine similarity. We decided for this correction to easily visualise highly significant results. For signature activities, cosine similarity, permutation testing, and cosine correction were applied in an identical manner. P-values corrected for the false discovery rate according to Benjamini and Hochberg.

Gene expression of DNA repair genes. We explored the activation of DNA repair pathways across 5,186 TCGA samples, which had extracted CIN signature activities and available gene expression data. We initially downloaded the list of genes involved in the different DNA repair pathways from the Reactome database (Fabregat et al., 2018, date of access: Nov. 5, 2021), and selected those genes differentially expressed between IHR and non-IHR samples by using a two-sided t-test (P-value cutoff of 0.01). IHR samples were those having a high activity of CX2, CX3 or CX5 signatures (threshold of scaled and centered signature activity >1.25). Then, Spearman correlations were computed between the activity of IHR signatures and the expression of DNA repair genes (Z-scores). All reported P-values have been corrected for multiple testing using the Benjamini-Hochberg method.

Testing for the influence of ethnicity. Black patients have been shown to experience higher rates of homologous recombination deficiency (HRD). We therefore explored whether the signatures differed in activity across different ethnic backgrounds as annotated by the TCGA. We found that there were substantial differences in signature activities with respect to the patients' ethnicities (FIG. 41), and that black patients have significantly higher activities in CX3 and CX5, both of which are implicated in impaired HR.

Testing the independence between CX2, CX3 and CX5 activity in BRCA1/2 mutants. We present three signatures of impaired homologous recombination (IHR): CX2, CX3 and CX5.

Their putative aetiologies were all derived in the same way, accumulating evidence from the signature pattern, gene associations and additional data. In particular, CX2 and CX5, like CX3, are not just associated with mutations in HR genes but also have copy number patterns indicative of defects in HR, which argues that they are specific markers of this process. To address the question whether CX2 and CX5 are independent forms of IHR, or are their associations driven by co-occurrence with CX3, we performed an association analysis between BRCA mutant cases and the activity of the three signatures (CX2, CX3, CX5), using a multivariate regression model which explicitly corrects for the effects of association with the other two signatures, cancer types and BRCA mutant cases. The results of this analysis are provided below, where the association between BRCA1/2 mutation status and signature activity for CX2, CX3 and CX5 was tested while correcting in each analysis for two of the signatures and cancer type. The WT BRCA1/2 was used as a benchmark against which the results were compared.
gBRCA1+LOH: CX2 (corrected for CX3 and CX5): t=2.2, p-value=0.031; CX5 (corrected for CX2 and CX3): t=3.6, p-value=0.00002; CX3 (corrected for CX2 and CX5): t=14.3, pvalue<2*10-16;
gBRCA2+LOH: CX2 (corrected for CX3 and CX5): t=−0.3, p-value=0.80; CX5 (corrected for CX2 and CX3): t=−0.2, p-value=0.81; CX3 (corrected for CX2 and CX5): t=10.8 3.0*10-09;
sBRCA1+LOH: CX2 (corrected for CX3 and CX5): t=2.9, p-value=0.00392; CX5 (corrected for CX2 and CX3): t=6.5, p-value=8.8*10-11; CX3 (corrected for CX2 and CX5): t=14.2, p-value<2*10-16;
sBRCA2+LOH: CX2 (corrected for CX3 and CX5): t=1.6, p-value=0.11; CX5 (corrected for CX2 and CX3): t=−0.2, p-value=0.85; CX3 (corrected for CX2 and CX5): t=8.7, p-value<2*10-16;
BRCA1 hyperm.: CX2 (corrected for CX3 and CX5): t=1.3, p-value=0.19; CX5 (corrected for CX2 and CX3): t=3.5, p-value=0.0004; CX3 (corrected for CX2 and CX5): t=6.2, p-value=6.6*0-10;
RAD51C hyperm.: CX2 (corrected for CX3 and CX5): t=1.1, p-value=0.28; CX5 (corrected for CX2 and CX3): t=−1.4, p-value=0.15; CX3 (corrected for CX2 and CX5): t=6.4, p-value=1.7*10-10.

The results of this analysis demonstrates that both CX2 and CX5 have significantly higher activities in BRCA mutant patients independent of CX3 activity levels and cancer type. This provides evidence that CX2 and CX5 are likely directly associated with BRCA mutant status and therefore potentially a result of impaired HR.

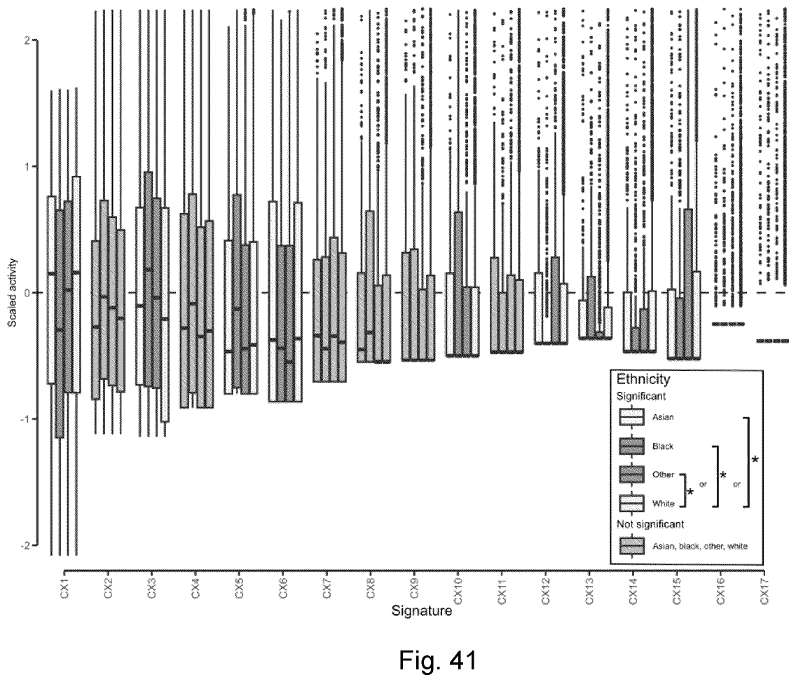

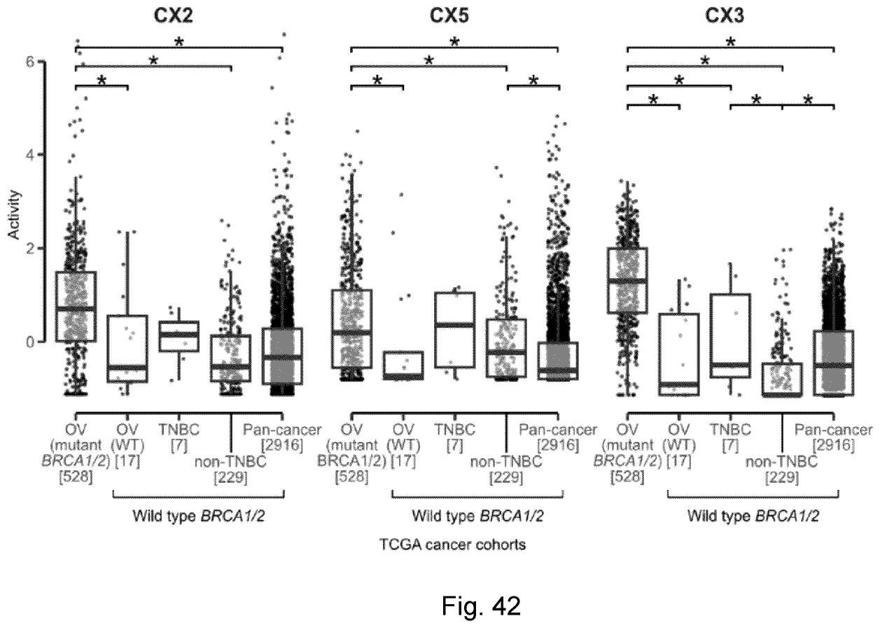

Testing for the influence of IHR signatures on triple-negative breast cancers. Triple-negative breast cancers (TNBC) are known to experience higher rates of homologous recombination deficiency (HRD). It is therefore of interest how the three impaired HR signatures (IHR, CX2, CX3, CX5) are distributed across TNBC and non-TNBC as well as to ovarian cancer samples, especially for BRCA1/2 and RAD51C wild type samples. We obtained TNBC status from Lehmann et al. and for each of the three signatures, tested differences in activities between breast (TNBC and non-TNBC) and ovarian (BRCA1/2 mutant and WT) and a control group consisting of other WT BRCA1/2 and RAD51 methylation negative samples in the TCGA cohort (FIG. 42). Sample sizes for OV WT (n=17) and TNBC (n=7) were small compared to OV mutant (n=528), non-TNBC (n=229) and control (n=2916), which limits the power of any comparison analyses. However, we do find that TNBC samples appear to have activities of CX2 and CX5 matching those of mutant ovarian cancer suggesting these may have a higher propensity to be affected by HRD than non-TNBC, which have activities similar to non-mutant OV and pan-cancer tumours.

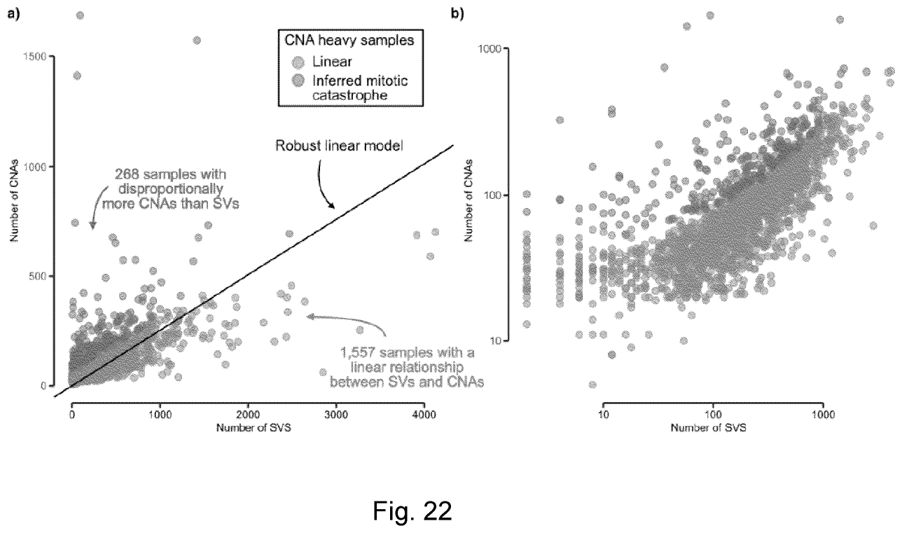

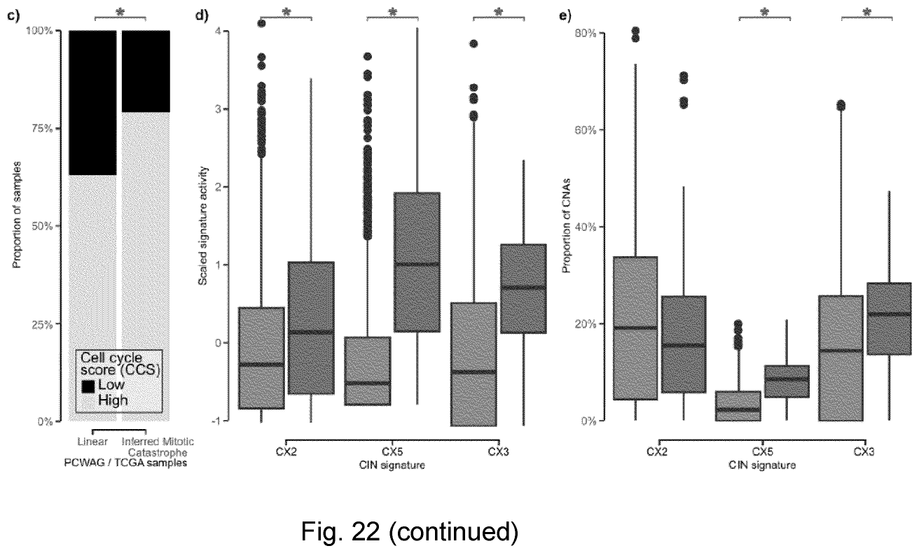

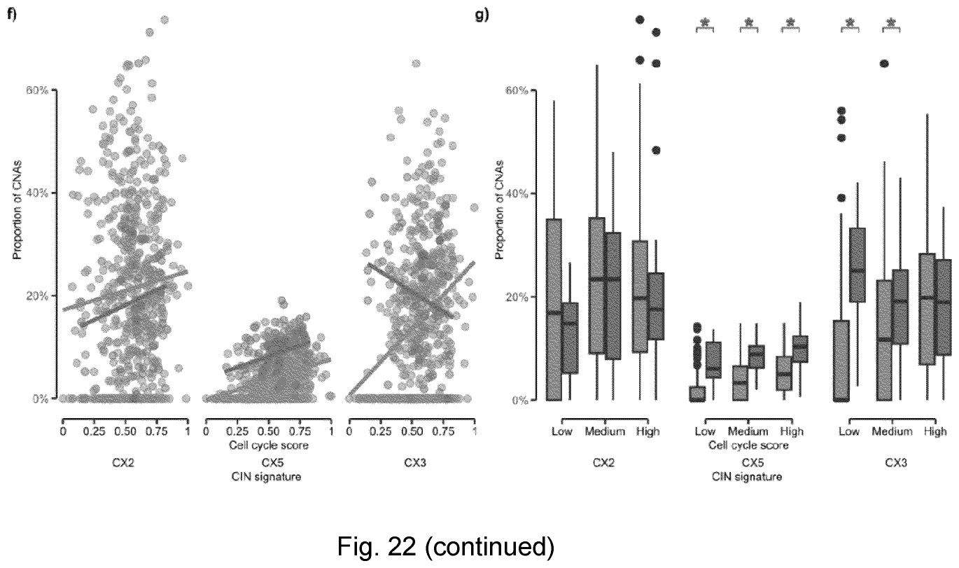

Implicating mitotic catastrophe in impaired HR. Samples with impaired HR signatures CX3 and CX5 experience additional replication stress and impaired DNA damage sensing. The more perturbed the cell cycle checkpoints and DNA repair pathways are, the easier copy number aberrations and structural variants manifest. However, we hypothesised that samples with an abundance of CNAs over SVs might accumulate those during mitosis which is influenced by how quick the cell cycle progresses indicated by replication stress and cell cycle activity and how strong double-strand break (DSB) repair mechanisms are impaired. Using the 1,898 PCAWG samples, we fitted a robust linear model to the number of CNAs and SVs. All samples with weights less than 1 did not fit well in the model and were deemed samples with inferred mitotic catastrophe (FIG. 22a-b). Indeed, in this group of samples with inferred mitotic catastrophe, high cell cycle scores (CCS) were enriched and significantly higher CX2, CX3 and CX5 contributions with a particular large effect size for CX5 and CX3 (FIG. 22c-d). Estimating the number of CNAs produced by each signature, the group of inferred mitotic samples also experienced significantly higher contributions from CX3 and CX5 (FIG. 22e). When splitting the samples by their cell cycle score (low, medium, high) and comparing these groups to the estimated number of CNAs produced by each of the IHR signatures, a fascinating picture emerges (FIG. 22f-g). CX2 shows no difference from the background (samples with no significant enrichment in CNAs over SVs). So the increase with cell cycle score is driven by not being able to repair the additional damage accumulating through a faster cell cycle. However, CX5 has constantly and significantly higher contributions to the samples with inferred mitotic catastrophe than the background, probably because a higher rate of replication stress is already present and overloading the repair systems. On the contrary, CX3 shows a negative trend for cell cycle speed. That is, cells are already maxed out in the number of CNAs they can repair due to impaired DNA damages and higher replication stress producing high rates of errors already at low cell cycle speed. Now with increasing cell cycle speed, other signatures (processes) become more active and produce more CNAs, CX3 relative contribution declines as the cells cannot tolerate more CNAs caused by CX3.

Therefore, we conclude that most of the excess CNAs produced by CX3 may be caused during the cell cycle potentially by higher replication stress but manifest during mitosis, causing a mitotic catastrophe.

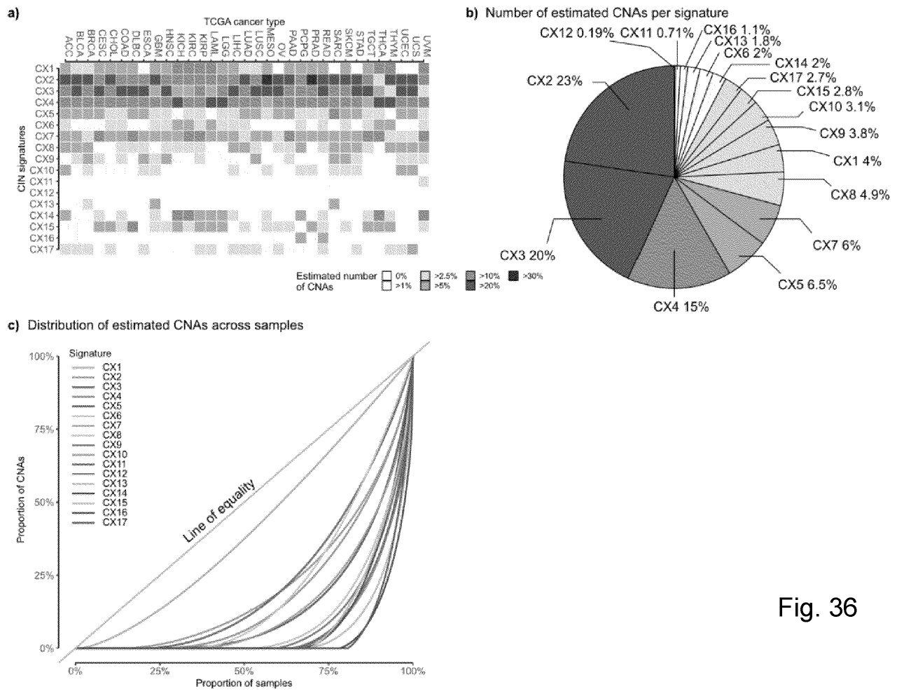

Estimation of CNAs produced by a signature. We fitted a linear model on the raw signature values to the number of CNAs in each sample. We set the intercept to zero, to only allow positive coefficients. Interaction terms between the signatures were avoided as they resulted in negative coefficients, indicating that the model overfitted. From the model we extracted the coefficients and multiplied with the raw signature values for each sample to get the estimates of CNAs produced by a signature (FIG. 36).

### Results

To determine putative causes underlying each of the 17 signatures, the inventors developed a data integration framework and assigned a confidence score to each signature aetiology based on the quality and extent of supporting data (FIG. 12). To propose aetiologies, we initially used signature definitions (i.e. the patterns of copy number change encoded by the signature, FIG. 8) and signature associations with known cancer driver mutations (FIG. 13a). We used these driver gene associations as markers for putative pathways involved in the aetiologies and assumed the same pathway deregulation for samples where no driver gene was mutated (similar to how BRCAness is defined in the absence of BRCA1/2 mutation, Lord & Ashworth, 2016). In many cases, the signature pattern was already suggestive of a mechanism (e.g. whole chromosome missegregation). Once a cause was proposed, we sought additional supporting data, including data from two additional patient cohorts and their clinical metadata (˜1,900 patients from the PCAWG project, ˜400 patients from the ICGC project); 5 types of mutational signatures (SBS, indel, DBS, ovarian copy number, rearrangement); 14 molecular features (somatic point mutations, gene expression, cell cycle score, aneuploidy score, whole-chromosome CNAs, tandem duplications, loss of heterozygosity (LOH), chromothripsis, kataegis, whole-genome duplication status, telomere length and elongation machinery activity, extrachromosomal DNA (ecDNA), centrosome amplification score (CA20)) and 11 DNA repair specific features (germline BRCA1/2 mutations, BRCA1 and RAD51C hypermethylation data, HRDetect response, Myriad myChoice score, TP53 inactivation score, telomeric imbalances score, large-scale state transition score, LOH score, DNA repair proficiency score, protein expression score for 23 DNA-damage repair genes, PCAWG structural variant with associated microhomologies). Here we provide a synthesis of the data supporting the putative aetiologies (summarized in Table 3).

TABLE 3
Aetiologies and prevalence of CIN signatures.

| | Prev. | | | |
|---|---|---|---|---|
| Sign | (%) | Putative cause | Conf | Pattern of Change |
| CX1 | 80 | Chromosome | 3 | Whole arm/chromosome changes |
| | | missegregation via | | |
| | | defective mitosis and/or | | |
| | | telomere dysfunction | | |
| CX2 | 69 | Impaired homologous | 3 | Clustered, short to medium sized |
| | | recombination (HR) | | segments |
| CX3 | 63 | Impaired HR with | 3 | Long segments with one copy |
| | | replication stress and | | change |
| | | impaired damage sensing | | |
| CX4 | 58 | PI3K/AKT-mediated | 3 | Short and long segments with two |
| | | toleration of whole- | | copies changed |
| | | genome duplication | | |
| CX5 | 56 | Impaired HR with | 3 | Clustered, medium to medium-long |
| | | replication stress | | sized segments in oscillating |
| | | | | chains |
| CX6 | 51 | Chromosome | 3 | Whole arm/chromosome changes |
| | | missegregation via | | |
| | | defective mitosis | | |
| CX7 | 50 | Unknown | 0 | Segments with small changepoints |
| CX8 | 38 | Replication stress | 3 | Low level amplifications |
| CX9 | 32 | Replication stress | 1 | Mid-level focal amplifications |
| CX10 | 29 | Impaired NHEJ with | 2 | Clustered short to medium sized |
| | | replication stress | | segments |
| CX11 | 28 | Replication stress | 3 | Clustered short segments |
| CX12 | 27 | Unknown | 0 | Short segments in long oscillating |
| | | | | chains |
| CX13 | 26 | Replication stress | 1 | High level clustered amplifications |
| CX14 | 26 | Chromosome | 2 | Whole arm/chromosome changes |
| | | missegregation via | | |
| | | defective mitosis | | |
| CX15 | 25 | Unknown | 0 | Clustered medium sized segments |
| CX16 | 17 | Unknown (Impaired DNA | 1 | Clustered short segments of two to |
| | | repair?) | | three copies |
| CX17 | 16 | Unknown (Replication | 2 | Clustered medium to medium-long |
| | | stress?) | | sized segments of one to two |
| | | | | copies |

Conf = confidence level indicated from 0 = low to 3 = high. Prev = pan cancer prevalence. Sign = signature.

Mitotic signatures: CX1, CX6 and CX14 all encoded patterns related to whole arm or whole chromosome changes and significantly correlated with direct counts of whole-chromosome changes (FIG. 14). This suggests putative causes resulting in chromosome missegregation during mitosis underlying these signatures. In agreement with this hypothesis, CX14 had significantly higher activity in tumours with inactivating mutations in CIC; CX1 with mutations in CIC, VHL and PBRM1; and CX6 with mutations in CUL1 and RAC1. CX1 and CX14 were linked with dysregulation of mitosis via significantly higher activity in tumours with inactivating mutations in CIC38,39 (FIG. 13a). CX1 was also associated with mitotic checkpoint weakening via inactivating mutations in VHL (Hell et al., 2014, Varela et al., 2011) and PBRM1 (Varela et al., 2011, Bihr et al., 43, Brownlee et al., 2014, Hodges et al., 2016). In addition to mitotic defects, structurally abnormal chromosomes can precipitate whole and partial chromosome missegregation (Pampalonat et al., 2010, Treff et al., 2011). Accordingly, significantly higher CX6 activity in tumours with CUL1 or RAC1 amplification suggested a role for impaired DNA damage response via SCF-complex dysregulation (Hannah & Zhou, 2009, Zhao et al., 2013, Chen et al., 2016, Li et al., 2020). Each of the three signatures correlated with downregulation of telomerase activity (FIG. 15b), with CX1 also being negatively correlated with telomere length (FIG. 15a) and associated with a lack of TERC and TERT amplification and expression (FIG. 15c-e). Therefore, telomere shortening may play a key role in the mechanisms underlying these signatures by increasing the frequency of abnormal chromosomes, mitotic nondisjunction and anaphase lagging (Pampalonat et al., 2010, Treff et al., 2011). Finally, CX1 positively correlated with the “clock-like” SBS1 signature (FIG. 13) suggesting these errors might also be mediated via a natural ageing process such as age-related telomere attrition (Li et al., 2020).

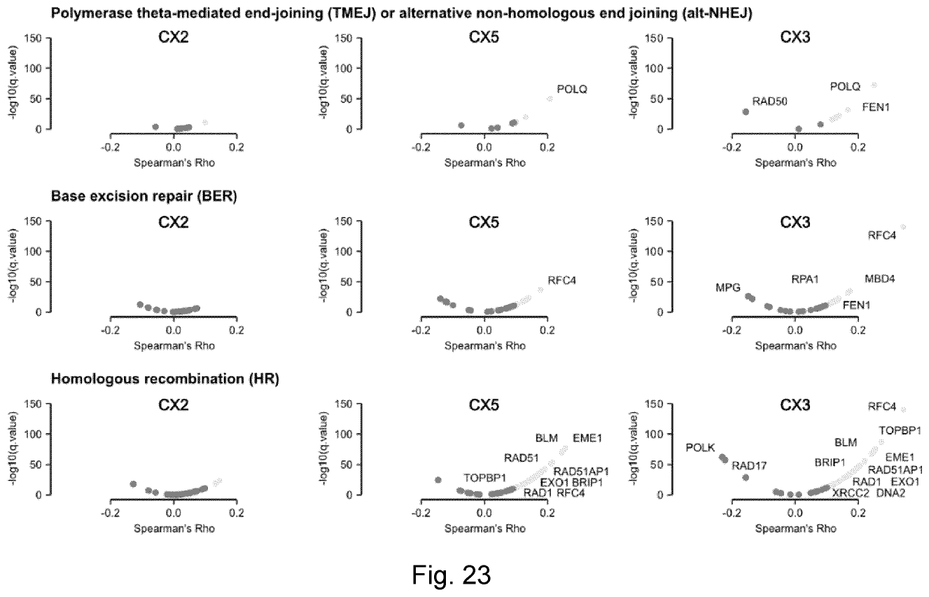

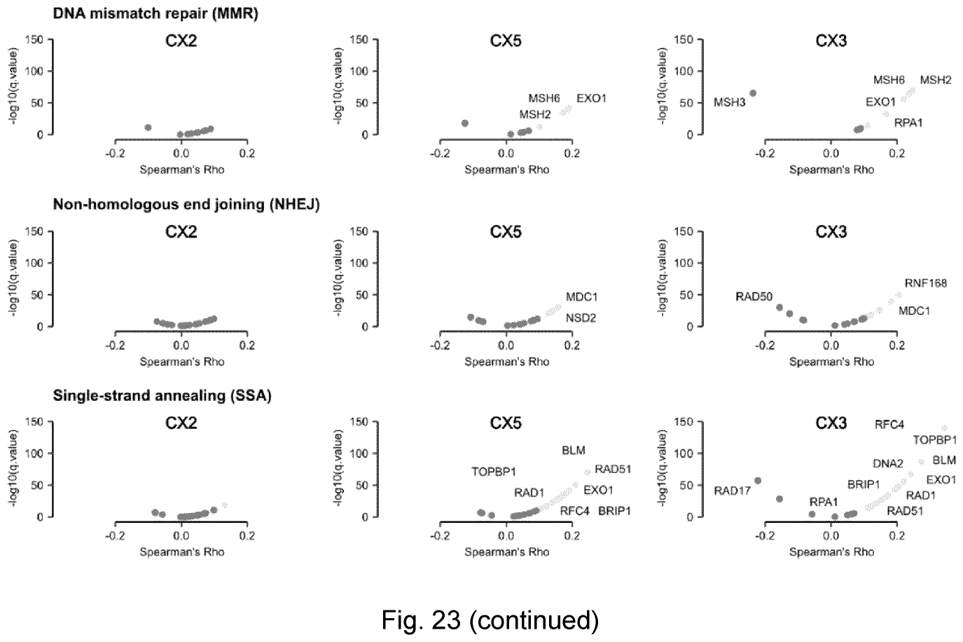

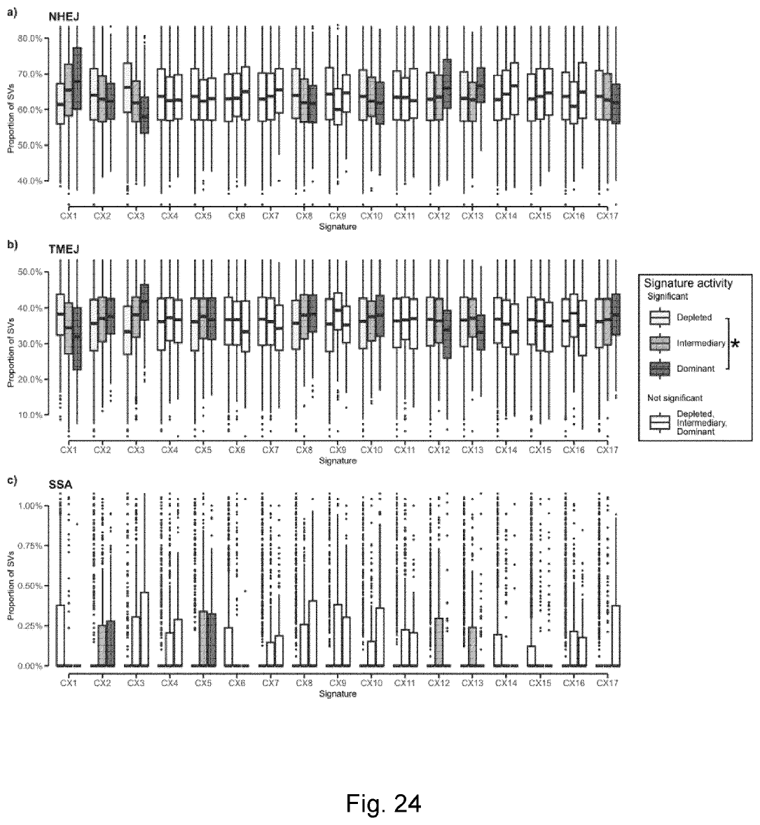

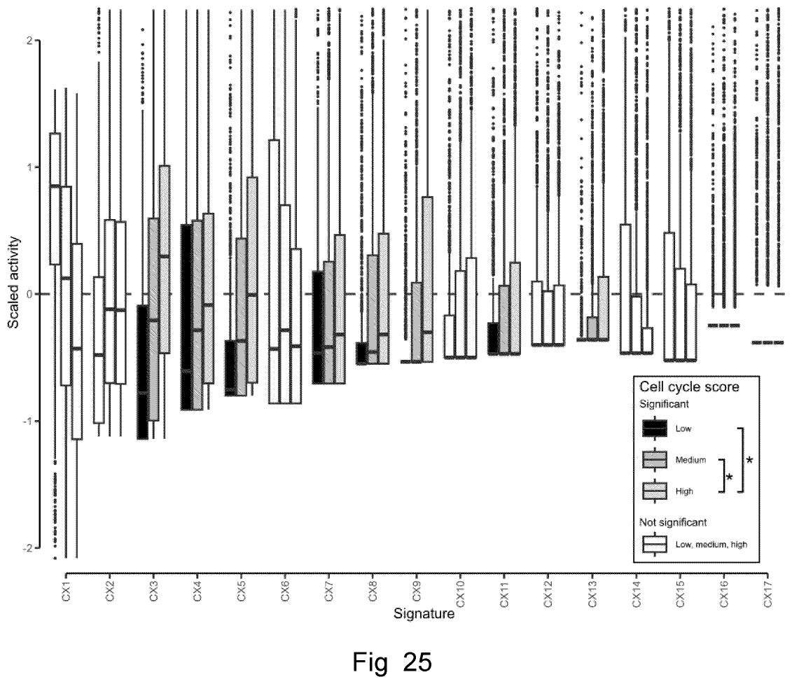

Signatures of impaired HR: CX2, CX3 and CX5 all exhibited patterns previously associated with impaired HR: CX2 showed a pattern of short, clustered changes associated with tandem-duplications; CX5 showed medium clustered, chained events associated with tandem duplication; and CX3 showed large, single copy changes with associated loss of heterozygosity. CX2, CX3 and CX5 were all observed at significantly higher levels in tumours with somatic BRCA1 mutation independently of each other, despite representing significantly different patterns of copy number changes (FIGS. 8, 13a, 16a). This suggests varying roles for disruption of homologous recombination (HR) as underlying causes (Powell et al. 2003). Several lines of evidence support the link between these signatures and HR: increases in CX2, CX3 and CX5 activity across germline mutated BRCA1 carriers (and BRCA2 carriers for CX3); higher activity in cases with methylated RAD51C11 (FIG. 16a); correlation with tandem duplication scores (Menghi et al., 2018) (FIG. 17); rearrangement signatures 1, 3 and 5 (Nik-Zainal et al., 2016) (FIG. 18); SBS signature 3 and ID signature 6 (Alexandrov et al., 2020); centrosome amplification score (Ogden et al., 2017) (FIG. 19); ovarian CN signatures 3 and 7 (Macintyre et al., 2018) (FIG. 20); association with loss of heterozygosity (Abkevich et al., 2012), chromothripsis (Piazza & Heyer, 2019) and kataegis (Guirouilh-Barbat et al., 2014) (FIG. 14); increased utilisation of non-homologous end joining (NHEJ), theta-mediated end joining (TMEJ) and single strand annealing (SSA) backup repair pathways visible as microhomologies at breakpoints (Stok et al., 2021, Hustedt & Durocher, 2017) (FIG. 21), as well as seven homologous recombination deficiency (HRD) metrics (Birkbak et al., 2011, Abkevich et al., 2012, Knijnenburg et al., 2018, Popova et al., 2012, Telli et al., 2016, Marquard et al., 2015) (FIG. 16d). The strength of these associations increased from CX2 to CX5 and to CX3. This suggested an increasing spectrum of CIN complexity associated with disruptions in HR mediated repair. Indeed, CX2 appears to be only associated with disruption of HR, whereas CX5 and CX3 have associations indicating the involvement of replication stress (via amplification and overexpression of MAPK1, PPP2R1A and U2AF1-Saavedra et al., 199, Primo et al., 2019, Perl et al., 2019, Palangat et al., 2019. Nguyen et al., 2018, Chen et al., 2018). The larger copy number changes observed for CX5 and CX3 suggest faster cell cycling and breaks carried through to mitosis, which was supported by strong correlation with cell cycle scores (FIG. 16b) and an increase in CNAs estimated to occur during mitosis (Schoonen et al., 2017) (FIG. 22, Methods). Further associations were observed for CX3, including missense mutations in ERCC2 and downregulation of key NER genes suggesting impaired nucleotide excision repair (NER) (FIG. 16c, FIG. 23), as well as TP53 mutation suggesting impaired damage sensing (FIG. 13a) (Li et al., 2019, Menon & Povirk 2014, Setton & Powell 2018). These CX3 associations are reminiscent of what has been termed BRCAness (Lord & Ashworth 2016) or HRD (Stok et al., 2021). However, CX5, and especially CX2, appear to represent a more moderate impairment of HR. Therefore we use the term impaired homologous recombination (IHR) for the aetiology underlying all three signatures rather than HRD.

Whole-genome duplication signature: CX4 encompassed a unique pattern of copy number change with neighbouring segments separated by 2 copy changes (FIG. 8), a pattern commonly used to define the presence of a whole-genome duplication (WGD) event (Dentro et al., 2021). CX4 was also associated with whole chromosome changes (FIG. 14a), a feature commonly observed in tetraploid cells due to increased mitotic errors (Prasad et al., 2021, Dewhurst et al. 2014). The specific WGD cause (endoreduplication, errors in cytokinesis, or cell fusion) was not evident from our data, however, this signature had high activity in tumours with PIK3R2, AKT1, and MAPK1 mutations suggesting tolerance to WGD may be mediated by PI3K/AKT activation (Berenjeno et al., 2017, Vanhaesebroeck et al., 2019, Darp et al. 202181-83) (FIG. 13a).

Signature of impaired non-homologous end joining: CX10, a signature of clustered copy number changes, had significantly higher activity in tumours with inactivating mutations in FBXW7, and correlated with FBXW7 mutant mediated tandem duplications class 1/2, suggesting a role for impaired non-homologous end joining (NHEJ) (Zhang et al., 2016 and 2019). A significant increase in the proportion of breakpoints with microhomologies in samples with this signature was indicative of a lack of blunt-end joining, a hallmark of NHEJ (FIG. 24a).

Correlation of the signature with FBXW7 mutant mediated tandem duplication class 1/254 and higher activity in tumours with PPP2R1A amplification, suggests these errors may occur in the context of replication fork collapse (Perl et al., 2019).

Signatures of amplification: CX8, CX9 and CX13 encoded patterns of low-, medium- and high-level amplifications, respectively. All three signatures were associated with increasing cell cycle score (Lundberg et al., 2020) (FIG. 25) and APOBEC mutagenesis (SBS2 and SBS13), and had higher activity in the context of amplification and overexpression of U2AF1, ERBB3 and MAPK1, suggesting replication stress as a putative cause (Cescon & Haibe-Kains, 2016, Kanu et al., 2016). Significantly higher CX8 activity in the context of amplification and overexpression of U2AF1, ERBB3 and MAPK1 implied R-loop formation, increased origin firing, premature replication fork termination, or asymmetric forks in combination with reliance on (error prone) NHEJ over HR as a putative mechanism (demonstrated via correlation with ID6 and ID8 (Alexandrov et al., 2020)) (Saavedra et al., 1999, Primo & Teixeira 2019, Nguyen et al., 2018, Chen et al., 2018). CX13 was strongly associated with ecDNA circularisation and amplification events (Kim et al., 2020) (FIG. 26), implying rolling circle amplification as the cause of the high copy number (Wang et al., 2021, Yan et al., 2020). However, the mechanism causing the ecDNA was not evident. An absence of associations with CX9 precluded in-depth identification of an underlying cause of these mid-level amplifications.

Clustered copy number signatures: CX11, CX16 and CX17 represented patterns indicative of clustered copy number changes. CX11 was associated with cell cycle score (FIG. 25) and ID signatures 1 and 258, suggesting replication stress as a putative cause. Significant increases in CX11 activity in the context of CDK4 amplification and overexpression (FIG. 13a) implied loss of cell cycle control as a possible cause of these clustered changes. Associations with CX16 and CX17 were widespread and indicated a number of possible aetiologies, however, given the available data we could not suggest a single putative aetiology. Correlation of CX16 and CX17 activities with SBS3 and ID6 signatures (Alexandrov et al. 2020), CX16 with tandem duplications (Menghi et al., 2018) and CX17 with RS1, RS3 and RS5 signatures (Nil-Zainal et al., 2016) implied a role for impaired HR (FIG. 13b, FIGS. 17-18). CX17 is distinct from CX16 due to its added association with R-loop formation via its high activity in U2AF1 amplified and overexpressed tumours (Nguyen et al., 2018, Chen et al., 2018). In addition, CX17 may have a role in WGD, due to its correlation with ovarian CNA signature 4 (Macintyre et al., 2018) (FIG. 20).

Cross-signature observations: Many covariates demonstrated associations with multiple signatures. Chromothripsis was linked with seven different signatures (FIG. 13), suggesting many potential aetiologies underpin these complex rearrangements. Replication stress was associated with six signatures, highlighting it as a major source of CIN. Different signatures showed a bias for occurrence before WGD (CX1, CX2, CX7, CX15) or after WGD (CX3, CX5, CX6, CX8, CX9, CX13, CX17) demonstrating the importance of WGD events in modulating CIN. Finally, signatures of APOBEC mutagenesis and kataegis were associated with six signatures, highlighting these as a common feature of CIN (Cescon & Haibe-Kains, 2016, Kanu et al., 2016, Nik-Zainal et al., 2012) (FIG. 13b, FIG. 14).

### Example 3—Drug Response Prediction and Drug Target Identification

In this example, the inventors investigated whether the activity of the copy number signatures identified in Example 1 can be predictive of drug response, and can identify drug targets that could be exploited to treat patients that show particular copy number signature activities.

### Methods

### See Examples 1 and 2

Calculating signature activities for cell lines. Cell line SNP 6.0 array CEL files were downloaded from the CCLE project (Ghandi et al., 2019) and were processed using Affymetrix Power Tools (APT; v2.11.2; standard options). Combined cell line log R and BAF data was split into sample-specific log R and BAF files. No germline samples were available for the identification of heterozygous SNPs in the cell line data, thus we ran the pipeline for SNP 6.0 without matched normal described above (see section “SNP 6.0 without matched normal” in the Methods of Example 1). In this pipeline, while the rescued SNPs allow for the identification of additional segments from their Log R, by construction their average BAF is never equal to 0 or 1 even in the presence of LOH. This biases purity estimates towards lower values and renders the copy-number fitting inaccurate. To circumvent this problem, we first fitted log R and BAF as per the ASCAT methodology (Van Loo et al. 2010), then searched the space of combinations of purity and ploidy around the same ploidy solutions, but between 95% and 100% purity and only solving for the total copy number using the Log R-only equation for each segment

$$log ( R ) = {log}_{2} \frac{{n}_{T} ρ + 2 ( 1 - ρ )}{ψ}$$

Where n_{T} is the total number of copies in the tumour, ρ is the purity and ψ is the average tissue ploidy. Akin to ASCAT, we picked the purity and ploidy combination that minimized the distance between nr and integer values. Only fitting the Log R and not the BAF in cell lines lead to more accurate values of the purity and thus the copy-number profiles. As fitting of absolute copy number to non-matched cell line data is inherently more challenging than other instances, all cell lines where ASCAT identified a plausible solution were visually inspected to determine fit suitability and excluded cell lines where absolute copy number profiles were incorrect or poorly fitted. We extracted signature activities for the 17 compendium signatures in 594 cancer cell lines. Extraction of the feature values from the copy number profiles derived from cell lines were performed as described above in section “Feature distributions and mixture modelling” in the Methods of Example 1. For identifying the signature activities, we used the linear combination decomposition function from YAPSA (R package, v1.12.0) and the 17 signatures of CIN.

CRISPR, RNAi and drug response data integration. Using the depmap portal we obtained essentiality scores of 17,645 genes from CRISPR knock-out screens, essentiality scores of 17,309 genes from RNAi screens, and area under dose response curve (AUC) values for 1,394 drugs across 481 human cancer cell lines from the PRISM screen (HTS002 screen) (see Table 1). After merging these data with cell line copy number profiles, we obtained a total of 382 cell lines with available copy number profiling and gene essentiality data from CRISPR and RNAi screens. From all of these, 297 also had drug response data from the PRISM screen.

Correlation of signatures with target gene KO, KD and drug inhibition. Kendall's Tau correlation was performed between CIN signature activities and gene essentiality scores from the genetic perturbation screens. Correlations were filtered for tau<−0.07 and q-value<0.05 after multiple testing corrections using the Benjamini-Hochberg method. This yielded 7,225 targets significantly associated with a signature from the CRISPR screen and 7,091 targets from the RNAi screen. These targets were then used to test correlation of the signature activity with matched drug inhibition of the target measured via AUC. After filtering (Kendall's tau<−0.1 and Benjamini-Hochberg corrected q-value<0.01) a total of 44 drug responses were correlated with signature activity via 40 targets. Conversely, those targets significantly linked to a CIN signature in both CRISPR and RNAi perturbation screens but without matched drug response represented putative novel targets for drug discovery. From an initial list of 319 targets overlapping in both the RNAi and CRISPR based correlation, 297 had unknown targeted therapies. After manually inspecting the target tractability (Mitsopoulos et al., 2021, Therneau et al., 2021) of these targets were considered druggable according to their structure or by ligand-based approaches, and after manual literature curation, 49 had a clear prior implication in CIN.

### Results

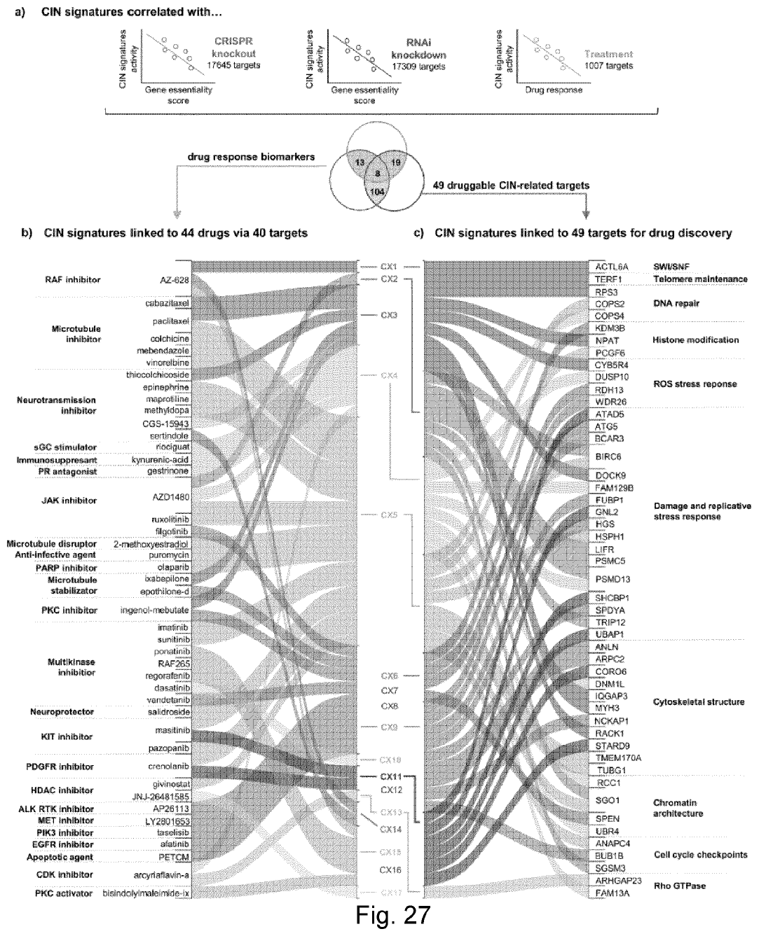

The putative signature aetiologies implicated canonical cancer pathways as some of the major drivers of CIN. Many of these pathways have been the focus of targeted therapy development. Therefore, given that the signatures identified in these examples can be readily measured in patient tumours, the inventors explored their utility for therapy response prediction and drug target identification. They integrated data from 297 cancer cell lines, including copy number profiling, genome-wide CRISPR knock-out screens93, genome-wide RNAi screens (McFarland et al., 2018) and the PRISM drug repurposing screen (Corsello et al., 2019). They assessed correlations between signature activities, gene essentiality, and sensitivity to drug perturbation of the gene (Methods, FIG. 27a).

They identified 40 genes where copy number signature activity was significantly correlated with both genetic and drug perturbation of the target (FIG. 27b, Table 4). Among these, several revealed promising new therapeutic avenues for targeting CIN. CX4 (associated with PI3K/AKT activation) was correlated with response to inhibition of CCND1 via arcyriaflavin-a (Hirakawa et al., 2017), which may indicate a therapeutic strategy for reversing tolerance to WGD. CX5, a signature of IHR, predicted response to olaparib via PARP1 inhibition. Given that this signature was also correlated with RNAi knockdown of PARP1, this may represent a biomarker specific to the inhibition of regular protein function rather than PARP trapping (Chaudhuri & Nussenzweig, 2017). CX9 (associated with replication stress) was correlated with response to multiple kinase inhibitors targeting genes involved in major mitogenic pathways (EGFR, JAK1, MET, PRKCA and PIK3CA), suggesting a multikinase inhibitor approach may be suitable for targeting replication stress. Correlation of CX13 (also associated with replication stress) with response to CDK4 inhibition, reinforced the interplay between replication stress and immune system activation (Uhbi & Brown, 2019), and potentially represents a biomarker-led approach for improving CDK4/6 inhibitor mediated tumour sensitisation to immune checkpoint blockade (Goel et al., 2017).

TABLE 4
Correlation values with drug responses identifying novel response biomarkers.

| CIN | | | | | | |
|---|---|---|---|---|---|---|
| sig. | Target | Drug | MOA | tau | p.value | q.value |
| CX1 | BRAF | AZ-628 | RAF inhibitor | −0.09858816 | 0.007504666 | 0.03189483 |
| CX2 | JAK1 | AZD1480 | JAK inhibitor | −0.095693663 | 0.014510294 | 0.049019852 |
| CX3 | TUBB | cabazitaxel | Microtubule | −0.106385994 | 0.003368922 | 0.006176357 |
| | | | inhibitor | | | |
| CX3 | PRKCG | ingenolmebutate | PKC activator | −0.107845376 | 0.003481497 | 0.006246242 |
| CX3 | TUBB | ixabepilone | Microtubule | −0.133560916 | 0.000349304 | 0.002364519 |
| | | | stabilizing agent | | | |
| CX3 | TUBA3C | ixabepilone | Microtubule | −0.133560916 | 0.000349304 | 0.004915251 |
| | | | stabilizing agent | | | |
| CX3 | TUBB | paclitaxel | Microtubule | −0.108740709 | 0.002807097 | 0.005479968 |
| | | | inhibitor | | | |
| CX3 | MAPT | paclitaxel | Microtubule | −0.108740709 | 0.002807097 | 0.006237993 |
| | | | inhibitor | | | |
| CX3 | GABRB2 | thiocolchicoside | Neurotransmission | −0.119393881 | 0.001765812 | 0.004888386 |
| | | | inhibitor | | | |
| CX4 | CCND1 | arcyriaflavin-a | CDK inhibitor | −0.115871886 | 0.002611232 | 0.022195472 |
| CX4 | JAK1 | AZD1480 | JAK inhibitor | −0.079097713 | 0.037347159 | 0.049019852 |
| CX4 | ADORA2A | CGS-15943 | Neurotransmission | −0.109033563 | 0.004937438 | 0.008713126 |
| | | | inhibitor | | | |
| CX4 | ESR1 | gestrinone | Progesterone | −0.13016269 | 0.001322309 | 0.004848467 |
| | | | receptor antagonist | | | |
| CX4 | GRIN2B | kynurenic-acid | Immunosuppressant | −0.109876338 | 0.005547193 | 0.007775501 |
| CX4 | GUCY1A3 | riociguat | Guanylate cyclase | −0.109488156 | 0.006158925 | 0.008129491 |
| | | | stimulant | | | |
| CX5 | HIF1A | 2-methoxyestradiol | Microtubule | −0.154289537 | 6.22201E−05 | 0.001368842 |
| | | | disrupting agent | | | |
| CX5 | JAK1 | AZD1480 | JAK inhibitor | −0.103932676 | 0.00808557 | 0.009487069 |
| CX5 | TUBB8 | colchicine | Microtubule | −0.120204356 | 0.00192476 | 0.004981731 |
| | | | inhibitor | | | |
| CX5 | TUBA1A | colchicine | Microtubule | −0.120204356 | 0.00192476 | 0.006205592 |
| | | | inhibitor | | | |
| CX5 | PDGFRA | crenolanib | PDGFR tyrosine | −0.125936063 | 0.000728383 | 0.004006105 |
| | | | kinase receptor | | | |
| | | | inhibitor | | | |
| CX5 | ADRA2B | epinephrine | Neurotransmission | −0.137808512 | 0.000631558 | 0.003705143 |
| | | | inhibitor | | | |
| CX5 | ADRB3 | epinephrine | Neurotransmission | −0.137808512 | 0.000631558 | 0.005150032 |
| | | | inhibitor | | | |
| CX5 | HDAC6 | givinostat | HDAC inhibitor | −0.113289991 | 0.002580071 | 0.005479968 |
| CX5 | PDGFRA | imatinib | Multikinase | −0.137767393 | 0.000321731 | 0.002359363 |
| | | | inhibition | | | |
| CX5 | ADRA2B | maprotiline | Neurotransmission | −0.123511394 | 0.001870664 | 0.004981731 |
| | | | inhibitor | | | |
| CX5 | PDGFRA | masitinib | KIT inhibitor | −0.115067215 | 0.002926801 | 0.005479968 |
| CX5 | TUBA1A | mebendazole | Microtubule | −0.120336731 | 0.004163938 | 0.007570797 |
| | | | inhibitor | | | |
| CX5 | ADRA2B | methyldopa | Neurotransmission | −0.102339987 | 0.007429171 | 0.009080097 |
| | | | inhibitor | | | |
| CX5 | PARP1 | olaparib | PARP inhibitor | −0.123774336 | 0.00108104 | 0.004669922 |
| CX5 | MAP4 | paclitaxel | Microtubule | −0.10956892 | 0.00268909 | 0.005479968 |
| | | | inhibitor | | | |
| CX5 | MAPT | paclitaxel | Microtubule | −0.10956892 | 0.00268909 | 0.006205592 |
| | | | inhibitor | | | |
| CX5 | PDGFRA | pazopanib | KIT inhibitor | −0.107989169 | 0.003887035 | 0.006246242 |
| CX5 | RPL10L | puromycin | Anti-infective agent | −0.150352958 | 8.0846E−05 | 0.002355167 |
| CX5 | TYK2 | ruxolitinib | JAK inhibitor | −0.127192296 | 0.00157879 | 0.006152913 |
| CX5 | APP | salidroside | Neuroprotective | −0.101163629 | 0.007209601 | 0.008935844 |
| | | | agent | | | |
| CX5 | PDGFRA | sunitinib | Multikinase | −0.101127822 | 0.008027756 | 0.009487069 |
| | | | inhibition | | | |
| CX5 | TUBB8 | vinorelbine | Microtubule | −0.13017525 | 0.000491525 | 0.003089586 |
| | | | inhibitor | | | |
| CX9 | EGFR | afatinib | EGFR inhibitor | −0.075360451 | 0.041604806 | 0.049019852 |
| CX9 | EGFR | AP26113 | ALK tyrosine | −0.071009838 | 0.040442816 | 0.049019852 |
| | | | kinase receptor | | | |
| | | | inhibitor | | | |
| CX9 | TUBB4B | epothilone-d | Microtubule | −0.075973603 | 0.04700107 | 0.049938637 |
| | | | stabilizing agent | | | |
| CX9 | JAK1 | filgotinib | JAK inhibitor | −0.119574654 | 0.003401119 | 0.006802238 |
| CX9 | PRKCA | ingenolmebutate | PKC activator | −0.119020007 | 0.001740103 | 0.004888386 |
| CX9 | MET | LY2801653 | MET inhibitor | −0.110581078 | 0.002817091 | 0.005479968 |
| CX9 | CASP3 | PETCM | Apoptotic agent | −0.107796514 | 0.004500795 | 0.006948596 |
| CX9 | PIK3CA | taselisib | PI3K inhibitor | −0.074240241 | 0.032935533 | 0.049019852 |
| CX9 | EGFR | vandetanib | Multikinase | −0.089725613 | 0.017478774 | 0.049019852 |
| | | | inhibition | | | |
| CX10 | FYN | dasatinib | Multikinase | −0.114874037 | 0.008034838 | 0.009487069 |
| | | | inhibition | | | |
| CX11 | PDGFRA | crenolanib | PDGFR tyrosine | −0.073587964 | 0.033226468 | 0.049019852 |
| | | | kinase receptor | | | |
| | | | inhibitor | | | |
| CX11 | PDGFRA | masitinib | KIT inhibitor | −0.072664281 | 0.043252811 | 0.049019852 |
| CX13 | CDK4 | arcyriaflavin-a | CDK inhibitor | −0.075457794 | 0.050691277 | 0.050691277 |
| CX14 | BRAF | AZ-628 | RAF inhibitor | −0.104823079 | 0.00628188 | 0.008129491 |
| CX14 | RAF1 | AZ-628 | RAF inhibitor | −0.104823079 | 0.00628188 | 0.008129491 |
| CX14 | HTR2A | sertindole | Neurotransmission | −0.105532212 | 0.005566552 | 0.007775501 |
| | | | inhibitor | | | |
| CX15 | BRAF | AZ-628 | RAF inhibitor | −0.118931527 | 0.005678276 | 0.009208016 |
| CX15 | MAPK1 | bisindolylmaleimide-ix | PKC inhibitor | −0.1145687 | 0.005905719 | 0.00932482 |
| CX15 | TEK | ponatinib | Multikinase | −0.122202839 | 0.003732668 | 0.007186788 |
| | | | inhibition | | | |
| CX15 | BRAF | RAF265 | Multikinase | −0.146100873 | 0.000794731 | 0.005150032 |
| | | | inhibition | | | |
| CX15 | BRAF | regorafenib | Multikinase | −0.129053702 | 0.002640493 | 0.006205592 |
| | | | inhibition | | | |
| CX15 | DDR2 | regorafenib | Multikinase | −0.129053702 | 0.002640493 | 0.006205592 |
| | | | inhibition | | | |
| CX15 | EPHA2 | regorafenib | Multikinase | −0.129053702 | 0.002640493 | 0.006205592 |
| | | | inhibition | | | |
| CX15 | TEK | regorafenib | Multikinase | −0.129053702 | 0.002640493 | 0.006205592 |
| | | | inhibition | | | |
| CX17 | HDAC2 | JNJ-26481585 | HDAC inhibitor | −0.137066263 | 0.003253841 | 0.006732085 |
| CX17 | HDAC4 | JNJ-26481585 | HDAC inhibitor | −0.137066263 | 0.003253841 | 0.006732085 |

CIN sig. = Number of CIN signature; Target = Name of the gene targeted by the drug; Drug = Name of the drug; MOA = Mode of action for each drug; tau = Kendall's Tau; p.value = p-value; q.value = Multiple testing corrected p-value (FDR, Benjamini-Hochberg).

Copy number signature correlations with gene essentiality scores, from both CRISPR and RNAi perturbation screens, identified 104 target genes with druggable structures (Mitsopoulos, 2021) that currently have no targeted therapies in the clinic. These represent putative synthetic lethal drug targets, 49 of which had evidence of being implicated in CIN-related mechanisms (FIG. 27c, Table 5). A number of these show promising links between the signature aetiology and potential consequence of target inhibition. CX1 activity was correlated with perturbation of ACTL6A (involved in SWI/SNF complex) and TERF1 (telomere maintenance), both of which are required for faithful chromosome segregation during mitosis (Pampalona et al., 2010, Brownlee et al., 2015). The combined dysregulation mitosis and telomere elongation machinery associated with CX1 suggests that inhibiting either one of these genes might be a promising therapeutic strategy by creating synthetic lethality. Indeed, inhibition of both genes has been previously suggested to induce cell lethality by generating excessive CIN (Kops et al., 2004). CX9 was correlated with BUB1B perturbation, a spindle assembly checkpoint (SAC) gene recently identified as therapeutically relevant in CIN-high cells measured via WGD status (Quinton et al., 2021) and an aneuploidy score (Cohen-Sharir et al., 2021). This association with CX9 suggests that SAC may play a crucial role in tolerating mid-level amplifications, and reducing levels of BUB1B may induce excessive and catastrophic chromosome missegregation (Janssen et al., 2009). Finally, CX11, which was strongly associated with CDK4 amplification, was correlated with inhibition of GLN2 which in turn impedes cyclin D1/CDK4 complex formation (Datta et al., 2015).

TABLE 5
Overview of novel drug targets.

| Sign | Biological mechanism | Genes | Tractability |
|---|---|---|---|
| CX1 | Cell adhesion | CDH4 | Druggable by Ligand |
| | | | Based |
| CX1 | Cytoskeletal structure | IQGAP3 | Druggable by Ligand |
| | | | Based |
| CX1 | Cytoskeletal structure | MYH3 | Druggable by Ligand |
| | | | Based |
| CX1 | Cytoskeletal structure | PPP1R16A | Druggable by Ligand |
| | | | Based |
| CX1 | Cytoskeletal structure | RACK1 | Druggable Structure |
| CX1 | DNA repair | RPS3 | Druggable Structure |
| CX1 | Histone modification | NPAT | Druggable by Ligand |
| | | | Based |
| CX1 | Metabolism | ECI2 | Druggable Structure |
| CX1 | Metabolism | EIF3E | Druggable Structure |
| CX1 | Metabolism | LPIN1 | Druggable by Ligand |
| | | | Based |
| CX1 | Metabolism | TNFAIP8L3 | Druggable Structure |
| CX1 | Replication stress | DOCK9 | Druggable Structure |
| CX1 | ROS stress | CYB5R4 | Druggable Structure |
| CX1 | SWI/SNF chromatin remodeling | ACTL6A | Druggable Structure |
| CX1 | Telomere maintenance | TERF1 | Druggable Structure |
| CX1 | Transcription regulation | DROSHA | Druggable Structure |
| CX1 | Transcription regulation | RPS7 | Druggable Structure |
| CX1 | Transcription regulation | TAF3 | Druggable Structure |
| CX2 | Cell cycle control | SGSM3 | Druggable by Ligand |
| | | | Based |
| CX2 | Chromatin architecture | SGO1 | Druggable by Ligand |
| | | | Based |
| CX2 | Metabolism | CYB5R3 | Druggable Structure |
| CX2 | Metabolism | PC | Druggable Structure |
| CX2 | Replication stress | LIFR | Druggable by Ligand |
| | | | Based |
| CX2 | Replication stress | PSMC5 | Druggable Structure |
| CX2 | Replication stress | TRIP12 | Druggable by Ligand |
| | | | Based |
| CX2 | Replication stress | BIRC6 | Druggable by Ligand |
| | | | Based |
| CX2 | Replication stress | PSMD13 | Druggable Structure |
| CX2 | Splicing | CDC40 | Druggable Structure |
| CX2 | Splicing | LSM7 | Druggable Structure |
| CX2 | Splicing | SNRPD1 | Druggable Structure |
| CX2 | Trafficking | SLC7A9 | Druggable Structure |
| CX2 | Transcription regulation | ABCA6 | Druggable by Ligand |
| | | | Based |
| CX2 | Unknown | ANKRD18A | Druggable by Ligand |
| | | | Based |
| CX4 | Chromatin architecture | RCC1 | Druggable Structure |
| CX4 | Chromatin architecture | UBR4 | Druggable by Ligand |
| | | | Based |
| CX4 | Cytoskeletal structure | DNM1L | Druggable Structure |
| CX4 | DNA repair | COPS4 | Druggable Structure |
| CX4 | Immune response | PLEKHO2 | Druggable by Ligand |
| | | | Based |
| CX4 | Metabolism | CBR4 | Druggable Structure |
| CX4 | Metabolism | NUP43 | Druggable Structure |
| CX4 | Replication stress | FAM129B/NIBAN2 | Druggable Structure |
| CX4 | Replication stress | PSMD13 | Druggable Structure |
| CX4 | ROS stress | DUSP10 | Druggable Structure |
| CX4 | Signalling by Rho GTPase | FAM13A | Druggable by Ligand |
| | | | Based |
| CX4 | Transcription regulation | TRMT10A | Druggable Structure |
| CX5 | Cell cycle control | ANAPC4 | Druggable Structure |
| CX5 | Chromatin architecture | SGO1 | Druggable by Ligand |
| | | | Based |
| CX5 | Cytoskeletal structure | TMEM170A | Druggable by Ligand |
| | | | Based |
| CX5 | Cytoskeletal structure | TUBG1 | Druggable Structure |
| CX5 | DNA repair | COPS2 | Druggable Structure |
| CX5 | Hemostasis | SERPINE2 | Druggable Structure |
| CX5 | Histone modification | PCGF6 | Druggable Structure |
| CX5 | Immune response | AP1S3 | Druggable Structure |
| CX5 | Metabolism | EXOSC3 | Druggable Structure |
| CX5 | Metabolism | MUC17 | Druggable by Ligand |
| | | | Based |
| CX5 | Mitochondrial biogenesis | LRRC46 | Druggable by Ligand |
| | | | Based |
| CX5 | Replication stress | HSPH1 | Druggable Structure |
| CX5 | Replication stress | BIRC6 | Druggable by Ligand |
| | | | Based |
| CX5 | Splicing | GTF2F1 | Druggable Structure |
| CX5 | Splicing | LARP7 | Druggable Structure |
| CX5 | Splicing | SNRNP70 | Druggable Structure |
| CX5 | Splicing | DHX8 | Druggable Structure |
| CX5 | Transcription regulation | INTS9 | Druggable Structure |
| CX6 | Angiogenesis | ENG | Druggable Structure |
| CX6 | Cell adhesion | FERMT2 | Druggable Structure |
| CX6 | Chromatin architecture | SPEN | Druggable by Ligand |
| | | | Based |
| CX6 | Histone modification | KDM3B | Druggable Structure |
| CX6 | Metabolism | TCP1 | Druggable Structure |
| CX6 | Replication stress | ATG5 | Druggable by Ligand |
| | | | Based |
| CX6 | ROS stress | RDH13 | Druggable by Ligand |
| | | | Based |
| CX6 | Trafficking | SPG7 | Druggable Structure |
| CX9 | Cell cycle control | BUB1B | Druggable Structure |
| CX9 | Cytoskeletal structure | ANLN | Druggable Structure |
| CX9 | Cytoskeletal structure | ARPC2 | Druggable Structure |
| CX9 | Cytoskeletal structure | NCKAP1 | Druggable Structure |
| CX9 | Membrane trafficking | VPS29 | Druggable Structure |
| CX9 | Metabolism | CELA2B | Druggable by Ligand |
| | | | Based |
| CX9 | Metabolism | EIPR1 | Druggable Structure |
| CX9 | Replication stress | BCAR3 | Druggable Structure |
| CX9 | Replication stress | FUBP1 | Druggable Structure |
| CX9 | Replication stress | HGS | Druggable Structure |
| CX9 | Replication stress | SPDYA | Druggable Structure |
| CX9 | ROS stress | WDR26 | Druggable by Ligand |
| | | | Based |
| CX9 | Trafficking | SLC9A3R1 | Druggable Structure |
| CX9 | Transcription regulation | DLX3 | Druggable Structure |
| CX9 | Transcription regulation | SBDS | Druggable Structure |
| CX11 | Calcium-sensitive signal | ENKUR | Druggable Structure |
| | transduction | | |
| CX11 | Cell adhesion | LAMA3 | Druggable by Ligand |
| | | | Based |
| CX11 | Cytoskeletal structure | CORO6 | Druggable Structure |
| CX11 | Cytoskeletal structure | STARD9 | Druggable by Ligand |
| | | | Based |
| CX11 | Immune response | C6 | Druggable Structure |
| CX11 | Metabolism | MCAT | Druggable Structure |
| CX11 | Metabolism | MMACHC | Druggable Structure |
| CX11 | Metabolism | SPC25 | Druggable by Ligand |
| | | | Based |
| CX11 | Metabolism | WAC | Druggable by Ligand |
| | | | Based |
| CX11 | Replication stress | ATAD5 | Druggable by Ligand |
| | | | Based |
| CX11 | Replication stress | GNL2 | Druggable by Ligand |
| | | | Based |
| CX11 | Replication stress | SHCBP1 | Druggable by Ligand |
| | | | Based |
| CX11 | Replication stress | UBAP1 | Druggable Structure |
| CX11 | Splicing | CRNKL1 | Druggable Structure |
| CX11 | Splicing | DHX8 | Druggable Structure |
| CX11 | Trafficking | ATP6V0D1 | Druggable Structure |
| CX11 | Transcription regulation | SUPT5H | Druggable Structure |
| CX13 | Immune response | HECTD1 | Druggable by Ligand |
| | | | Based |
| CX13 | Membrane trafficking | MICU1 | Druggable Structure |
| CX13 | Metabolism | NUP98 | Druggable Structure |
| CX13 | Metabolism | REXO2 | Druggable Structure |
| CX13 | Signalling by Rho GTPase | ARHGAP23 | Druggable by Ligand |
| | | | Based |

Sign = Number of CIN signature; Biological mechanism = Summary of role of the gene; Genes = Name of the gene; Tractability = Details about the of a target.

### Example 4—Copy Number Signatures Identify Impaired Homologous Recombination and Predict Platinum Response

In this example, the inventors investigated in more detail the copy number signatures identified in Example 2 as likely to be indicative of impaired homologous recombination, and whether these could be predictive of response to platinum-based therapy.

### Methods

### See Examples 1-3

Preparation of survival data. Clinical metadata for the TCGA cohort were downloaded from the TCGA website: https://portal.gdc.cancer.gov/. Clinical metadata for the ICGC breast cancer cohort was from the original publication (Nik-Zainal et al., 2016). Data for the PCAWG OV-AU and ESAD-UK were obtained from synapse (ICGC/TCGA Pan-Cancer Analysis of Whole Genomes Consortium, 2020). Age was converted into years and split into 4 groups: [0, 53), [53, 62), [62, 76), [76+). Stages were simplified by omitting additional stage identifier after the initial roman letter identifier (e.g. Stage IIA1 became Stage II). Stage IS was treated as NA. We added some missing clinical stages supplied by Liu et al. 2018.

Survival analysis. In order to study the relationship between the signature activities, clinical classifiers and survival of patients, we deployed a univariate (Kaplan-Meier estimates) and a multivariate method (Cox proportional hazard model). Kaplan-Meier estimates (function survfit) and Cox proportional hazard models (function coxph) were performed using survival (Therneau, 2021, R package, v3.1-11) and survminer (Drawing Survival Curves using ggplot2′ [R package survminer version 0.4.9]. (2021); R package, v0.4.8). For the Kaplan-Meier estimates a logrank test was performed. For the Cox proportional hazard models we included the covariates stage and age category, except for HRDetect classification on the TCGA-OV cohort where we only corrected for age and not stage since there were not enough samples in each stage for the model to converge (n=40).

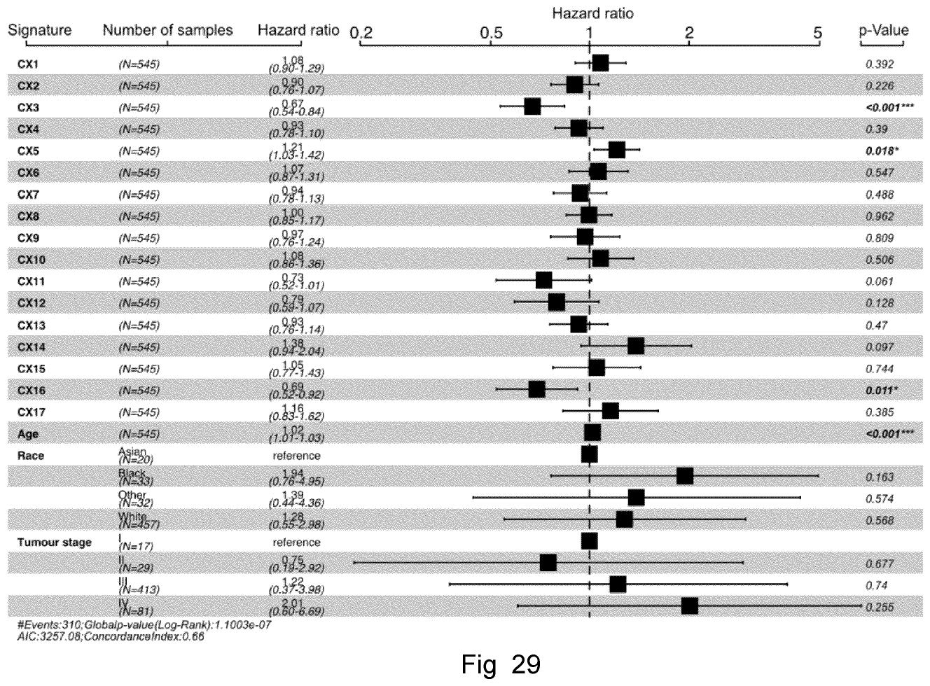

Clinical classifier based on CX2/CX3 signature activity. From our work on the signature aetiologies, we found that CX2 has no significant relation to predicting platinum-sensitivity in TCGA ovarian cancer patients but CX3 does (FIG. 29). We devised a clinical classifier exploiting exactly this relation on the 36 germline BRCA1 mutant samples of the TCGA-OV cohort that had an additional loss of heterozygosity at the BRCA1 locus. For each sample, we normalised their 17 signature activities to 1. Then we centered the mean and scaled the variance of the signatures CX2 and CX3 across the 36 samples. This scaling and centering is the first part of the classifier. The second part of the classifier is checking which signature has a higher value and classify accordingly: if CX2 activity is higher we predicted platinum-resistance and if CX3 activity is higher then we predicted platinum-sensitivity. Applying this classifier to new samples means that their CX2 and CX3 signature activities are evaluated relatively to germline BRCA1 mutant samples. The scaling worked well for ovarian and breast cancer samples but had to be redone for the oesophageal samples. We applied the CX2/CX3 classifier to the whole ovarian cohort and found that 27% of the TCGA OV cohort might be platinum sensitive. Extending this classifier pan-cancer, we estimate that at most 8% of all tumours might be sensitive.

Comparison with HRDetect and Myriad myChoice classifier. HRDetect values were taken from Davies et al. 2017 and Degasperi et al. 2020. As in the original publication, the threshold of 0.7 was used to identify positive samples (Davies et al). Myriad myChoice classifier is based on the HRD score (Telli et al., 2016). Positive samples were samples with scores above or equal to 42. HRD scores were taken from Knijnenburg et al. 2018 and complemented by data from Marquard et al. 2015 for samples with NA values.

Clinical classifier based on three impaired HR signatures. In order to test our clinical classifier based on CX2 and CX3 alone, we deployed three additional clinical classifiers taking into account all three impaired HR signatures (CX2, CX3, CX5). The three classifiers produced hazard ratios for the resistant or early death group at 0.62, 0.57 and 0.67 which were inferior to the 0.54 hazard ratio produced by the CX2/CX3 classifier.

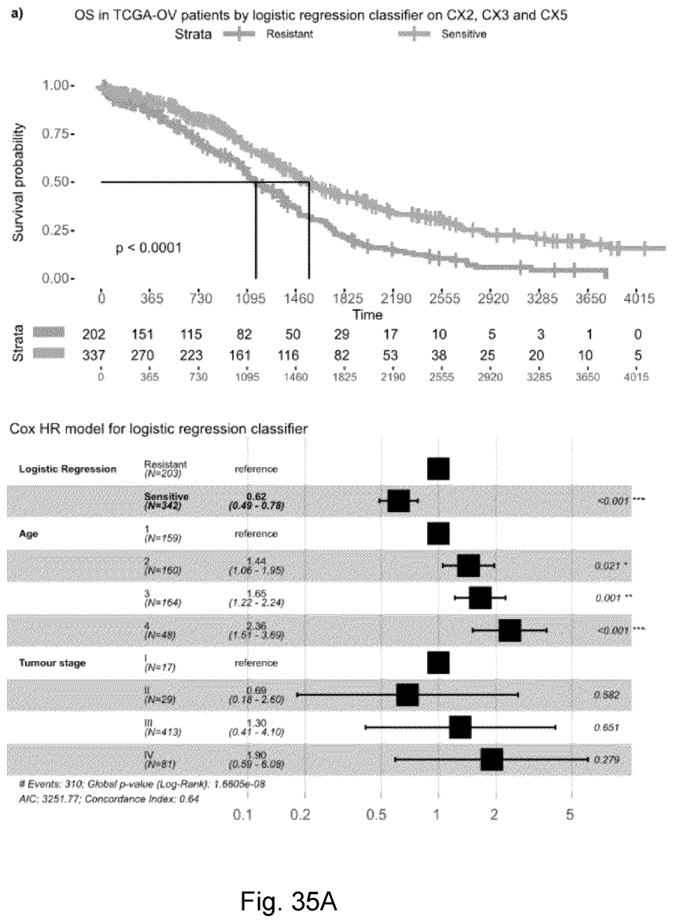

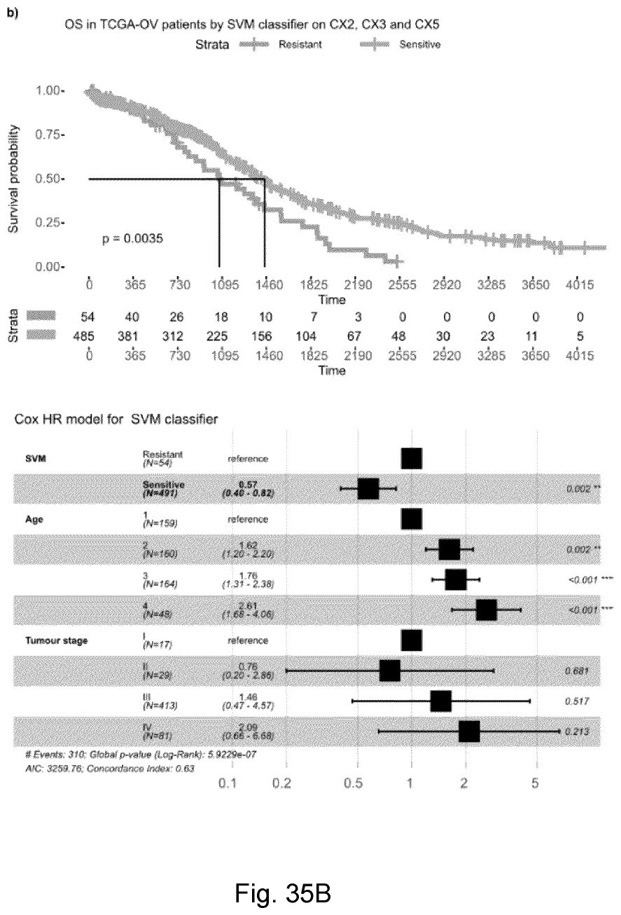

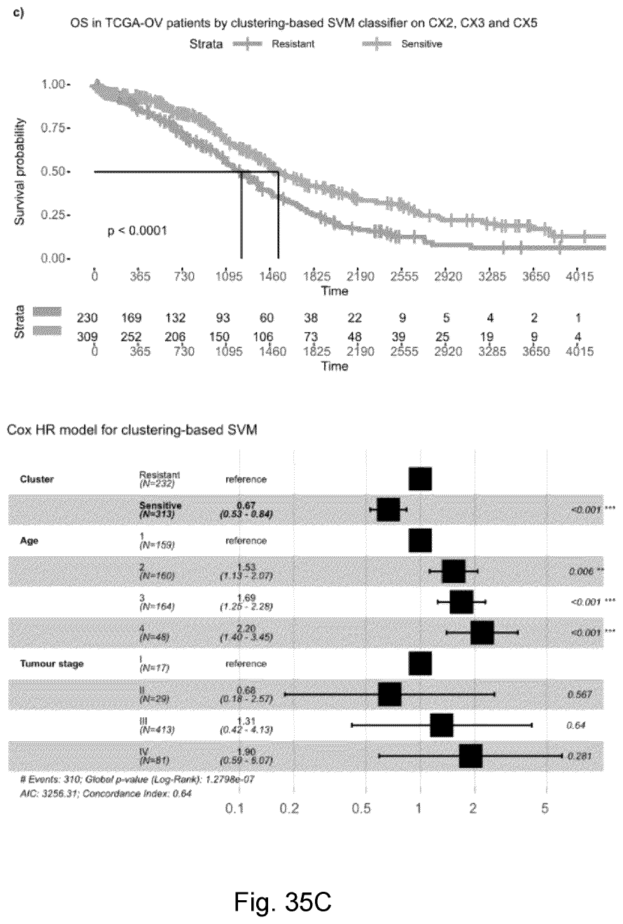

Logistic regression on survival outcome. First, we extracted the germline BRCA1 samples from the ovarian TCGA cohort and scaled the activities for the three impaired HR signatures. Then we trained a logistic regression using the function glm (R core package, v.3.6.1) on the scaled signatures to predict the outcome of the survival data (death). Then we scaled the signature activities of all TCGA ovarian cancer samples according to the scale and variance from the germline BRCA1 samples and tested the logistic regression classifier by producing a Kaplan-Meier estimate and a Cox proportional hazard model (FIG. 35, top row). Support vector machine (svm) on survival outcome The second classifier was a support vector machine (svm). We trained the model on the same data as the logistic regression and CX2/CX3 classifiers: scaled impaired HR signature activities from the germline BRCA1 samples of the TCGA ovarian cancer cohort. During training to predict survival outcome (death) we avoided overfitting by ten-fold cross validation. Afterwards, the activities of the complete TCGA-OV cohort were scaled according to the germline BRCA1 sample scaling with the svm predicting an early death based (FIG. 35, middle row).

Support vector machine (svm) on hierarchical clustering. The third classifier was an svm predicting the class membership of two groups produced by hierarchical clustering on the scaled signature activities of the germline BRCA1 samples of the TCGA ovarian cancer cohort. A ten-fold cross validation was used as a regularisation method. The classifier was then applied to the complete TCGA-OV cohort which was again scaled according to the activities of the germline BRCA1 samples (FIG. 35, bottom row).

### Results

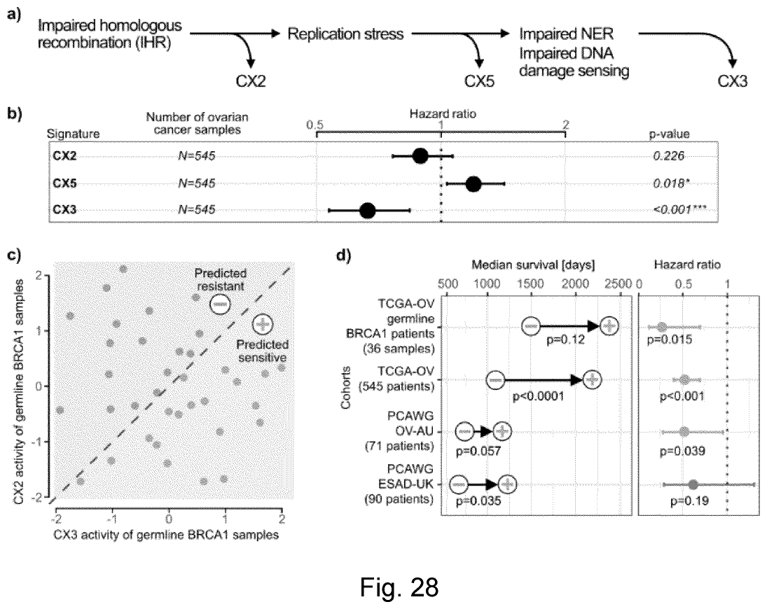

The aetiologies of the 3 impaired HR signatures described in Example 2 indicated a model of increasing CIN complexity (FIG. 28a). IHR alone gives rise to CX2, a signature of small copy number changes indicative of tandem duplication. IHR plus replication stress causes CX5, involving larger CNAs. Finally, IHR plus replication stress, impaired damage sensing and impaired NER, gives rise to CX3 with the largest CNAs that are strongly associated with LOH. Our results did not reveal if the different levels of complexity developed in a stepwise manner or by independent processes.

Disruption of both HR and NER have been shown to confer sensitivity to platinum-based chemotherapy (Konstantinopoulos et al., 2015, Martin et al., 2008). Given only CX3 is associated with disruption of NER, we hypothesised our IHR signatures may demonstrate differing abilities to predict platinum sensitivity. As ovarian cancer patients are routinely treated with platinum-based chemotherapy, we were able to test the ability of all three signatures to predict overall survival, and hence platinum sensitivity, using a Cox-proportional hazards model (FIG. 28b, FIG. 29). CX2 showed no association with platinum sensitivity, CX5 was predictive of resistance and CX3 predictive of sensitivity.

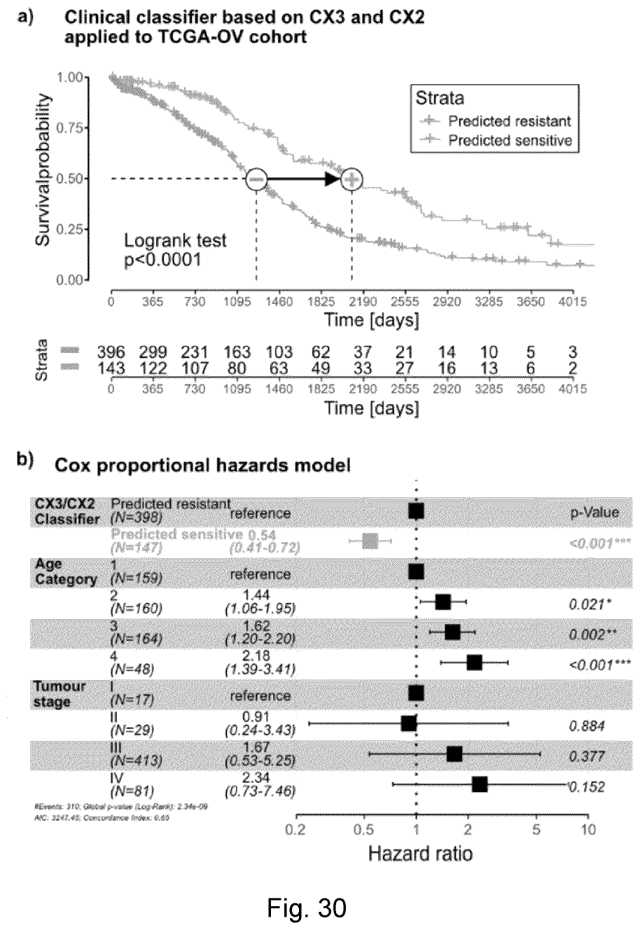

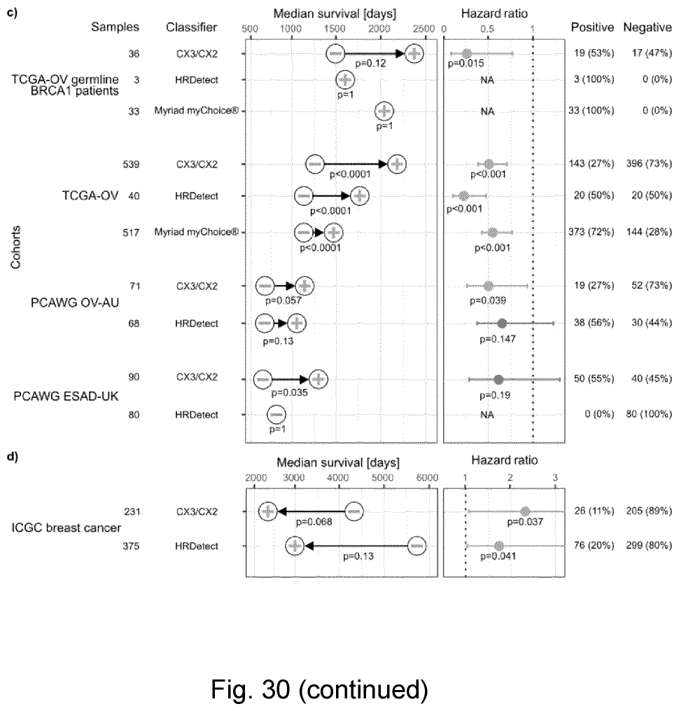

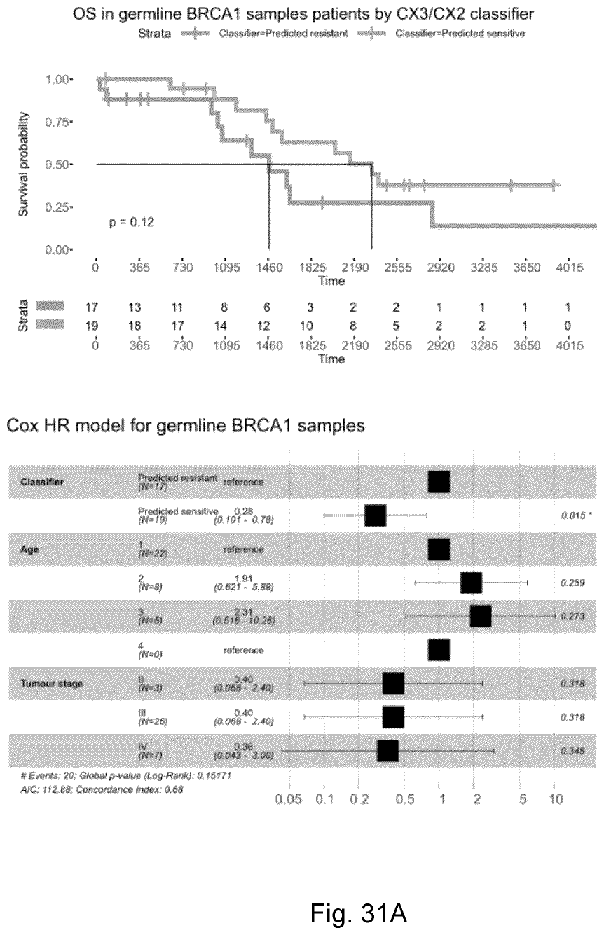

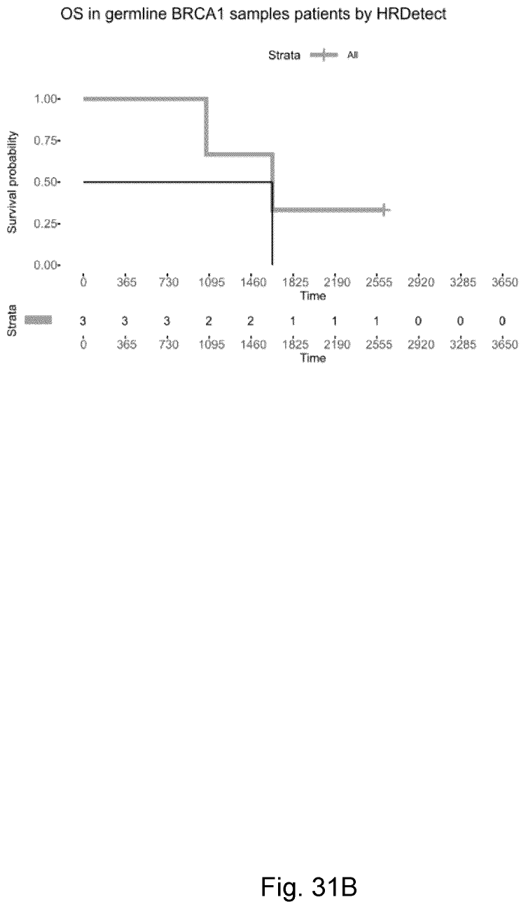

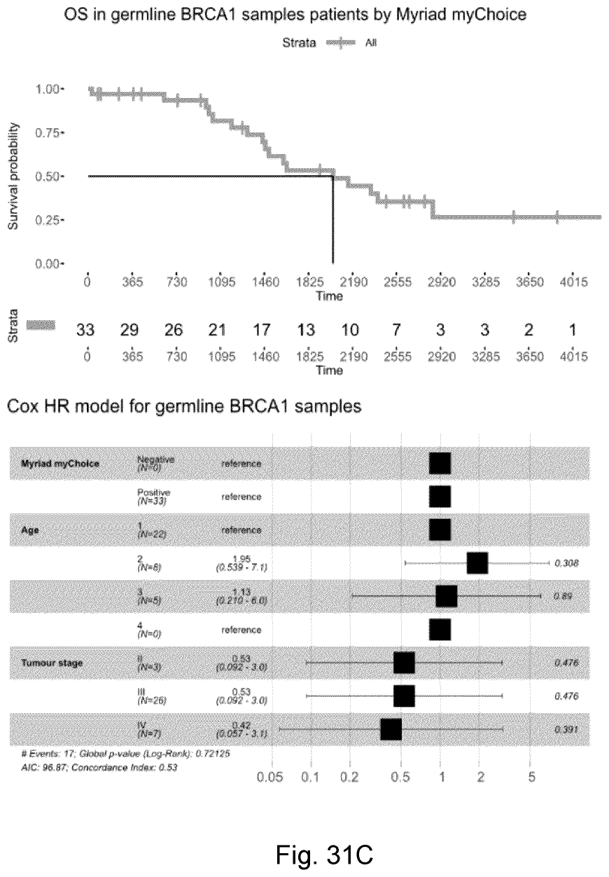

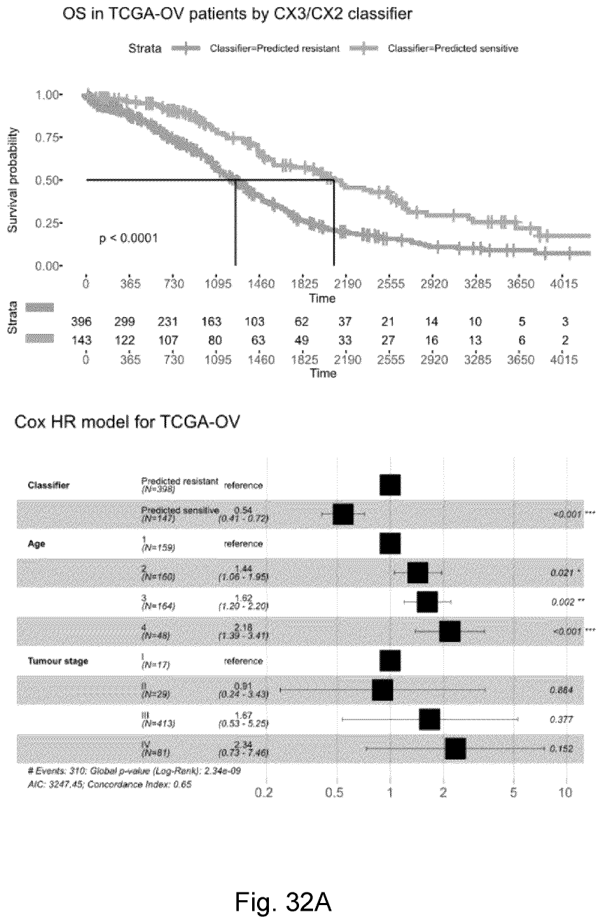

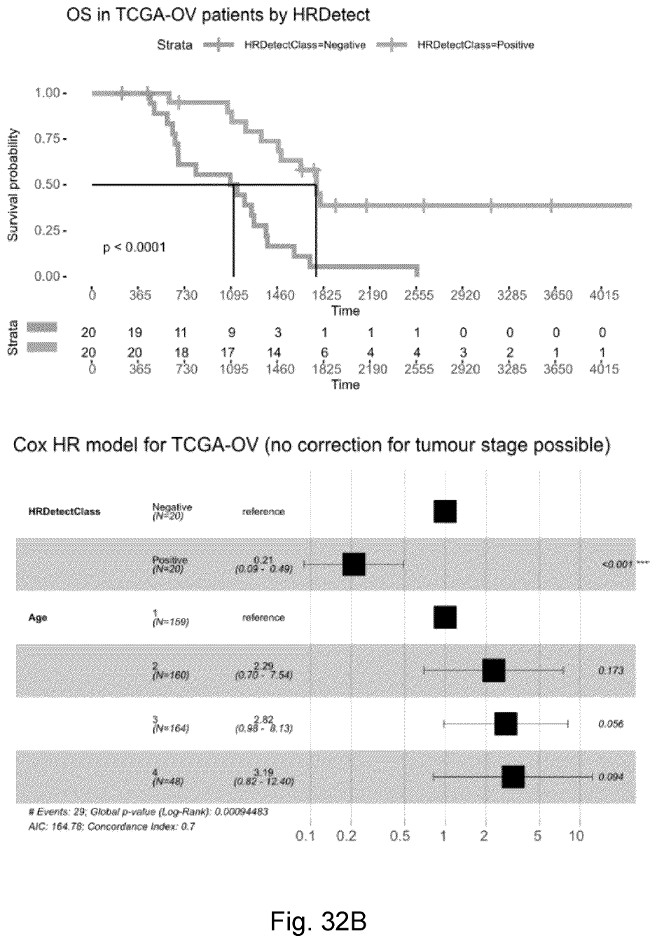

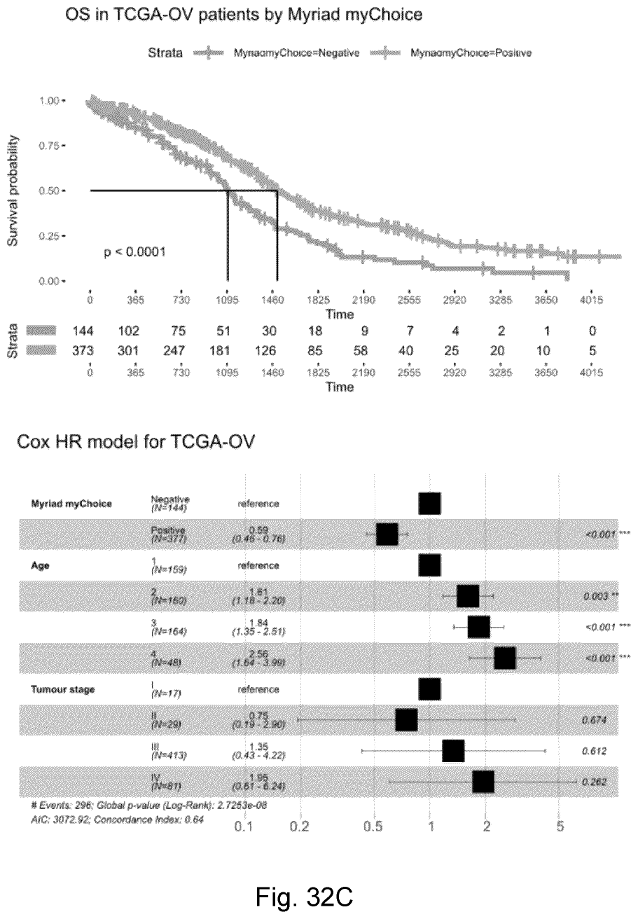

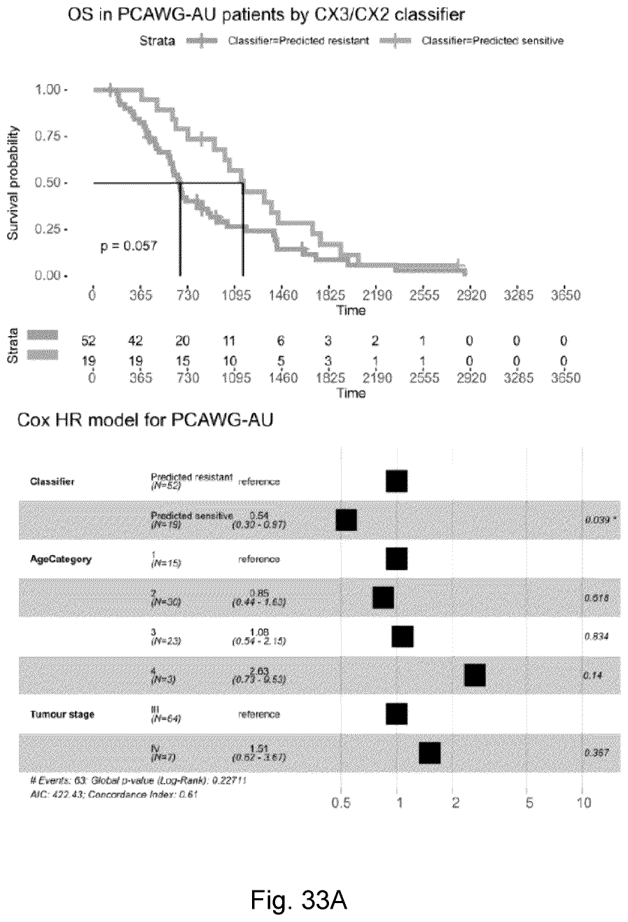

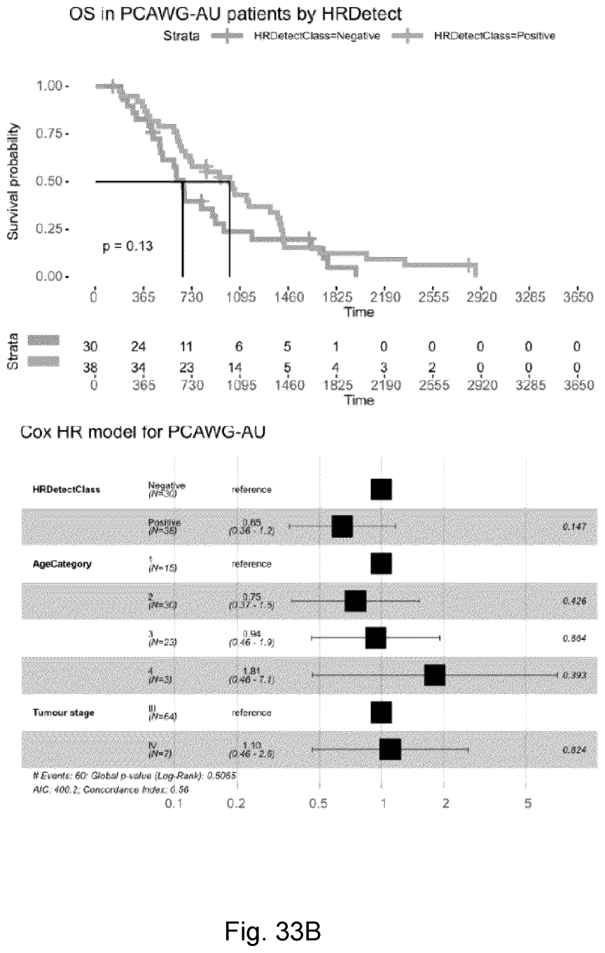

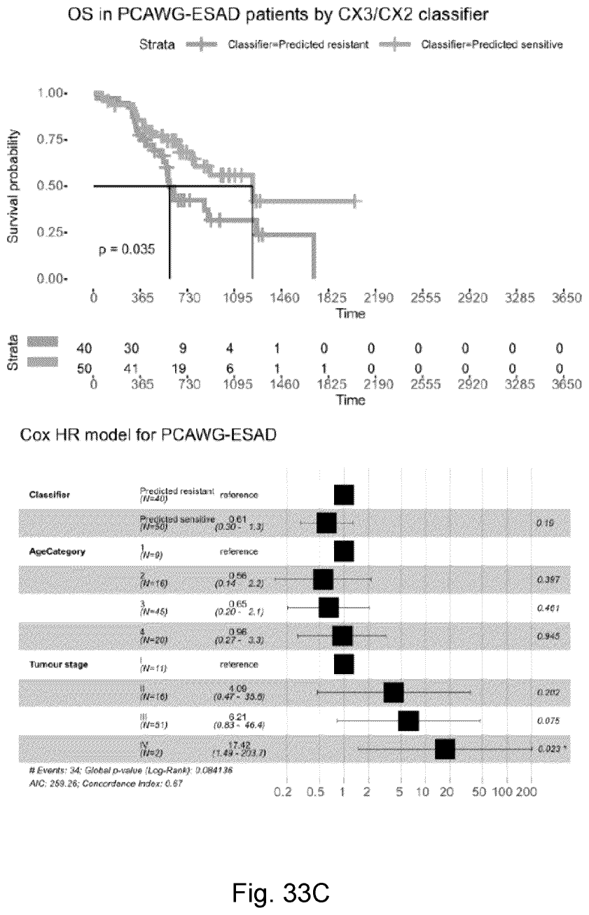

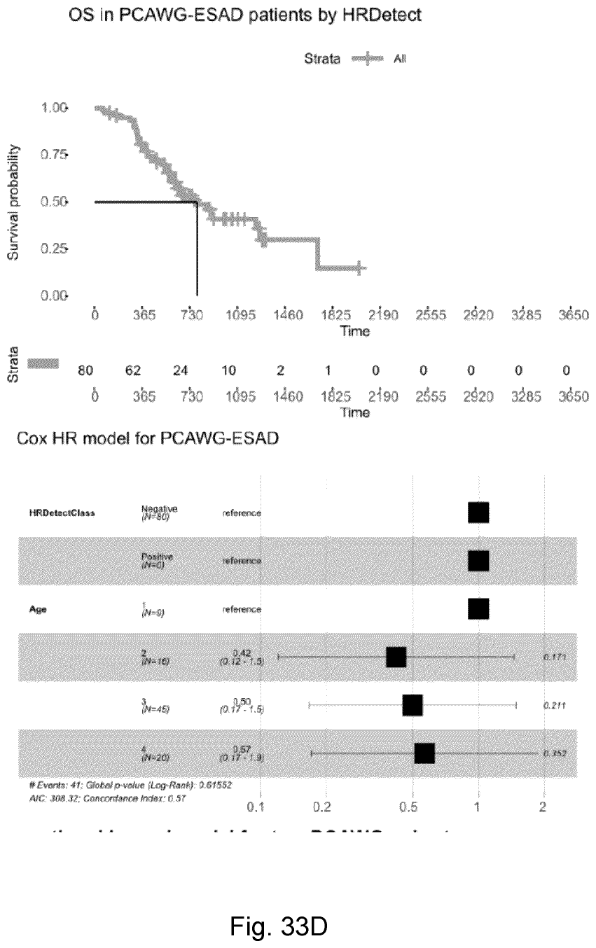

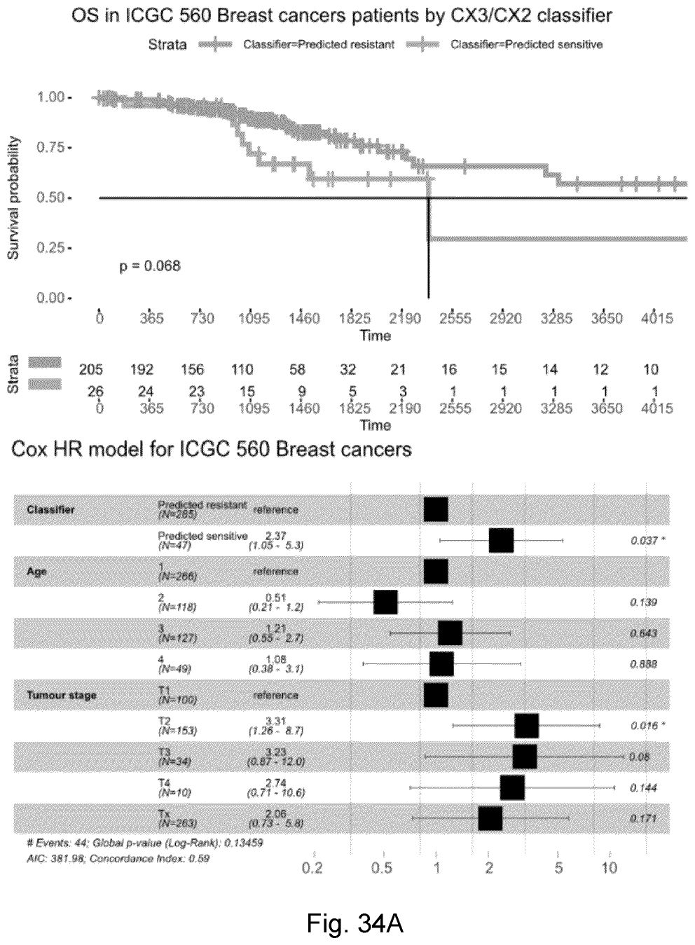

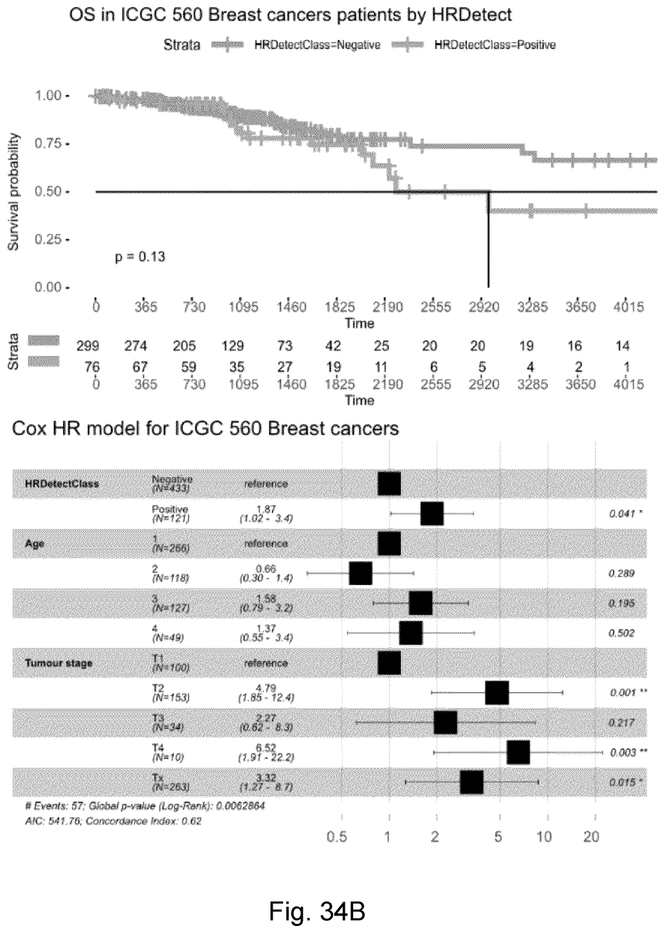

Given that the IHR signatures were able to dissect platinum response, we further hypothesised that they could be used in combination to provide better predictors of platinum sensitivity. Given that CX2 was not predictive, we used it as a baseline for measuring IHR-related genomic changes, and required CX3 activity to exceed it, resulting in a simple classification rule: “if CX3 activity is greater than CX2 activity, then predict sensitivity” (FIG. 28c). Despite its simplicity, this classifier was able to distinguish significant overall survival separation within BRCA1 germline mutant ovarian cancers, across the entire TCGA ovarian cohort, an independent validation cohort, and across an oesophageal cancer cohort (also routinely treated with platinum-based chemotherapy) (FIG. 28d, FIGS. 30-34). Other classifiers using all 3 IHR signatures, including more complex machine learning methods, did not outperform this decision rule (FIG. 35). Furthermore, this simple classifier had comparable performance and in some cases superior performance to more complex state-of-the-art HRD predictors across ovarian, oesophageal and breast cancer cohorts (FIG. 30c-d). Applying the CX2/CX3 classifier to the whole TCGA ovarian cohort estimated 27% of tumours to be platinum sensitive. Applying the classifier pan-cancer, we estimate that 8% of all tumours might be sensitive.

### Example 5—Predicting Sensitivity to Platinum-Based Chemotherapy

In this example, the inventors further validated the findings described in Example 4 in relation to prediction of sensitivity to platinum-based chemotherapy, in an independent cohort of ovarian cancer patients.

### Results

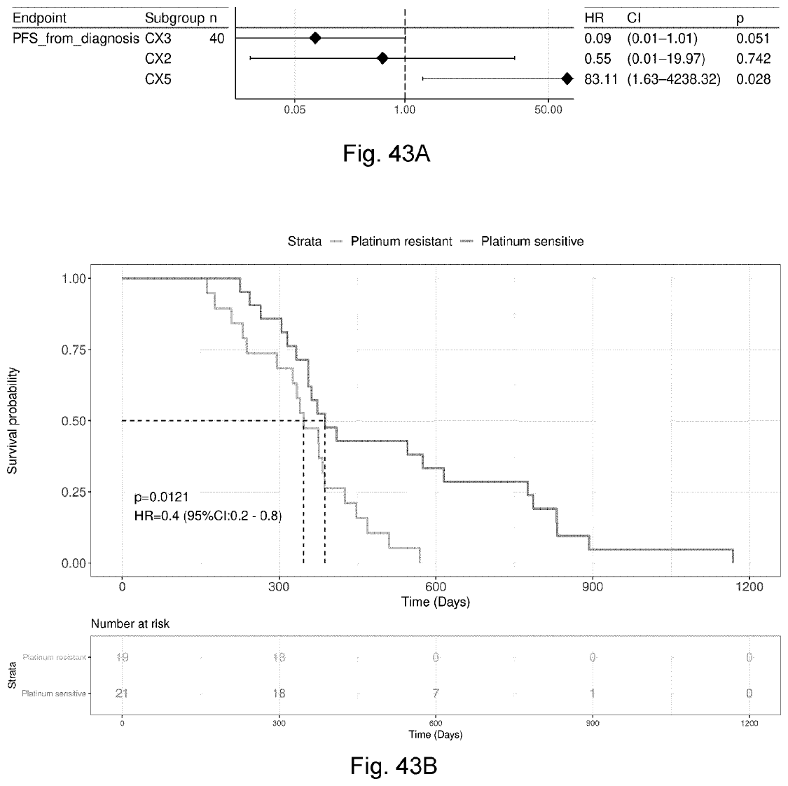

Using CIN signatures quantified across an independent cohort of 40 high-grade serous ovarian cancer patients treated with platinum-based chemotherapy, the inventors validated the ability of CX2, CX3 and CX5 to predict sensitivity to platinum-based chemotherapy treatment. They trained cox proportional hazards models to predict the progression free survival interval after first-line treatment for all three signatures, and the CX3/CX2 classifier described in Example 4. As described in Example 4, it was found that CX2 was not predictive of sensitivity, CX5 predicted resistance and CX3 predicted sensitivity (FIG. 43A). In addition, the CX3/CX2 classifier described in Example 4 was also predictive of sensitivity to platinum-based chemotherapy (FIG. 43B).

### Methods

Ethical approval and clinical sample collection. Clinical data and samples for the patients were collected as part of the prospective Cambridge Translational Cancer Research Ovarian Study 04 (CTCROV04) approved by the Institutional Ethics Committee (REC08/H0306/61). Patients provided written, informed consent for participation in this study and for the use of their donated tissue for the laboratory studies carried out in this work.

Sample processing for tissues. FFPE tissue blocks were cut as 8 μm sections and tumour-enriched regions were recovered by macrodissection based on regions marked on an adjacent haematoxylin-and-eosin-stained section by the study pathologist. DNA was extracted from 3-10 sections using QIAmp DNA Micro kit (Qiagen) with the following modification to the original protocol: an additional incubation step with Buffer ATL at 95.C for 15 minutes was introduced before adding proteinase K. The paraffin was removed using a xylene/ethanol method.

DNA sequencing. Whole-genome sequence libraries were prepared from 75 ng DNA using SMARTer Thruplex DNA-Seq (Takara) protocol. DNA from each sample was sheared on Covaris LE220 (Covaris): duty cycle-30%, intensity-5.0, bursts per sec-50, duration-120 sec, peak incident power-180, temperature 20° C., water level-4. All samples underwent 5 PCR cycles. Library quality and quantity were assessed with D5000 on 4200 Tapestation according to the supplier's recommendations. Libraries were then pooled together and sequenced using PE-50 mode on NovaSeq SP aiming for 10 million reads per sample.

Absolute copy-number fitting. Reads were aligned against the human genome assembly GRCh37 using BWA-MEM (Li 2013, arXiv: 1303.3997). Duplicates were marked using Picard (github.com/broadinstitute/picard) and relative copy number was computed using QDNAseq (Scheinin et al. Genome Res. 2014 December; 24 (12): 2022-32.) with a bin size of 30 kb. Absolute tumour copy number (the number of chromosome copies of each DNA segment in the tumour cells in a sample) was computed for every bin across each sample. Each segmented relative copy number bin estimate j was transformed from relative copy number (rCN) to absolute copy number (aCN) as follows:

$${aCN}_{j} = \frac{1}{purity} \cdot ( \frac{{rCN}_{j}}{d} - 2 \times ( 1 - purity ) ) - 2$$

where purity is the fraction of tumour cells in the sample, and d is a constant proportional to the read depth, which is computed from the mean relative copy number of the sample, r, and the average absolute copy number of the tumour cells in the sample, ploidy.

$$d = \frac{r}{( ploidy \times purity + 2 \times ( 1 - purity ) )}$$

Both purity and ploidy were unobserved in the data and were estimated using a grid search of purities ranging from [0.05,1] in 0.01 increments and ploidies ranging from [1.8,8] in increments of 0.1, minimising the following mean squared error:

$${e}_{purity , ploidy} = \frac{1}{J} \cdot {\sum }_{j = 1}^{J} {( aCN - round ( aCN ) )}^{2}$$

Purity/ploidy values were excluded from consideration if they resulted in a fit which showed greater than 10 megabases of the genome with homozygous loss. Fits were removed which did not show at least one genomic segment at every integer copy number state from 1 to ploidy.

Signature calling. Pre-identified copy number features such as copy number segment size, magnitude of copy number change, and frequency of copy number changes are extracted from the copy number profiles. These features are already compositionally associated with pancancer signatures as described above. As a result, by comparing the feature breakdown for an individual copy number profile to a matrix of signature-feature associations, it is possible to generate the signature breakdown for a sample. This signature breakdown gives exposures to each of the 17 pancancer signatures for the sample. In order to improve the reliability of the signature exposures each of the 17 signatures was used with a threshold, where values below the threshold are set to 0 to avoid false positives in signature exposure. These thresholds are defined in advance of the present cohort-specific analysis, using Markov-Chain Monte Carlo methods to identify the variability in each signature as a result of random noise in the copy number features. In this analysis, all downstream survival models used thresholded signature exposures. Finally, to compare exposures between signatures, each set of raw signatures for the samples were centred and scaled according to the distribution of the signature over all samples in the presently analysed cohort. By scaling the samples in this way, it is possible to compare exposures in the same sample by their relative magnitude in the cohort.

Calculating progression free survival (PFS) for platinum treatment. PFS was calculated following the CA125 definitions of progression in 1st Line Therapy, agreed by the Gynecologic Cancer InterGroup (GCIG) in 2005 (gcigtrials.org/system/files/CA%20125%20Definitions%20Agreed%20to%20by%20GCIG%20-%20November%202005.pdf). Patients were sorted into three separate categories based on their CA125 levels over the course of the Platinum 1st line treatment; Category A patients begin with ‘abnormal’ readings pre-treatment, that then fall into the normal range during treatment; Category B patients begin with abnormal readings that never normalise; and Category C patients begin in the normal range. In these definitions, the normal range was chosen to be 0-35 CA125, and the abnormal range 35+, following NICE guidance (nice.org.uk/guidance/cg122/resources/ovarian-cancer-recognition-and-initial-management-pdf-35109446543557). These categories then defined the patient-specific CA125 progression threshold. In categories A and C, progression occurs on the 1st CA125 reading that has at least twice the normal limit, or 70, provided that there is a consecutive reading of twice the normal limit recorded at least one week after the initial reading. In category B, the progression criteria are the same, with the exception that the progression limit is increased to be at least twice the lowest CA125 reading in the treatment line. In many patients, CA125 levels will continue to rise during the early platinum treatments, due to the lag between treatment administration and effect. To avoid false early progressions, CA 125 readings were only eligible for progression if they occurred after the last treatment in the line. There remain some cases where CA125 readings fail to reach the threshold for progression before the next line of therapy begins. In these cases the beginning of the next line is chosen as the date of progression. Using the progression date calculated for each patient, the progression-free survival was defined as the number of days from the date of diagnosis to the date of progression.

Survival analysis. Cox proportional-hazards models were generated in R using the coxph function from the survival package. Progression-free survival (PFS) was then predicted using various combinations of pancancer signature exposures. Signatures CX2, CX3 and CX5 were used individually, and in addition a classifier was made by comparing CX3 to CX2, where both signatures were centred and scaled to allow for comparison. As described above, in this example a scaled CX3>scaled CX2 was taken to indicate sensitivity, and a scaled CX3≤ scaled CX2 was taken to indicate resistance. To further confirm the results, covariates were also introduced to the cox model to test for significance. When including stratified age at diagnosis and tumour stage into the cox model, neither covariate showed significance in predicting survival, and neither changed the significance of the signature predictors.

### Example 6—Discussion

Examples 1-3 present a robust analysis framework for chromosomal instability in human cancers built on a pan-cancer analysis across 33 cancer types, and Examples 4-5 demonstrate its practical utility. This resource advances the field in two ways: it untangles CIN according to characteristic genomic patterns and underlying causes, and defines copy number signatures as new biomarkers to quantitatively measure different types of CIN. The approach described herein complements previous landscape studies of the genetic consequences of CIN (Network, TCGAR 2008, 2011, 2017, Zack et al., 2013), which generally focused on recurrent somatic copy number events at individual loci that are likely to reflect selective pressures (Zack et al., 2013). In contrast, copy number signatures (Macintyre et al., 2018, Steele et al., 2019) uncover mechanistic biases in the patterns of alterations across all chromosomes.

To maximise sample size, we used SNP 6.0 technology data from the TCGA collection. This technology is well established for copy number analysis, but has lower resolution than whole-genome sequencing. As further WGS data becomes available there will be an opportunity to further refine the signatures and increase their resolution. However, in their current form, we have demonstrated that the signatures are widely applicable across technologies, including inexpensive assays like shallow WGS that can be easily applied in a clinical setting to formalin-fixed tumour material (Scheinin et al., 2014). However, it is important to note that the bulk-sequenced samples we analysed do not show dynamics of CIN. The approach may be adapted to multiple samples or single cells from the same patient to show how patterns of CIN change over time. Further work may also be performed to quantify copy number signature activity at specific genomic loci, as the method described in these examples supports signature quantification at a whole-genome level.

The 17 copy number signatures (Tables 6 and 7) and their aetiologies provide a valuable resource for furthering our understanding of CIN. For example, CX1 represents the most prevalent type of CIN across tumours: chromosome missegregation. CX1 aetiology analysis pointed at multiple different mitotic defects giving rise to this signature. This suggests that, despite diversity in the potential causes of mitotic defects, these all result in the same change in genome structure (Bakhoum & Cantley, 2018). These missegregation events typically result in large copy number changes, potentially disrupting the function of many genes, however, our signature analysis reveals that these changes only represent, on average, 4% of the total number of copy number changes observed in a tumour (FIG. 36). In contrast, CX2 accounts for 23% of the copy number changes observed in a tumour. This highlights the power of our compendium of signatures to quantify and disentangle the causes, and functional impact that different types of CIN have on tumour genomes. Our results also highlight the potential of our signatures to improve the treatment of patients with extreme CIN tumours. Platinum-based chemotherapy is currently considered a broad spectrum cytotoxic chemotherapy and is routinely used to treat cancers with extreme CIN. However, here we showed that platinum response can be robustly dissected using different signatures of IHR. By developing our IHR signatures into a companion diagnostic assay, platinum-based therapies could potentially be administered in a more targeted manner, allowing resistant patients to avoid its toxic side effects, and healthcare systems to reduce the cost burden of ineffectual treatment. Similarly for other signatures, our analysis of drug response across cell lines reinforces their potential to be developed into companion diagnostics for improved patient stratification during clinical trials.

In summary, the signature compendium presented here is an important resource to guide future studies into a deeper understanding of the origins and diversity of CIN and how to therapeutically target different CIN types.

TABLE 6
Definition of the 43 mixture components used.

| Feature | Mean | SD | Weight | Number |
|---|---|---|---|---|
| segsize | 38420.7697 | 15438.8759 | 0.0150893 | 1 |
| segsize | 86319.107 | 30243.0733 | 0.02246956 | 2 |
| segsize | 171234.256 | 55096.0468 | 0.03478786 | 3 |
| segsize | 427454 | 174237 | 0.1237 | 4 |
| segsize | 918939.408 | 260126.652 | 0.07107799 | 5 |
| segsize | 1494667.9 | 404499.004 | 0.07395141 | 6 |
| segsize | 2496039.63 | 608098.846 | 0.09346463 | 7 |
| segsize | 3898217.97 | 583841.597 | 0.0566778 | 8 |
| segsize | 4908317.82 | 398896.482 | 0.01339669 | 9 |
| segsize | 6371179 | 985163 | 0.0659 | 10 |
| segsize | 8780945 | 1086234 | 0.0506 | 11 |
| segsize | 13435586 | 1439442 | 0.0414 | 12 |
| segsize | 18665397 | 1664701 | 0.0331 | 13 |
| segsize | 22913723.2 | 1028090.2 | 0.01095836 | 14 |
| segsize | 30753807 | 2370747.45 | 0.01373617 | 15 |
| segsize | 36812267.4 | 1799660.32 | 0.01285096 | 16 |
| segsize | 44866788 | 4250506.18 | 0.02235963 | 17 |
| segsize | 57831002.6 | 3574456.9 | 0.01970078 | 18 |
| segsize | 68086495.8 | 6023604.95 | 0.01683004 | 19 |
| segsize | 81015695.9 | 3149494.22 | 0.01317039 | 20 |
| segsize | 93268268 | 10492824.6 | 0.01645642 | 21 |
| segsize | 154120037 | 27901372.7 | 0.02569139 | 22 |
| changepoint | 0.10594582 | 0.06312023 | 0.06094551 | 1 |
| changepoint | 0.34283504 | 0.1572276 | 0.11805556 | 2 |
| changepoint | 1.00818588 | 0.2528096 | 0.37043868 | 3 |
| changepoint | 1.40754134 | 0.3988254 | 0.06601043 | 4 |
| changepoint | 2.20266882 | 0.4656683 | 0.2117235 | 5 |
| changepoint | 3.20155742 | 0.32865165 | 0.02131326 | 6 |
| changepoint | 4.11442601 | 0.49429328 | 0.02397049 | 7 |
| changepoint | 5.13850887 | 1.16026304 | 0.01390753 | 8 |
| changepoint | 6.93357762 | 1.76527488 | 0.01834703 | 9 |
| changepoint | 9.96341765 | 2.61954362 | 0.01218381 | 10 |
| bp10MB | 3.90E−08 | NA | 0.75713738 | 1 |
| bp10MB | 1.04971801 | NA | 0.23064104 | 2 |
| bp10MB | 5.43989834 | NA | 0.01222158 | 3 |
| bpchrarm | 0.05683922 | NA | 0.47183552 | 1 |
| bpchrarm | 1.9260777 | NA | 0.38270107 | 2 |
| bpchrarm | 7.06398355 | NA | 0.12115462 | 3 |
| bpchrarm | 18.1504596 | NA | 0.02221902 | 4 |
| bpchrarm | 46.6651445 | NA | 0.00208977 | 5 |
| osCN | 0.24540037 | NA | 0.91590854 | 1 |
| osCN | 2.42532595 | NA | 0.08006416 | 2 |
| osCN | 9.36453963 | NA | 0.00402731 | 3 |

Feature = One of the five fundamental features of copy number aberrations; Mean = If SD is present, then this is the mean of the Gaussian component, otherwise mean of the Poisson component; SD = If present, standard deviation of the Gaussian component; Weight = Weight in the mixture identified by the converged model; Number = Number of component used as identifiers.

TABLE 7
Signature definitions normalised per signature.

| | CX1 | CX2 | CX3 | CX4 | CX5 | CX6 | CX7 | CX8 | CX9 |
|---|---|---|---|---|---|---|---|---|---|
| segsize1 | 0 | 0 | 0.00016184 | 0 | 0.00216351 | 0 | 0 | 0 | 0.0104853 |
| segsize2 | 0 | 0.00484254 | 0.0014391 | 1.12E−12 | 1.31E−24 | 0 | 0 | 0 | 0.00886383 |
| segsize3 | 1.80E−42 | 0.02408467 | 0.00146289 | 0.01971736 | 1.10E−17 | 0 | 0 | 0 | 0.01954622 |
| segsize4 | 4.93E−05 | 0.07132246 | 1.98E−27 | 0.05888886 | 0.03046283 | 0 | 3.62E−19 | 1.82E−11 | 0.04760496 |
| segsize5 | 0 | 0.06398659 | 0 | 0.04537172 | 0.05376763 | 0 | 0 | 0.01192221 | 0.04544527 |
| segsize6 | 1.34E−15 | 0.05328949 | 5.88E−17 | 0.03511257 | 0.06703374 | 0 | 0 | 1.71E−10 | 0.02003537 |
| segsize7 | 0.00124638 | 0.0370812 | 0.00874625 | 0.01889691 | 0.07095821 | 0 | 0 | 0 | 2.74E−14 |
| segsize8 | 0.00073204 | 0.0152153 | 0.01220894 | 0.00685698 | 0.04356222 | 0 | 0 | 0 | 0 |
| segsize9 | 0.00026715 | 0.00661238 | 0.00999693 | 0.00107158 | 0.02570263 | 0 | 4.00E−45 | 0 | 0 |
| segsize10 | 0.00018658 | 0.00666403 | 0.01963967 | 0.00298085 | 0.03470504 | 0 | 0 | 0 | 0 |
| segsize11 | 0 | 0.00517994 | 0.02605192 | 0.00494172 | 0.03068329 | 0 | 0 | 0 | 0 |
| segsize12 | 0 | 0.00298202 | 0.02996957 | 0.00669632 | 0.02035681 | 0 | 0 | 0 | 0 |
| segsize13 | 0.00059621 | 0.00083885 | 0.02290195 | 0.00663182 | 0.01009959 | 0 | 0 | 0 | 0.00706606 |
| segsize14 | 0.00089838 | 5.19E−24 | 0.01547621 | 0.00545447 | 0.00384132 | 0 | 8.62E−43 | 8.28E−32 | 0.00861475 |
| segsize15 | 0.00189299 | 0 | 0.02348316 | 0.0139198 | 8.81E−09 | 2.23E−27 | 0.0077465 | 0.00800351 | 0.01225905 |
| segsize16 | 0.00179258 | 0 | 0.01022332 | 0.00507305 | 2.84E−35 | 0.09072743 | 0 | 0.00171533 | 0.00745238 |
| segsize17 | 0.00245739 | 0 | 0.01477938 | 0.00358163 | 0 | 0.09977945 | 0 | 0.00918182 | 0.01981539 |
| segsize18 | 0.00191146 | 0 | 0.00731191 | 0.00159002 | 0 | 0.0789592 | 0 | 0.00514497 | 0.01251594 |
| segsize19 | 0.00134138 | 0 | 0.00583115 | 0.00117343 | 0 | 0.09459047 | 0 | 0.00271298 | 0.01093206 |
| segsize20 | 0.00108847 | 0 | 0.00181984 | 0.00018712 | 0 | 0.12590562 | 0 | 0 | 0.00503965 |
| segsize21 | 0.00275101 | 0 | 0.00316785 | 5.46E−30 | 0 | 0.18223962 | 0 | 0.00254837 | 0.01103619 |
| segsize22 | 0.00320638 | 0 | 0 | 0 | 0 | 0.3277982 | 0 | 0 | 0 |
| changepoint1 | 0.00714553 | 0 | 0.00570593 | 0.00644497 | 0 | 1.52E−28 | 0.37146604 | 0 | 0 |
| changepoint2 | 0.0018116 | 0.00231429 | 0.02066105 | 0 | 0.02592285 | 0 | 0.6207804 | 0 | 0 |
| changepoint3 | 0.0057141 | 0.30264735 | 0.06298824 | 0 | 0 | 0 | 0 | 0 | 0 |
| changepoint4 | 0.00395169 | 0.1590432 | 0.04408744 | 0.14370395 | 4.89E−07 | 6.36E−29 | 0 | 0 | 0 |
| changepoint5 | 0.00081806 | 0.00130316 | 0.01884735 | 0.44735542 | 2.87E−22 | 0 | 0 | 0 | 0 |
| changepoint6 | 0 | 0 | 0.00113433 | 0.04626792 | 0.00572015 | 0 | 0 | 0.25772804 | 0.08034599 |
| changepoint7 | 0 | 0 | 2.48E−07 | 0.013576 | 0.00060475 | 0 | 0 | 0.26241368 | 0.08338061 |
| changepoint8 | 7.51E−07 | 2.87E−05 | 0.00014151 | 0.01335823 | 0.00187002 | 0 | 0 | 0.23063518 | 0.08601107 |
| changepoint9 | 5.55E−06 | 0.00021389 | 6.04E−11 | 0.00397518 | 0.00156376 | 0 | 0 | 0.16147758 | 0.08433821 |
| changepoint10 | 3.72E−23 | 4.57E−05 | 1.20E−42 | 0.00120084 | 0.00066237 | 0 | 0 | 0.04651625 | 0.11725483 |
| bp10MB1 | 0.6028781 | 0.01707379 | 0.20746246 | 1.65E−11 | 2.13E−07 | 0 | 0 | 0 | 4.12E−39 |
| bp10MB2 | 0.23454046 | 0.0713407 | 0.23615567 | 7.28E−09 | 0.08124033 | 0 | 0 | 0 | 0 |
| bp10MB3 | 0.00353574 | 0.0774666 | 0.01810788 | 0.04959495 | 0.05605663 | 0 | 0 | 0 | 0.01163367 |
| bpchrarm1 | 0.07670944 | 0 | 8.65E−12 | 0 | 0 | 0 | 0 | 0 | 0 |
| bpchrarm2 | 0.03989853 | 0 | 0.03907297 | 0 | 0 | 0 | 0 | 0 | 0 |
| bpchrarm3 | 0.0012317 | 0.02368954 | 0.03015067 | 0.00867143 | 0.00737851 | 0 | 0 | 0 | 0.02564305 |
| bpchrarm4 | 0 | 0.00697882 | 0 | 0.00188251 | 0.01475545 | 0 | 0 | 0 | 0 |
| bpchrarm5 | 0 | 0.00102692 | 0 | 0 | 0 | 0 | 0 | 0 | 3.10E−06 |
| osCN1 | 0.00134109 | 4.71E−25 | 0.08072928 | 0 | 0.33415312 | 0 | 0 | 0 | 0.26237002 |
| osCN2 | 0 | 0.03703466 | 0.02008309 | 0.02243513 | 0.07673459 | 0 | 0 | 0 | 0.00230712 |
| osCN3 | 0 | 0.00769325 | 0 | 0.00338728 | 0 | 0 | 7.01E−06 | 0 | 0 |

| | CX10 | CX11 | CX12 | CX13 | CX14 | CX15 | CX16 | CX17 |
|---|---|---|---|---|---|---|---|---|
| segsize1 | 0.04909098 | 0.1532556 | 0.54287195 | 0 | 0 | 0 | 0.1941291 | 0.00302181 |
| segsize2 | 0.06240538 | 0.13370365 | 0.29501578 | 0 | 0 | 1.36E−34 | 0.05313293 | 0.00644556 |
| segsize3 | 0.11545427 | 0.15966316 | 0.13973829 | 0 | 0 | 1.17E−37 | 0.01458414 | 0.01131112 |
| segsize4 | 0.15082364 | 0.13743967 | 0 | 0 | 0 | 1.17E−19 | 0.00763752 | 0.01833861 |
| segsize5 | 0.04282543 | 0.0128697 | 0 | 0 | 8.89E−14 | 0.00675661 | 0.00751893 | 0.0256733 |
| segsize6 | 5.97E−16 | 0 | 0 | 0 | 0.00657832 | 0.0178896 | 0.00581555 | 0.02397868 |
| segsize7 | 0 | 0 | 0 | 0 | 7.36E−13 | 0.03367993 | 0.00871748 | 0.02118697 |
| segsize8 | 0 | 0 | 0 | 0 | 0.0094351 | 0.02610913 | 0.0124591 | 0.01962675 |
| segsize9 | 0 | 0 | 0 | 0 | 0.00603125 | 0.0174711 | 0.01134712 | 0.01003373 |
| segsize10 | 0 | 0 | 0 | 0 | 2.76E−43 | 0.02926329 | 0.01537962 | 0.01600513 |
| segsize11 | 1.14E−22 | 0 | 0 | 0 | 0 | 0.0344689 | 0.01131485 | 0.02253518 |
| segsize12 | 0.00109233 | 0 | 0 | 0 | 3.72E−26 | 0.03380073 | 0.00971938 | 0.02412889 |
| segsize13 | 7.82E−15 | 0 | 0 | 0 | 0 | 0.02264792 | 0.0054729 | 0.02672835 |
| segsize14 | 4.00E−44 | 0 | 0 | 0 | 3.08E−27 | 0.01624692 | 0.00434368 | 0.01647804 |
| segsize15 | 1.26E−36 | 0 | 0 | 0 | 0.00032232 | 0.02507739 | 0 | 0.01759323 |
| segsize16 | 0 | 0 | 0 | 0 | 0.03878736 | 0.00745051 | 0.00031091 | 0.00921888 |
| segsize17 | 0 | 0 | 0 | 0 | 0.03632237 | 0.0128698 | 0 | 0.01182435 |
| segsize18 | 0.00076324 | 0 | 0 | 0 | 0.03332187 | 0.00290087 | 0 | 0.01573012 |
| segsize19 | 0 | 0 | 0 | 0 | 0.05069218 | 0.00447889 | 0 | 0.01058943 |
| segsize20 | 0 | 0 | 0 | 0 | 0.0896177 | 2.54E−06 | 0 | 1.24E−35 |
| segsize21 | 0 | 0 | 0 | 0 | 0.07083435 | 0.0040517 | 0 | 7.90E−11 |
| segsize22 | 0 | 0 | 0 | 0 | 0.24536963 | 0 | 0 | 0 |
| changepoint1 | 0.03802524 | 0.01231251 | 0 | 0 | 0.19935879 | 0.05083028 | 0.0028998 | 0 |
| changepoint2 | 0 | 0 | 0 | 0 | 0.21332453 | 0.10116892 | 0 | 0 |
| changepoint3 | 4.66E−34 | 0 | 0 | 0 | 1.41E−12 | 0.07578269 | 0 | 0.02823178 |
| changepoint4 | 0.05424671 | 2.27E−31 | 0 | 0 | 1.35E−10 | 0.03476157 | 0.0379787 | 0.07109328 |
| changepoint5 | 0.1715475 | 3.48E−10 | 0 | 0 | 9.78E−33 | 3.03E−07 | 0.15329279 | 0.13414173 |
| changepoint6 | 0.02771987 | 0 | 0 | 0 | 0 | 0 | 0.0856759 | 0.03689786 |
| changepoint7 | 0.01198916 | 0 | 0 | 0 | 0 | 0 | 0.01279051 | 0.02626175 |
| changepoint8 | 0.00962178 | 0.00010044 | 0 | 0 | 0 | 4.96E−05 | 0.01413791 | 0.02258389 |
| changepoint9 | 0.00366384 | 3.63E−05 | 0 | 0.10281373 | 8.66E−39 | 0.00014521 | 0.00837755 | 0.01363431 |
| changepoint10 | 0 | 0.0001242 | 0 | 0.8870933 | 2.85E−33 | 0 | 0.00330837 | 0.01301523 |
| bp10MB1 | 1.94E−08 | 4.59E−22 | 0 | 0 | 6.09E−23 | 3.17E−10 | 0.05867745 | 5.26E−08 |
| bp10MB2 | 6.05E−17 | 6.33E−37 | 0 | 0 | 1.03E−28 | 0.1804205 | 0.0632623 | 0.10061176 |
| bp10MB3 | 0.07684001 | 0.06936349 | 2.14E−24 | 0 | 0 | 0.03141776 | 0.0679324 | 0.03703585 |
| bpchrarm1 | 0.00200804 | 0 | 0 | 0 | 1.08E−08 | 0 | 0 | 0 |
| bpchrarm2 | 0 | 0 | 0 | 0 | 3.59E−24 | 0.04683046 | 0 | 0.01812208 |
| bpchrarm3 | 0.00325985 | 0.01924726 | 1.00E−45 | 0 | 0 | 0.02757227 | 0.01662104 | 0.03150475 |
| bpchrarm4 | 0.01738671 | 0.00648727 | 0 | 0 | 0 | 1.29E−12 | 0.01380378 | 0.00069697 |
| bpchrarm5 | 0.00097641 | 0.00238764 | 0.00059188 | 0.0100929 | 0 | 0 | 0.00041607 | 0 |
| osCN1 | 0.06969012 | 0.20960216 | 0 | 0 | 0 | 0.12358192 | 0.04201865 | 0.11868507 |
| osCN2 | 0.08435372 | 0.08032654 | 0.01664069 | 0 | 0 | 0.03227263 | 0.04668589 | 0.03591845 |
| osCN3 | 0.00621559 | 0.00308044 | 0.00514142 | 0 | 4.33E−06 | 0 | 0.01023775 | 0.00111711 |

Columns = Mixture components; Rows = Signatures. Cells = Weight.

TABLE 8
Change in signature activities for mutations
in high-confidence genes (see FIG. 23)

| signature | gene | rho | p. value | q. value |
|---|---|---|---|---|
| CX2 | KPNA2 | 0.174546587239601 | 1.13E−22 | 3.09E−20 |
| CX2 | EME1 | 0.147191918329808 | 1.88E−13 | 2.57E−10 |
| CX2 | BLM | 0.132697157890139 | 9.24E−08 | 8.44E−06 |
| CX2 | POLK | −0.127854335264968 | 2.65E−08 | 1.81E−04 |
| CX2 | FANCD2 | 0.127134000356769 | 4.32E−06 | 2.37E−04 |
| CX2 | CCNH | −0.118598509410718 | 1.14E−03 | 5.20E−02 |
| CX2 | MAD2L2 | 0.117125482995099 | 2.86E−04 | 1.12E−01 |
| CX2 | CCNA2 | 0.109892555684754 | 2.25E−01 | 7.71E+00 |
| CX2 | NEIL2 | −0.106082266566602 | 2.01E+00 | 6.10E+01 |
| CX2 | GPS1 | 0.104223271349443 | 5.67E+00 | 1.55E+02 |
| CX2 | FAAP100 | 0.102333299244932 | 1.60E+01 | 3.99E+02 |
| CX2 | DDB2 | −0.101097982244208 | 3.12E+01 | 7.13E+02 |
| CX2 | POLQ | 0.100794219506909 | 3.68E+01 | 7.75E+02 |
| CX2 | MSH3 | −0.100363216335258 | 4.63E+00 | 9.06E+02 |
| CX3 | ACTL6A | 0.358101500252626 | 2.30E−142 | 6.30E−140 |
| CX3 | RFC4 | 0.343094534754342 | 7.16E−129 | 9.81E−127 |
| CX3 | KPNA2 | 0.280974590501962 | 1.72E−80 | 1.57E−78 |
| CX3 | TOPBP1 | 0.273485441745686 | 1.99E−75 | 1.36E−73 |
| CX3 | ISY1 | 0.267329044081555 | 1.10E−67 | 6.01E−66 |
| CX3 | CCNH | −0.260609301087006 | 4.34E−67 | 1.98E−65 |
| CX3 | EME1 | 0.254007632704388 | 5.43E−63 | 2.12E−61 |
| CX3 | POLQ | 0.250947976387581 | 3.92E−61 | 1.34E−59 |
| CX3 | RAD51AP1 | 0.247450784103156 | 4.85E−59 | 1.48E−57 |
| CX3 | MSH2 | 0.245647034179157 | 5.66E−58 | 1.55E−56 |
| CX3 | BLM | 0.241215760884101 | 2.17E−55 | 5.40E−54 |
| CX3 | MSH3 | −0.237471647465788 | 3.01E−53 | 6.87E−52 |
| CX3 | MSH6 | 0.235262933200862 | 5.30E−53 | 1.12E−50 |
| CX3 | POLK | −0.231809053186526 | 4.44E−50 | 8.68E−49 |
| CX3 | RAD17 | −0.222677049659681 | 3.80E−45 | 6.94E−44 |
| CX3 | EXO1 | 0.219305962850724 | 2.21E−43 | 3.79E−42 |
| CX3 | POLR2H | 0.216782774170516 | 4.44E−42 | 7.16E−41 |
| CX3 | UBA7 | −0.214308525472241 | 8.11E−41 | 1.23E−39 |
| CX3 | UBE2T | 0.212242604422656 | 8.91E−40 | 1.29E−38 |
| CX3 | XPC | −0.209384610605298 | 2.36E−38 | 3.23E−37 |
| CX3 | FANCI | 0.208689452821377 | 5.19E−38 | 6.77E−37 |
| CX3 | FANCB | 0.211987278743076 | 8.82E−37 | 1.10E−36 |
| CX3 | RNF168 | 0.205135652366604 | 2.80E−36 | 3.34E−35 |
| CX3 | RUVBL1 | 0.204104601229527 | 8.80E−36 | 1.00E−34 |
| CX3 | RAD1 | 0.202862647817713 | 3.46E−35 | 3.79E−34 |
| CX3 | CHEK2 | 0.196893226303822 | 2.21E−33 | 2.33E−31 |
| CX3 | DNA2 | 0.195347777709329 | 1.14E−31 | 1.15E−30 |
| CX3 | BRIP1 | 0.193201054872836 | 1.08E−31 | 1.06E−29 |
| CX3 | CDK7 | −0.189625028780851 | 4.34E−29 | 4.10E−28 |
| CX3 | POLR2D | 0.188863377918079 | 9.44E−29 | 8.62E−28 |
| CX3 | GTF2H4 | 0.187485255664576 | 3.82E−28 | 3.38E−27 |
| CX3 | FANCE | 0.184497650354278 | 7.62E−27 | 6.52E−26 |
| CX3 | XRCC2 | 0.18321406442592 | 2.71E−26 | 2.25E−25 |
| CX3 | MDC1 | 0.181239395938412 | 1.88E−25 | 1.51E−24 |
| CX3 | KDM4B | −0.180472471710647 | 3.96E−25 | 3.10E−24 |
| CX3 | CCNA2 | 0.174755292041162 | 9.27E−23 | 7.05E−22 |
| CX3 | MBD4 | 0.174553329809904 | 1.12E−22 | 8.29E−22 |
| CX3 | FANCD2 | 0.174436212201088 | 1.25E−22 | 9.01E−22 |
| CX3 | RAD51 | 0.17178249965808 | 1.47E−21 | 1.03E−20 |
| CX3 | RPA1 | 0.167467385143061 | 7.43E−20 | 5.09E−19 |
| CX3 | FEN1 | 0.167285006129595 | 8.75E−20 | 5.85E−19 |
| CX3 | RAD51C | 0.165784756934059 | 3.33E−19 | 2.17E−18 |
| CX3 | XRCC3 | 0.163902099166434 | 1.76E−18 | 1.12E−17 |
| CX3 | CHEK1 | 0.161839883335458 | 1.06E−17 | 6.59E−17 |
| CX3 | GEN1 | 0.161020956325629 | 2.15E−17 | 1.31E−16 |
| CX3 | CDK2 | 0.160967443746372 | 2.25E−17 | 1.34E−17 |
| CX3 | CLSPN | 0.160885407239125 | 2.41E−17 | 1.41E−16 |
| CX3 | USP1 | 0.159587089477009 | 7.34E−17 | 4.19E−16 |
| CX3 | TIMELESS | 0.158358912301934 | 2.08E−16 | 1.17E−15 |
| CX3 | RAD50 | −0.157104586145 | 6.00E−16 | 3.29E−15 |
| CX3 | RHNO1 | 0.153081190211884 | 1.68E−14 | 9.02E−14 |
| CX3 | FANCA | 0.152368216545033 | 3.00E−14 | 1.58E−13 |
| CX3 | GPS1 | 0.152225999200225 | 3.37E−14 | 1.74E−14 |
| CX3 | POLI | −0.150913601666601 | 9.75E−14 | 4.95E−13 |
| CX3 | FAAP24 | 0.150236999772154 | 1.68E−13 | 8.36E−14 |
| CX3 | MPG | −0.149615116346125 | 2.76E−13 | 1.35E−12 |
| CX3 | UBE2B | −0.147988103385166 | 1.00E−13 | 4.83E−12 |
| CX3 | POLR2K | 0.146984745425436 | 2.21E−12 | 1.04E−11 |
| CX3 | UBE2V2 | 0.146849829852224 | 2.46E−12 | 1.14E−11 |
| CX3 | RNF8 | 0.145088861121525 | 9.67E−12 | 4.42E−11 |
| CX3 | PRKDC | 0.144529258613787 | 1.49E−11 | 6.69E−11 |
| CX3 | SIRT6 | −0.144370807374535 | 1.68E−11 | 7.44E−11 |
| CX3 | YY1 | 0.140143903317123 | 4.14E−10 | 1.80E−09 |
| CX3 | ATR | 0.137312381444243 | 3.35E−09 | 1.44E−08 |
| CX3 | PARP2 | 0.137039915934063 | 4.09E−09 | 1.72E−08 |
| CX3 | NEIL2 | −0.136638112175456 | 5.48E−09 | 2.28E−08 |
| CX3 | MUTYH | 0.136401843922046 | 6.51E−09 | 2.66E−08 |
| CX3 | DTL | 0.132905224569568 | 7.98E−08 | 3.21E−07 |
| CX3 | PPIE | 0.131637582936782 | 1.95E−07 | 7.73E−07 |
| CX3 | PARP1 | 0.127536783202478 | 3.29E−06 | 1.29E−06 |
| CX3 | ABRAXAS1 | −0.126892701124869 | 5.08E−06 | 1.96E−06 |
| CX3 | ERCC3 | 0.125851836553035 | 1.02E−05 | 3.89E−05 |
| CX3 | DCLRE1B | 0.124134109845651 | 4.60E−05 | 1.72E−05 |
| CX3 | RFC2 | 0.122467758935659 | 9.54E−05 | 3.49E−04 |
| CX3 | DDB2 | −0.120785607861041 | 2.83E−04 | 1.01E−03 |
| CX3 | BRCA1 | 0.120314700693923 | 3.83E−04 | 1.34E−03 |
| CX3 | RFC3 | 0.119452422245374 | 6.63E−04 | 2.30E−03 |
| CX3 | XPA | −0.118472369763365 | 1.23E−03 | 4.22E−03 |
| CX3 | POLE2 | 0.118286527586263 | 1.39E−03 | 4.69E−03 |
| CX3 | NEIL3 | 0.118130024946529 | 1.53E−03 | 5.11E−03 |
| CX3 | NSD2 | 0.117742864007102 | 1.95E−03 | 6.43E−03 |
| CX3 | SUMO2 | 0.117633424737776 | 2.09E−03 | 6.81E−03 |
| CX3 | MRE11 | 0.116665744215006 | 3.81E−03 | 1.23E−03 |
| CX3 | PCLAF | 0.116181468787627 | 5.14E−03 | 1.64E−02 |
| CX3 | SPRTN | 0.115215123304563 | 9.32E−03 | 2.93E−02 |
| CX3 | INO80 | −0.11405024551787 | 1.89E−03 | 5.83E−02 |
| CX3 | MAD2L2 | 0.114008358753863 | 1.94E−02 | 5.92E−02 |
| CX3 | COPS3 | 0.11385575160354 | 2.13E−02 | 6.42E−02 |
| CX3 | RAD9A | 0.113515961588227 | 2.62E−02 | 7.79E−02 |
| CX3 | ERCC8 | −0.111846359360869 | 7.11E−02 | 2.07E−01 |
| CX3 | POLD3 | 0.110778289317967 | 1.34E−01 | 3.86E−02 |
| CX3 | RFC5 | 0.110432713978613 | 1.64E−01 | 4.68E−01 |
| CX3 | DCLRE1C | 0.109730929878079 | 2.47E−01 | 6.98E−01 |
| CX3 | PRPF19 | 0.108492152771422 | 5.07E−02 | 1.42E+00 |
| CX3 | FAAP100 | 0.108153558880693 | 6.16E−01 | 1.71E+00 |
| CX3 | AQR | −0.105814849904717 | 2.33E−01 | 6.39E+00 |
| CX3 | RAD18 | 0.105553343416755 | 2.70E+00 | 7.32E+00 |
| CX3 | UFD1 | 0.104680168488432 | 5.95E+00 | 1.60E+01 |
| CX3 | FANCL | 0.103622121648537 | 7.90E+00 | 2.10E+01 |
| CX3 | FANCG | 0.101824461147853 | 3.06E+01 | 7.98E+01 |
| CX5 | KPNA2 | 0.268540520649596 | 3.60E−72 | 9.86E−71 |
| CX5 | EME1 | 0.258121263507938 | 1.57E−65 | 2.15E−63 |
| CX5 | BLM | 0.247696730962602 | 3.47E−61 | 3.17E−57 |
| CX5 | FANCD2 | 0.218322292530115 | 7.16E−43 | 4.91E−41 |
| CX5 | RAD51AP1 | 0.216686984239325 | 4.98E−42 | 2.73E−40 |
| CX5 | RAD51 | 0.212036341879433 | 1.13E−39 | 5.16E−38 |
| CX5 | ACTL6A | 0.209901494660953 | 1.31E−38 | 5.12E−37 |
| CX5 | POLQ | 0.209100052129507 | 3.26E−38 | 1.11E−36 |
| CX5 | CCNA2 | 0.196491050749643 | 3.38E−32 | 1.03E−30 |
| CX5 | EXO1 | 0.19131077142358 | 7.68E−30 | 2.10E−28 |
| CX5 | MSH6 | 0.187126977228267 | 5.48E−28 | 1.37E−26 |
| CX5 | UBA7 | −0.186100082381673 | 1.54E−27 | 3.52E−26 |
| CX5 | FANCI | 0.185925969880079 | 1.83E−27 | 3.86E−26 |
| CX5 | BRIP1 | 0.180796011262587 | 2.89E−25 | 5.66E−24 |
| CX5 | RFC4 | 0.179331523834718 | 1.19E−24 | 2.18E−23 |
| CX5 | CCNH | −0.177112427594232 | 1.00E−23 | 1.71E−22 |
| CX5 | CLSPN | 0.176025325408029 | 2.80E−23 | 4.52E−22 |
| CX5 | TIMELESS | 0.174218187086022 | 1.53E−22 | 2.33E−21 |
| CX5 | MSH2 | 0.173689352754412 | 2.51E−22 | 3.62E−21 |
| CX5 | TOPBP1 | 0.172296280258316 | 9.15E−22 | 1.25E−20 |
| CX5 | RAD1 | 0.16307557317659 | 3.62E−18 | 4.72E−18 |
| CX5 | MDC1 | 0.159617324269922 | 7.15E−17 | 8.91E−16 |
| CX5 | GEN1 | 0.15709336740407 | 6.05E−16 | 7.21E−15 |
| CX5 | NSD2 | 0.156890901885734 | 7.18E−17 | 8.19E−15 |
| CX5 | DNA2 | 0.154404185664498 | 5.67E−15 | 6.22E−14 |
| CX5 | UBE2T | 0.152645600050427 | 2.40E−14 | 2.53E−15 |
| CX5 | BRCA2 | 0.151052934756212 | 8.71E−14 | 8.84E−13 |
| CX5 | CDK2 | 0.149150904228892 | 4.00E−13 | 3.83E−12 |
| CX5 | RAD51C | 0.149134102878965 | 4.05E−13 | 3.83E−12 |
| CX5 | CHEK2 | 0.147914162729173 | 1.06E−12 | 9.72E−12 |
| CX5 | POLK | −0.145624532783208 | 6.39E−12 | 5.64E−11 |
| CX5 | XRCC2 | 0.144369940137911 | 1.68E−11 | 1.44E−10 |
| CX5 | FANCB | 0.146831601260132 | 5.78E−11 | 4.80E−10 |
| CX5 | NEIL3 | 0.142007616925539 | 1.02E−11 | 8.23E−10 |
| CX5 | DDB2 | −0.141623917338517 | 1.36E−10 | 1.07E−09 |
| CX5 | RFC3 | 0.140947152695218 | 2.27E−10 | 1.73E−09 |
| CX5 | BRCA1 | 0.140418015487719 | 3.37E−10 | 2.50E−10 |
| CX5 | PRKDC | 0.139570846510885 | 6.35E−10 | 4.58E−09 |
| CX5 | MPG | −0.138130859323131 | 1.84E−09 | 1.29E−08 |
| CX5 | YY1 | 0.13735623803447 | 3.25E−09 | 2.22E−08 |
| CX5 | POLR2D | 0.133931692396071 | 3.85E−09 | 2.57E−07 |
| CX5 | PARP2 | 0.13271887930849 | 9.10E−08 | 5.94E−07 |
| CX5 | XPA | −0.132015816497301 | 1.49E−07 | 9.51E−07 |
| CX5 | MAD2L2 | 0.131359726391016 | 2.36E−07 | 1.47E−06 |
| CX5 | UBE2V2 | 0.131186196484237 | 2.67E−07 | 1.62E−06 |
| CX5 | RAD18 | 0.129618701414547 | 7.91E−07 | 4.71E−06 |
| CX5 | RNF168 | 0.129119148129969 | 1.12E−06 | 6.51E−06 |
| CX5 | POLE2 | 0.128834806960382 | 1.36E−06 | 7.74E−06 |
| CX5 | MSH3 | −0.12420349344477 | 3.06E−05 | 1.71E−04 |
| CX5 | POLR2K | 0.123179426244564 | 6.00E−05 | 3.29E−04 |
| CX5 | XRCC3 | 0.123110553028364 | 6.27E−05 | 3.37E−04 |
| CX5 | RFC5 | 0.121971960623789 | 1.32E−04 | 6.94E−04 |
| CX5 | NEIL1 | −0.121390178932415 | 1.92E−04 | 9.92E−04 |
| CX5 | XPC | −0.121329071537011 | 2.00E−04 | 1.01E−03 |
| CX5 | RUVBL1 | 0.118799241098382 | 1.00E−03 | 4.91E−03 |
| CX5 | FANCM | 0.118110930354584 | 1.55E−03 | 7.44E−03 |
| CX5 | NEIL2 | −0.117913823145134 | 1.75E−03 | 8.27E−03 |
| CX5 | FANCE | 0.116487870202006 | 4.26E−03 | 1.98E−02 |
| CX5 | FAAP24 | 0.115636622883777 | 7.19E−03 | 3.29E−02 |
| CX5 | DTL | 0.113222991338831 | 3.12E−03 | 1.40E−01 |
| CX5 | SIRT6 | −0.113202276754411 | 3.16E−02 | 1.40E−01 |
| CX5 | USP1 | 0.110361611801357 | 1.71E−01 | 7.44E−01 |
| CX5 | GPS1 | 0.108681681502468 | 4.55E−01 | 1.95E+00 |
| CX5 | POLM | −0.107057025277239 | 1.15E+00 | 4.87E+00 |
| CX5 | RHNO1 | 0.106057867744267 | 2.03E+00 | 8.31E+00 |
| CX5 | POLR2B | 0.104844202206742 | 4.01E+00 | 1.62E+01 |
| CX5 | DCLRE1B | 0.103964845970537 | 8.44E+00 | 3.35E+01 |
| CX5 | RPA1 | 0.103059368521553 | 1.08E+01 | 4.22E+01 |
| CX5 | FANCA | 0.101629996563475 | 2.34E+00 | 8.92E+01 |
| CX5 | PARP1 | 0.101647998635443 | 2.32E+01 | 8.92E+01 |

### REFERENCES

1. Schimmel, J., van Schendel, R., den Dunnen, J. T. & Tijsterman, M. Templated Insertions: A Smoking Gun for Polymerase Theta-Mediated End Joining. Trends Genet. 35, 632-644 (2019).
2.Smimov, P. et al. PharmacoGx: an R package for analysis of large pharmacogenomic datasets. Bioinformatics vol. 32 1244-1246 (2016).
3. Liu, J. et al. An Integrated TCGA Pan-Cancer Clinical Data Resource to Drive High-Quality Survival Outcome Analytics. Cell 173, 400-416.e11 (2018).
4. Therneau, T. M. A Package for Survival Analysis in R. https://CRAN.R-project.org/package=survival (2021).
5. Guirouilh-Barbat, J., Lambert, S., Bertrand, P. & Lopez, B. S. Is homologous recombination really an error-free process? Frontiers in Genetics vol. 5 (2014).
6. Taylor, A. M. et al. Genomic and Functional Approaches to Understanding Cancer Aneuploidy. Cancer Cell 33, 676-689.e3 (2018).
7. Pitroda, S. P. et al. DNA repair pathway gene expression score correlates with repair proficiency and tumor sensitivity to chemotherapy. Sci. Transl. Med. 6, 229ra42 (2014).
8. Ben-David, U. & Amon, A. Context is everything: aneuploidy in cancer. Nat. Rev. Genet. 21, 44-62 (2020).
9. Network, T. C. G. A. R. & The Cancer Genome Atlas Research Network. Comprehensive genomic characterization defines human glioblastoma genes and core pathways. Nature vol. 455 1061-1068 (2008).
10. Network, T. C. G. A. R. & The Cancer Genome Atlas Research Network. Integrated genomic characterization of oesophageal carcinoma. Nature vol. 541 169-175 (2017).
11. Network, T. C. G. A. R. & The Cancer Genome Atlas Research Network. Integrated genomic analyses of ovarian carcinoma. Nature vol. 474 609-615 (2011).
12. Oliva-Rico, D. & Herrera, L. A. Regulated expression of the lncRNA TERRA and its impact on telomere biology. Mech. Ageing Dev. 167, 16-23 (2017).
13. Heaphy, C. M. et al. Altered telomeres in tumors with ATRX and DAXX mutations. Science 333, 425 (2011).
14. Dyer, M. A., Qadeer, Z. A., Valle-Garcia, D. & Bernstein, E. ATRX and DAXX: Mechanisms and Mutations. Cold Spring Harb. Perspect. Med. 7, (2017).
15. Almeida, B. P. D., Vieira, A. F., Paredes, J., Bettencourt-Dias, M. & Barbosa-Morais, N. L. Pan-cancer association of a centrosome amplification gene expression signature with genomic alterations and clinical outcome. PLOS Computational Biology vol. 15 e1006832 (2019).
16. Bakhoum, S. F. & Cantley, L. C. The Multifaceted Role of Chromosomal Instability in Cancer and Its Microenvironment. Cell 174, 1347-1360 (2018).
17. Raghvendra Vishwakarma, K. J. M. Chromosome Instability; Implications in Cancer Development, Progression, and Clinical Outcomes. Cancers 12, (2020).
18. Vargas-Rondón, N., Villegas, V. E. & Rondón-Lagos, M. The Role of Chromosomal Instability in Cancer and Therapeutic Responses. Cancers 10, (2018).
19. Bakhoum, S. F. et al. Chromosomal instability drives metastasis through a cytosolic DNA response. Nature 553, 467-472 (2018b).
20. Maechler, M. sfsmisc: Utilities from ‘Seminar fuer Statistik’ ETH Zurich. (2020).
21. Bakhoum, S. F., Danilova, O. V., Kaur, P., Levy, N. B. & Compton, D. A. Chromosomal instability substantiates poor prognosis in patients with diffuse large B-cell lymphoma. Clin. Cancer Res. 17, 7704-7711 (2011).
22. Lee, A. J. X. et al. Chromosomal instability confers intrinsic multidrug resistance. Cancer Res. 71, 1858-1870 (2011).
23. Benjamini, Y. & Hochberg, Y. Controlling the False Discovery Rate: A Practical and Powerful Approach to Multiple Testing. J. R. Stat. Soc. Series B Stat. Methodol. 57, 289-300 (1995).
24. Kumar, P. et al. ATAC-seq identifies thousands of extrachromosomal circular DNA in cancer and cell lines. Sci Adv 6, eaba2489 (2020).
25. Gu, X., Yu, J., Chai, P., Ge, S. & Fan, X. Novel insights into extrachromosomal DNA: redefining the onco-drivers of tumor progression. J. Exp. Clin. Cancer Res. 39, 215 (2020).
26. Ding, Z. et al. Estimating telomere length from whole genome sequence data. Nucleic Acids Res. 42, e75 (2014).
27. Venables, W. N. & Ripley, B. D. Modern Applied Statistics with S. (2002).
28. Birkbak, N. J. et al. Paradoxical relationship between chromosomal instability and survival outcome in cancer. Cancer Res. 71, 3447-3452 (2011).
29. Davies, H. et al. HRDetect is a predictor of BRCA1 and BRCA2 deficiency based on mutational signatures. Nat. Med. 23, 517-525 (2017).
30. Cohen-Sharir, Y. et al. Aneuploidy renders cancer cells vulnerable to mitotic checkpoint inhibition. Nature 590, 486-491 (2021).
31. Macintyre, G. et al. Copy number signatures and mutational processes in ovarian carcinoma. Nat. Genet. (2018) doi: 10.1038/s41588-018-0179-8.
32. Steele, C. D. et al. Undifferentiated Sarcomas Develop through Distinct Evolutionary Pathways. Cancer Cell 35, 441-456.e8 (2019).
33. Van Loo, P. et al. Allele-specific copy number analysis of tumors. Proc. Natl. Acad. Sci. U.S.A. 107, 16910-16915 (2010).
34. Kim, J. et al. Somatic ERCC2 mutations are associated with a distinct genomic signature in urothelial tumors. Nat. Genet. 48, 600-606 (2016).
35. Tan, V. Y. F. & Févotte, C. Automatic relevance determination in nonnegative matrix factorization with the β-divergence. IEEE Trans. Pattern Anal. Mach. Intell. 35, 1592-1605 (2013).
36. Hannah, J. & Zhou, P. Regulation of DNA damage response pathways by the cullin-RING ubiquitin ligases. DNA Repair 8, 536-543 (2009).
37. Zhao, J. et al. SCF E3 ligase F-box protein complex SCF (FBXL19) regulates cell migration by mediating Rac1 ubiquitination and degradation. FASEB J. 27, 2611-2619 (2013).
38. Chittaranjan, S. et al. Loss of CIC promotes mitotic dysregulation and chromosome segregation defects. bioRxiv 533323 (2019) doi: 10.1101/533323.
39. Varela, I. et al. Exome sequencing identifies frequent mutation of the SWI/SNF complex gene PBRM1 in renal carcinoma. Nature 469, 539-542 (2011).
40. Bihr, S. et al. Expression and Mutation Patterns of PBRM1, BAP1 and SETD2 Mirror Specific Evolutionary Subtypes in Clear Cell Renal Cell Carcinoma. Neoplasia 21, 247-256 (2019).
41. Luke, B. & Lingner, J. TERRA: telomeric repeat-containing RNA. EMBO J. 28, 2503-2510 (2009).
42. Hell, M. P., Duda, M., Weber, T. C., Moch, H. & Krek, W. Tumor suppressor VHL functions in the control of mitotic fidelity. Cancer Res. 74, 2422-2431 (2014).
43. Pampalona, J., Soler, D., Genescà, A. & Tusell, L. Whole chromosome loss is promoted by telomere dysfunction in primary cells. Genes Chromosomes Cancer 49, 368-378 (2010).
44. Treff, N. R., Su, J., Taylor, D. & Scott, R. T., Jr. Telomere DNA deficiency is associated with development of human embryonic aneuploidy. PLOS Genet. 7, e1002161 (2011).
45. Powell, S. N. & Kachnic, L. A. Roles of BRCA1 and BRCA2 in homologous recombination, DNA replication fidelity and the cellular response to ionizing radiation. Oncogene 22, 5784-5791 (2003).
46. Menghi, F. et al. The Tandem Duplicator Phenotype Is a Prevalent Genome-Wide Cancer Configuration Driven by Distinct Gene Mutations. Cancer Cell 34, 197-210.e5 (2018).
47. Nik-Zainal, S. et al. Landscape of somatic mutations in 560 breast cancer whole-genome sequences. Nature 534, 47-54 (2016).
48. Alexandrov, L. B. et al. The repertoire of mutational signatures in human cancer. Nature 578, 94-101 (2020).
49. Ogden, A., Rida, P. C. G. & Aneja, R. Prognostic value of CA20, a score based on centrosome amplification-associated genes, in breast tumors. Sci. Rep. 7, 262 (2017).
50. Abkevich, V. et al. Patterns of genomic loss of heterozygosity predict homologous recombination repair defects in epithelial ovarian cancer. Br. J. Cancer 107, 1776-1782 (2012).
51. Piazza, A. & Heyer, W.-D. Homologous Recombination and the Formation of Complex Genomic Rearrangements. Trends Cell Biol. 29, 135-149 (2019).
52. Knijnenburg, T. A. et al. Genomic and Molecular Landscape of DNA Damage Repair Deficiency across The Cancer Genome Atlas. Cell Rep. 23, 239-254.e6 (2018).
53. Popova, T. et al. Ploidy and large-scale genomic instability consistently identify basal-like breast carcinomas with BRCA1/2 inactivation. Cancer Res. 72, 5454-5462 (2012).
54. Telli, M. L. et al. Homologous Recombination Deficiency (HRD) Score Predicts Response to Platinum-Containing Neoadjuvant Chemotherapy in Patients with Triple-Negative Breast Cancer. Clin. Cancer Res. 22, 3764-3773 (2016).
55. Saavedra, H. I., Fukasawa, K., Conn, C. W. & Stambrook, P. J. MAPK mediates RAS-induced chromosome instability. J. Biol. Chem. 274, 38083-38090 (1999).
56. Primo, L. M. F. & Teixeira, L. K. DNA replication stress: oncogenes in the spotlight. Genet. Mol. Biol. 43, e20190138 (2019).
57. Li, Q. et al. ERCC2 Helicase Domain Mutations Confer Nucleotide Excision Repair Deficiency and Drive Cisplatin Sensitivity in Muscle-Invasive Bladder Cancer. Clin. Cancer Res. 25, 977-988 (2019).
58. Menon, V. & Povirk, L. Involvement of p53 in the repair of DNA double strand breaks: multifaceted Roles of p53 in homologous recombination repair (HRR) and non-homologous end joining (NHEJ). Subcell. Biochem. 85, 321-336 (2014).
59. Setton, J. S. & Powell, S. N. A new role for a tumor-suppressing protein. eLife vol. 7 (2018).
60. Lord, C. J. & Ashworth, A. BRCAness revisited. Nat. Rev. Cancer 16, 110-120 (2016).
61. Stok, C., Kok, Y. P., van den Tempel, N. & van Vugt, M. A. T. M. Shaping the BRCAness mutational landscape by alternative double-strand break repair, replication stress and mitotic aberrancies. Nucleic Acids Res. (2021) doi: 10.1093/nar/gkab151.
62. Dentro, S. C. et al. Characterizing genetic intra-tumor heterogeneity across 2,658 human cancer genomes. Cell 184, 2239-2254.e39 (2021).
63. Prasad, K. et al. Whole-genome duplication shapes the aneuploidy landscape of human cancers. bioRxiv 2021.05.05.442712 (2021) doi: 10.1101/2021.05.05.442712.
64. Dewhurst, S. M. et al. Tolerance of whole-genome doubling propagates chromosomal instability and accelerates cancer genome evolution. Cancer Discov. 4, 175-185 (2014).
65. Davoli, T. & de Lange, T. The Causes and Consequences of Polyploidy in Normal Development and Cancer. (2011) doi: 10.1146/annurev-cellbio-092910-154234.
66. Berenjeno, I. M. et al. Oncogenic PIK3CA induces centrosome amplification and tolerance to genome doubling. Nat. Commun. 8, 1773 (2017).
67. Zhang, Q. et al. FBXW7 Facilitates Nonhomologous End-Joining via K63-Linked Polyubiquitylation of XRCC4. Mol. Cell 61, 419-433 (2016).
68. Perl, A. L. et al. Protein phosphatase 2A controls ongoing DNA replication by binding to and regulating cell division cycle 45 (CDC45). J. Biol. Chem. 294, 17043-17059 (2019).
69. Lundberg, A. et al. A pan-cancer analysis of the frequency of DNA alterations across cell cycle activity levels. Oncogene 39, 5430-5440 (2020).
70. Cescon, D. W. & Haibe-Kains, B. DNA replication stress: a source of APOBEC3B expression in breast cancer. Genome Biol. 17, 202 (2016).
71. Kanu, N. et al. DNA replication stress mediates APOBEC3 family mutagenesis in breast cancer. Genome Biol. 17, 185 (2016).
72. Chen, L. et al. The Augmented R-Loop Is a Unifying Mechanism for Myelodysplastic Syndromes Induced by High-Risk Splicing Factor Mutations. Mol. Cell 69, 412-425.e6 (2018).
73. Nguyen, H. D. et al. Spliceosome Mutations Induce R Loop-Associated Sensitivity to ATR Inhibition in Myelodysplastic Syndromes. Cancer Res. 78, 5363-5374 (2018).
74. Kim, H. et al. Extrachromosomal DNA is associated with oncogene amplification and poor outcome across multiple cancers. Nat. Genet. 52, 891-897 (2020).
75. Nik-Zainal, S. et al. Mutational processes molding the genomes of 21 breast cancers. Cell 149, 979-993 (2012).
76. Meyers, R. M. et al. Computational correction of copy number effect improves specificity of CRISPR-Cas9 essentiality screens in cancer cells. Nat. Genet. 49, 1779-1784 (2017).
77. McFarland, J. M. et al. Improved estimation of cancer dependencies from large-scale RNAi screens using model-based normalization and data integration. Nat. Commun. 9, 4610 (2018).
78. Corsello, S. M. et al. Non-oncology drugs are a source of previously unappreciated anti-cancer activity. bioRxiv 730119 (2019) doi: 10.1101/730119.
79. Hirakawa, T., Nasu, K., Aoyagi, Y., Takebayashi, K. & Narahara, H. Arcyriaflavin a, a cyclin D1-cyclin-dependent kinase4 inhibitor, induces apoptosis and inhibits proliferation of human endometriotic stromal cells: a potential therapeutic agent in endometriosis. Reprod. Biol. Endocrinol. 15, 53 (2017).
80. Ray Chaudhuri, A. & Nussenzweig, A. The multifaceted roles of PARP1 in DNA repair and chromatin remodelling. Nat. Rev. Mol. Cell Biol. 18, 610-621 (2017).
81. Ubhi, T. & Brown, G. W. Exploiting DNA Replication Stress for Cancer Treatment. Cancer Res. 79, 1730-1739 (2019).
82. Goel, S. et al. CDK4/6 inhibition triggers anti-tumour immunity. Nature 548, 471-475 (2017).
83. Mitsopoulos, C. et al. canSAR: update to the cancer translational research and drug discovery knowledgebase. Nucleic Acids Res. 49, D1074-D1082 (2021).
84. Brownlee, P. M., Meisenberg, C. & Downs, J. A. The SWI/SNF chromatin remodelling complex: Its role in maintaining genome stability and preventing tumourigenesis. DNA Repair 32, 127-133 (2015).
85. Kops, G. J. P., Foltz, D. R. & Cleveland, D. W. Lethality to human cancer cells through massive chromosome loss by inhibition of the mitotic checkpoint. Proc. Natl. Acad. Sci. U.S.A 101, 8699-8704 (2004).
86. Quinton, R. J. et al. Whole-genome doubling confers unique genetic vulnerabilities on tumour cells. Nature (2021) doi: 10.1038/s41586-020-03133-3.
87. Janssen, A., Kops, G. J. P. L. & Medema, R. H. Elevating the frequency of chromosome missegregation as a strategy to kill tumor cells. Proc. Natl. Acad. Sci. U.S.A 106, 19108-19113 (2009).
88. Datta, D. et al. Nucleolar GTP-binding Protein-1 (NGP-1) Promotes G1 to S Phase Transition by Activating Cyclin-dependent Kinase Inhibitor p21 Cip1/Waf1. J. Biol. Chem. 290, 21536-21552 (2015).
89. Thompson, L. H. & Schild, D. Homologous recombinational repair of DNA ensures mammalian chromosome stability. Mutat. Res. 477, 131-153 (2001).
90. Schoonen, P. M. et al. Progression through mitosis promotes PARP inhibitor-induced cytotoxicity in homologous recombination-deficient cancer cells. Nat. Commun. 8, 15981 (2017).
91. Konstantinopoulos, P. A., Ceccaldi, R., Shapiro, G. I. & D'Andrea, A. D. Homologous Recombination Deficiency: Exploiting the Fundamental Vulnerability of Ovarian Cancer. Cancer Discov. 5, 1137-1154 (2015).
92. Martin, L. P., Hamilton, T. C. & Schilder, R. J. Platinum resistance: the role of DNA repair pathways. Clin. Cancer Res. 14, 1291-1295 (2008).
93. Favero F, et al. Sequenza: allele-specific copy number and mutation profiles from tumor sequencing data. Ann Oncol. 2015 January; 26 (1): 64-70.
94. Zack, T. I. et al. Pan-cancer patterns of somatic copy number alteration. Nat. Genet. 45, 1134-1140 (2013).
95. Wong, D. & Yip, S. Making heads or tails—the emergence of capicua (CIC) as an important multifunctional tumour suppressor. J. Pathol. 250, 532-540 (2020).
96. Brownlee, P. M. et al. BAF180 promotes cohesion and prevents genome instability and aneuploidy. Cell Rep. 6, 973-981 (2014).
97. Hodges, C., Kirkland, J. G. & Crabtree, G. R. The Many Roles of BAF (mSWI/SNF) and PBAF Complexes in Cancer. Cold Spring Harb. Perspect. Med. 6, (2016).
98. Chen, L., Liu, T., Tu, Y., Rong, D. & Cao, Y. Cul1 promotes melanoma cell proliferation by promoting DEPTOR degradation and enhancing cap-dependent translation. Oncol. Rep. 35, 1049-1056 (2016).
99. Li, Q. et al. Rac1 activates non-oxidative pentose phosphate pathway to induce chemoresistance of breast cancer. Nat. Commun. 11, 1456 (2020).
100. Shammas, M. A. Telomeres, lifestyle, cancer, and aging. Curr. Opin. Clin. Nutr. Metab. Care 14, 28-34 (2011).
101. Hustedt, N. & Durocher, D. The control of DNA repair by the cell cycle. Nat. Cell Biol. 19, 1-9 (2017).
102. Marquard, A. M. et al. Pan-cancer analysis of genomic scar signatures associated with homologous recombination deficiency suggests novel indications for existing cancer drugs. Biomark Res 3, 9 (2015).103. Palangat, M. et al. The splicing factor U2AF1 contributes to cancer progression through a noncanonical role in translation regulation. Genes Dev. 33, 482-497 (2019).
104. Vanhaesebroeck, B. et al. Perspective: Potential Impact and Therapeutic Implications of Oncogenic PI3K Activation on Chromosomal Instability. Biomolecules 9, (2019).
105. Darp, R., Vittoria, M. A., Ganem, N. J. & Ceol, C. J. Oncogenic BRAF Induces Whole-Genome Doubling Through Suppression of Cytokinesis. bioRxiv 2021.04.08.439023 (2021)
106. Yan, Y. et al. Current understanding of extrachromosomal circular DNA in cancer pathogenesis and therapeutic resistance. J. Hematol. Oncol. 13, 124 (2020).
107. Wang, Y., Huang, R., Zheng, G. & Shen, J. Small ring has big potential: insights into extrachromosomal DNA in cancer. Cancer Cell Int. 21, 236 (2021).
108. Beroukhim, R. et al. The landscape of somatic copy-number alteration across human cancers. Nature 463, 899-905 (2010).
109. Tate, J. G. et al. COSMIC: the Catalogue Of Somatic Mutations In Cancer. Nucleic Acids Res. 47, D941-D947 (2019).
110. Gerstung, M. et al. The evolutionary history of 2,658 cancers. Nature 578, 122-128 (2020).
111. Ghandi, M. et al. Next-generation characterization of the Cancer Cell Line Encyclopedia. Nature 569, 503-508 (2019).
112. Wang, Y. K. et al. Genomic consequences of aberrant DNA repair mechanisms stratify ovarian cancer histotypes. Nat. Genet. 49, 856-865 (2017).
113. Maxwell, K. N. et al. BRCA locus-specific loss of heterozygosity in germline BRCA1 and BRCA2 carriers. Nat. Commun. 8, 319 (2017).
114. Cancer Genome Atlas Research Network. Integrated genomic analyses of ovarian carcinoma. Nature 474, 609-615 (2011).
115. ICGC/TCGA Pan-Cancer Analysis of Whole Genomes Consortium. Pan-cancer analysis of whole genomes. Nature 578, 82-93 (2020).
116. Degasperi, A. et al. A practical framework and online tool for mutational signature analyses show inter-tissue variation and driver dependencies. Nat Cancer 1, 249-263 (2020).
117. Li, Y. et al. Patterns of somatic structural variation in human cancer genomes. Nature 578, 112-121 (2020).
118. Ellrott, K. et al. Scalable Open Science Approach for Mutation Calling of Tumor Exomes Using Multiple Genomic Pipelines. Cell Syst 6, 271-281.e7 (2018).
119. Barthel, F. P. et al. Systematic analysis of telomere length and somatic alterations in 31 cancer types. Nat. Genet. 49, 349-357 (2017).
120. Bailey, M. H. et al. Comprehensive Characterization of Cancer Driver Genes and Mutations. Cell 174, 1034-1035 (2018).
121. Jassal, B. et al. The reactome pathway knowledgebase. Nucleic Acids Res. 48, D498-D503 (2020).
122. Tan, V. Y. F. & Févotte, C. Automatic relevance determination in nonnegative matrix factorization with the B-divergence. IEEE Trans. Pattern Anal. Mach. Intell. 35, 1592-1605 (2013).
123. Weaver, B. A. A. & Cleveland, D. W. Does aneuploidy cause cancer? Curr. Opin. Cell Biol. 18, 658-667 (2006).
124. Frankish, A. et al. GENCODE reference annotation for the human and mouse genomes. Nucleic Acids Res. 47, D766-D773 (2019).
125. Huebschmann, D., Gu, Z. & Schlesner, M. YAPSA: Yet Another Package for Signature Analysis. (2019).
126. Cimini, D. Merotelic kinetochore orientation, aneuploidy, and cancer. Biochim. Biophys. Acta 1786, 32-40 (2008).
127. Lens, S. M. A. & Medema, R. H. Cytokinesis defects and cancer. Nat. Rev. Cancer 19, 32-45 (2019).
128. Mitelman, F., Johansson, B. & Mertens, F. Mitelman Database of Chromosome Aberrations and Gene Fusions in Cancer. https://mitelmandatabase.isb-cgc.org.
129. Carter, S. L. et al. Absolute quantification of somatic DNA alterations in human cancer. Nat. Biotechnol. 30, 413-421 (2012).
130. Cisyk, A. L. et al. Characterizing the Prevalence of Chromosome Instability in Interval Colorectal Cancer. Neoplasia 17, 306-316 (2015).
131. Harbour, J. W. Eye cancer: unique insights into oncogenesis: the Cogan Lecture. Invest. Ophthalmol. Vis. Sci. 47, 1736-1745 (2006).
132. Etemadmoghadam, D. et al. Synthetic lethality between CCNE1 amplification and loss of BRCA1. Proc. Natl. Acad. Sci. U.S.A 110, 19489-19494 (2013).
133. Murnane, J. P. Telomere dysfunction and chromosome instability. Mutat. Res. Fundam. Mol. Mech. Mutagen. 730, 28-36 (2012).
134. Zakov, S. & Bafna, V. Reconstructing breakage fusion bridge architectures using noisy copy numbers. J. Comput. Biol. 22, 577-594 (2015).
135. Korbel, J. O. & Campbell, P. J. Criteria for inference of chromothripsis in cancer genomes. Cell 152, 1226-1236 (2013).
136. Malhotra, A. et al. Breakpoint profiling of 64 cancer genomes reveals numerous complex rearrangements spawned by homology-independent mechanisms. Genome Res. 23, 762-776 (2013).
137. Verhaak, R. G. W., Bafna, V. & Mischel, P. S. Extrachromosomal oncogene amplification in tumour pathogenesis and evolution. Nat. Rev. Cancer 1 (2019).
138. Birkbak, N. J. et al. Telomeric allelic imbalance indicates defective DNA repair and sensitivity to DNA-damaging agents. Cancer Discov. 2, 366-375 (2012).
139. Ryland, G. L. et al. Loss of heterozygosity: What is it good for? BMC Med. Genomics 8, 45 (2015).
140. Umbreit, N. T. et al. Mechanisms generating cancer genome complexity from a single cell division error. Science 368, (2020).
141. Ng, C. K. Y. et al. The role of tandem duplicator phenotype in tumour evolution in high grade serous ovarian cancer. J. Pathol. 226, 703-712 (2012).
142. Jonsdottir, A. B. et al. Tetraploidy in BRCA2 breast tumours. Eur. J. Cancer 48, 305-310 (2012).
143. Pradhan, M. et al. Gross genomic alterations and gene expression profiles of high-grade serous carcinoma of the ovary with and without BRCA1 inactivation. BMC Cancer 10, 493 (2010).
144. Chien, J. et al. TP53 mutations, tetraploidy and homologous recombination repair defects in early stage high-grade serous ovarian cancer. Nucleic Acids Res. 43, 6945-6958 (2015).
145. Nichols, C. A. et al. Loss of heterozygosity of essential genes represents a widespread class of potential cancer vulnerabilities. Nat. Commun. 11, 1-14 (2020).
146. Macintyre, G., YIstra, B. & Brenton, J. D. Sequencing Structural Variants in Cancer for Precision Therapeutics. Trends Genet. 32, 530-542 (2016).
147. Kashima, Y. et al. Single-cell sequencing techniques from individual to multiomics analyses. Exp. Mol. Med. 52, 1419-1427 (2020).
148. Lahnemann, D. et al. Eleven grand challenges in single-cell data science. Genome Biology vol. 21 53 (2020).
149. Blei, D. M. & Jordan, M. I. Variational inference for Dirichlet process mixtures. Bayesian Anal. 1, 121-143 (2006).
150. Grün, B. & Leisch, F. FlexMix Version 2: Finite Mixtures with Concomitant Variables and Varying and Constant Parameters. Journal of Statistical Software, Articles 28, 1-35 (2008).
151. Martin, A., Quinn, K. & Park, J. H. MCMCpack: Markov Chain Monte Carlo in R. Journal of Statistical Software, Articles 42, 1-21 (2011).
152. Scrucca, L., Fop, M., Murphy, T. B. & Raftery, A. E. mclust 5: Clustering, Classification and Density Estimation Using Gaussian Finite Mixture Models. R J. 8, 289-317 (2016).
153. Scheinin, I. et al. DNA copy number analysis of fresh and formalin-fixed specimens by shallow whole-genome sequencing with identification and exclusion of problematic regions in the genome assembly. Genome Res. 24, 2022-2032 (2014).
154. R Core Team. R: A Language and Environment for Statistical Computing.
155. Olshen, A. B., Venkatraman, E. S., Lucito, R. & Wigler, M. Circular binary segmentation for the analysis of array-based DNA copy number data. Biostatistics 5, 557-572 (2004).
156. Seshan, V. E. & Olshen, A. DNAcopy: DNA copy number data analysis. (2019).
157. Auton, A. et al. A global reference for human genetic variation. Nature 526, 68-74 (2015).
158. Wedge, D., Van Loo, P. & Dentro, S. Battenberg: Battenberg subclonal copy number caller. (2021).
159. Fabregat, A. et al. Reactome graph database: Efficient access to complex pathway data. PLOS Comput. Biol. 14, e1005968 (2018).
160. Hunt, S. E. et al. Ensembl variation resources. Database 2018, (2018).
161. Yates, A. D. et al. Ensembl 2020. Nucleic Acids Res. 48, D682-D688 (2020).
162. Durinck, S. et al. BioMart and Bioconductor: a powerful link between biological databases and microarray data analysis. Bioinformatics 21, 3439-3440 (2005).
163. Durinck, S., Spellman, P. T., Birney, E. & Huber, W. Mapping identifiers for the integration of genomic datasets with the R/Bioconductor package biomaRt. Nat. Protoc. 4, 1184-1191 (2009).
164. Cancer Genome Atlas Research Network. Comprehensive genomic characterization defines human glioblastoma genes and core pathways. Nature 455, 1061-1068 (2008).
165. Boersma, V. et al. MAD2L2 controls DNA repair at telomeres and DNA breaks by inhibiting 5′end resection. Nature 521, 537-540 (2015).
166. Isono, M. et al. BRCA1 Directs the Repair Pathway to Homologous Recombination by Promoting 53BP1 Dephosphorylation. Cell Rep. 18, 520-532 (2017).

All references cited herein are incorporated herein by reference in their entirety and for all purposes to the same extent as if each individual publication or patent or patent application was specifically and individually indicated to be incorporated by reference in its entirety.

The specific embodiments described herein are offered by way of example, not by way of limitation. Various modifications and variations of the described compositions, methods, and uses of the technology will be apparent to those skilled in the art without departing from the scope and spirit of the technology as described. Any sub-titles herein are included for convenience only, and are not to be construed as limiting the disclosure in any way. Unless context dictates otherwise, the descriptions and definitions of the features set out above are not limited to any particular aspect or embodiment of the invention and apply equally to all aspects and embodiments which are described. Throughout the specification and claims, the following terms take the meanings explicitly associated herein, unless the context clearly dictates otherwise. The phrase “in one embodiment” as used herein does not necessarily refer to the same embodiment, though it may. Furthermore, the phrase “in another embodiment” as used herein does not necessarily refer to a different embodiment, although it may. Thus, as described below, various embodiments of the invention may be readily combined, without departing from the scope or spirit of the invention. It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means for example +/−10%. Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps. Other aspects and embodiments of the invention provide the aspects and embodiments described above with the term “comprising” replaced by the term “consisting of” or “consisting essentially of”, unless the context dictates otherwise. The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof.

1. A method of characterising a DNA sample obtained from a tumour, the method including the steps of:
(a) obtaining a tumour copy number profile for the sample;
(b) quantifying a set of copy number features of the copy number profile, wherein a copy number feature is a metric that characterises a copy number event in a copy number profile, and wherein the set of features does not comprise the absolute copy number of segments in the copy number profile; and
(c) determining exposure to one or more signatures of chromosomal instability based on the quantified features, wherein the signatures of chromosomal instability have been obtained by quantifying the set of copy number features in a plurality of tumour samples, and identifying one or more mutational signatures likely to result in the copy number profiles of the plurality of tumour samples.

2. The method of claim 1, wherein quantifying a set of copy number features of the copy number profile comprises quantifying, for each copy number event in the copy number profile, one or more features selected from: segment size, copy number change-point, breakpoint count per predetermined length of sequence and/or per chromosome arm, and number of segments with oscillating copy number, optionally wherein the set of copy number features includes all of the following features: segment size, breakpoint count per predetermined length of sequence, copy number change-point, breakpoint count per chromosome arm, and number of segments with oscillating copy number, optionally wherein the set of copy number features consists of said features or said features and one or more allele-specific features.

3. The method of claim 1 or claim 2, wherein quantifying the set of copy number features comprises:
using unrounded copy number segments; and/or
collapsing and merging near diploid segments to a diploid state, wherein near diploid segments are segments that have a copy number within a predetermined distance from 2, optionally wherein the predetermined distance is 0.1.

4. The method of any preceding claim, wherein quantifying the set of copy number features comprises quantifying a feature selected from segment size and copy number changepoint, and wherein said feature is not quantified for diploid segments.

5. The method of any preceding claim, wherein quantifying the set of copy number features comprises obtaining one or more summarised measures for each copy number feature across the copy number profile, optionally wherein the one or more summarised measures comprise the sum over all copy number events for which a feature has been quantified of the posterior probabilities of each feature value belonging to each of a set of predetermined distributions and/or wherein the one or more summarised measures have been identified empirically by quantifying the copy number features in a plurality of tumour samples.

6. The method of claim 5, wherein the set of predetermined distributions for a copy number feature is a set of Gaussian distributions for any feature that is quasi-continuous, and/or wherein the set of predetermined distributions is a set of Poisson distributions for any count feature.

7. The method of any preceding claim, wherein determining exposure to one or more signatures of chromosomal instability based on the quantified features comprises identifying the values of E that satisfy:
$$PbC \approx E \times SbC \qquad ( Equation 1 )$$
where E is a vector of size n comprising coefficients E_{1, . . . , n} where Eᵢ is the exposure to signature i; PbC is a vector of size c, each element in the vector representing a summarised measure across the copy number profile associated with one of the copy number features; and SbC is a matrix of size c by n, each value representing the weight of a summarised measured C in a signature i.

8. The method of any preceding claim, wherein the signatures of chromosomal instability have been obtained by identifying the values of SbC and E that satisfy:
$$PbC \approx E \times SbC \qquad ( Equation 1 )$$
where E is a matrix of size n by p, each element in the matrix representing the exposure to a signature in the copy number profile of one of the plurality of tumour samples; PbC is a matrix of size c by p, each element in the matrix representing a summarised measure associated with one of the copy number features, wherein the summarised measured is obtained for the copy number profile of one of the plurality of tumour samples; and SbC is a matrix of size c by n, each value representing the weight of a summarised measured C in a signature/, optionally wherein the values of E and SbC are obtained by non-negative matrix factorisation.

9. The method of any of claims 5 to 8, wherein the summarised measures comprise the sum over all copy number events for which a feature has been quantified of the posterior probabilities of each feature value belonging to each of a set of predetermined distributions, wherein the predetermined distributions are the distributions defined by the parameters in Table 6, or corresponding distributions obtained by fitting mixture models to summarised measures of the set of copy number features obtained for a plurality of tumour samples, and/or wherein the signatures are those defined in Table 7 or corresponding signatures obtained by quantifying the set of copy number features in a plurality of tumour samples, and identifying one or more mutational signatures likely to result in the copy number profiles of the plurality of tumour samples.

10. The method of any preceding claim, wherein the plurality of tumour samples comprise tumour samples from a plurality of types of tumours, and the one or more signatures have been obtained by combining:
a first set of signatures obtained by quantifying the set of copy number features in the plurality of tumour samples, and identifying one or more mutational signatures likely to result in the copy number profiles of the plurality of tumour samples; and
one or more further sets of signatures obtained by quantifying the set of copy number features in a plurality of tumour samples from a respective tumour type for each further set of signatures, and identifying one or more mutational signatures likely to result in the copy number profiles of the plurality of tumour samples from the respective tumour type.

11. The method of claim 10, wherein the plurality of sets of signatures have been combined by removing signatures in the one or more further sets that have a similarity to any signature in the first set above a predetermined threshold and/or removing signatures in the one or more further sets that have a similarity to any signature in another of the one or more further sets above a predetermined threshold, and/or removing signatures in the one or more further sets that can be obtained by a linear combination of signatures in the first set.

12. The method of any preceding claim, wherein the one or more signatures of chromosomal instability comprise signatures associated with one or more processes causing chromosomal instability, and exposure to the one or more signatures is indicative of the presence of the respective process(es) in the sample and/or wherein the method comprises determining that a signature is present in the sample if the exposure to the signature in the sample is above a signature specific threshold, optionally wherein the signature-specific threshold has been identified using a simulated background distribution of signature exposure for the respective signature,
optionally wherein the one or more signatures comprise one or more signatures selected from: one or more signatures associated with chromosome missegregation, optionally chromosome missegregation via defective mitosis and/or telomere dysfunction, one or more signatures associated with impaired homologous recombination, one or more signatures associated with tolerance of whole genome duplication, one or more signatures associated with impaired non-homologous end joining, one or more signatures associated with replication stress, and one or more signature associated with impaired DNA damage sensing.

13. The method of any preceding claim, wherein the sample has been obtained from a subject who has bene diagnosed as having cancer and/or wherein the plurality of tumour samples comprise tumour samples from one or more of a plurality of types of tumours, optionally wherein the cancer and/or the plurality of tumour types are selected from: ovarian cancer, breast cancer, endometrial cancer, kidney cancer, lung cancer, pancreatic cancer, liver cancer, oesophagus cancer, stomach cancer, head and neck cancer, brain cancer, colon cancer, pancreatic cancer, prostate cancer, bladder cancer, cervical cancer, leukemia, lymphoma, testicular cancer, thyroid cancer, melanoma, adrenal cancer, bowel cancer, sarcoma, thymoma, neuroendocrine tumour, and bile duct cancer.

14. The method of any preceding claim, wherein the sample is a tumour sample or a liquid biopsy sample, and/or wherein the method further comprises one or more of obtaining the sample from a subject who has been diagnosed as having cancer, obtaining sequence data from the sample, determining a copy number profile from sequence data obtained from the sample, obtaining a matched germline sample, obtaining sequence data from a matched germline sample, and providing to a user one or more of: the exposure to the one or more signatures, a value derived therefrom, values for one or more copy number features, and a determination of whether one or more processes causing chromosomal instability are likely to be present in the sample.

15. A method of characterising the processes causing chromosomal instability occurring in a plurality of types of cancers, the method including the steps of:
(a) obtaining a tumour copy number profile for a plurality of tumour samples from each of the plurality of types of cancers;
(b) quantifying a set of copy number features of the copy number profiles, wherein a copy number feature is a metric that characterises a copy number event in a copy number profile, and wherein the set of features does not comprise the absolute copy number of segments in the copy number profile; and
(c) identifying one or more mutational signatures likely to result in the copy number profiles of the plurality of tumour samples, optionally wherein the method further has the features of any of claims 1 to 14.

16. A method of predicting whether a subject with cancer is likely to respond to a therapy that targets a particular gene, the method comprising: characterising a DNA sample obtained from a tumour of the subject, using the method of any of claims 1 to 14, as having a high or low exposure to one or more signatures of chromosomal instability associated with response to inhibition of the gene, wherein if the sample is characterised as having a high exposure to said signature, the subject is likely to respond to the therapy.

17. The method of claim 16, wherein the therapy inhibits CCND1 and the signature is a signature associated with tolerance to whole genome duplication or PI3K/AKT-mediated tolerance of whole genome duplication (such as e.g. CX4 in Table 7 or a corresponding signature), wherein the therapy inhibits PARP1 and the signature is a signature associated with impaired homologous recombination (such as e.g. CX5 in Table 7 or a corresponding signature), wherein the therapy inhibits a kinase in a mitogenic pathway (such as EGFR, JAK1, MET, PRKCA, PI3KCA) and the signature is a signature associated with replication stress (optionally wherein the signature is further indicative of focal amplifications; such as e.g. CX9 in Table 7 or a corresponding signature), wherein the therapy inhibits CDK4 and the signature is a signature associated with replication stress (optionally wherein the signature is further indicative of clustered amplifications, such as e.g. CX13 in Table 7 or a corresponding signature).

18. The method of claim 16 or claim 17, wherein a signature of chromosomal instability is considered to be associated with response to inhibition of the gene if the exposure to the signature is significantly correlated with the effect of perturbation of the gene and/or wherein a sample is considered to have a high exposure to the signature if exposure to the signature is higher than the expected exposure for the signature in a set of samples that do not respond to the perturbation of the gene.

19. A method of identifying a drug target for the treatment of a cancer, the method comprising:
characterising a plurality of DNA samples, using the method of any of claims 1 to 14, wherein the plurality of DNA samples comprise samples obtained from a tumour or a tumour cell line in which the drug target has been the subject of inhibition and for which response to inhibition of the drug target has been quantified, and
determining whether one or more signatures of chromosomal instability are associated with response to inhibition of the drug target, wherein the presence of a signature of chromosomal instability associated with response to inhibition of the drug target is indicative of the drug target being usable for the treatment of a cancer in which the signature is active.

20. The method of claim 19, further comprising identifying and optionally providing a drug that targets the drug target.

21. A method of predicting whether a subject with cancer is likely to respond to a platinum-based therapy, the method comprising: characterising a sample obtained from a tumour in the subject, using the method of any of claims 1 to 14, as having a high or low exposure to a first signature (CX3) associated with impaired homologous recombination plus replication stress, impaired damage sensing and impaired nucleotide excision repair, wherein if the sample is characterised as having a high exposure to said signature, the subject is likely to respond to platinum-based therapy, or characterising a sample obtained from a tumour in the subject, using the method of any of claims 1 to 14, as having a high or low exposure to a first signature (CX5) associated with impaired homologous recombination plus replication stress, wherein if the sample is characterised as having a high exposure to said signature, the subject is not likely to respond to platinum-based therapy.

22. The method of claim 21, wherein the sample is considered to have a high exposure to the first signature if exposure to the first signature is higher than exposure to a second signature (CX2) associated with impaired homologous recombination alone, and/or wherein the sample is considered to have a low exposure to the first signature if exposure to the first signature is lower than exposure to a second signature (CX2) associated with impaired homologous recombination alone, and/or wherein the sample is considered to have a high exposure to the first signature if exposure to the first signature is higher than exposure to the first signature in a control sample or set of samples, and/or
wherein the cancer is ovarian cancer, and/or
wherein characterising a sample obtained from a tumour in the subject, using the method of any of claims 1 to 14, as having a high or low exposure to a first and/or second signature comprises normalising the exposures for each signature, optionally wherein normalising comprises scaling signature exposures using the parameters of a distribution of the exposure to the respective signatures in a cohort of samples, and/or
wherein a tumour copy number profile for the sample comprises absolute copy numbers determined by obtaining a relative copy number at each of a plurality of genomic locations and determining an absolute copy number for the respective locations based on the relative copy number, the mean relative copy number of the sample, the tumour cell ploidy in the sample and the tumour purity of the sample.

23. A platinum-based therapy for use in a method of treatment of cancer in a subject, the method comprising: (i) determining whether a DNA sample obtained from said subject is likely to respond to platinum-based therapy using a method according to claim 21 or claim 22; and (ii) administering the platinum-based therapy to said subject if the DNA sample is determined to be likely to respond to platinum-based therapy.

24. A method or platinum-based therapy for use according to any of claims 21 to 23, wherein the subject has been diagnosed as having or being at risk of having ovarian cancer or oesophagal cancer.

25. A system comprising:
a processor; and
a computer readable medium comprising instructions that, when executed by the processor, cause the processor to perform the steps of the method of any of claims 1 23.
